SEQUENCE LISTING (43 sequences)

SEQ ID NO: 1 - DNA, 20 bp, Artificial Sequence
  Description of Artificial Sequence Synthetic oligonucleotide
agagcatcggaccgaagcgg

SEQ ID NO: 2 - DNA, 20 bp, Artificial Sequence
  Description of Artificial Sequence Synthetic oligonucleotide
gggggagaacatactcgggg

SEQ ID NO: 3 - DNA, 20 bp, Artificial Sequence
  Description of Artificial Sequence Synthetic oligonucleotide
cccaggttgctagggcttgg

SEQ ID NO: 4 - DNA, 701 bp, Unknown
  Description of Unknown CMV promoter sequence
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaaccctt

SEQ ID NO: 5 - DNA, 249 bp, Unknown
  Description of Unknown U6 promoter sequence
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 6 - protein, 11 aa, Herpes simplex virus
DALDDFDLDML

SEQ ID NO: 7 - DNA, 20 bp, Artificial Sequence
  Description of Artificial Sequence Synthetic oligonucleotide
atcgcctgaaacttgtccgg

SEQ ID NO: 8 - DNA, 20 bp, Artificial Sequence
  Description of Artificial Sequence Synthetic oligonucleotide
cgaaagggtgtgaaagaggg

SEQ ID NO: 9 - DNA, 20 bp, Artificial Sequence
  Description of Artificial Sequence Synthetic oligonucleotide
tggggaaggtaaagcggcga

SEQ ID NO: 10 - DNA, 18 bp, Artificial Sequence
  Description of Artificial Sequence Synthetic oligonucleotide
agagcatcggaccgaagc

SEQ ID NO: 11 - DNA, 20 bp, Artificial Sequence
  Description of Artificial Sequence Synthetic oligonucleotide
gcttgagcaatttcggaccc

SEQ ID NO: 12 - DNA, 20 bp, Artificial Sequence
  Description of Artificial Sequence Synthetic oligonucleotide
tgtgtctcttgctggtaccg

SEQ ID NO: 13 - DNA, 20 bp, Artificial Sequence
  Description of Artificial Sequence Synthetic oligonucleotide
tgagcctgtgccagaggata

SEQ ID NO: 14 - DNA, 22 bp, Artificial Sequence
  Description of Artificial Sequence Synthetic oligonucleotide
tcaactttgattgccaagtgca

SEQ ID NO: 15 - DNA, 20 bp, Artificial Sequence
  Description of Artificial Sequence Synthetic oligonucleotide
gagcagttctggaaccaacc

SEQ ID NO: 16 - DNA, 20 bp, Artificial Sequence
  Description of Artificial Sequence Synthetic oligonucleotide
ttgaggccgaagagagatgt

SEQ ID NO: 17 - DNA, 20 bp, Artificial Sequence
  Description of Artificial Sequence Synthetic oligonucleotide
ccttgtggaatttgggtcat

SEQ ID NO: 18 - DNA, 21 bp, Artificial Sequence
  Description of Artificial Sequence Synthetic oligonucleotide
tcaaatgcaggcacttagaat

SEQ ID NO: 19 - DNA, 22 bp, Artificial Sequence
  Description of Artificial Sequence Synthetic oligonucleotide
caaggaaaaagagaagcaagga

SEQ ID NO: 20 - DNA, 20 bp, Artificial Sequence
  Description of Artificial Sequence Synthetic oligonucleotide
attttaatggctggctttgg

SEQ ID NO: 21 - DNA, 25 bp, Artificial Sequence
  Description of Artificial Sequence Synthetic oligonucleotide
ttttagtttaggttgttagggtttg

SEQ ID NO: 22 - DNA, 25 bp, Artificial Sequence
  Description of Artificial Sequence Synthetic oligonucleotide
taaaaaaacacctcaaattttaccc

SEQ ID NO: 23 - DNA, 20 bp, Artificial Sequence
  Description of Artificial Sequence Synthetic oligonucleotide
tcatcctccttggaaacccg

SEQ ID NO: 24 - DNA, 20 bp, Artificial Sequence
  Description of Artificial Sequence Synthetic oligonucleotide
gtcatcgcccaaccagtaca

SEQ ID NO: 25 - DNA, 20 bp, Artificial Sequence
  Description of Artificial Sequence Synthetic oligonucleotide
agcagcagcaatggacttcg

SEQ ID NO: 26 - DNA, 23 bp, Artificial Sequence
  Description of Artificial Sequence Synthetic oligonucleotide
agaaatacaggatggaggatggt

SEQ ID NO: 27 - DNA, 20 bp, Artificial Sequence
  Description of Artificial Sequence Synthetic oligonucleotide
aagcgcttcctcctcattgg

SEQ ID NO: 28 - DNA, 20 bp, Artificial Sequence
  Description of Artificial Sequence Synthetic oligonucleotide
aaagcacctcaggttttgcc

SEQ ID NO: 29 - DNA, 20 bp, Artificial Sequence
  Description of Artificial Sequence Synthetic oligonucleotide
agctgttgattggctgcttt

SEQ ID NO: 30 - DNA, 20 bp, Artificial Sequence
  Description of Artificial Sequence Synthetic oligonucleotide
ttcaaattccgcccactaaa

SEQ ID NO: 31 - DNA, 20 bp, Artificial Sequence
  Description of Artificial Sequence Synthetic oligonucleotide
cacctcccagcttcactctc

SEQ ID NO: 32 - DNA, 20 bp, Artificial Sequence
  Description of Artificial Sequence Synthetic oligonucleotide
ctgcgggttcatctagttcc

SEQ ID NO: 33 - DNA, 20 bp, Artificial Sequence
  Description of Artificial Sequence Synthetic oligonucleotide
acaaccagcattcgatccat

SEQ ID NO: 34 - DNA, 23 bp, Artificial Sequence
  Description of Artificial Sequence Synthetic oligonucleotide
gctgtcggaattgggtactgttt

SEQ ID NO: 35 - DNA, 23 bp, Artificial Sequence
  Description of Artificial Sequence Synthetic oligonucleotide
gctgtcggaattgggtactgttg

SEQ ID NO: 36 - DNA, 20 bp, Artificial Sequence
  Description of Artificial Sequence Synthetic oligonucleotide
aactcttacttggcgctccc

SEQ ID NO: 37 - DNA, 20 bp, Artificial Sequence
  Description of Artificial Sequence Synthetic oligonucleotide
ctgtccatcgctaagctccc

SEQ ID NO: 38 - DNA, 20 bp, Artificial Sequence
  Description of Artificial Sequence Synthetic oligonucleotide
aatcccatcaccatcttcca

SEQ ID NO: 39 - DNA, 19 bp, Artificial Sequence
  Description of Artificial Sequence Synthetic oligonucleotide
ctccatggtggtgaagacg

SEQ ID NO: 40 - DNA, 11 bp, Unknown
  Description of Unknown CDKL5 gDNA sequence
atctcmaacag

SEQ ID NO: 41 - DNA, 13 bp, Unknown
  Description of Unknown CA5B gDNA sequence
acaagcraaaccc

SEQ ID NO: 42 - DNA, 11 bp, Artificial Sequence
  Description of Artificial Sequence Synthetic oligonucleotide
atctcaaacag

SEQ ID NO: 43 - DNA, 13 bp, Artificial Sequence
  Description of Artificial Sequence Synthetic oligonucleotide
acaagcraaaccc

(19) United States
(12) Patent Application Publication
(10) Pub. No.: US 2022/0389393 A1
(43) Pub. Date: Dec. 8, 2022

(54) COMPOSITIONS AND METHODS FOR EDITING OF THE CDKL5 GENE

(71) Applicant: The Regents of the University of California, Oakland, CA (US)

(72) Inventors: Julian Halmai, Davis, CA (US); Kyle Fink, Davis, CA (US); Peter Deng, Davis, CA (US)

(21) Appl. No.: 17/770,258

(22) PCT Filed: Oct. 21, 2020
(86) PCT No.: PCT/US2020/056726

§ 371 Date: Apr. 19, 2022

Related U.S. Application Data
(60) Provisional application No. 62/924,141, filed on Oct. 21, 2019.
(60) Provisional application No. 62/925,731, filed on Oct. 24, 2019.

(51) Int. Cl. C12N 9/02; C12N 9/22; C12N 15/11; C12N 15/90; C12N 5/00; A61K 38/46; A61K 31/7088

(52) U.S. Cl. CPC C12N 9/0071; C12Y 114/11; C12N 9/22; C12N 15/11; C12N 15/907; C12N 5/00; A61K 38/465; A61K 31/7088; C12N 2310/20; C12N 2800/80

(57) ABSTRACT

A gene editing system is provided that comprises a first nucleotide molecule encoding a dCas9-Ten-Eleven Translocation methylcytosine dioxygenase 1 catalytic domain (TET1CD) fusion protein; and a second nucleotide molecule encoding at least one small guide RNA (sgRNA), comprising: a scaffold region and a spacer region, wherein the spacer region hybridizes to a nucleotide sequence complementary to a target sequence adjacent to a 5′-end of a protospacer adjacent motif (PAM), and wherein the target sequence and the PAM are located within 1 kilobase (kb) of the transcriptional start site (TSS) of the CDKL5 gene. Methods of making and using the system are further described herein.

### CROSS-REFERENCE TO RELATED APPLICATIONS

This application claims priority to U.S. Provisional Application No. 62/924,141, filed Oct. 21, 2019, and U.S. Provisional Application No. 62/925,731, filed Oct. 24, 2019, the entire contents of each of which are incorporated herein by reference.

### STATEMENT OF U.S. FEDERALLY FUNDED RESEARCH

This invention was made with government support under Grant No. P30 CA093373 awarded by the National Cancer Institute; and under Grant Nos. NCRR C06-RR12088, S10 OD018223, S10 RR12964, S10 RR 026825, and 1S100D010786-01 awarded by the National Institute of Health. The government has certain rights in the invention.

### BACKGROUND

The following description of the background of the present technology is provided simply as an aid in understanding the present technology and is not admitted to describe or constitute prior art to the present technology. Throughout and within this disclosure technical and patent literature is referenced by an Arabic numeral or an identifying citation. The complete bibliographic citation for the literature referenced by an Arabic numeral can be found immediately preceding the claims.

Epigenetics is the study of mitotically and/or meiotically stable but reversible modifications to nucleotides or higher order chromatin structure that can alter expression patterns of genes in the absence of changes to the underlying DNA sequence (1). These modifications occur on multiple levels, such as 5-methyl-cytosine (5-meC) DNA methylation, post-translational modifications of histones bound by protein domains that serve as epigenetic writers, readers and erasers and noncoding RNAs that assist in the recruitment of chromatin modifying proteins to DNA (2). These epigenetic layers dynamically dictate the three-dimensional organization of the genome within the nuclear ultrastructure and orchestrate local accessibility for the eukaryotic transcriptional machinery (3). Because of this, epigenetic signatures play a crucial role in dictating cellular identity during development and throughout life in response to the environment (1), correlate with aging (4) and are linked to disease (5), for instance, Rett syndrome (RTT) and CDKL5 deficiency disorder (CDD), two rare X-linked developmental brain disorders associated with epigenetic modification. The neurodevelopmental disorder CDKL5 deficiency is caused by de novo mutations in the CDKL5 gene on the X chromosome (30). Due to random X-chromosome inactivation (XCI), females affected by the disorder form a mosaic of tissue with cells expressing either the mutant or wild type allele (31). Phenotypic variation observed between females in families with RTT are also ascribed to differences in X-inactivation patterns.

Accordingly, there is a need to improve our understanding of XCI and reactivation of X-linked genes, and a need for targeted approaches that result in specific gene reactivation. Targeted DNA demethylation of genes on the X chromosome would allow for a directed assessment of the causal role between DNA methylation and gene expression on the inactive X chromosome. Furthermore, the presence of coding SNPs that exist in clonally-derived female cell lines provides an allele-specific model to study escape from XCI induced by targeted epigenetic remodelling. There is also a need for potential therapeutic approaches that activate a silenced wild type allele of a gene such as CDKL5 in cells expressing the loss-of-function mutant allele.

This disclosure satisfies these needs and provides related advantages as well.

### SUMMARY OF THE DISCLOSURE

The process of XCI epigenetically regulates the amount of transcriptionally active X-chromatin in somatic tissue as a dosage compensation mechanism to ensure equal expression levels of X-linked genes in males and females (6). In female somatic cells, one X chromosome randomly becomes inactive and is cytologically manifested during interphase as a perinuclear heterochromatic Barr body, which is then clonally maintained through mitosis (7, 8). This mechanism is mediated by the long noncoding RNA X-inactive specific transcript (XIST) expressed from the inactive X chromosome in cis (9), which serves as a guiding factor to tether Polycomb proteins for gene silencing to target sites on the X-chromatin (10). XIST induces the formation of repressive heterochromatin through histone deacetylation (11), DNA methylation of CpG-island (CGI) promoters (12), di- and trimethylation of histone 3 at lysine 9 (H3K9me2/3) (13), the deposition and spreading of H3K27me3 across the inactive X-chromatin (14) and the H2A histone variant macroH2A (15).

Gene expression data suggests there is an estimated 15-30% of human X-linked genes that escape XCI (16) at an arbitrary transcriptional threshold of 10% of the active allele (17). The level of escape from XCI is variable between genes and individuals (16), demonstrates tissue heterogeneity (18) and increases with age (19). X-escapees have a distinct epigenetic signature from genes that are subject to XCI, including enrichment of active and depletion of repressive histone marks, and generally reduced levels of DNA methylation near regulatory elements (17). In particular, the degree of CGI promoter 5meC DNA methylation has been demonstrated to be highly correlative with XCI (12, 20).

In line with the idea that DNA methylation forms an epigenetic barrier on the inactive X chromosome, the most potent X-reactivation to date has been achieved by treatment with 5-azacytidine, a global DNA hypomethylating agent in combination with X-wide genetic ablation of XIST (21). In addition, pharmacological and genetic screens aiming to identify trans-acting factors promoting XCI have identified the maintenance DNA methyltransferase DNMT1 as a key player in XCI (22, 23). However, previous studies aiming to elucidate the mechanism of XCI-escape, such as the aforementioned small molecule approaches, utilized untargeted approaches. While these studies have provided a significant foundation of knowledge, in particular demonstrating the importance of DNA methylation in our understanding of X-reactivation, the global side-effects of these types of approaches limit the study of specific gene reactivation.

Until recently, the lack of targeted approaches by which epigenetics can be modified has limited the studies of XCI mechanisms. With the availability of the RNA-guided clustered regularly interspaced palindromic repeats (CRISPR) system, catalytically inactive dCas9 fused to epigenetic effector domains has become the method of choice for targeted rewriting of the epigenome to further elucidate the causality between epigenetic marks and gene expression (24, 25). In particular, dCas9 fusions with the catalytic domain of ten-eleven translocation dioxygenase 1 (TET1) have gained prominence as a candidate to precisely demethylate gene promoters or enhancers for multiple gene targets (26-29). Synthetically inducing a gene escape from XCI via DNA methylation editing of a gene promoter using a dCas9 fusion proteins for targeted DNA demethylation has the potential for providing a much needed therapy for at least X-linked developmental brain disorders.

Building on these discoveries, Applicant provides the following aspects and disclosures.

In one aspect, the present disclosure provides a gene editing system comprising, or consisting essentially of or yet further consisting of: (i) a first nucleotide molecule encoding a dCas9-Ten-Eleven Translocation methylcytosine dioxygenase 1 catalytic domain (TET1CD) fusion protein, and (ii) a second nucleotide molecule encoding at least one single guide RNA (sgRNA), comprising, or consisting essentially of, or yet further consisting of a scaffold region and a spacer region; wherein the spacer region hybridizes to a nucleotide sequence complementary to a target sequence adjacent to a 5′-end of a protospacer adjacent motif (PAM); and wherein the target sequence and the PAM are located within about 1 kilobase (kb) of the transcriptional start site (TSS) of the cyclin dependent kinase-like 5 (CDKL5) gene.

In some embodiments, the spacer region comprises, or consists essentially of, or yet further consists of a spacer sequence provided in Table 1.

In some embodiments, the gene editing system further comprises a third nucleotide molecule encoding a dCas9 protein fused to at least one transcriptional activator.

In some embodiments, the at least one transcriptional activator fused to the dCas9 protein that comprises, or consists essentially of or consists of VP64 or a fragment thereof.

In some embodiments, the target sequence for the sgRNA comprises, or consists essentially of, or consist of one or more of AGAGCATCGGACCGAAGCGG, GGGGGAGAACATACTCGGGG, and/or CCCAGGTTGCTAGGGCTTGG.

In some embodiments, the at least one sgRNA comprises a first sgRNA, a second sgRNA, and a third sgRNA, wherein the target sequence for the first sgRNA comprises or consists essentially of, or yet further consists of AGAGCATCGGACCGAAGCGG, wherein the target sequence for the second sgRNA comprises or consists essentially of, or yet further consists of GGGGGAGAACATACTCGGGG, and wherein the target sequence for the third sgRNA comprises or consists essentially of, or yet further consists of CCCAGGTTGCTAGGGCTTGG.

In some embodiments, the first nucleotide molecule, the second nucleotide molecule, and the third nucleotide molecule are integrated into one or more viral or plasmid vectors.

In some embodiments, the viral vector is a selected from the group of a lentiviral vector, an adeno-associated viral (AAV) vector, or an adenoviral vector.

In one aspect, the disclosure provides a kit comprising the system as described herein and optional instructions for use in the methods as described herein.

In one aspect, the disclosure provides a host cell comprising the gene editing system.

In some embodiments, the host cell comprises a prokaryotic or a eukaryotic cell.

In some embodiments, the host cell comprises a mammalian or a human cell. In another aspect, the mammalian or host cell is a stem cell or progenitor cell, e.g., a iPSC, an embryonic stem cell or a stem cell with the capacity to differentiate into a specific lineage, e.g., neuronal lineage.

In some embodiments, the host cell as described herein has reduced CDKL5 gene expression and/or reduced DNA methylation in the CDKL5 promoter region.

In some embodiments, the host cell is a cultured cell or a primary cell.

In some embodiments, the host cell further comprising a therapeutic molecule.

In one aspect, the disclosure provides a pharmaceutical composition comprising the gene editing system, the vectors or the host cell comprising the gene editing system.

In some embodiments, the pharmaceutical composition comprises a carrier.

In some embodiments, the pharmaceutical composition comprises a pharmaceutically acceptable carrier or excipient.

In one aspect, the disclosure provides a method for increasing CDKL5 gene expression in a cell or subject in need thereof comprising or consists essentially of, or yet further consists of administering to the subject the gene editing system or the pharmaceutical composition comprising or consists essentially of, or yet further consists of the gene editing system.

In some embodiments, DNA methylation in a CDKL5 promoter region of the subject is methylated or hypermethylated, and in one aspect as compared to a non-silenced X-chromosome.

In some embodiments, the CDKL5 promoter region is located on a silenced X-chromosomal allele of the subject.

In some embodiments, the subject has been diagnosed with CDKL5 deficiency disorder (CDD).

In some embodiments, a cell is isolated from a subject having been diagnosed with CDD.

In some embodiments, the cell is a neuronal cell.

In some embodiments, the gene editing system or the pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

In some embodiments, the subject to be treated is a mammal.

In some embodiments, the mammal is a non-human fetus, an infant, a juvenile, or an adult.

In some embodiments, a biological sample from the subject is analyzed for CDKL5 gene expression, prior to and/or after treatment.

In some embodiments, CDKL5 gene expression is analyzed by quantitative PCR using exon-spanning primers for CDKL5 and for the reference gene GAPDH. Exemplary primer oligonucleotides for analyzing CDKL5 gene expression are provided in Table 1.

In one aspect, the disclosure provides a method for treating or preventing CDD in a subject in need thereof comprising administering to the subject the gene editing system or the pharmaceutical composition comprising the gene editing system. In one aspect, a biological system is analyzed for CDKL5 gene expression prior to or after treatment.

In some embodiments, DNA methylation in a CDKL5 promoter region of the subject is reduced, in one aspect, as compared to wild-type gene.

In some embodiments, the CDKL5 promoter region is located on a silenced X-chromosomal allele of the subject.

In some embodiments, the gene editing system or pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

In some embodiments, the subject is a mammal. In some embodiments, the mammal is a non-human fetus, an infant, a juvenile, or an adult.

In some embodiments, genomic DNA isolated from the subject is analyzed for targeted DNA methylation.

In some embodiments, targeted DNA methylation is analyzed by bisulfite-sequencing PCR. Exemplary primers for bisulfite-sequencing PCR are provided in Table 1.

In one aspect, the disclosure provides a vector encoding a sgRNA, wherein the sgRNA comprises a scaffold region and a spacer region, wherein the spacer region hybridizes to a nucleotide sequence complementary to a target sequence comprising, or consisting essentially of, or yet further consisting of one or more of AGAGCATCGGACCGAAGCGG, and/or GGGGGAGAACATACTCGGGG, and/or CCCAGGTTGCTAGGGCTTGG.

In some embodiments, the spacer region comprises a spacer sequence provided in Table 1.

In some embodiments, the vector encodes a first sgRNA and a second sgRNA; wherein the first sgRNA and the second sgRNA each comprise (a) a scaffold region and (b) a spacer region that hybridizes to a nucleotide sequence complementary to a target sequence; and wherein: (i) the target sequence of the first sgRNA comprises or consists essentially of, or yet further consists of AGAGCATCGGACCGAAGCGG, and the target sequence of the second sgRNA comprises or consists essentially of, or yet further consists of GGGGGAGAACATACTCGGGG; (ii) the target sequence of the first sgRNA comprises or consists essentially of, or yet further consists of AGAGCATCGGACCGAAGCGG, and the target sequence of the second sgRNA comprises or consists essentially of, or yet further consists of CCCAGGTTGCTAGGGCTTGG; or (iii) the target sequence of the first sgRNA comprises or consists essentially of, or yet further consists of GGGGGAGAACATACTCGGGG, and the target sequence of the second sgRNA comprises or consists essentially of, or yet further consists of CCCAGGTTGCTAGGGCTTGG.

In some embodiments, the vector encodes a first sgRNA, a second sgRNA, and a third sgRNA, wherein the first sgRNA, the second sgRNA, and the third sgRNA each comprise (a) a scaffold region and (b) a spacer region that hybridizes to a nucleotide sequence complementary to a target sequence, wherein the target sequence of the first sgRNA comprises or consists essentially of, or yet further consists of AGAGCATCGGACCGAAGCGG, wherein the target sequence of the second sgRNA comprises or consists essentially of, or yet further consists of GGGGGAGAACATACTCGGGG, and wherein the target sequence of the third sgRNA comprises or consists essentially of, or yet further consists of CCCAGGTTGCTAGGGCTTGG.

In some embodiments, the vector further comprises a nucleotide molecule encoding a dCas9-TET1CD fusion protein.

In some embodiments, the vector further comprises a nucleotide molecule encoding a dCas9 protein fused to at least one transcriptional activator.

In some embodiments, the vector further comprises a first nucleotide molecule encoding a dCas9-TET1CD fusion protein and a second nucleotide molecule encoding a dCas9 protein fused to at least one transcriptional activator.

In some embodiments, the transcriptional activator fused to the dCas9 protein comprises VP64 or a fragment thereof. In some embodiments, the vector is a viral vector or a plasmid vector.

In some embodiments, the viral vector is a lentiviral vector, an AAV vector, or an adenoviral vector.

In one aspect, the disclosure provides a host cell comprising the vector.

In one aspect, the disclosure provides a pharmaceutical composition comprising the vector or the host cell comprising the vector.

In some embodiments, the pharmaceutical composition comprises a carrier.

In some embodiments, the pharmaceutical composition comprises a pharmaceutically acceptable carrier or excipient.

### BRIEF DESCRIPTION OF THE DRAWINGS

FIGS. 1A-G show the programmable transcription of the CDKL5 gene.

FIG. 1A shows a schematic illustrating the University of California Santa Cruz (UCSC) genome browser snapshot of the target sites of six sgRNAs directed against the CDKL5 promoter on the X-chromosome (Xp22.13). FIG. 1A further shows DNase hypersensitive sites and H3K4me3, which are often found near promoters derived from ENCODE. Sense sgRNAs are 2, 6, and antisense sgRNAs are 1, 3, 4, and 5.

FIG. 1B shows a bar graph illustrating CDKL5 mRNA fold change relative to mock-treated cells in U87MG cells determined by RT-qPCR resulting from programmable transcription using a dCas9-no effector (dC) or dCas9-VP64 (dC-V) in combination with different pools of three to six sgRNAs targeted to the CDKL5 promoter 48 hours after transient transfection. #Significantly different from dCas9 sgRNAs 1-3, n=3 independent experiments, Tukey's HSD, p<0.05.

FIG. 1C shows a bar graph illustrating CDKL5 mRNA fold change relative to mock-treated cells in BE2C cells determined by RT-qPCR resulting from programmable transcription using dCas9-no effector or dCas9-VP64 co-expressed with sgRNAs 1-3 48 hours after transient transfection.

FIG. 1D shows a bar graph illustrating CDKL5 mRNA fold change relative to mock-treated cells in Lenti-X 293T determined by RT-qPCR resulting from programmable transcription using dCas9-no effector or dCas9-VP64 co-expressed with sgRNAs 1-3 48 hours after transient transfection. #Significantly different from dCas9 sgRNAs 1-3, n=3 independent experiments, Student t-test p<0.05.

FIG. 1E shows a bar graph illustrating Male-female expression differences in CDKL5 compared to a known X chromosome Inactivation (XCI) escape gene CA5B across 27 GTEx tissues.

FIG. 1F shows a bar graph illustrating the analysis of XCI status of CDKL5 compared to genes showing variable expression from the inactive X-chromosome using scRNA-seq from previously published data. #Significantly different from CA5B, p<0.05.

FIG. 1G shows a Sanger sequencing of genomic DNA and cDNA from SH-SY5Y illustrating that CDKL5 showed mono-allelic expression of a SNP, in contrast to an escape gene, CA5B, which showed expression from the escape allele.

FIGS. 2A-E show targeted reactivation of CDKL5 from the inactive X allele.

FIG. 2A shows a schematic illustrating the targeted reactivation of CDKL5 on the X-chromosome using a coding SNP in the CDKL5 gene.

FIG. 2B shows graphs illustrating a flow sort of cells purified to stably express dCas9 or dCas9-VP64 fused to a GFP via a T2A peptide or dCas9-TET1CD-P2A-BFP.

FIG. 2C shows a bar graph illustrating allele specific read counts for the mRNA expression of the active (Xa) or inactive (Xi) CDKL5 allele of mock-treated SH-SY5Y or after constitutive expression of dCas9 effector domains dCas9 (dC), dCas9-VP64 (dC-V), dCas9-TET1CD (dC-T) or a combination of dCas9-VP64 and dCas9-TET1CD (dC-V+dC-T) and sgRNAs 1-3 after 21 days post-transduction. #Significantly different from mock-treated, ‡significantly different from dCas9, n=3 independent experiments, Tukey's HSD, p<0.05.

FIG. 2D shows a bar graph illustrating the relative Xi CDKL5 mRNA expression of mock-treated or stably transduced SH-SY5Y relative to CDKL5 Xa mRNA expression of mock-treated cells as determined by allele-specific RT-qPCR after 21 days post-transduction. #Significantly different from dC, ‡significantly different from dC-V, †significantly different from dC-T, n=3 independent experiments, Tukey's HSD, p<0.05.

FIG. 2E shows a bar graph illustrating the relative Xa CDKL5 mRNA expression in mock-treated and stably transduced SH-SY5Y cells determined by allele-specific RT-qPCR after 21 days post-transduction. #Significantly different from mock-treated, †significantly different from dCas9, n=3 independent experiments, Tukey's HSD, all p<0.05.

FIGS. 3A-E show that dCas9-TET1CD caused removal of DNA methylation from the CDKL5 CGI promoter.

FIG. 3A shows a schematic illustrating a UCSC genome browser snapshot of the target sites of sgRNAs 1-3 directed against the CDKL5 promoter on Xp22.13 and a large CpG Island (>1 kb) spanning the transcriptional start site of CDKL5. The black box represents a >200 bp region assessed for targeted DNA methylation changes containing 24 individual CpG dinucleotides (drawn to scale).

FIG. 3B shows a scatter plot illustrating 5-methylcytosine levels in a CpG context (5meCG) over total CpG context as assessed by targeted bisulfite sequencing across 11 CpG dinucleotides in mock-treated cells or cells transduced to constitutively express dCas9-no effector (dC) or dCas9 fused to either VP64 (dC-V) or TET1CD (dC-T), a combination thereof (dC-V+dC-T) or a catalytically inactive TET1CD (dC-dT). X-axis depicts the individual CpG position relative to the amplicon (not drawn to scale).

FIG. 3C shows a bar graph illustrating the mean 5-methylcytosine levels in a CpG context over all 11 CpG dinucleotides in all treatment groups. #Significantly different from mock-treated cells, ‡significantly different from dCas9, †significantly different from dC-dT, ¥significantly different from dC-T, n=3 independent experiments, Tukey's HSD, all p<0.05.

FIG. 3D shows a scatter plot illustrating 5-methylcytosine levels in a CpG context (5meCG) over total CpG context as assessed by targeted bisulfite sequencing across CpG dinucleotides 12-24 in mock-treated cells or cells transduced to constitutively express dCas9-no effector (dC) or dCas9 fused to either VP64 (dC-V) or TET1CD (dC-T), a combination thereof (dC-V+dC-T) or a catalytically inactive TET1CD (dC-dT). X-axis depicts the individual CpG position relative to the amplicon (not drawn to scale).

FIG. 3E shows a bar graph illustrating the mean 5-methylcytosine levels in a CpG context over all 12 CpG dinucleotides in all treatment groups from FIG. 3D, n=3 independent experiments.

FIG. 3F shows a scatter plot of the combination of data from FIG. 3B and FIG. 3D, illustrating 5-methylcytosine levels in a CpG context (5meCG) over total CpG context as assessed by targeted bisulfite sequencing across CpG dinucleotides 1-24 in mock-treated cells or cells transduced to constitutively express dCas9-no effector (dC) or dCas9 fused to either VP64 (dC-V) or TET1CD (dC-T), a combination thereof (dC-V+dC-T) or a catalytically inactive TET1CD (dC-dT). X-axis depicts the individual CpG position relative to the amplicon (not drawn to scale).

FIG. 3G shows a bar graph illustrating the mean 5-methylcytosine levels in a CpG context over all 24 CpG dinucleotides in all treatment groups from FIG. 3E. #significantly different from mock-treated cells, ‡significantly different from dCas9, †significantly different from dC-dT, ¥significantly different from dC-T, n=3 independent experiments, Tukey's HSD, all p<0.05.

FIGS. 4A-F shows the depletion of the XCI hallmark histone modification H3K27me3.

FIG. 4A shows a University of California Santa Cruz (UCSC) genome browser snapshot of the target sites of sgRNAs 1-3 directed against the CDKL5 promoter on Xp22.13 and H3K27me3 peaks derived from ENCODE. Black boxes show the regions assessed by ChIP-qPCR

FIG. 4B shows a bar graph illustrating input normalized H3K27me enrichment levels determined by ChIP-qPCR in region A of the CDKL5 promoter in mock-treated cells or cells transduced to constitutively express dCas9-no effector (dC) or dCas9 fused to either VP64 (dC-V) or TET1CD (dC-T).

FIG. 4C shows a bar graph illustrating input normalized H3K27me enrichment levels determined by ChIP-qPCR in region B of the CDKL5 promoter.

FIG. 4D shows a bar graph illustrating input normalized H3K27me enrichment levels determined by ChIP-qPCR in region C of the CDKL5 promoter.

FIG. 4E shows a bar graph illustrating input normalized H3K27me enrichment levels determined by ChIP-qPCR in the promoter of the nearest neighboring gene, SCML2.

FIG. 4F shows a bar graph illustrating input normalized H3K27me enrichment levels determined by ChIP-qPCR in the promoter of a distal gene, MECP2, that serves as a negative control. #Significantly different from mock-treated cells, n=3 independent experiments, p<0.05.

FIGS. 5A-K show global DNA hypomethylation due to constitutive dCas9-TET1CD expression.

FIG. 5A shows a scatter plot illustrating 32 CpG positions shown with their respective location on the X-chromosome (hg19) from the 850K MethylationEPIC array across the CDKL5 promoter were used to assess gene-wide changes in DNA methylation levels represented as changes in the beta value of the TSS200, TSS1500, 5′UTR and gene body of CDKL5. In particular, FIG. 5A shows reduced DNA methylation levels in the TSS1500 and TSS200 region of cells transduced with dCas9-TET1CD found after the transduction with dCas9-no effector (dC), dCas9-TET1CD (dC-T) and a catalytically inactive TET1CD (dC-dT). The line above TSS1500 demonstrates the sgRNA binding sites in the CDKL5 promoter. *illustrates significantly differentially methylated positions for further assessment.

FIG. 5B shows a bar graph illustrating side-by-side assessment of significantly differentially methylated positions in the CDKL5 promoter with a mean difference in beta value of <0.05. #Significantly different from dC, †significantly different from dC-dT, n=2 independent experiments, FDR <5%.

FIG. 5C shows an histogram illustrating the number of genes by the number of significantly hypomethylated sites of dCas9-TET1CD transduced cells when compared to dCas9 or a catalytically inactive TET1 fused to dCas9 demonstrates that the majority of genes shows only a single probe falling within the respective promoter region.

FIG. 5D shows a bar graph illustrating side-by-side assessment of significantly differentially methylated positions in the COL9A3 promoter with a mean difference in beta value of <0.05. #significantly different from dC, \significantly different from dC-dT, n=2 independent experiments, FDR <5%.

FIG. 5E shows a Venn diagram illustrating shared genes between dCas9-TET1CD and dCas9 or a catalytically inactive TET1CD mutant, and shows an overlap of 48 genes between the two groups.

FIG. 5F shows a flow chart diagram representing the analysis pipeline for genome-wide methylation effects of dCas9-TET1CD, starting from a total number of probes, down to significantly differentially methylated sites and ultimately differentially methylated genes.

FIG. 5G shows QC analysis of 850K Methylation EPIC data illustrating a dendogram demonstrating that biological replicates clustered together and controls showed different hierarchies than dCas9-TET1CD.

FIG. 5H shows density plots of beta value distribution before and after normalization with preprocessNoob and preprocessFunNorm of the data of FIG. 5G.

FIG. 5I shows the total probe statistics by feature of the data of FIG. 5G.

FIG. 5J shows total number of hypermethylated differentially methylated positions by feature of the data of FIG. 5G.

FIG. 5K shows the total number of hypomethylated differentially methylated positions by feature of the data of FIG. 5G.

FIGS. 6A-E show Off-target analysis of CRISPR/dCas9 effectors by RNA-seq.

FIG. 6A shows a volcano plot illustrating significance (FDR adjusted p value) versus fold change for differential DESeq2 expression analysis of mock-treated, dCas9-VP64 (dC-V), dCas9-TET1CD (dC-T) or dCas9-VP64 and dCas9-TET1CD (dC-V+dC-T) guided by sgRNAs 1-3 to the CDKL5 promoter compared to a dCas9-no effector control (dC). Differentially expressed genes are illustrated by black dots (FDR <1%, log fold change >1), predicted CRISPR off-target sites are highlighted in blue and the CDKL5 target gene is highlighted in green. The number of downregulated genes is shown in the upper left of each panel, and the number of upregulated genes is shown in the upper right of each panel.

FIG. 6B shows a Venn diagram illustrating the overlap of differentially expressed genes between all conditions and the putative off-target list, and shows that a single gene, CNTNAP2, was shared between all four groups as a putative off-target.

FIG. 6C shows a Venn diagram illustrating the overlap between differentially expressed genes and differentially methylated positions identified in a comparison between dCas9-TET1CD and dCas9 and potential CRISPR off-targets.

FIG. 6D shows a bar graph illustrating the validation of the differentially expressed gene, CNTNAP, by RT-qPCR, and shows the relative CNTNAP2 mRNA levels in SH-SY5Y determined by RT-qPCR after constitutive expression of dCas9 (dC), dCas9-VP64 (dC-V), dCas9-TET1CD (dC-T) or a combination of dCas9-VP64 and dCas9-TET1CD (dC-V+dC-T) and sgRNAs 1-3 after 21 days post-transduction. #significantly different from dCas9, n=3 independent experiments, Tukey's HSD, p<0.05. #significantly different from dCas9 sgRNAs 1-3, n=3 independent experiments, Student t-test p<0.05.

FIG. 6E shows a bar graph illustrating the validation of the differentially expressed gene, HHIPL1, by RT-qPCR, and shows the relative HHIPL1 mRNA levels in SH-SY5Y determined by RT-qPCR after constitutive expression of dCas9 (dC) or dCas9-TET1CD (dC-T) and sgRNAs 1-3 after 21 days posttransduction. #significantly different from dCas9 sgRNAs 1-3, n=3 independent experiments, Student's t-test, p<0.05.

FIG. 7 shows a schematic illustrating a model of the programmable transcription of the CDKL5 gene using Cas9 effector domain fused to epigenetic effector domains from VSP64 or ten-eleven translocation dioxygenase 1 (TET1). In particular, DNA methylation editing of the CDKL5 promoter using a dCas9-TET1 fusion protein for targeted DNA demethylation resulted in a significant increase in allele-specific expression of the inactive allele of CDKL5 and a significant reduction in methylated CpG dinucleotides in the CGI core promoter. Moreover, while dCas9-VSP64 fusion protein had no effect alone, co-expression of dCas9-TET1 and a dCas9-VP64 transactivator has a synergistic effect on the reactivation of the inactive CDKL5 allele to levels above 60% of the active allele.

### DETAILED DESCRIPTION

Embodiments according to the present disclosure are described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting. Throughout and within this disclosure various technical and patent publications are references by a citation or an Arabic numeral. The full bibliographic citations for each reference identified by an Arabic numeral is found in the reference section, immediately preceding the claims.

It is to be appreciated that certain aspects, modes, embodiments, variations and features of the present methods are described below in various levels of detail in order to provide a substantial understanding of the present technology. The definitions of certain terms as used in the specification are provided below. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination. The term consisting of intends the recited elements and any additional elements that do not materially change of the function of the recited element or elements.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

The practice of the present technology employs, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; Herzenberg et al. eds (1996) Weir's Handbook of Experimental Immunology, 5th edition; and/or more recent editions thereof.

### Definitions

As used herein, the singular forms “a,” “an” and “the” are intended to include the plural forms as well, unless the context clearly indicates otherwise.

As used herein, the term “about,” when referring to a measurable value such as an amount or concentration and the like, is meant to encompass variations of 20%, 10%, 5%, 1%, 0.5%, or even 0.1% of the specified amount.

As used herein, the terms or “acceptable,” “effective,” or “sufficient” when used to describe the selection of any components, ranges, dose forms, etc. disclosed herein intend that said component, range, dose form, etc. is suitable for the disclosed purpose.

As used herein, the term “adeno-associated virus” or “AAV” refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the gene editing systems, host cells, pharmaceutical compositions, vectors, and methods disclosed herein include any of the 11 or 12 serotypes, e.g., AAV2, AAV5, and AAV8, or variant serotypes, e.g. AAV-DJ. The AAV structural particle is composed of 60 protein molecules made up of VP1, VP2 and VP3. Each particle contains approximately 5 VP1 proteins, 5 VP2 proteins and 50 VP3 proteins ordered into an icosahedral structure.

As used herein, the term “administering” a compound or composition to a subject means delivering the compound to the subject. “Administering” includes prophylactic administration of the compound or composition (i.e., before the disease and/or one or more symptoms of the disease are detectable) and/or therapeutic administration of the composition (i.e., after the disease and/or one or more symptoms of the disease are detectable). The methods of the present technology include administering one or more compounds or agents.

If more than one compound is to be administered, the compounds may be administered together at substantially the same time, and/or administered at different times in any order.

Also, the compounds of the present technology may be administered before, concomitantly with, and/or after administration of another type of drug or therapeutic procedure (e.g., surgery).

As used herein, “ameliorate,” “ameliorating,” and the like, as used herein, refer to inhibiting, relieving, eliminating, or slowing progression of one or more symptoms.

As used herein, “and/or” refers to and encompasses any and all possible combinations of one or more of the associated listed items, as well as the lack of combinations when interpreted in the alternative (“or”).

As used herein, the term “aptamer” as used herein refers to single stranded DNA or RNA molecules that can bind to one or more selected targets with high affinity and specificity. Non-limiting exemplary targets include by are not limited to proteins or peptides.

As used herein, the term “Cas9” refers to a CRISPR-associated, RNA-guided endonuclease such as Streptococcus pyogenes Cas9 (spCas9) and orthologs and biological equivalents thereof. Biological equivalents of Cas9 include but are not limited to C2c1 from Alicyclobacillus acideterrestris and Cpf1 (which performs cutting functions analogous to Cas9) from various bacterial species including Acidaminococcus spp. and Francisella novicida U112. Cas9 may refer to an endonuclease that causes double stranded breaks in DNA, a nickase variant such as a RuvC or HNH mutant that causes a single stranded break in DNA, as well as other variations such as deadCas-9 or dCas9, which lack endonuclease activity. Cas9 may also refer to “split-Cas9” in which CAs9 is split into two halves—C-Cas9 and N-Cas9—and fused with a two intein moieties. See, e.g., U.S. Pat. No. 9,074,199 B1; Zetsche et al., Nat Biotechnol. 33(2):139-42 (2015); Wright et al., PNAS 112(10) 2984-89 (2015).

As used herein, the term “cell” or “host cell” may refer to either a prokaryotic or eukaryotic cell, optionally obtained from a subject or a commercially available source.

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guide RNA. A CRISPR system can be used to cause double stranded or single stranded breaks in a target polynucleotide. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359, and Hsu et al., Cell 156(6): 1262-1278 (2014).

As used herein, the term “comprising” is intended to mean that the compositions and methods include the recited elements, but do not exclude others.

As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

As used herein, the term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the route of administration, and the physical delivery system in which it is carried.

In some embodiments, “effective amount” or “therapeutically effective amount” refers to a quantity sufficient to achieve a desired therapeutic and/or prophylactic effect, e.g., an amount which results in the full or partial amelioration of disease or disorders or symptoms associated with mitochondrial dysfunction, neurological disease, lack of energy, glycolytic process dysfunction or cellular respiration related dysfunction in a subject in need thereof. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. A person of ordinary skill in the art will be able to determine appropriate dosages depending on these and other factors. The compositions can also be administered in combination with one or more additional compounds. Multiple doses may be administered. Additionally or alternatively, multiple therapeutic compositions or compounds may administered. In the methods described herein, the compounds may be administered to a subject having one or more signs or symptoms of a disease or disorder described herein.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “endonuclease” refers to any suitable endonuclease enzyme protein or a variant thereof that will be specifically directed by the selected guide polynucleotide to enzymatically knock-out the target sequence of the guide polynucleotide.

As used herein, the term “variant thereof,” as used with respect to an endonuclease, refers to the referenced endonuclease in its enzymatically functional form expressed in any suitable host organism or expression system and/or including any modifications to enhance the enzymatic activity of the endonuclease.

In some embodiments of the present disclosure, a suitable endonuclease includes a CRISPR-associated sequence 9 (Cas9) endonuclease or a variant thereof, a CRISPR-associated sequence 13 (Cas13) endonuclease or a variant thereof, CRISPR-associated sequence 6 (Cas6) endonuclease or a variant thereof, a CRISPR from Prevotella and Francisella 1 (Cpf1) endonuclease or a variant thereof, or a CRISPR from Microgenomates and Smithella 1 (Cms1) endonuclease or a variant thereof. In some embodiments of the present disclosure, a suitable endonuclease includes a Streptococcus pyogenes Cas9 (SpCas9), a Staphylococcus aureus Cas9 (SaCas9), a Francisella novicida Cas9 (FnCas9), or a variant thereof. Variants may include a protospacer adjacent motif (PAM) SpCas9 (xCas9), high fidelity SpCas9 (SpCas9-FIF1), a high fidelity SaCas9, or a high fidelity FnCas9.

In some embodiments of the present disclosure, the endonuclease comprises a Cas fusion nuclease comprising a Cas9 protein or a variant thereof fused with a Fok1 nuclease or variant thereof. Variants of the Cas9 protein of this fusion nuclease include a catalytically inactive Cas9 (e.g., dead Cas9). In some embodiments of the present disclosure, the endonuclease may be a Cas9, Cas1 3, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM1 0 152, Rhodococcus erythropolis PR4, Rhodococcus jostii RFIA1, Rhodococcus opacus B4 (uid36573), Acidothermus cellulolyticus 11B, Arthrobacter chlorophenolicus A6, Kribbella flavida (DSM 17836, uid43465), Thermomonospora curvata (DSM431 83), Bifidobacterium dentium Bd1, Bifidobacterium longum DJO10A, Slackia heliotrinireducens (DSM 20476), Persephonella marina EX H1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei1 9 1, uncultured Termite group 1 bacterium phylotype Rs D 17, Fibrobacter succinogenes S85, Bacillus cereus (ATCC 10987), Listeria innocua, Lactobacillus casei, Lactobacillus rhamnosus GG, Lactobacillus salivarius UCC1 18, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS1 0565, Streptococcus gallolyticus UCN34 (uid46061), Streptococcus gordonii Challis subst CH1, Streptococcus mutans NN2025 (uid46353), Streptococcus mutans, Streptococcus pyogenes M 1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS61 80, Streptococcus pyogenes MGAS31 5, Streptococcus pyogenes SSI-1, Streptococcus pyogenes MGAS1 0750, Streptococcus pyogenes NZ1 3 1, Streptococcus thermophiles CNRZ1 066, Streptococcus thermophiles LMD-9, Streptococcus thermophiles LMG 1831 1, Clostridium botulinum A3 Loch Maree, Clostridium botulinum B Eklund 17B, Clostridium botulinum Ba4 657, Clostridium botulinum F Langeland, Clostridium cellulolyticum H 10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus moniliformis (DSM 121 12), Bradyrhizobium BTAil, Nitrobacter hamburgensis X14, Rhodopseudomonas palustris BisB1 8, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B51 0 (uid46085), Rhodospirillum rubrum (ATCC 11170), Diaphorobacter TPSY (uid29975), Verminephrobacter eiseniae EFO1-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 8 1116, Campylobacter jejuni, Campylobacter lari RM21 00, Helicobacter hepaticus, Wolinella succinogenes, Tolumonas auensis DSM 9 187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U112, Francisella tularensis holarctica, Francisella tularensis FSC 198, Francisella tularensis, Francisella tularensis WY96-3418, or Treponema denticola (ATCC 35405).

As used herein, the terms “equivalent” or “biological equivalent” are used interchangeably when referring to a particular molecule, biological, or cellular material and intend those having minimal homology while still maintaining desired structure or functionality.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

As used herein, the term “guide polynucleotide” refers to a polynucleotide having a “synthetic sequence” capable of binding the corresponding endonuclease enzyme protein (e.g., Cas9) and a variable target sequence capable of binding the genomic target (e.g., a nucleotide sequence found in an exon of a target gene). In some embodiments of the present disclosure, a guide polynucleotide is a guide ribonucleic acid (gRNA). In some embodiments, the variable target sequence of the guide polynucleotide is any sequence within the target that is unique with respect to the rest of the genome and is immediately adjacent to a Protospacer Adjacent Motif (PAM). The exact sequence of the PAM sequence may vary as different endonucleases require different PAM sequences.

As used herein, “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

As used herein, “hybridization” or “hybridizes” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6× saline-sodium citrate (“SSC”) to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M sodium chloride (“NaCl”) and 15 mM citrate buffer. It is understood that equivalents of SSC using other buffer systems can be employed.

As used herein, the term “isolated” as used herein refers to molecules or biologicals or cellular materials being substantially free from other materials.

As used herein, the term “lentivirus” refers to a member of the class of viruses associated with this name and belonging to the genus lentivirus, family Retroviridae. While some lentiviruses are known to cause diseases, other lentivirus are known to be suitable for gene delivery. See, e.g., Tomas et al. (2013) Biochemistry, Genetics and Molecular Biology: “Gene Therapy—Tools and Potential Applications,” ISBN 978-953-51-1014-9, DOI: 10.5772/52534.

As used herein, the terms “nucleic acid sequence,” “nucleotide sequence,” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “organ” a structure which is a specific portion of an individual organism, where a certain function or functions of the individual organism is locally performed and which is morphologically separate. Non-limiting examples of organs include the skin, blood vessels, cornea, thymus, kidney, heart, liver, umbilical cord, intestine, nerve, lung, placenta, pancreas, thyroid and brain.

As used herein, the term “ortholog” is used in reference of another gene or protein and intends a homolog of said gene or protein that evolved from the same ancestral source.

Orthologs may or may not retain the same function as the gene or protein to which they are orthologous. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

As used herein, “prevention,” “prevents,” or “preventing” of a disorder or condition refers to a compound that, in a statistical sample, reduces the occurrence of the disorder, symptom, or condition in the treated sample relative to a control subject, or delays the onset of one or more symptoms of the disorder or condition relative to the control subject.

As used herein, the term “promoter” as used herein refers to any sequence that regulates the expression of a coding sequence, such as a gene. refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. An inducible promoter is one which is capable of being induced by a molecule or a factor added to the cell or expressed in the cell. An inducible promoter may still produce a basal level of transcription in the absence of induction, but induction typically leads to significantly more production of the protein. Promoters can also be tissue specific. A tissue specific promoter allows for the production of a protein in a certain population of cells that have the appropriate transcriptional factors to activate the promoter.

Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include CDKL5 promoter, SCML2 promoter, COL9A3 promoter, MECP2, CMV promoter and U6 promoter, the phosphoglycerate kinase 1 (PGK) promoter; SSFV, CMV, MNDU3, SV40, Efla, UBC and CAGG. Non-limiting exemplary promoter sequences are provided herein below:

CMV Promoter

ATACGCGTTGACATTGATTATTGACTAGTTATTAATAGTAATCAATTACGG GGTCATTAGTTCATAGCCCATATATGGAGTTCCGCGTTACATAACTTACGGTAAA TGGCCCGCCTGGCTGACCGCCCAACGACCCCCGCCCATTGACGTCAATAATGACG TATGTTCCCATAGTAACGCCAATAGGGACTTTCCATTGACGTCAATGGGTGGAGT ATTTACGGTAAACTGCCCACTTGGCAGTACATCAAGTGTATCATATGCCAAGTAC GCCCCCTATTGACGTCAATGACGGTAAATGGCCCGCCTGGCATTATGCCCAGTAC ATGACCTTATGGGACTTTCCTACTTGGCAGTACATCTACGTATTAGTCATCGCTAT TACCATGGTGATGCGGTTTTGGCAGTACATCAATGGGCGTGGATAGCGGTTTGAC TCACGGGGATTTCCAAGTCTCCACCCCATTGACGTCAATGGGAGTTTGTTTTGGC ACCAAAATCAACGGGACTTTCCAAAATGTCGTAACAACTCCGCCCCATTGACGC AAATGGGCGGTAGGCGTGTACGGTGGGAGGTCTATATAAGCAGAGCTCGTTTAG TGAACCGTCAGATCGCCTGGAGACGCCATCCACGCTGTTTTGACCTCCATAGAAG ACACCGGGACCGATCCAGCCTCCGGACTCTAGAGGATCGAACCCTT, or a biological equivalent thereof.

U6 Promoter

GAGGGCCTATTTCCCATGATTCCTTCATATTTGCATATACGATACAAGGCT GTTAGAGAGATAATTAGAATTAATTTGACTGTAAACACAAAGATATTAGTACAA AATACGTGACGTAGAAAGTAATAATTTCTTGGGTAGTTTGCAGTTTTAAAATTAT GTTTTAAAATGGACTATCATATGCTTACCGTAACTTGAAAGTATTTCGATTTCTTG GCTTTATATATCTTGTGGAAAGGACGAAACACC, or a biological equivalent thereof.

A number of effector elements are disclosed herein for use in these vectors; e.g., a tetracycline response element (e.g., tetO), a tet-regulatable activator, T2A, VP64, RtA, KRAB, and a miRNA sensor circuit. The nature and function of these effector elements are commonly understood in the art and a number of these effector elements are commercially available. Non-limiting exemplary sequences thereof are disclosed herein and further description thereof is provided herein below.

As used herein, the term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, “protospacer adjacent motif” (PAM) refers to a short nucleotide sequence adjacent to a target sequence (protospacer) that is recognized (targeted) by a sgRNA/Cas endonuclease system described herein. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long. The PAM sequence plays a key role in target recognition by licensing sgRNA base pairing to the protospacer sequence (Szczelkun et al., Proc. Natl. Acad. Sci. U.S.A 111: 9798-803 (2014)).

As used herein, the term “recombinant expression system” refers to a genetic construct for the expression of certain genetic material formed by recombination.

As used herein, the term “sgRNA” or “single guide RNA” as used herein refers to the guide RNA sequences used to target specific genes for correction employing the CRISPR technique. Techniques of designing sgRNAs and donor therapeutic polynucleotides for target specificity are well known in the art. For example, Doench et al., Nature Biotechnology 32(12):1262-7 (2014), Mohr et al., FEBS J. 283: 3232-38 (2016), and Graham et al., Genome Biol. 16:260 (2015). sgRNA comprises or alternatively consists essentially of, or yet further consists of a fusion polynucleotide comprising CRISPR RNA (crRNA; i.e., a scaffold region) and trans-activating CRIPSPR RNA (tracrRNA; i.e., a spacer region); or a polynucleotide comprising crRNA (i.e., a scaffold region) and tracrRNA (i.e., a spacer region). In some aspects, a sgRNA is synthetic (Kelley et al., J of Biotechnology 233:74-83 (2016).

As used herein, the terms “subject,” “individual,” or “patient” can be an individual organism, a vertebrate, a mammal, or a human. “Mammal” includes a human, non-human mammal, non-human primate, murine (e.g., mouse, rat, guinea pig, hamster), ovine, bovine, ruminant, lagomorph, porcine, caprine, equine, canine, feline, avis, etc. In any embodiment herein, the mammal is feline or canine. In any embodiment herein, the mammal is human.

As used herein, “target sequence” refers to a nucleotide sequence adjacent to a 5′-end of a protospacer adjacent motif (PAM). Being “adjacent” herein means being within 1 to 8 nucleotides of the site of reference, including being “immediately adjacent,” which means that there is no intervening nucleotides between the immediately adjacent nucleotide sequences and the immediately adjacent nucleotide sequences are within one nucleotide of each other.

As used herein, “target site” refers to a site of the target sequence including both the target sequence and its complementary sequence, for example, in double stranded nucleotides. The target site described herein may mean a nucleotide sequence hybridizing to a sgRNA spacer region, a complementary nucleotide sequence of the nucleotide sequence hybridizing to a sgRNA spacer region, and/or a nucleotide sequence adjacent to the 5′-end of a PAM. Full complementarity of a sgRNA spacer region with a target site is not necessarily required, provided there is sufficient complementarity to cause hybridization and promote formation of a CRISPR complex. A target sequence or target site may comprise any polynucleotide, such as DNA or RNA polynucleotides. In some embodiments, a target sequence or target site is located in the nucleus or cytoplasm of a cell. In some embodiments, the target sequence or target site may be within an organelle of a eukaryotic cell, for example, mitochondrion or chloroplast.

As used herein, the term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected) or a group of tissues making up an organ or part or region of the body of an organism. The tissue may comprise a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prevention or prophylaxis.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. A clone is a line of cells that is genetically identical to the originating cell; in this case, a stem cell.

A population of cells intends a collection of more than one cell that is identical (clonal) or non-identical in phenotype and/or genotype. A substantially homogenous population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers.

As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller (2006) The Promise of Stem Cells for Neural Repair, Brain Res. Vol. 1091(1):258-264; Pluchino et al. (2005) Neural Stem Cells and Their Use as Therapeutic Tool in Neurological Disorders, Brain Res. Brain Res. Rev., Vol. 48(2):211-219; and Goh, et al. (2003) Adult Neural Stem Cells and Repair of the Adult Central Nervous System, J. Hematother. Stem Cell Res., Vol. 12(6):671-679.

As use herein, the term “differentiation” describes the process whereby an unspecialized cell acquires the features of a specialized cell such as a heart, liver, or muscle cell. “directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, the term “differentiates or differentiated” defines a cell that takes on a more committed (“differentiated”) position within the lineage of a cell. “Dedifferentiated” defines a cell that reverts to a less committed position within the lineage of a cell. Induced pluripotent stem cells are examples of dedifferentiated cells.

As used herein, the “lineage” of a cell defines the heredity of the cell, i.e. its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100 (Suppl. 1) 11911-6.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

As used herein, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques.

Another type of vector is a viral vector, wherein virally-derived DNA or RNA sequences are present in the vector for packaging into a virus (e.g., retroviruses, replication defective retroviruses, adenoviruses, replication defective adenoviruses, lentiviruses, replication defective lentiviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous viral expression vectors include retroviruses, replication defective retroviruses, adenoviruses, replication defective adenoviruses, lentiviruses, replication defective lentiviruses, and adeno-associated viruses.

It is to be inferred without explicit recitation and unless otherwise intended, that when the present disclosure relates to a polypeptide, protein, polynucleotide or antibody, a fragement an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, or a hydroxyl group(s) it can form salts with metals, such as alkali and earth alkali metals (e.g. Na*, Li*, K*, Ca2+, Mg2+, Zn²⁺), ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Modes for Carrying Out the Disclosure Gene Editing Systems

The disclosure provides a gene editing systems comprising, or alternatively consisting essentially of, or yet further consisting of: (i) a first nucleotide molecule encoding a dCas9-Ten-Eleven Translocation methylcytosine dioxygenase 1 catalytic domain (TET1CD) fusion protein; and (ii) a second nucleotide molecule encoding at least one small guide RNA (sgRNA). In some embodiment, the second nucleotide molecule encoding at least one small guide RNA (sgRNA) comprises, or consists essentially of, or consisting of a scaffold region and a spacer region. In some embodiments, the scaffold region is an amino acid sequence that is necessary for dCas9 binding to the gRNA (addgene.org/guides/crispr/). In some embodiments, the spacer region hybridizes to a nucleotide sequence that is complementary to a target sequence adjacent to a 5′-end of a protospacer adjacent motif (PAM). In some embodiments, the target sequence and the PAM are located at least about 2 or about 1 kilobase (kb), at least about 1.5 kb, at least about 1 kb, at least about 0.9 kb, at least about 0.8 kb, at least about 0.7 kb, at least about 0.6 kb, at least about 0.5 kb, at least about 0.4 kb, at least about 0.3 kb, at least about 0.2 kb, at least about 0.1 kb from the transcriptional start site (TSS) of the CDKL5 gene. While the target sequence and the PAM are in one aspect located can be located at least about 1 kb from the transcriptional start site, it is apparent to the skilled artisan that other ranges are within the scope of this invention, e.g., the target sequence and the PAM are located from about 2 kb, or from about 1 kb to about 0.1 kb.

In some embodiments, the first nucleotide molecule encoding a dCas9-Ten-Eleven Translocation methylcytosine dioxygenase 1 catalytic domain (TET1CD) fusion protein; and (ii) the second nucleotide molecule encoding at least one small guide RNA (sgRNA) induce DNA demethylation of CpGs (GC islands or region) at positions of at least about −1500, at least about −1000, at least about −500, at least about −200, at least about −148, at least about −66 and, at least about −19 relative to transcription start site.

In some embodiments, the first nucleotide and second nucleotide molecules permit the transcriptional reprogramming of a gene promoter by precisely demethylating gene promoters or enhancers for desired gene targets. Thus, in one aspect, as described herein, is a method for transcriptionally reprogramming a gene promoter in a cell in need thereof, by inserting into the cell, the system as disclosed herein. In some embodiments, DNA is methylated at 5-cytosine (5mC), and such methylation silence gene expression and is important for genomic imprinting, regulation of gene expression, chromatic architecture organization, and cell-fate determination. In some embodiments, gene demythylation is associated with gene activation and occurs either via passive demethylation or through the oxidation of the methyl group. In some embodiments, demethylation via oxidation is mediated by TET (ten-eleven translocation) dioxygenases that oxidizes 5 methyl cytosine (5mC) to 5-hydroxymethylcytosine (5-hmC), which is a critical step in the ultimate removal of the methyl group.

In some embodiments, the full-length TET1 protein comprises typical features of 20G-Fe(II) oxygenases, including conservation of residues predicted to be important for coordination of the cofactors Fe(II) and 20G. The full-length TET1 protein has 2136 amino acids, and comprises an N-terminal a helix followed by a continuous series of p strands, typical of the double-stranded 0 helix (DSBH) fold of the 20G-Fe(II) oxygenases, a unique conserved cysteine-rich region (amino acids 1418-1610 of the full-length human TET1 protein; MIM:607790; ENSG00000138336) that is contiguous with the N terminus of the DSBH region (amino acids 1611-2074), a CXXC-type zinc-binding domain (amino acids 584-624 of the full-length human TET1 protein) domain, binuclear Zn-chelating domain, and three bipartite nuclear localization signals (NLS) (66, 68). In some embodiments, TET1 catalytic domain (TET1CD) comprises, or consists essentially of, or consisting of amino acids 1418 to 2136 of the full-length TET1 protein, and encompasses the conserved cysteine-rich region and the DSBH domain (68). In some embodiments, the DSBH domain of the catalytic domain construct comprises a nuclear localization (NLS) sequence. In some embodiments, the DSBH domain of the catalytic domain construct does not comprise a NLS sequence.

In some embodiments, the dCas9-TET1 fusion protein facilitates the targeted demethylation of gene targets (24-29). In particular, dCas9-TET1 facilitates the targeted demethylation of gene targets selected from the group consisting of CDK5L, SCML2 (Scm Polycomb Group Protein Like 2), COL9A3, or Methyl-CpG Binding Protein 2 (MECP) as shown in the Examples below. In some embodiments, both (i) a first nucleotide molecule encoding a dCas9-Ten-Eleven Translocation methylcytosine dioxygenase 1 catalytic domain (TET1CD) fusion protein and (ii) a second nucleotide molecule encoding at least one small guide RNA (sgRNA), are required to target dCas9-Tetl to a specific locus to demethylate DNA without altering the DNA sequence.

In some embodiments, the dCas9 is a catalytically inactive Cas9 nuclease from the Clustered regularly interspaced palindromic repeats (CRISPR), a type II bacterial adaptive immune system that has been modified to target the dCas9 to a desired genomic loci using sequence-specific guide RNAs for genome editing. In some embodiments, the desired genomic loci include any genes, optionally CDK5L, SCML2 (Scm Polycomb Group Protein Like 2), COL9A3, or Methyl-CpG Binding Protein 2 (MECP). In some embodiments, CDKL5 sgRNAs 20-bp spacer sequences are selected within at least about about 1 kb or about 2 kb, at least about 1.5 kb, at least about 1 kb, at least about 0.9 kb, at least about 0.8 kb, at least about 0.7 kb, at least about 0.6 kb, at least about 0.5 kb, at least about 0.4 kb, at least about 0.3 kb, at least about 0.2 kb, at least about 0.1 kb of the CDKL5 TSS (chrX:18,443,725, hg19) using the CRISPR/Cas9 and TALEN online tool for genome editing, CHOPCHOP. In some embodiments, guide RNAs (sgRNAs) span DNase I hypersensitive sites and H3K4me3 peaks of the CDKL5 promoter within at least about 2 kb, at least about 1.5 kb, at least about 1 kb, at least about 0.9 kb, at least about 0.8 kb, at least about 0.7 kb, at least about 0.6 kb, at least about 0.5 kb, at least about 0.4 kb, at least about 0.3 kb, at least about 0.2 kb, at least about 0.1 kb of window on either side of the CDKL5 transcriptional start site. In some embodiments, the second nucleotide molecule encoding at least one small guide RNA (sgRNA) used to create target-specific sgRNA expression vectors are listed in Table 1.

In some embodiments, the targeted sequence is a sequence in the gene promoter. The targeted sequence or a fragment thereof hybridizes to the corresponding gRNA. In one embodiment, the targeted sequence hybridizes to the corresponding gRNA without any mismatches. In another embodiment, the targeted sequence hybridizes to the corresponding gRNA with 1, 2, 3, 4, 5, 6, 7, 8, 9, 10 or more mismatches. Based on the targeted sequence, the gRNA sequence can be determined. In one embodiment, a gRNA comprises, or consists essentially of, or yet further consists of a sequence complement to a targeted sequence, such as those as disclosed herein, or an equivalent that is capable of binding to the same targeted sequence but comprises 1, 2, 3, 4, 5, 6, 7, 8, 9, 10 or more mismatches. In another embodiment, a gRNA comprises, or consists essentially of, or yet further consists of a sequence reverse-complement to a targeted sequence, such as those as disclosed herein, or an equivalent that is capable of binding to the same targeted sequence but comprises 1, 2, 3, 4, 5, 6, 7, 8, 9, 10 or more mismatches. In yet another embodiment, a gRNA comprises, or consists essentially of, or yet further consists of a sequence reverse to a targeted sequence, such as those as disclosed herein, or an equivalent that is capable of binding to the same targeted sequence but comprises 1, 2, 3, 4, 5, 6, 7, 8, 9, 10 or more mismatches.

In one aspect, this disclosure provides a third nucleotide molecule encoding a dCas9 protein fused to at least one transcriptional activator. In some embodiments, the fusion protein comprising the deactivated CRISPR-associated protein 9 (dCas9) with at least one tandem repeat of the transcriptional activator herpes simplex virus VP16 (i.e.VP64) induces transcriptional activation of endogenous of an endogenous gene. In some embodiments, the at least one transcriptional activator comprises VP64 or a biologically active fragment of VP16. Transcription factors act through a DNA-binding domain that localizes a protein to a specific site within the genome and through accessory effector domains that either activate or repress transcription at or near that site. Effector domains, such as the activation domain the herpes simplex virus VP16 (66) and the repression domain Kruppel-associated box (KRAB), are modular and retain their activity when they are fused to other DNA-binding proteins. In some embodiments, VP64 is the activation domain VP16 In some embodiments, VP64 is a recombinant tetrameric repeat of comprising the minimal activation domain VP64. In some embodiments, the activation domain of VP16 comprises amino acids 413-489 of the VP16 protein (66). In some embodiments, the recombinant tetrameric repeat of VP16's minimal activation domain comprises, or consists essentially of, or yet further consists of the amino acid sequence DAL DDFDLDMIL (66) In some embodiments, a third nucleotide molecule encoding a dCas9 protein fused to at least one of dCas9-VP64, VP64-p65-Rta triparte fusion (addgene.org/99670/), and or SunTag. SunTag is a novel protein scaffold/tagging system with a repeating peptide array for signal amplification in gene expression.

In some embodiment, dCas9-VP64 fusion protein upregulates genes in an unmethylated chromatin context. In some embodiment combination of dCas9-VP64 fusion protein and dCas9-TET1CD shows a synergistic effect resulted in a greater than 60% expression of an inactive allele (i.e. silence allele). In some embodiments, expression of dCas9-VP64 fusion protein alone does not significantly increase the reactivation levels of the inactive allele. In some embodiments, dual expression of dCas9-VP64 fusion protein and dCas9-TET1CD resulted in the fewest number of differentially expressed genes in RNAseq analysis.

In some embodiments, gene activation requires several sgRNAs. In some embodiments, gene activation requires six sgRNAs. In some embodiments, gene activation requires at least about, 1-10, 1-5, 1-6, 1-3, 3-6, or 4-6 sgRNAs. In some embodiments, the target sequence for the sgRNA comprises or consists essentially of or consist of one or more of: AGAGCATCGGACCGAAGCGG, GGGGGAGAACATACTCGGGG, CCCAGGTTGCTAGGGCTTGG, ATCGCCTGAAACTTGTCCGG, CGAAAGGGTGTGAAAGAGGG, and/or TGGGGAAGGTAAAGCGGCGA. In some embodiments, the target sequence for the sgRNA comprises or consists essentially of or consist of AGAGCATCGGACCGAAGC. In some embodiments, the target sequence for the sgRNA comprises or consists essentially of or consist of GGGGGAGAACATACTCGGGG.

In some embodiments, the target sequence for the sgRNA comprises or consists essentially of or consist of CCCAGGTTGCTAGGGCTTGG.

In some embodiments, the second nucleotide molecule encoding at least one small guide RNA (sgRNA) comprises or consists essentially of or consist of at least three sgRNAs.

In some embodiments, the second nucleotide molecule encoding at least one small guide RNA (sgRNA) comprises a first sgRNA, a second sgRNA, and a third sgRNA. In some embodiments, the target sequence for the first sgRNA comprises or consists essentially of or consist of AGAGCATCGGACCGAAGCGG. In some embodiments, the target sequence for the second sgRNA comprises or consists essentially of or consist of GGGGGAGAACATACTCGGGG. In some embodiments, the target sequence for the third sgRNA comprises or consists essentially of or consist of CCCAGGTTGCTAGGGCTTGG.

In some embodiments, the target sequence for the first sgRNA comprises or consists essentially of or consist of one or more of AGAGCATCGGACCGAAGCGG, GGGGGAGAACATACTCGGGG, and/or CCCAGGTTGCTAGGGCTTGG.

In one aspect, the present disclosure provides a gene editing system comprising, or consisting essentially of or yet further consisting of: a first nucleotide molecule encoding a dCas9-Ten-Eleven Translocation methylcytosine dioxygenase 1 catalytic domain (TET1CD) fusion protein, wherein the dCas9-TET1 fusion protein facilitates the targeted demethylation of a gene target selected from the group consisting of CDK5L, SCML2, COL9A3, or MECP. and a second nucleotide molecule encoding at least one single guide RNA (sgRNA), comprising, or consisting essentially of, or yet further consisting of a scaffold region and a spacer region; wherein the spacer region hybridizes to a nucleotide sequence complementary to a target sequence adjacent to a 5′-end of a protospacer adjacent motif (PAM); and wherein the target sequence and the PAM are located within about 2 or aboutl kilobase (kb) and ranges as described herein of the transcriptional start site (TSS) of the cyclin dependent kinase-like 5 (CDKL5) gene, and wherein the target sequence for the first sgRNA comprises or consists essentially of AGAGCATCGGACCGAAGCGG, the target sequence for the second sgRNA comprises or consists essentially of or consists of GGGGGAGAACATACTCGGGG, and the target sequence for the third sgRNA comprises or consists essentially of or consists of CCCAGGTTGCTAGGGCTTGG.

In some embodiments, the spacer region comprises, or consists essentially of, or yet further consists of a spacer sequence provided in Table 1.

In some embodiments, the gene editing system further comprises a third nucleotide molecule encoding a dCas9 protein fused to at least one transcriptional activator.

In some embodiments, the at least one transcriptional activator fused to the dCas9 protein that comprises, or consists essentially of or consists of VP64 or a fragment thereof.

In some embodiments, the target sequence for the sgRNA comprises, or consists essentially of, or consist of one or more of AGAGCATCGGACCGAAGCGG, GGGGGAGAACATACTCGGGG, and/or CCCAGGTTGCTAGGGCTTGG.

In some embodiments, the at least one sgRNA comprises a first sgRNA, a second sgRNA, and a third sgRNA, wherein the target sequence for the first sgRNA comprises or consists essentially of, or yet further consists of AGAGCATCGGACCGAAGCGG, wherein the target sequence for the second sgRNA comprises or consists essentially of, or yet further consists of GGGGGAGAACATACTCGGGG, and wherein the target sequence for the third sgRNA comprises or consists essentially of, or yet further consists of CCCAGGTTGCTAGGGCTTGG.

In some embodiments, the first nucleotide molecule, the second nucleotide molecule, and the third nucleotide molecule are integrated into one or more viral or plasmid vectors.

In some embodiments, the viral vector is a selected from the group of a lentiviral vector, an adeno-associated viral (AAV) vector, or an adenoviral vector.

In one aspect, the disclosure provides a kit comprising the system as described herein and optional instructions for use in the methods as described herein.

In one aspect, the disclosure provides a host cell comprising the gene editing system.

In one aspect, the disclosure provides a pharmaceutical composition comprising the gene editing system, the vectors or the host cell comprising the gene editing system.

In some embodiments, the pharmaceutical composition comprises a carrier.

In some embodiments, the pharmaceutical composition comprises a pharmaceutically acceptable carrier or excipient.

Vector Systems

In one aspect, the present disclosure provides is a vector comprising, or alternatively consisting essentially of, or yet further consisting of one or more of the nucleotide molecule(s) as disclosed herein. In one embodiment, provided is a vector comprising, or consisting essentially of, or yet further consisting of a nucleotide molecule(s) as disclosed herein or its complement or an equivalent of each thereof. Such equivalent hybridize to the same targeted sequence or encodes the same protein. In one embodiment, a nucleotide molecule(s) or a vector as provided herein may further comprises another sequence, such as one or more of a sequence identified above and/or listed as a feature in the tables or figures.

In some embodiments, the first nucleotide molecule, the second nucleotide molecule, and the third nucleotide molecule are inserted into and comprised as part of one or more viral or plasmid vectors. In some embodiments, the second nucleotide molecule encoding at least one small guide RNA (sgRNAs) is inserted into, incorporated or cloned into a sgRNA expression vector. In some embodiments, the second nucleotide molecule encoding at least one small guide RNA (sgRNAs) is cloned into a viral vector. In some embodiments, the viral vector is selected from the group of retroviral vectors, adenovirus vectors, adeno-associated virus vectors, or alphavirus vectors. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof. Further details as to modern methods of vectors for use in gene transfer may be found in, for example, Kotterman et al. (2015) Viral Vectors for Gene Therapy: Translational and Clinical Outlook Annual Review of Biomedical Engineering 17. In some embodiments, the viral vector is a selected from the group of a lentiviral vector, an adeno-associated viral (AAV) vector, or an adenoviral vector.

In some embodiments, the viral vector is a lentiviral vector. In some embodiments, the lentiviral vector is an optimized lentiviral sgRNA cloning vector with MS2 loops at tetraloop and stemloop 2 and EFla-puro resistance marker.

In one aspect, the present disclosure provides a vector encoding a sgRNA. In some embodiments, the sgRNA comprises, or consists essentially of, or yet further consists of a scaffold region and a spacer region. In some embodiments, the spacer region hybridizes to a nucleotide sequence that is complementary to a target sequence comprising, or consisting essentially of, or yet further consisting of one or more of GGGGGAGAACATACTCGGGG, AGAGCATCGGACCGAAGCGG, CCCAGGTTGCTAGGGCTTGG, ATCGCCTGAAACTTGTCCGG, CGAAAGGGTGTGAAAGAGGG, and/or TGGGGAAGGTAAAGCGGCGA. In some embodiments, the spacer region hybridizes to a nucleotide sequence that is complementary to a target sequence comprising or consisting essentially of, or yet further consisting GGGGGAGAACATACTCGGGG. In some embodiments, the spacer region hybridizes to a nucleotide sequence that is complementary to a target sequence comprising or consisting essentially of, or yet further consisting AGAGCATCGGACCGAAGCGG. In some embodiments, the spacer region hybridizes to a nucleotide sequence that is complementary to a target sequence comprising or consisting essentially of, or yet further consisting CCCAGGTTGCTAGGGCTTGG. In some embodiments, the spacer region hybridizes to a nucleotide sequence that is complementary to a target sequence comprising or consisting essentially of, or yet further consisting ATCGCCTGAAACTTGTCCGG. In some embodiments, the spacer region hybridizes to a nucleotide sequence that is complementary to a target sequence comprising or consisting essentially of, or yet further consisting CGAAAGGGTGTGAAAGAGGG. In some embodiments, the spacer region hybridizes to a nucleotide sequence that is complementary to a target sequence comprising or consisting essentially of, or yet further consisting TGGGGAAGGTAAAGCGGCGA.

In one aspect, the present disclosure provides a vector encoding a first sgRNA and a second sgRNA. In some embodiments, the first sgRNA comprises or consisting essentially of, or yet further consisting a scaffold region and a spacer region, and the spacer region of the first sgRNA hybridizes to a nucleotide sequence complementary to a target sequence comprising or consisting essentially of, or yet further consisting AGAGCATCGGACCGAAGCGG In some embodiments, the second sgRNA comprises or consisting essentially of, or yet further consisting a scaffold region and a spacer region, and the spacer region of the second sgRNA hybridizes to a nucleotide sequence complementary to a target sequence comprising or consisting essentially of, or yet further consisting GGGGGAGAACATACTCGGGG.

In some embodiments, the vector encodes a first sgRNA and a second sgRNA. In some embodiments, the first sgRNA comprises or consists essentially of, or yet further consists a scaffold region and a spacer region and the spacer region of the first sgRNA hybridizes to a nucleotide sequence complementary to a target sequence comprising or consisting essentially of, or yet further consisting of AGAGCATCGGACCGAAGCGG. In some embodiments, the second sgRNA comprises or consists essentially of, or consists of a scaffold region and a spacer region, and the spacer region of the second sgRNA hybridizes to a nucleotide sequence complementary to a target sequence comprising or consisting essentially of, or consisting of CCCAGGTTGCTAGGGCTTGG.

In some embodiments, a vector encodes a first sgRNA and a second sgRNA. In some embodiments, the first sgRNA comprises or consists essentially of, or consists of a scaffold region and a spacer region and the spacer region of the first sgRNA hybridizes to a nucleotide sequence complementary to a target sequence comprising or consisting essentially of, or consisting of GGGGGAGAACATACTCGGGG. In some embodiments, the second sgRNA comprises or consists essentially of, or consists of a scaffold region and a spacer region, and the spacer region of the second sgRNA hybridizes to a nucleotide sequence complementary to a target sequence comprising or consisting essentially of, or consisting of CCCAGGTTGCTAGGGCTTGG.

In some embodiments, a vector encodes a first sgRNA, a second sgRNA, and a third sgRNA. In some embodiments, the first sgRNA comprises or consists essentially of, or consists of a scaffold region and a spacer region and the spacer region of the first sgRNA hybridizes to a nucleotide sequence complementary to a target sequence comprising or consisting essentially of, or consisting of AGAGCATCGGACCGAAGCGG. In some embodiments, the second sgRNA comprises or consists essentially of, or consists of a scaffold region and a spacer region, and the spacer region of the second sgRNA hybridizes to a nucleotide sequence complementary to a target sequence comprising or consisting essentially of, or consisting of GGGGGAGAACATACTCGGGG. In some embodiments, the third sgRNA comprises or consists essentially of, or consists of a scaffold region and a spacer region, and the spacer region of the third sgRNA hybridizes to a nucleotide sequence complementary to a target sequence comprising or consisting essentially of, or consisting of CCCAGGTTGCTAGGGCTTGG.

In some embodiments, a vector encodes a sgRNA and the sgRNA comprises or consists essentially of, or consists of a scaffold region and a spacer region, and the spacer region hybridizes to a nucleotide sequence complementary to a target sequence comprising or consisting essentially of, or consisting of AGAGCATCGGACCGAAGCGG.

In one aspect, the present disclosure provides a vector encoding a first sgRNA, a second sgRNA, and/or a third sgRNA and further comprises a nucleotide molecule encoding a dCas9-TET1CD fusion protein. In some embodiments, the vector encoding a first sgRNA, a second sgRNA, and/or a third sgRNA and further comprises or consists essentially of, or consists of a nucleotide molecule encoding a dCas9 protein fused to at least one transcriptional activator. In some embodiments, the transcriptional activator comprises VP64 or a biologically equivalent fragment thereof. In some embodiments, VP64 is the activation domain VP16. In some embodiments, VP64 is a recombinamt tetrameric repeat of comprising the minimal activation domain VP64. In some embodiments, the activation domain of VP16 comprises amino acids 413-489 of the VP16 protein. In another embodiments, the recombinant tetrameric repeat of VP16's minimal activation domain comprises or consists essentially of, or consists of the amino acid sequence DALDDFDLDML.

In some embodiments, the vector further comprises or consists essentially of, or consists of a first nucleotide molecule encoding a dCas9-TET1CD fusion protein and a second nucleotide molecule encoding a dCas9 protein fused to at least one transcriptional activator. In some embodiments, the transcriptional activator comprises VP64 or a fragment thereof. In some embodiments, VP64 fragment comprises the activation domain VP16. In some embodiments, VP64 is a recombinant tetrameric repeat of comprising or consisting essentially of or yet further consisting of the minimal activation domain VP64. In some embodiments, the activation domain of VP16 comprises or consists essentially of, or consists of amino acids 413-489 of the VP16 protein. In another embodiments, the recombinant tetrameric repeat of VP16's minimal activation domain comprises or consists essentially of, or consists of the amino acid sequence DALDDFDLDML. In some embodiments, the vector is a viral vector or a plasmid vector. In some embodiments, the viral vector is a lentiviral vector, an AAV vector, or an adenoviral vector.

In a further aspect, the systems, nucleotides, nucleic acids or host cells as described herein are detectably labeled for research or other use. Detectable labels such as radionucleotides and fluorescent labels are commercially available and widely used.

Host Cells

The present disclosure provides an isolated or engineered host cell comprising any one or more of the polynucleotides, gene editing systems and/or any one or more of the vectors as disclosed herein. In some embodiments, the host cell produces the gene editing system, the nucleotide molecule(s) and/or the vector(s). Additionally or alternatively, the host cell is an insect cell, a mammalian cell, or a bacterial cell. In some embodiment, the host cell is selected from a stem cell, an embryonic stem cell (that in one aspect is from an established cultured cell line), a progenitor cell, an IPSC, a neuronal progenitor cell, a neuronal stem cell or a stem or progenitor cell with the ability to differentiate into a neuron. The host cell can also be an egg, a sperm, a zygote, or a germline cell. In yet a further embodiment, the host cell is a mammalian cell. In one aspect the cell is a culture or primary cell from a non-human host or subject. In one aspect, the cell is a cell in need of genetic correction, e.g., a cell with inactive gene expression, as described herein. In a further aspect, the cell is a neuronal cell with dysfunctional gene expression. The cells are useful in cell assay systems and therapies as described herein.

In some embodiments, the nucleotide molecule is engineered to one or more of the chromosome(s) or chromosome sites of the host cell. In some embodiments, the host cell comprises homozygous polynucleotides. In another embodiment, the host cell comprises a heterozygous polynucleotide. In some aspects and/or embodiments of the disclosure herein, the nucleotide molecule is engineered to one or more of the chromosome(s) or chromosome site(s) of the mammalian cell.

In some embodiments, the host cell comprises gene editing systems comprising, or alternatively consisting essentially of, or yet further consisting of: (i) a first nucleotide molecule encoding a dCas9-Ten-Eleven Translocation methylcytosine dioxygenase 1 catalytic domain (TET1CD) fusion protein; and (ii) a second nucleotide molecule encoding at least one small guide RNA (sgRNA). In some embodiment, the second nucleotide molecule encoding at least one small guide RNA (sgRNA) that comprises a scaffold region and a spacer region. In some embodiment, the spacer region hybridizes to a nucleotide sequence that is complementary to a target sequence adjacent to a 5′-end of a protospacer adjacent motif (PAM). In some embodiments, the target sequence and the PAM are located at least 1 kilobase (kb) from the transcriptional start site (TSS) of the CDKL5 gene. In some embodiments, the first nucleotide molecule encoding a dCas9-Ten-Eleven Translocation methylcytosine dioxygenase 1 catalytic domain (TET1CD) fusion protein; and (ii) the second nucleotide molecule encoding at least one small guide RNA (sgRNA) induce DNA demethylation of CpGs (GC islands or region) at positions of at least about −1500, at least about −1000, at least about −500, at least about −200, at least about −148, at least about −66 and, at least about −19 relative to transcription start site.

In one aspect, the present disclosure provides a host cell expressing a third nucleotide molecule encoding a dCas9 protein fused to at least one transcriptional activator. In some embodiments, the fusion protein comprising the deactivated CRISPR-associated protein 9 (dCas9) with at least one tandem repeat of the transcriptional activator herpes simplex virus VP16 (i.e. VP64) induces transcriptional activation of endogenous of an endogenous gene. In some embodiments, the at least one transcriptional activator comprises VP64 or a fragment thereof. Transcription factors act through a DNA-binding domain that localizes a protein to a specific site within the genome and through accessory effector domains that either activate or repress transcription at or near that site. Effector domains, such as the activation domain the herpes simplex virus VP16 (4) and the repression domain Kruppel-associated box (KRAB), are modular and retain their activity when they are fused to other DNA-binding proteins. In some embodiments, VP64 is the activation domain VP16. In some embodiments, VP64 is a recombinant tetrameric repeat of comprising the mimimal activation domain VP64. In some embodiments, the activation domain of VP16 comprises amino acids 413-489 of the VP16 protein. In another embodiments, the recombinant tetrameric repeat of VP16's minimal activation domain comprises the amino acid sequence DALDDFDLDML

In some embodiment, dCas9-VP64 fusion protein upregulates genes in the host cell in an unmethylated chromatin context. In some embodiment combination of dCas9-VP64 fusion protein and dCas9-TET1CD shows a synergistic effect resulted in a greater than 60% expression of an inactive allele (i.e. silence allele) in the host cell. In some embodiments, expression of dCas9-VP64 fusion protein alone does not significantly increase the reactivation levels of the inactive allele. In some embodiments, dual expression of dCas9-VP64 fusion protein and dCas9-TET1CD resulted in the fewest number of differentially expressed genes in RNAseq analysis.

In some embodiments, the host cell further expresses several sgRNAs. In some embodiments, the host cell expresses six sgRNAs. In some embodiments, the host cell expresses at least about, 1-10, 1-5, 1-6, 1-3, 3-6, or 4-6 sgRNAs. In some embodiments, the host cell expresses a target sequence that is complementary to a sgRNA sequence selected from the group consisting of AGAGCATCGGACCGAAGCGG, GGGGGAGAACATACTCGGGG, CCCAGGTTGCTAGGGCTTGG, ATCGCCTGAAACTTGTCCGG, CGAAAGGGTGTGAAAGAGGG, and TGGGGAAGGTAAAGCGGCGA. In some embodiments, the target sequence for the sgRNA comprises AGAGCATCGGACCGAAGC. In some embodiments, the target sequence for the sgRNA comprises GGGGGAGAACATACTCGGGG. In some embodiments, the target sequence for the sgRNA comprises CCCAGGTTGCTAGGGCTTGG.

In some embodiments, the host cell expresses a second nucleotide molecule encoding at least one small guide RNA (sgRNA) that comprises at least three sgRNAs. In some embodiments, the host cell expresses a second nucleotide molecule encoding at least one small guide RNA (sgRNA) that comprises a first sgRNA, a second sgRNA, and a third sgRNA. In some embodiments, the target sequence for the first sgRNA comprises AGAGCATCGGACCGAAGCGG. In some embodiments, the target sequence for the second sgRNA comprises GGGGGAGAACATACTCGGGG. In some embodiments, the target sequence for the third sgRNA comprises CCCAGGTTGCTAGGGCTTGG. In some embodiments, the target sequence for the first sgRNA comprises AGAGCATCGGACCGAAGCGG, the target sequence for the second sgRNA comprises GGGGGAGAACATACTCGGGG, and the target sequence for the third sgRNA comprises CCCAGGTTGCTAGGGCTTGG.

In one aspect, the present disclosure provides a host cell genetically engineered to express a vector comprising, or alternatively consisting essentially of, or yet further consisting of one or more of the nucleotide molecule(s) as disclosed herein. In one embodiment, provided is a vector comprising, or consisting essentially of, or yet further consisting of a nucleotide molecule(s) as disclosed herein or its complement or an equivalent of each thereof. Such equivalent hybridize to the same targeted sequence or encodes the same protein. In one embodiment, a nucleotide molecule(s) or a vector as provided herein may further comprises another sequence, such as one or more of a sequence listed as a feature in the drawings.

In some embodiments, the host cell expresses a first nucleotide molecule, the second nucleotide molecule, and the third nucleotide molecule are cloned into one or more viral or plasmid vectors. In some embodiments, the second nucleotide molecule encoding at least one small guide RNA (sgRNAs) is cloned into a sgRNA expression vector. In some embodiments, the second nucleotide molecule encoding at least one small guide RNA (sgRNAs) is cloned into a viral vector. In some embodiments, the viral vector is selected from the group consisting of retroviral vectors, adenovirus vectors, adeno-associated virus vectors, and alphavirus vectors. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene. Further details as to modern methods of vectors for use in gene transfer may be found in, for example, Kotterman et al. (2015) Viral Vectors for Gene Therapy: Translational and Clinical Outlook Annual Review of Biomedical Engineering 17. In some embodiments, the viral vector is a selected from the group of a lentiviral vector, an adeno-associated viral (AAV) vector, or an adenoviral vector, e.g., an Addgene plasmid available under 73797 or an equivalent thereof. In some embodiments, the viral vector is a lentiviral vector. In some embodiments, the lentiviral vector is an optimized lentiviral sgRNA cloning vector with MS2 loops at tetraloop and stemloop 2 and EFla-puro resistance marker.

In one aspect, the present disclosure provides a host cell engineered to express a vector encoding a sgRNA. In some embodiments, the sgRNA comprises a scaffold region and a spacer region. In some embodiments, the spacer region hybridizes to a nucleotide sequence that is complementary to a target sequence comprising or consisting essentially of or consisting of GGGGGAGAACATACTCGGGG, AGAGCATCGGACCGAAGCGG, CCCAGGTTGCTAGGGCTTGG, ATCGCCTGAAACTTGTCCGG, CGAAAGGGTGTGAAAGAGGG, or TGGGGAAGGTAAAGCGGCGA. In some embodiments, the spacer region hybridizes to a nucleotide sequence that is complementary to a target sequence comprising or consisting essentially of or consisting of GGGGGAGAACATACTCGGGG. In some embodiments, the spacer region hybridizes to a nucleotide sequence that is complementary to a target sequence comprising or consisting essentially of or consisting of AGAGCATCGGACCGAAGCGG. In some embodiments, the spacer region hybridizes to a nucleotide sequence that is complementary to a target sequence comprising or consisting essentially of or consisting of CCCAGGTTGCTAGGGCTTGG. In some embodiments, the spacer region hybridizes to a nucleotide sequence that is complementary to a target sequence comprising ATCGCCTGAAACTTGTCCGG. In some embodiments, the spacer region hybridizes to a nucleotide sequence that is complementary to a target sequence comprising or consisting essentially of or consisting of CGAAAGGGTGTGAAAGAGGG. In some embodiments, the spacer region hybridizes to a nucleotide sequence that is complementary to a target sequence comprising or consisting essentially of or consisting of TGGGGAAGGTAAAGCGGCGA.

In one aspect, the present disclosure provides a host cell engineered to express a vector encoding a first sgRNA and a second sgRNA. In some embodiments, the host cell expresses a first sgRNA that comprises a scaffold region and a spacer region, and the spacer region of the first sgRNA hybridizes to a nucleotide sequence complementary to a target sequence comprising or consisting essentially of or consisting of AGAGCATCGGACCGAAGCGG. In some embodiments, the host cell expresses a second sgRNA that comprises a scaffold region and a spacer region, and the spacer region of the second sgRNA hybridizes to a nucleotide sequence complementary to a target sequence comprising or consisting essentially of or consisting of GGGGGAGAACATACTCGGGG.

In some embodiments, the host cell expresses a vector that encodes a first sgRNA and a second sgRNA. In some embodiments, the host cell expresses a first sgRNA that comprises a scaffold region and a spacer region and the spacer region of the first sgRNA hybridizes to a nucleotide sequence complementary to a target sequence comprising or consisting essentially of or consisting of AGAGCATCGGACCGAAGCGG. In some embodiments, the host cell expresses a second sgRNA that comprises a scaffold region and a spacer region, and the spacer region of the second sgRNA hybridizes to a nucleotide sequence complementary to a target sequence comprising or consisting essentially of or consisting of CCCAGGTTGCTAGGGCTTGG.

In some embodiments, the host cell expresses a vector that encodes a first sgRNA and a second sgRNA. In some embodiments, the host cell expresses a first sgRNA comprises a scaffold region and a spacer region and the spacer region of the first sgRNA hybridizes to a nucleotide sequence complementary to a target sequence comprising or consisting essentially of or consisting of GGGGGAGAACATACTCGGGG. In some embodiments, the host cell expresses a second sgRNA comprises a scaffold region and a spacer region, and the spacer region of the second sgRNA hybridizes to a nucleotide sequence complementary to a target sequence comprising or consisting essentially of or consisting of CCCAGGTTGCTAGGGCTTGG.

In some embodiments, the host cell expresses a vector that encodes a first sgRNA, a second sgRNA, and a third sgRNA. In some embodiments, the first sgRNA comprises a scaffold region and a spacer region and the spacer region of the first sgRNA hybridizes to a nucleotide sequence complementary to a target sequence comprising or consisting essentially of or consisting of AGAGCATCGGACCGAAGCGG. In some embodiments, the host cell expresses a second sgRNA comprises a scaffold region and a spacer region, and the spacer region of the second sgRNA hybridizes to a nucleotide sequence complementary to a target sequence comprising or consisting essentially of or consisting of GGGGGAGAACATACTCGGGG. In some embodiments, the host cell expresses a third sgRNA comprises a scaffold region and a spacer region, and the spacer region of the third sgRNA hybridizes to a nucleotide sequence complementary to a target sequence comprising or consisting essentially of or consisting of CCCAGGTTGCTAGGGCTTGG.

In some embodiments, the host cell expresses a vector that encodes a sgRNA and the sgRNA comprises a scaffold region and a spacer region, and the spacer region hybridizes to a nucleotide sequence complementary to a target sequence comprising or consisting essentially of or consisting of AGAGCATCGGACCGAAGCGG.

In one aspect, the present disclosure provides the host cell engineered to express a vector encoding a first sgRNA, a second sgRNA, and/or a third sgRNA and further comprises or consists essentially of or consists of a nucleotide molecule encoding a dCas9-TET1CD fusion protein. In some embodiments, the host cell expresses a vector encoding a first sgRNA, a second sgRNA, and/or a third sgRNA and further comprises a nucleotide molecule encoding a dCas9 protein fused to at least one transcriptional activator. In some embodiments, the transcriptional activator comprises VP64 or a biologically active fragment thereof. In some embodiments, the biologically active fragment of VP64 is the activation domain VP16. In some embodiments. VP64 is a recombinant tetrameric repeat of comprising the minimal activation domain VP64. In some embodiments, the activation domain of VP16 comprises or consists essentially of or consists of amino acids 413-489 of the VP16 protein. In another embodiments, the recombinant tetrameric repeat of VP16's minimal activation domain comprises or consists essentially of or consists of the amino acid sequence DALDDFDLDML.

In some embodiments, the host cell expresses a vector that further comprises or consists essentially of or consists of a first nucleotide molecule encoding a dCas9-TET1CD fusion protein and a second nucleotide molecule encoding a dCas9 protein fused to at least one transcriptional activator. In some embodiments, the transcriptional activator comprises or consists essentially of or consists VP64 or a biologically active fragment thereof. In some embodiments, the biologically active fragment of VP64 is the activation domain VP16. In some embodiments, VP64 is a recombinant tetrameric repeat of comprising or consisting essentially of or consisting of the minimal activation domain VP64. In some embodiments, the activation domain of VP16 comprises or consists essentially of or consists amino acids 413-489 of the VP16 protein. In another embodiments, the recombinant tetrameric repeat of VP16's minimal activation domain comprises or consists essentially of or consists of the amino acid sequence DALDDFDLDML In some embodiments, the vector is a viral vector or a plasmid vector. In some embodiments, the viral vector is a lentiviral vector, an AAV vector, or an adenoviral vector.

In one aspect, the present disclosure provides for a pharmaceutical composition comprising an isolated or engineered host cell comprising any one or more of the polynucleotides, systems, vectors or host cells alone or in combination with each other and optionally additional therapeutic agents, and a carrier, optionally a pharmaceutically acceptable carrier or excipient. In some embodiments, the host cell produces the gene editing system, the nucleotide molecule(s) and/or the vector(s).

In some embodiments, the pharmaceutical composition comprises a host cell comprising a gene editing systems comprising, or alternatively consisting essentially of, or yet further consisting of: (i) a first nucleotide molecule encoding a dCas9-Ten-Eleven Translocation methylcytosine dioxygenase 1 catalytic domain (TET1CD) fusion protein; and (ii) a second nucleotide molecule encoding at least one small guide RNA (sgRNA). In some embodiment, the second nucleotide molecule encoding at least one small guide RNA (sgRNA) that comprises a scaffold region and a spacer region. In some embodiments, the composition comprises a carrier, optionally a pharmaceutically acceptable carrier or excipient. In some embodiment, the spacer region hybridizes to a nucleotide sequence that is complementary to a target sequence adjacent to a 5′-end of a protospacer adjacent motif (PAM). In some embodiments, the target sequence and the PAM are located at least 1 kilobase (kb) from the transcriptional start site (TSS) of the CDKL5 gene. In some embodiments, the first nucleotide molecule encoding a dCas9-Ten-Eleven Translocation methylcytosine dioxygenase 1 catalytic domain (TET1CD) fusion protein; and (ii) the second nucleotide molecule encoding at least one small guide RNA (sgRNA) induce DNA demethylation of CpGs (GC islands or region) at positions of at least about −1500, at least about −1000, at least about −500, at least about −200, at least about −148, at least about −66 and, at least about −19 relative to transcription start site.

In one aspect, the present disclosure provides a composition comprising a host cell as described herein and expressing a third nucleotide molecule encoding a dCas9 protein fused to at least one transcriptional activator. In some embodiments, the fusion protein comprising the deactivated CRISPR-associated protein 9 (dCas9) with at least one tandem repeat of the transcriptional activator herpes simplex virus VP16 (i.e.VP64) induces transcriptional activation of endogenous of an endogenous gene. In some embodiments, the at least one transcriptional activator comprises VP64 or a biologically active fragment thereof. Transcription factors act through a DNA-binding domain that localizes a protein to a specific site within the genome and through accessory effector domains that either activate or repress transcription at or near that site. Effector domains, such as the activation domain the herpes simplex virus VP16 (4) and the repression domain Kruppel-associated box (KRAB), are modular and retain their activity when they are fused to other DNA-binding proteins. In some embodiments, VP64 is the activation domain VP16. In some embodiments, VP64 is a recombinant tetrameric repeat of comprising the minimal activation domain VP64. In some embodiments, the activation domain of VP16 comprises amino acids 413-489 of the VP16 protein. In another embodiments, the recombinant tetrameric repeat of VP16's minimal activation domain comprises the amino acid sequence DALDDFDL_DML.

In some embodiments, the composition comprises a host cell as described herein that further expresses several sgRNAs, also as described herein. In some embodiments, the host cell expresses six sgRNAs. In some embodiments, the host cell expresses at least about, 1-10, 1-5, 1-6, 1-3, 3-6, or 4-6 sgRNAs. In some embodiments, the host cell expresses a target sequence that is complementary to a sgRNA sequence selected from the group of AGAGCATCGGACCGAAGCGG, GGGGGAGAACATACTCGGGG, CCCAGGTTGCTAGGGCTTGG, ATCGCCTGAAACTTGTCCGG, CGAAAGGGTGTGAAAGAGGG, or TGGGGAAGGTAAAGCGGCGA. In some embodiments, the target sequence for the sgRNA comprises or consists essentially of or consists of AGAGCATCGGACCGAAGC. In some embodiments, the target sequence for the sgRNA comprises or consists essentially of or consists or GGGGGAGAACATACTCGGGG. In some embodiments, the target sequence for the sgRNA comprises or consists essentially of or consists or CCCAGGTTGCTAGGGCTTGG.

In some embodiments, the pharmaceutical composition comprises a host cell as described herein that expresses a second nucleotide molecule encoding at least one small guide RNA (sgRNA) that comprises or consists essentially of or consists of at least three sgRNAs. In some embodiments, the host cell expresses a second nucleotide molecule encoding at least one small guide RNA (sgRNA) that comprises a first sgRNA, a second sgRNA, and a third sgRNA. In some embodiments, the target sequence for the first sgRNA comprises or consists essentially of or consists or AGAGCATCGGACCGAAGCGG. In some embodiments, the target sequence for the second sgRNA comprises or consists essentially of or consists or GGGGGAGAACATACTCGGGG. In some embodiments, the target sequence for the third sgRNA comprises or consists essentially of or consists or CCCAGGTTGCTAGGGCTTGG. In some embodiments, the target sequence for the first sgRNA comprises or consists essentially of or consists or AGAGCATCGGACCGAAGCGG, the target sequence for the second sgRNA comprises or consists essentially of or consists or GGGGGAGAACATACTCGGGG, and the target sequence for the third sgRNA comprises or consists essentially of or consists or CCCAGGTTGCTAGGGCTTGG.

In one aspect, the present disclosure provides a composition comprising a host cell as described herein genetically engineered to express a vector comprising, or alternatively consisting essentially of, or yet further consisting of one or more of the nucleotide molecule(s) as disclosed herein. In one embodiment, provided is a vector comprising, or consisting essentially of, or yet further consisting of a nucleotide molecule(s) as disclosed herein or its complement or an equivalent of each thereof. Such equivalent hybridize to the same targeted sequence or encodes the same protein. In one embodiment, a nucleotide molecule(s) or a vector as provided herein may further comprises another sequence, such as one or more of a sequence listed as a feature in the drawings.

In some embodiments, the composition comprises a host cell that expresses a first nucleotide molecule, the second nucleotide molecule, and the third nucleotide molecule are cloned into one or more viral or plasmid vectors. In some embodiments, the second nucleotide molecule encoding at least one small guide RNA (sgRNAs) is cloned into a sgRNA expression vector. In some embodiments, the second nucleotide molecule encoding at least one small guide RNA (sgRNAs) is cloned into a viral vector. In some embodiments, the viral vector is selected from the group consisting of retroviral vectors, adenovirus vectors, adeno-associated virus vectors, and alphavirus vectors.

In one aspect, the present disclosure provides a composition comprising a host cell engineered to express a vector encoding a sgRNA. In some embodiments, the sgRNA comprises a scaffold region and a spacer region. In some embodiments, the spacer region hybridizes to a nucleotide sequence that is complementary to a target sequence comprising or consisting essentially of or consisting of GGGGGAGAACATACTCGGGG, AGAGCATCGGACCGAAGCGG, CCCAGGTTGCTAGGGCTTGG, ATCGCCTGAAACTTGTCCGG, CGAAAGGGTGTGAAAGAGGG, or TGGGGAAGGTAAAGCGGCGA. In some embodiments, the spacer region hybridizes to a nucleotide sequence that is complementary to a target sequence comprising or consisting essentially of or consisting of GGGGGAGAACATACTCGGGG. In some embodiments, the spacer region hybridizes to a nucleotide sequence that is complementary to a target sequence comprising or consisting essentially of or consisting of AGAGCATCGGACCGAAGCGG.

In some embodiments, the spacer region hybridizes to a nucleotide sequence that is complementary to a target sequence comprising or consisting essentially of or consisting of CCCAGGTTGCTAGGGCTTGG. In some embodiments, the spacer region hybridizes to a nucleotide sequence that is complementary to a target sequence comprising or consisting essentially of or consisting of ATCGCCTGAAACTTGTCCGG. In some embodiments, the spacer region hybridizes to a nucleotide sequence that is complementary to a target sequence comprising or consisting essentially of or consisting of CGAAAGGGTGTGAAAGAGGG.

In some embodiments, the spacer region hybridizes to a nucleotide sequence that is complementary to a target sequence comprising or consisting essentially of or consisting of TGGGGAAGGTAAAGCGGCGA.

In one aspect, the present disclosure provides a composition comprising a host cell engineered to express a vector encoding a first sgRNA and a second sgRNA. In some embodiments, the host cell expresses a first sgRNA that comprises a scaffold region and a spacer region, and the spacer region of the first sgRNA hybridizes to a nucleotide sequence complementary to a target sequence comprising or consisting essentially of or consisting of AGAGCATCGGACCGAAGCGG. In some embodiments, the host cell expresses a second sgRNA that comprises a scaffold region and a spacer region, and the spacer region of the second sgRNA hybridizes to a nucleotide sequence complementary to a target sequence comprising or consisting essentially of or consisting of GGGGGAGAACATACTCGGGG.

In some embodiments, the composition comprisese host cell that expresses a vector that encodes a first sgRNA and a second sgRNA. In some embodiments, the host cell expresses a first sgRNA that comprises a scaffold region and a spacer region and the spacer region of the first sgRNA hybridizes to a nucleotide sequence complementary to a target sequence comprising or consisting essentially of or consisting of AGAGCATCGGACCGAAGCGG. In some embodiments, the host cell expresses a second sgRNA that comprises a scaffold region and a spacer region, and the spacer region of the second sgRNA hybridizes to a nucleotide sequence complementary to a target sequence comprising or consisting essentially of or consisting of CCCAGGTTGCTAGGGCTTGG.

In some embodiments, the composition comprises a host cell that expresses a vector that encodes a first sgRNA and a second sgRNA. In some embodiments, the host cell expresses a first sgRNA comprises a scaffold region and a spacer region and the spacer region of the first sgRNA hybridizes to a nucleotide sequence complementary to a target sequence comprising or consisting essentially of or consisting of GGGGGAGAACATACTCGGGG. In some embodiments, the host cell expresses a second sgRNA comprises a scaffold region and a spacer region, and the spacer region of the second sgRNA hybridizes to a nucleotide sequence complementary to a target sequence comprising or consisting essentially of or consisting of CCCAGGTTGCTAGGGCTTGG.

In some embodiments, the composition comprises a host cell expresses a vector that encodes a first sgRNA, a second sgRNA, and a third sgRNA. In some embodiments, the first sgRNA comprises a scaffold region and a spacer region and the spacer region of the first sgRNA hybridizes to a nucleotide sequence complementary to a target sequence comprising or consisting essentially of or consisting of AGAGCATCGGACCGAAGCGG. In some embodiments, the host cell expresses a second sgRNA comprises a scaffold region and a spacer region, and the spacer region of the second sgRNA hybridizes to a nucleotide sequence complementary to a target sequence comprising or consisting essentially of or consisting of GGGGGAGAACATACTCGGGG. In some embodiments, the host cell expresses a third sgRNA comprises a scaffold region and a spacer region, and the spacer region of the third sgRNA hybridizes to a nucleotide sequence complementary to a target sequence comprising or consisting essentially of or consisting of CCCAGGTTGCTAGGGCTTGG.

In some embodiments, the composition comprises a host cell expresses a vector that encodes a sgRNA and the sgRNA comprises a scaffold region and a spacer region, and the spacer region hybridizes to a nucleotide sequence complementary to a target sequence comprising or consisting essentially of or consisting of AGAGCATCGGACCGAAGCGG.

In one aspect, the present disclosure provides a composition comprising a host cell engineered to express a vector encoding a first sgRNA, a second sgRNA, and/or a third sgRNA and further comprises a nucleotide molecule encoding a dCas9-TET1CD fusion protein. In some embodiments, the host cell expresses a vector encoding a first sgRNA, a second sgRNA, and/or a third sgRNA and further comprises a nucleotide molecule encoding a dCas9 protein fused to at least one transcriptional activator. In some embodiments, the transcriptional activator comprises VP64 or a biologically active fragment thereof. In some embodiments, the biologically active fragment of VP64 is the activation domain VP16. In some embodiments, VP64 is a recombinant tetrameric repeat of comprising the minimal activation domain VP64. In some embodiments, the activation domain of VP16 comprises or consists essentially of or consists of amino acids 413-489 of the VP16 protein. In another embodiments, the recombinant tetrameric repeat of VP16's minimal activation domain comprises or consists essentially of or consists of the amino acid sequence DALDDFDLDML.

In some embodiments, the composition comprises a host cell that expresses a vector that further comprises a first nucleotide molecule encoding a dCas9-TET1CD fusion protein and a second nucleotide molecule encoding a dCas9 protein fused to at least one transcriptional activator. In some embodiments, the transcriptional activator comprises or consisting essentially of or consisting of VP64 or a biologically active fragment thereof. In some embodiments, VP64 fragment comprises, or consists essentially thereof or consists of the activation domain VP16 In some embodiments, VP64 is a recombinant tetrameric repeat of comprising the minimal activation domain VP64. In some embodiments, the activation domain of VP16 comprises amino acids 413-489 of the VP16 protein. In another embodiments, the recombinant tetrameric repeat of VP16's minimal activation domain comprises or consists essentially of or consists of the amino acid sequence DALDDFDLDML. In some embodiments, the vector is a viral vector or a plasmid vector.

In some embodiments, the viral vector is a lentiviral vector, an AAV vector, or an adenoviral vector. In some embodiments, the composition comprises a carrier, optionally a pharmaceutically acceptable carrier or excipient.

Cell Assay Systems

The vectors, gene editing systems and host cells can be used as in vitro assays systems to test new therapies or additions to the vectors, gene editing systems or host cells as described herein. Thus, in one aspect, provided herein is a method for increasing a gene expression such as a CDKL5 gene expression in a cell, comprising inserting into the cell the vectors and/or gene editing systems as described above. In one aspect, the gene expression is lower than wildtype expression due to reduced DNA methylation of the CDKL5 promoter region. Although CDKL5 is used as an example of such as system, one skill in the art can apply the principles of this system to other genes wherein DNA methylation is reduced, and/or the promoter region is located on a silenced X-chromosomal allele of the cell. The cells can be samples isolated from subjects suspected of containing defective gene expression and/or a commercially available or laboratory generated cell line. The host cell can be a prokaryotic or a eukaryotic cell, non-limiting examples of such include an insect cell, a mammalian cell, or a bacterial cell. In some embodiment, the host cell is selected from an egg, a sperm, a zygote, or a germline cell. In yet a further embodiment, the host cell is a mammalian cell. In one aspect, the cell is a cell in need of genetic correction, e.g., a cell with dysfunctional gene expression, as described herein. In a further aspect, the cell is a neuronal cell with dysfunctional gene expression.

One of skill of the art can generate the host cell system with a cell or cells from a subject to determine if the therapy is useful for the subject. In additional or alternatively, additional therapies can be tested for combination therapy.

The insertion of the vectors and/or gene editing system can be in vitro, ex vivo or in vivo. When used in an animal, it can serve as an animal model to assay for combination therapies.

Therapeutic and Diagnostic Methods

The present disclosure provides a gene editing system comprising a first nucleotide encoding a dCas9-ten-Eleven Translocation methylcytosine dioxygenase 1 catalytic domain (TET1CD) fusion protein and a second nucleotide encoding at least one small guide RNA (sgRNA) for targeting a nucleotide complementary sequence located within aboutl kilobase of the transcritptional start site (TSS) of the CDKL5 gene. Additional alterations and modifications to the systems are provided herein.

A significant number of X-linked genes escape from X chromosome inactivation and are associated with a distinct epigenetic signature. One epigenetic modification that strongly correlates with X-escape is reduced DNA methylation in promoter regions. Applicant created an artificial escape system by editing DNA methylation on the promoter of CDKL5, a gene causative for an infantile epilepsy, from the silenced X-chromosomal allele in human neuronal-like cells. The artificial system comprises a fusion of the catalytic domain of TET1 to dCas9 that is targeted to the CDKL5 promoter using three guide RNAs. This artificial system caused significant reactivation of the inactive CDKL5 allele in combination with removal of methyl groups from CpG dinucleotides. The artificial system also was further enhanced with co-expression of dCas9-TET1 fusion protein and a fusion protein comprising dCas9 and theVP64 transactivator. Together, these two dCas9 fusion proteins exhibited a synergistic effect on the reactivation of the inactive allele to levels above 60% of the active allele (FIG. 7). Applicant further used a multi-omics assessment to determine potential off-targets on the transcriptome and methylome, and found that synergistic delivery of dCas9 effectors is highly selective for the target site. Unexpectely, the application of this artificial system elucidated a causal role for reduced DNA methylation with escape from X chromosome inactivation. This novel artificial system has great potential for those suffering from X-linked disorders.

In particular, defects in epigenetics modification of ions channel in the nervous system are linked to Rett syndrome (RTT) and cyclin-dependent kinase-like 5 (CDKL5) deficiency disorder (CDD). RTT and CDKL5 deficiency disorder are two X-linked developmental brain disorders with overlapping but distinct phenotypic features. Mutations in the X-linked gene encoding methyl-CpG-binding protein 2 (MECP2) account for 90-95% of the case of classic Rett syndrome, and mutations in the X-linked gene encoding CDKL5 account from some cases of atypical RTT that manifest with early refractory epilepsy.

The neurodevelopmental disorder CDKL5 deficiency is caused by de novo mutations in the CDKL5 gene on the X chromosome (30). Due to random XCI, females affected by the disorder form a mosaic of tissue with cells expressing either the mutant or wild type allele (31). A potential therapeutic approach might be to activate the silenced wild type CDKL5 allele in cells expressing the loss-of-function mutant allele. Applicants synthetically induced escape of CDKL5 from the inactive X chromosome in the neuronal-like cell line SH-SY5Y via a DNA methylation editing of the CDKL5 promoter using a dCas9-TET1 fusion protein for targeted DNA demethylation. This artificial system/synthetic induction of CDKL5 escape from XCI, resulted in a significant increase in allele-specific expression of the inactive CDKL5 allele and correlated with a significant reduction in methylated CpG dinucleotides in the CGI core promoter.

The present disclosure demonstrates that dCas9-TET1 has a synergistic effect with the dCas9-VP64, thereby further increasing transcript levels from the inactive allele. The disclosure also provides describes whole-transcriptomic and genome-wide methylation data that illustrate the specificity of the novel artificial system for one target gene (CDKL5). As such, the disclosure demonstrates that loss of DNA methylation is crucial for inducing escape from the inactive X chromosome, and illustrates a novel therapeutic avenue for treatment subjects suffering from X-linked disorders generally.

In one aspect, the present disclosure provides a method for increasing CDKL5 gene expression in a cell or subject in need thereof comprising or consisting essentially of or consisting of administering to the cell or subject a system of gene editing systems comprising, or alternatively consisting essentially of, or yet further consisting of: (i) a first nucleotide molecule encoding a dCas9-Ten-Eleven Translocation methylcytosine dioxygenase 1 catalytic domain (TET1CD) fusion protein; and (ii) a second nucleotide molecule encoding at least one small guide RNA (sgRNA). In some embodiment, the second nucleotide molecule encoding at least one small guide RNA (sgRNA) that comprises a scaffold region and a spacer region. In some embodiment, the spacer region hybridizes to a nucleotide sequence that is complementary to a target sequence adjacent to a 5′-end of a protospacer adjacent motif (PAM). In some embodiments, the target sequence and the PAM are located at least 1 kilobase (kb) from the transcriptional start site (TSS) of the CDKL5 gene.

In one aspect, the present disclosure provides a method for increasing CDKL5 gene expression in a cell or subject in need thereof comprising or consisting essentially of or consisting of administering to the cell or subject a system of gene editing further comprising, a third nucleotide molecule encoding a dCas9 protein fused to at least one transcriptional activator. In some embodiments, the transcriptional activator comprises VP64 or a biologically active fragment thereof. In some embodiments, VP64 is the activation domain VP16. In some embodiments, VP64 is a recombinant tetrameric repeat of comprising or consisting essentially of or consisting of the minimal activation domain VP64. In some embodiments, the activation domain of VP16 comprises or consists essentially of or consists of amino acids 413-489 of the VP16 protein. In another embodiments, the recombinant tetrameric repeat of VP16's minimal activation domain comprises or consists essentially of or consists of the amino acid sequence DALDDFDLDML

In some embodiments, a method for increasing CDKL5 gene expression in a cell or subject in need thereof comprising or consisting essentially of or consisting of administering to the cell or subject a system of gene editing further comprising a sgRNA. In some embodiments, the system of gene editing system comprises at least about, 1-10, 1-5, 1-6, 1-3, 3-6, or 4-6 sgRNAs. In some embodiments, the system of gene editing system comprises consists essentially of or consists of a sgRNA selected from the group of AGAGCATCGGACCGAAGCGG, GGGGGAGAACATACTCGGGG, CCCAGGTTGCTAGGGCTTGG, ATCGCCTGAAACTTGTCCGG, CGAAAGGGTGTGAAAGAGGG, or TGGGGAAGGTAAAGCGGCGA. In some embodiments, the gene editing system comprises or consists essentially of or consists of a sgRNA with a sequence set forth as AGAGCATCGGACCGAAGC. In some embodiments, the gene editing system comprises or consists essentially of or consists of a sgRNA with a sequence set forth as GGGGGAGAACATACTCGGGG. In some embodiments, the gene editing system comprises a sgRNA with a sequence set forth as CCCAGGTTGCTAGGGCTTGG.

In some embodiments, the second nucleotide molecule encoding at least one small guide RNA (sgRNA) comprises at least three sgRNAs. In some embodiments, the second nucleotide molecule encoding at least one small guide RNA (sgRNA) comprises a first sgRNA, a second sgRNA, and a third sgRNA. In some embodiments, the target sequence for the first sgRNA comprises or consists essentially of or consists of AGAGCATCGGACCGAAGCGG. In some embodiments, the target sequence for the second sgRNA comprises or consists essentially of or consists of GGGGGAGAACATACTCGGGG. In some embodiments, the target sequence for the third sgRNA comprises or consists essentially of or consists of CCCAGGTTGCTAGGGCTTGG.

In some embodiments, the target sequence for the first sgRNA comprises or consists essentially of or consists of AGAGCATCGGACCGAAGCGG, the target sequence for the second sgRNA comprises or consists essentially of or consists of GGGGGAGAACATACTCGGGG, and the target sequence for the third sgRNA comprises or consists essentially of or consists of CCCAGGTTGCTAGGGCTTGG.

In one aspect, the present disclosure provides a method for increasing CDKL5 gene expression in a cell or a subject in need thereof comprising administering to the cell or subject a pharmaceutical composition of the present disclosure. In some embodiments, administering to a subject a gene editing system or the pharmaceutical composition of the present invention reduces DNA methylation in a CDKL5 promoter region of the subject. In some embodiments, the CDKL5 promoter region is located on a silenced X-chromosomal allele of the subject. In some embodiments, the subject in need for increasing CDKL5 gene expression has been diagnosed with CDKL5 deficiency disorder (CDD). In some embodiments, the subject is a mammal or mammalian cell. In some embodiments, the mammal is a non-human fetus, an infant, a juvenile, or an adult.

In some embodiments, the system or pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

In one aspect, the present disclosure provides a method for treating or preventing CDD in a cell or subject in need thereof comprising administering to the cell or subject a gene editing system or the pharmaceutical composition of the present invention. In some embodiments, administering to a subject a gene editing system or the pharmaceutical composition of the present invention reduces DNA methylation in a CDKL5 promoter region of the subject. In some embodiments, the CDKL5 promoter region is located on a silenced X-chromosomal allele of the subject. In some embodiments, the subject is a mammal. In some embodiments, the mammal is a non-human fetus, an infant, a juvenile, or an adult. In some embodiments, the system or pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

Kits

In one aspect, the present invention provides a kit comprising or consisting essentially of, or yet further consisting of any one or more of the gene editing system, the vector, the host cell or the compositions and an optional instruction for use in activating a silenced X-chomosomal allele in a subject in need thereof. In some embodiments, the kit is used for increasing CDKL5 gene expression in a subject in need thereof. In some embodiments, the kit is used for treating or preventing CDD in a subject in need thereof. In some embodiments, a kit comprising the gene editing system of the present invention and optional instructions for use as described herein.

The following examples are provided to illustrate but not limit the aspects of this disclosure.

### EXAMPLES

The examples herein are provided to illustrate advantages of the present technology and to further assist a person of ordinary skill in the art with preparing and/or using the compounds of the present technology. The examples herein are also presented in order to more fully illustrate the preferred aspects of the present technology. The examples should in no way be construed as limiting the scope of the present technology. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Material and Methods

Cloning ofsgRNAs. For the cloning of CDKL5 sgRNAs 20-bp spacer sequences were selected within ±1 kb of the CDKL5 TSS (chrX:18,443,725, hg19) using the online tool CHOPCHOP (Montague et al., Nucleic Acids Res. 42:W401-7 (2014)). For transient transfection experiments, sgRNAs were cloned into a sgRNA expression vector (Addgene plasmid #73797) following a previously published protocol (Mali et al., Science 339:823-826. (2013)). For transductions, sgRNAs were cloned into a lentiviral expression vector (Addgene plasmid #73797) as previously described (Joung et al., Nat. Protoc. 12:828-863 (2017)). Spacer sequences used to create target-specific sgRNA expression vectors are listed in Table 1. All constructs were sequence confirmed by Sanger sequencing (Genewiz, Inc, South Plainfield, N.J., USA) and chromatograms were analysed using SnapGene software (from GSL Biotech; available at snapgene.com).

TABLE 1
Spacer Sequences Used to Create Target-Specific sgRNA Expression Vectors.

| Oligonucleotide Name | Function | 5′−>3′ Sequence |
|---|---|---|
| CDKL5 sgRNA1 | spacer sequence | AGAGCATCGGACCGAAGCGG |
| CDKL5 sgRNA2 | spacer sequence | GGGGGAGAACATACTCGGGG |
| CDKL5 sgRNA3 | spacer sequence | CCCAGGTTGCTAGGGCTTGG |
| CDKL5 sgRNA4 | spacer sequence | ATCGCCTGAA ACTTGTCCGG |
| CDKL5 sgRNA5 | spacer sequence | CGAAAGGGTGTGAAAGAGGG |
| CDKL5 sgRNA6 | spacer sequence | TGGGGAAGGTAAAGCGGCGA |
| rsl808_gDNA_F | Sanger sequencing | GCTTGAGCAATTTCGGACCC |
| rsl808_gDNA_R | Sanger sequencing | TGTGTCTCTTGCTGGTACCG |
| rs35478150_gDNA_F | Sanger sequencing | TGAGCCTGTGCCAGAGGATA |
| rs35478150_gDNA_R | Sanger sequencing | TCAACTTTGATTGCCAAGTGCA |
| rs35478150_cDNA_F | Sanger sequencing | GAGCAGTTCTGGAACCAACC |
| rs35478150_cDNA_R | Sanger sequencing | TTGAGGCCGAAGAGAGATGT |
| rsl808_cDNA_F | Sanger sequencing | CCTTGTGGAATTTGGGTCAT |
| rsl808_cDNA_R | Sanger sequencing | TCAAATGCAGGCACTTAGAAT |
| CDKL5_AmpSeqF | Amplicon sequencing | CAAGGAAAAAGAGAAGCAAGGA |
| CDKL5_AmpSeqR | Amplicon sequencing | ATTTTAATGGCTGGCTTTGG |
| CDKL5_BSS_F | Amplicon sequencing | TTTTAGTTTAGGTTGTTAGGGTTTG |
| CDKL5_BSS_R | Amplicon sequencing | TAAAAAAACACCTCAAATTTTACCC |
| CDKL5_ChIP_AF | ChIP-qPCR | TCATCCTCCTTGGAAACCCG |
| CDKL5_ChIP_AR | ChIP-qPCR | GTCATCGCCCAACCAGTACA |
| CDKL5_ChIP_BF | ChIP-qPCR | AGCAGCAGCAATGGACTTCG |
| CDKL5_ChIP_BR | ChIP-qPCR | AGAAATACAGGATGGAGGATGGT |
| CDKL5_ChIP_CF | ChIP-qPCR | AAGCGCTTCCTCCTCATTGG |
| CDKL5_ChIP_CR | ChIP-qPCR | AAAGCACCTCAGGTTTTGCC |
| MECP2_ChIP_F | ChIP-qPCR | AGCTGTTGATTGGCTGCTTT |
| MECP2_ChIP_R | ChIP-qPCR | TTCAAATTOCGCCCACTAAA |
| ChIP_SCML2F | ChIP-qPCR | CACCTCCCAGCTTCACTCTC |
| ChIP_SCML2R | ChIP-qPCR | CTGCGGGTTCATCTAGTTCC |
| CDKL5_common_F | ChIP-qPCR | ACAACCAGCATTCGATCCAT |
| CDKL5_A_allele_R | ChIP-qPCR | GCTGTCGGAATTGGGTACTGTTT |
| CDKL5_C_allele_R | ChIP-qPCR | GCTGTCGGAATTGGGTACTGTTG |
| CDKL5_qPCR_F | RT-qPCR | AACTCTTACTTGGCGCTCCC |
| CDKL5_qPCR_R | RT-qPCR | CTGTCCATCGCTAAGCTCCC |
| GAPDH_qPCR_F | RT-qPCR | AATCCCATCACCATCTTCCA |
| GAPDH_qPCR_R | RT-qPCR | CTCCATGGTGGTGAAGACG |

Transient transfection experiments. U87MG (ATCC, Manassas, Va.) and Lenti-X 293T (Takara Bio USA, Inc., Mountain View, Calif.) were grown in media containing high-glucose DMEM supplemented with 1% L-glutamine (Thermo Fisher Scientific, Waltham, Mass.) and 10% HyClone heat-inactivated FBS (Thermo Fisher Scientific). BE(2)C (ATCC) cells were grown in DME/F12 (Thermo Fisher Scientific) supplemented with 1% L-glutamine and 10% HyClone heat-inactivated FBS. For gene expression modulation experiments, cells per well were grown to 80% confluency and transfected within 24 hours of plating using Lipofectamine 3000 (Life Technologies) following the manufacturer's instructions with 3 ul of Lipofectamine 3000 reagent diluted in 500 ul Opti-MEM reduced serum media (Thermo Fisher Scientific). Transfections were performed in 12-well plates using either a mock-treatment (diluted transfection reagent) or 700 ng dCas9 expression vector (Fuw-dCas9-Tet1CD-P2A-BFP, Addgene plasmid #108245; Fuw-dCas9-Tet1CD_IM, Addgene plasmid #84479; pLV hUbC-dCas9-T2A-GFP, Addgene plasmid #53191; pLV hUbC-dCas9 VP64-T2A-GFP, Addgene plasmid #53192) and 300 ng of equimolar pooled sgRNA expression vectors. Transfection medium was replaced 24 hours post-transfection with complete growth medium.

48 hours post-transfection, cells were rinsed in 1×DPBS (Thermo) and lysed in the well using TriZol (Ambion, Austin, Tex.). Total RNA was extracted using the Direct-zol RNA Miniprep kit (Zymo Research, Irvine, Calif.) and 500 ng RNA was reverse transcribed using RevertAid First Strand cDNA Synthesis Kit according to the manufacturer's instructions using random hexamer primers. Real-time PCR was performed in triplicate with 20 ng of cDNA per reaction and PowerUp SYBR Green Master Mix (Thermo Fisher Scientific) using the StepOne Plus Real Time PCR system (Thermo Fisher Scientific) and the StepOne Plus software was used to extract raw CT values. Gene expression analysis was performed with GAPDH as a reference gene in three biological replicates using exon-spanning primers for CDKL5 and GAPDH. All primer oligonucleotides used in this study are listed in Supplementary Table 1. Fold change of gene expression was calculated as the delta delta CT between GAPDH and CDKL5 transcript levels normalized to Mock-treated relative CDKL5 transcript levels as the reference.

Integrative XCI status analysis of CDKL5. In order to determine the XCI status of CDKL5, publicly available data from GTEx (gtexportal.org) was used to determine the sex-biased expression using 27 GTEx v6p tissues and blood dendritic cells from a female of Asian ancestry (24A) to assess XCI status of CDKL5 (16). Publicly available microarray data was also used to identify a single nucleotide polymorphism (SNP) in the CDKL5 gene of SH-SHY5Y (Krishna et al., BMC Genomics 15:1154 (2014)). Genomic DNA was isolated from SH-SY5Y using the Quick-gDNA MiniPrep kit (Zymo Research). Total RNA was extracted using the Direct-zol RNA Miniprep kit (Zymo Research) and 500 ng RNA was reverse transcribed using RevertAid First Strand cDNA Synthesis Kit (Thermo Fisher Scientific). The presence of the coding SNPs rs34567810 in CDKL5 and rs1808 in the escape gene CA5B was confirmed via Sanger sequencing (Genewiz, Inc) of both genomic DNA and RNA. Chromatograms were analysed using SnapGene software (GSL Biotech).

Lentivirus production and purification. To produce lentiviral particles as described before (Pollock et al., Mol. Ther. 24:965-977 (2016)), a total of 50 million Lenti-X 293T cells were seeded into two T-225 flasks per viral packaging the day before transfection in high glucose DMEM supplemented with 10% fetal bovine serum and 1% L-glutamine. For each flask 25 μg of dC, dCV, dCT or sgRNA expression vector, 5 μg of pMD2.G (envelope, Addgene plasmid #12259), and 25 μg of psPAX2 (gag/pol, Addgene plasmid #12260) were complexed with 140 ul using TransIT-293 (Mirus, Madison, Wis.) according to the manufacturer's recommendation in OPTI-mem. 48 hours after transfection, media was changed to 15 mL of UltraCULTURE medium (Lonza, Basel, Switzerland).

Vector supernatants were collected 72 hours post-transfection. Supernatant is initially centrifuged at 1500 rpm to clarify media and then concentrated by centrifugation at 3,000 rpm using Centricon-Plus-70-Centrifugal-Filter-Units (MilliPoreSigma, Burlington, Mass.). Viral aliquots were stored at −80° C. Virus for the expression of dCas9 effectors was titered by transduction of Lenti-X 293T cells and analysed by flow cytometry for expression of GFP and BFP. All flow cytometry analyses were performed on the BD Fortessa at the UC Davis Flow Cytometry Shared Resource Core. Viral titers for the expression of sgRNAs were determined by using the qPCR lentivirus titration kit (Applied Biological Materials Inc., Richmond, BC). SH-SY5Y (ATCC) cells were grown in DME/F12 media containing 20% FBS and 1% L-glut. SH-SY5Y cells were seeded on 6-well plates at a density of 300,000 cells per well and co-transduced with equimolar levels of dCas9 lentiviral particles equivalent to one Lenti-X 293T and a volume of dCas9 lentivirus equivalent to one Lenti-X 293T transducing unit and 5×107 IU of each sgRNA expression vector in combination with 2.5 μg/ml protamine sulfate (Fresenius Kabi, Lake Zurick, Ill.). For cells co-transduced with dCas9-VP64 and dCas9-TET1CD lentiviral volumes equivalent to 0.5 Lenti-X 293T transducing units each were used. Cells were sorted 5 days post-transduction at passage 11 for expression of GFP and/or BFP using the Influx cell sorter at the UC Davis Flow Cytometry Shared Resource Core (Sacramento, Calif.) and further expanded for 3-4 passages for subsequent analysis.

Targeted X-reactivation analysis. SH-SY5Y cells from each FACS-isolated treatment group and unsorted cells were seeded at a density of 300,000 cells per well in 6-well plates and allowed to grow until approximately 70% confluency. Cells were then rinsed in 1× DPBS and lysed in the well using TriZol (Ambion). Total RNA was extracted using the Direct-zol RNA Miniprep kit (Zymo Research) and 500 ng RNA was reverse transcribed using RevertAid First Strand cDNA Synthesis Kit (Thermo Fisher Scientific). For X-reactivation analysis, 100 ng of cDNA from stable SH-SY5Y lines was used for PCR amplification using Phusion High Fidelity Mastermix (New England Biolabs, Ipswich, Mass.). Each forward primer contained a unique 5-bp barcode sequence at the 5′ end for multiplexing (Supplementary Table 1). All amplicons were gel extracted and purified using the Zymo Gel DNA Recovery kit (Zymo Research) and pooled at equal concentrations for Illumina sequencing.

Amplicon sequencing was performed by the CCIB DNA Core Facility at Massachusetts General Hospital (Cambridge, Mass.). Forward and reverse reads of raw sequencing data were merged into a single long read using FLASH2 and barcodes were demultiplexed using FASTX at the beginning or end of the sequence read, allowing for a single mismatch each, yielding a mean read depth of >10,000 reads per sample. Processed FASTQ files were then analysed for frequency of reads containing the reactivated C allele for the coding SNP rs35478150 identified in exon 16 of the CDKL5 gene with the grep function over the total number of matched reads, yielding the reactivation frequency. Allele-specific RT-qPCR was performed as described above using a common forward primer and allele-specific reverse primers for the same coding SNP as analysed by amplicon sequencing (Table 1). Reactivation percentage was calculated as the percentage of relative Xi CDKL5 expression over relative Xa CDKL5 expression from mock-treated cells, normalized to GAPDH.

Targeted DNA demethylation analysis. Genomic DNA from transduced and mock-treated cells was isolated using the Quick-gDNA MiniPrep kit (Zymo Research). Bisulfite conversion was performed using the EZ DNA Methylation-Lightning Kit (Zymo Research) following the manufacturer's instructions. Primers for bisulfite-sequencing PCR were designed using MethPrimer with default settings (Li and Dahiya, Bioinformatics 18:1427-1431 (2002)) and unique 5-bp barcode sequences were added at the 5′ end for multiplexing (Table 1). 100 ng of bisulfite converted DNA was used for PCR amplification with ZymoTaq polymerase (Zymo Research) and the 238-bp amplicon was purified with the QIAquick PCR Purification Kit (Qiagen, Hilden, Germany) and submitted for amplicon sequencing. Amplicon sequencing was performed by the CCIB DNA Core Facility at Massachusetts General Hospital (Cambridge, Mass.) and further processed as described above. Alignment of processed FASTQ files and read mapping to a 238 bp reference amplicon was performed using Bismark with default settings (Krueger et al., Bioinformatics 27:1571-1572 (2011)). Further analysis and methylation calling of sorted BAM files was performed using CGMapTools (Guo et al., Bioinformatics 34:381-387 (2018)).

Chromatin immunoprecipitation (ChIP) and ChIP-qPCR. ChIP was performed as previously described (O'Geen et al., Epigenetics Chromatin 12:26 (2019)). Mock-treated and transduced cells were cross-linked 3-4 passages after FACS as described above in 1% formaldehyde for 10 min at room temperature and the reaction was stopped with 0.125 M glycine. Cross-linked cells were lysed with ChIP lysis buffer (5 mM PIPES pH8, 85 mM KCl, 1% Igepal) with a protease inhibitor (PI) cocktail (Roche). Nuclei were collected by centrifugation at 2000 rpm for 5 min at 4° C. and lysed in nuclei lysis buffer (50 mM Tris pH8, 10 mM EDTA, 1% SDS) supplemented with PI cocktail. Chromatin was fragmented using the Bioruptor Pico (Diagenode, Denville, N.J.) and diluted with 5 volumes RIPA buffer (50 mM Tris pH 7.6, 150 mM NaCl, 1 mM EDTA pH8, 1% Igepal, 0.25% deoxycholic acid).

ChIP enrichment was performed by incubation with 3 μg H3K27me3 antibody (ab6002, Abcam, Cambridge, UK) or 2 μg normal rabbit IgG (ab46540, Abcam) for 16 h at 4° C. Immune complexes were bound to 20 μl magnetic protein A/G beads (Biorad, Hercules, Calif.) for 2 h at 4° C. Beads were washed 2× with RIPA (Thermo Fisher Scientific) and 3× with ChIP wash buffer (100 mM Tris pH8, 500 mM LiCl, 1% deoxycholic acid). The final wash was performed in ChIP wash buffer with 150 mM NaCl. Cross-links were then reversed by heating beads in 100 μl ChIP elution buffer (50 mM NaHCO₃, 1% SDS) overnight at 65° C., and DNA was purified using the QIAquick PCR Purification Kit (Qiagen, Hilden, Germany). ChIP-qPCR was performed with PowerUp SYBR Green Master Mix (Thermo Fisher Scientific) using the StepOne Plus Real Time PCR system (Thermo Fisher Scientific) and the StepOne Plus software was used to extract raw CT values. ChIP enrichment was calculated relative to input samples using the delta CT method.

Whole-genome methylation analysis by Infinium MethylationEPIC array. Whole genome methylation analysis was performed following (O'Geen et al., supra). Briefly, 300,000 cells for each treatment group were seeded in 6-well plates and allowed to grow to approximately 70% confluency. Genomic DNA from transduced and mock-treated cells in biological duplicates was isolated using the Quick-gDNA MiniPrep kit (Zymo Research) and 500 ng submitted for bisulfite conversion and Illumina's Infinium MethylationEPIC BeadChip array by the Vincent J. Coates Genomics Sequencing Laboratory (Berkeley, Calif.). The minfi package (Aryee et al., Bioinformatics 30:1363-1369 (2014); Fortin et al., Bioinformatics 33:558-560 (2017)) was used to extract two channel raw data (RGChannelSet) from the IDAT files at the probe level for all 850,000 probes. The RGChannelSet was used for background subtraction using preprocessNoob (Triche et al., Nucleic Acids Res. 41:e90 (2013)) followed by preprocessFunnorm (Fortin et al., Genome Biol. 15:503 (2014)) to normalize the samples. Beta values for each site (beta=M/(M+U), where M and U denote the methylated and unmethylated signals) were extracted from the GenomicRatioSet, which is the data organized by the CpG locus level mapped to the genome. The ChAMP package (Tian et al., Bioinformatics 33:3982-3984 (2017)) was used to filter probes using default settings with filterXY set to false. The limma function within ChAMP was then used (Smyth et al., Stat. Appl. Genet. Mol. Biol. 3, Article3 (2004), Wettenhall et al., Bioinformatics, 20:3705-3706 (2004)) to detect differentially methylated positions at default settings and merged the output file with the individual FunNorm beta values. In order to determine differentially methylated promoter regions, CpG sites were selected for cgi.feat. TSS200-island and TSS1500-island and a mean difference in beta value of ±0.05. Differentially methylated genes were defined as genes with at least 3 differentially methylated positions in the promoter. Venn diagrams were generated using bioinformatics.psb.ugent.be/.

RNA-Seq Library Preparation and Analysis. Global changes to transcription were assessed using RNA-Seq. Briefly, 300,000 cells for each treatment group were seeded in 6-well plates and allowed to grow to approximately 70% confluency. Cells were then rinsed in 1×DPBS and lysed in the well using TriZol (Ambion). Total RNA was extracted using the Direct-zol RNA Miniprep kit (Zymo Research). RNA was quantified with Nanodrop and 1 ug of RNA was used for each library. RNA libraries were generated using the NEBNext Ultra II RNA Library Prep kit (NEB) following manufacturer's instructions. Libraries were multiplexed and pooled for a single lane of sequencing on a HiSeq4000. Sequencing reads were de-multiplexed and aligned to the Hg38 reference genome with STAR Universal Aligner version 2.5.3a using the following settings: Indexed Reference Genome: Ensembl reference genome and annotation files for Hg38 release 77 were downloaded and complied into a single file, Genome was indexed using the following arguments “STAR—runMode genomeGenerate--runThreadN 12-genomeDir/STAR_INDEX_HG38--genomeFastaFiles GRCh38_r77.all.fa--sjdbGTFfile Homo_sapiens.GRCh38.77.gtf-sjdbOverhang 149”; Sample Read Alignment: alignment of each sample's reads was performed with the following arguments: “STAR--runThreadN 24-genomeDir/STAR_INDEX_HG38--outFileNamePrefix/STAR/SampleName_--outSAMtype BAM SortedByCoordinate--outWigType bedGraph--quantMode TranscriptomeSAM GeneCounts--readFilesCommand zcat--readFilesIn Sample-R1.fastq.gz Sample-R2.fastq.gz”. Differential Expression (DE) analysis was performed with DESeq2 (Love et al., Genome Biol. 15:550 (2014)) software in R Studio. First, gene count files were combined into a single file. Then, normalization and DE analysis were performed using a dCas9 control. DE gene lists from pairwise comparisons were exported into .csv files and utilized for GO term analysis using DAVID (david.ncifcrf.gov). Volcano plots were generated using ggplot2 software in R studio.

Off-target analysis. Off-target analysis of CRISPR sgRNAs was performed using the CasOFFinder tool (www.rgenome.net/cas-offinder/) (Bae et al., Bioinformatics 30:1473-1475 (2014)). Briefly, 20 bp spacer sequences for the three top sgRNA candidates without PAM sequences were used as the query using hg38 as the reference genome for canonical SpCas9 PAM sites. The algorithm was executed using 3 or less mismatches and DNA and RNA bulge sizes of 1. In order to extend the list from off-target sites to potential off-target genes, genes in a ±5 kb window were included using the Table Browser function of the UCSC Genome Browser. The list of off-target genes was then overlapped with all differentially expressed genes from the three conditions as well as differentially methylated probes from the dCas9-TET1CD comparison with dCas9 catalytically inactive TET1.

Statistical analysis. Statistical analyses were performed in Prism 8 (GraphPad Software, San Diego, Calif.) and in R Studio 3.6.0. Statistics are presented as the mean f SD. Targeted assessments were performed in biological triplicates. Genome-wide assessment were performed in triplicates unless otherwise noted. Between-group differences were analysed using a One-way analysis of variance (ANOVA). When appropriate, a Tukey's post hoc test was performed. Statistical differences between the means of two groups were determined using an independent samples t Test. The p value cut-off for all targeted analyses was set at 0.05 for all analyses. Statistical analyses of differentially methylated sites were performed using the limma function embedded in ChAMP in R Studio 3.6.0. The null hypothesis was rejected for tests with FDR <5%. Statistical analyses of differentially expressed genes was performed using DESeq2 in R Studio 3.6.0. The null hypothesis was rejected for tests with FDR <1%.

### Example 2: Programmable Transcription of the CDKL5 Gene

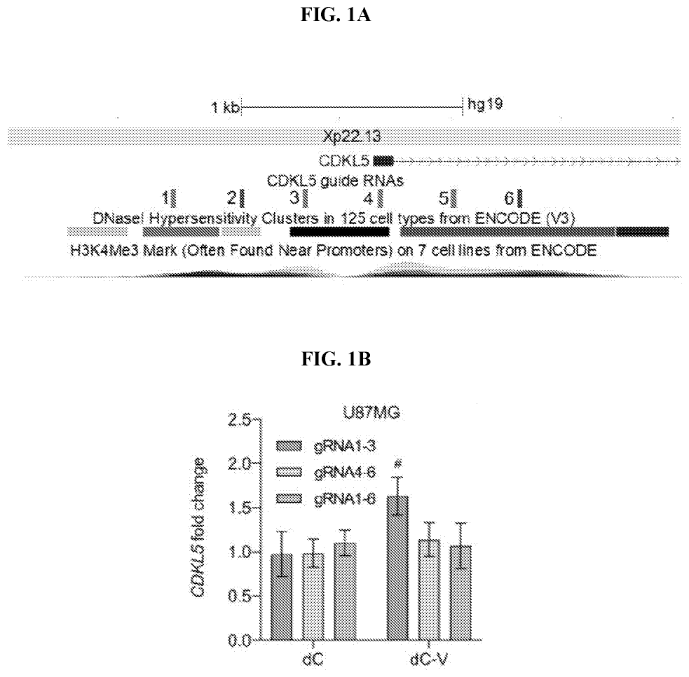

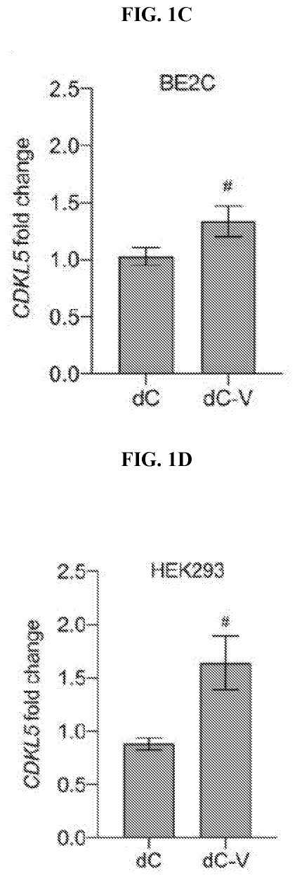

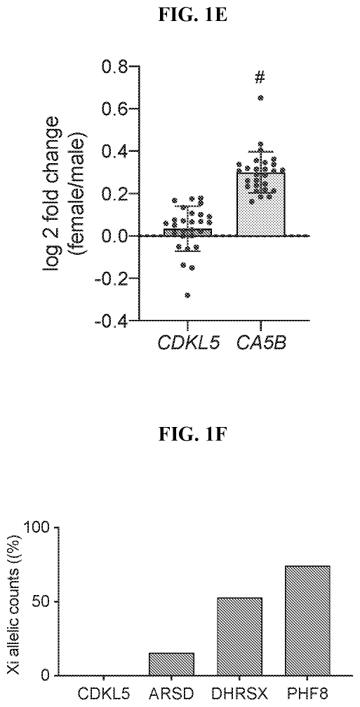

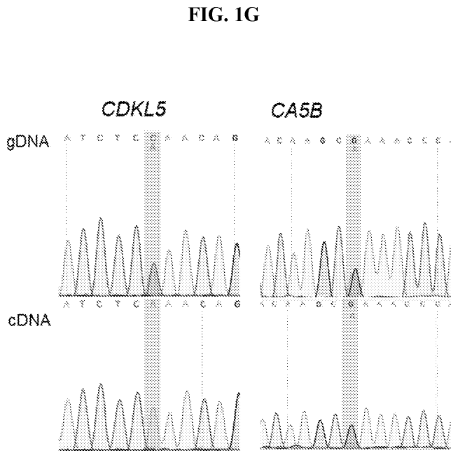

To investigate whether the CDKL5 gene is amenable to transcriptional reprogramming via dCas9 effector domains, U87MG cells were transiently co-transfected with dCas9 constructs and gRNA expression vectors. In particular, a dCas9-VP64 expression plasmid (dC-V) was used for the co-transfection. Plasmid expressing dCas9 without effector domain was used as a control (dC). 6 individual guide RNAs were design to span DNase I hypersensitive sites and H3K4me3 peaks of the CDKL5 promoter within a ±1 kb window on either side of the CDKL5 transcriptional start site (FIG. 1A). Because several guide RNAs are required for gene activation with dC-V, several combinations of 3-6 guide RNAs were tested. RT-qPCR was performed and significant activation of CDKL5 expression with the combination of guide RNAs 1-3 paired with dC-V targeting a region upstream of the transcriptional start site was observed (FIG. 1B). In particular, CDKL5 expression increased 1.6-fold in U87MG cells when compared to dC (p=0.023). However, no significant difference between dC and dC-V was observed with cells transfected with guide RNAs 1-6 or guide RNAs 4-6 (FIG. 1B). In concordance with U87MG cells, transfection with guide RNAs 1-3 and dC-V showed a 1.3-fold upregulation of CDKL5 in BE2C cells (p=0.0112, FIG. 1C) and a 1.6-fold upregulation of CDKL5 in HEK293 (p=0.0424, FIG. 1D), when compared to cells transfected with dC. Therefore, these results demonstrated the identification of a cis-regulatory element in the CDKL5 promoter that allows for programmable transcription.

### Example 3: dCas9-TET1CD Significantly Reactivated Silenced CDKL5 Expression

Due to the lack of informative allele-specific polymorphisms in either U87MG, BE2C, and HEK cell lines, bi-allelic mRNA activation in female SH-SY5Y cells was examined in order to assess whether the increase in gene expression was due to superactivation of the active CDKL5 allele, reactivation of the silenced allele, or a combination of both. Comparative analysis across several GTEx tissues demonstrated that CDKL5 did not display female-biased expression, which served as a proxy for X-chromosome Inactivation (XCI) status when compared to the known escape gene CA5B (FIG. 1E). Analysis of XCI using pre-existing scRNA-seq data to assess allele-specific expression from lymphoblastoid cells further revealed that CDKL5 is monoallelically expressed only from the active X chromosome (FIG. 1F). In order to distinguish between the active and the inactive CDKL5 allele, the presence of a SNP (rs35478150) in the coding region of the CDKL5 gene in SH-SY5Y cells was determined. Sanger sequencing confirmed monoallelic expression of the active CDKL5 A allele and silencing of the C allele. Expression of a polymorphic site in CA5B was also examined, and the results showed bi-allelic expression from the active and escape allele (FIG. 1H).

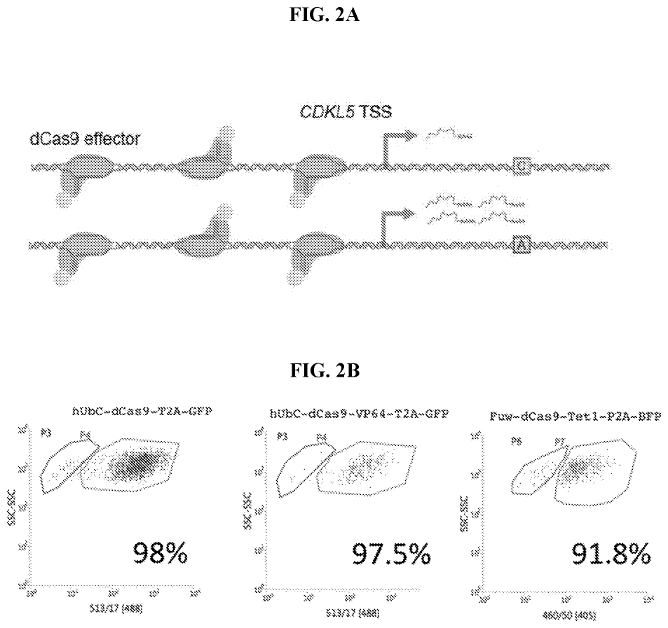

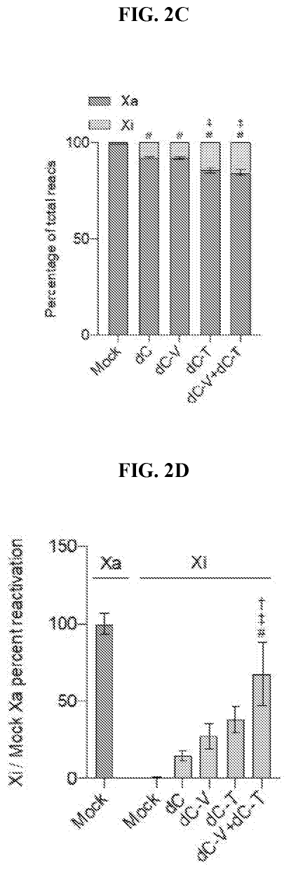

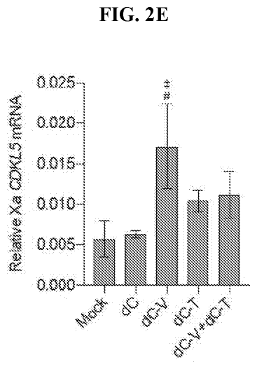

Due to the importance of methylated CGI promoters in XCI, the role of a dCas9-TET1CD fusion protein for DNA methylation editing (dC-T) was investigated. In order to determine X chromosome reactivation efficiency, allele-specific activation facilitated by dC-V or dC-T was evaluated (FIG. 2A). SH-SY5Y cells were transduced with lentiviral particles encoding the dC fusion proteins and the three guide RNAs. dCas9 expression plasmids also encoded in-frame fluorescent markers GFP (dC and dC-V) or BFP (dC-T). Three days following transduction, transduced cells were selected by FACS based on the respective fluorescent marker (FIG. 2B).

To determine reactivation of the silenced CDKL5 allele with high sensitivity, amplicon-based targeted RNA-sequencing was performed. Targeting of dC to CDKL5 was sufficient to significantly reactivate expression of the silenced allele by greater than 11-fold to 8% of total allelic reads compared to mock-treated cells (p<0.0001; FIG. 2C). Transcriptional reprogramming using dC-V targeted to the CDKL5 promoter did not show a significant increase when compared to dC. Strikingly, cells transduced with dC-T showed a 20.7-fold increase when compared to mock (p<0.0001) and a significant 1.8-fold increase above dC (p<0.0001), leading to reactivation levels of up to 14.5% of total expression (FIG. 2C). Since dC-V increases total CDKL5 mRNA in other cell lines tested, a determination of whether multiplexing dCas9-VP64 and dCas9-TET1CD (dC-T+dC-V) to the same locus further potentiates CDKL5 reactivation was made. However, no significant difference was observed between dC-T and co-transduction of dC-T+dC-V in increasing the proportion of allelic reads derived from the inactive allele.

Due to the fact that the observed allelic reads via amplicon sequencing are a ratio of active versus silenced CDKL5 expression, allele-specific RT-qPCR was performed in order to compare reactivation levels from the inactive allele to the active allele baseline expression in SH-SY5Y (FIG. 2D). Similar to amplicon sequencing data, no expression of the inactive allele was observed above background (<1% Xi/Xa mock). Expression levels in cells transduced with dC of 14.8% Xi/Xa mock in cells treated with dC was observed (FIG. 2D). No statistically significant increase in Xi/Xa mock expression over dC was observed in cells treated with dC-V (27.3%) or dC-T (38.2%) (FIG. 2D). However, SH-SY5Y cells that were co-transduced with dC-V+dC-T showed a statistically significant increase of reactivation from the inactive allele (67.4%) when compared to dC (p=0.0004), dC-V (p=0.042) and dC-T (p=0.038) (FIG. 2D). These findings showed that SH-SY5Y that have been treated with the dCas9 effector domains reached close to equal bi-allelic expression due to a synergistic effect of dC-V and dC-T on the previously silent, reactivated allele.

For the expression of the active CDKL5 allele, no significant difference between dC and mock-treated cells was observed (FIG. 2E). Moreover, dC-V significantly upregulated mRNA expression from the active allele by 3.0-fold when compared to mock (p=0.0052) or 2.7-fold when compared to dC (p=0.0073; FIG. 2E). No significant difference was observed between mock cells versus dC-T or dC-V+dC-T and dC versus dC-T or dC-V+dC-T. Noticeably, targeting dC-T to the CDKL5 promoter did not significantly modulate active CDKL5 mRNA levels.

### Example 4: dCT Significantly Reduced DNA Methylation

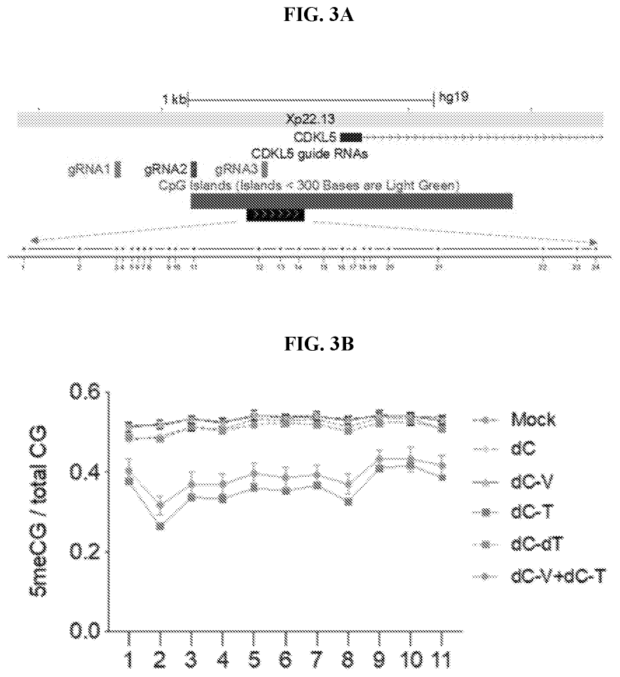

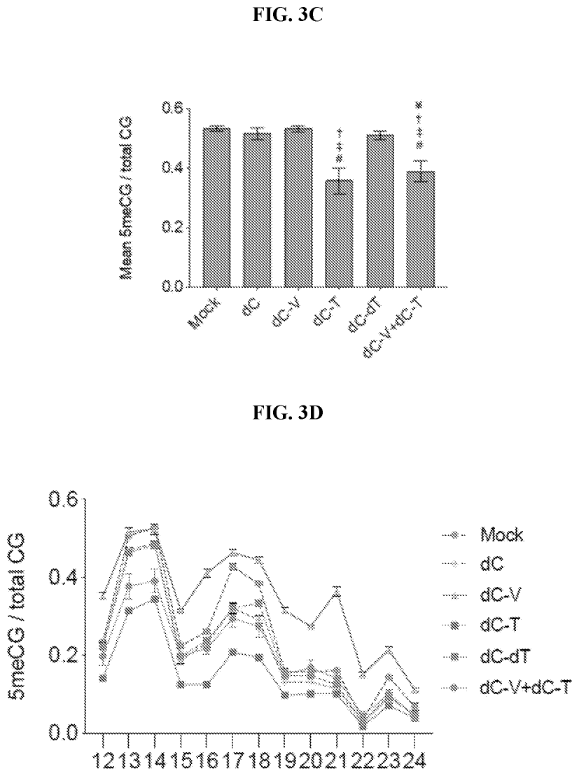

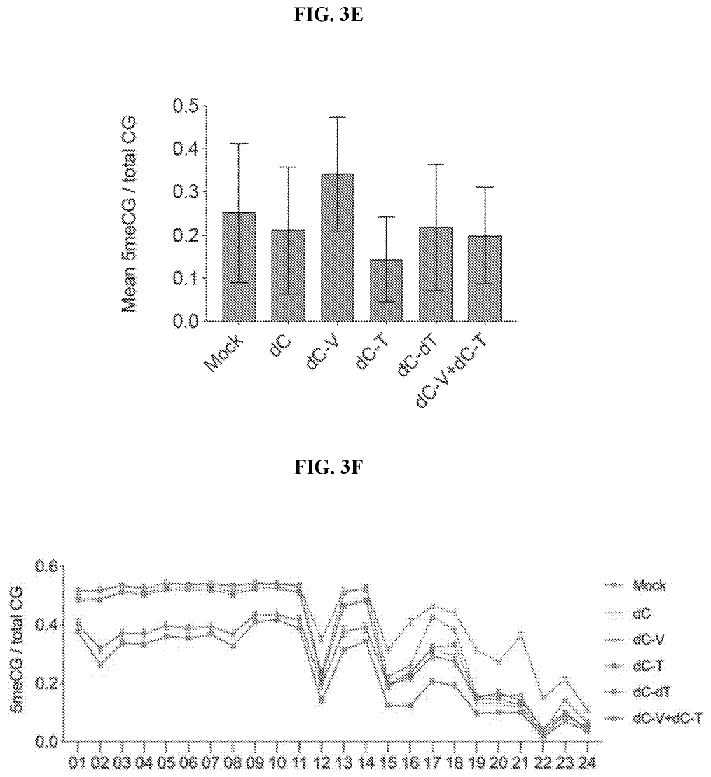

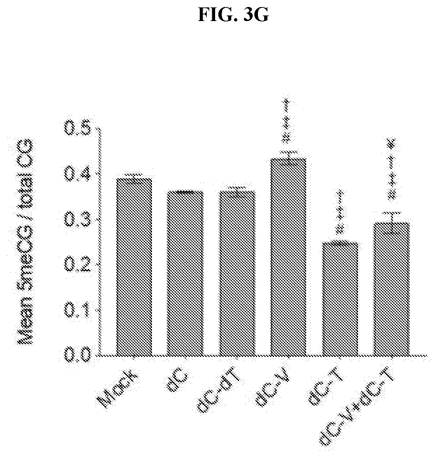

The status of XCI highly correlates to promoter CGI methylation. Due to the differences in targeted reactivation between effector domains, targeted bisulfite amplicon sequencing was performed in the CDKL5 core promoter region in order to identify the role of differential DNA methylation in X-reactivation between groups (FIG. 3A). PCR-based amplicons that allowed the measurement of the ratio of 5-meCG/totalCG at 24 CpG individual dinucleotides in the CDKL5 core promoter by deep sequencing was generated (FIGS. 3B, D-E). Due to the lack of a polymorphism in the promoter region, biallelic CpG methylation was assessed, assuming that DNA methylation was primarily present on the XCI silenced allele. Two segments of DNA methylation that were demarcated by a dip in methylation at CpG dinucleotide position 12 were observed. The first segment showed that the CDKL5 promoter was partially methylated in mock-treated SH-SY5Y (53% 5-meCG/CG±0.9%, FIG. 3B). The second segment showed a decreased baseline DNA methylation level and more variability of 5-meCG/CG (25.4% 5-meCG/CG 16.8%, FIGS. 3D-E), suggesting that partial methylation of the core region containing the first 11 CpGs was critical for regulation of CDKL5 transcription. Amplicon sequencing of bisulfite converted genomic DNA revealed the mean 5-meCG/totalCG ratio across the first 11 CpG sites was 53.3% in mock and 51.6% in dC transduced cells (FIG. 3C). A 17.5% decrease of 5-meCG/CG in cells transduced with dC-T compared to mock-treated cells (p<0.0001) and a 15.9% decrease to dC (p<0.0001) was observed. This effect was due to the catalytic activity of dC-T, since a catalytically inactive TET1 mutant (dC-dT) fails to disrupt methylation at 51% 5-meCG/CG (p<0.0001). The combinatorial treatment of dC-T+dC-V also showed a significant reduction in methylation levels of 14.3% compared to mock-treated cells (p<0.0001) and 12.6% compared to dC (p<0.0001). However, the combination of dC-T and dC-V had significantly higher levels of methylation when compared to dC-T alone. The combination of dC-T and dC-V had the greatest increase of the inactive allele. This might be due to TET1 achieving a level of demethylation that allowed for gene transcription. In fact, the addition of dC-V actually significantly decreased the amount of demethylation, indicating, that dC-V did not contribute to DNA demethylation as a mechanism of transcriptional activation.

### Example 5: Targeted Loss of Repressive H3K27Me3 in the CDKL5 Promoter

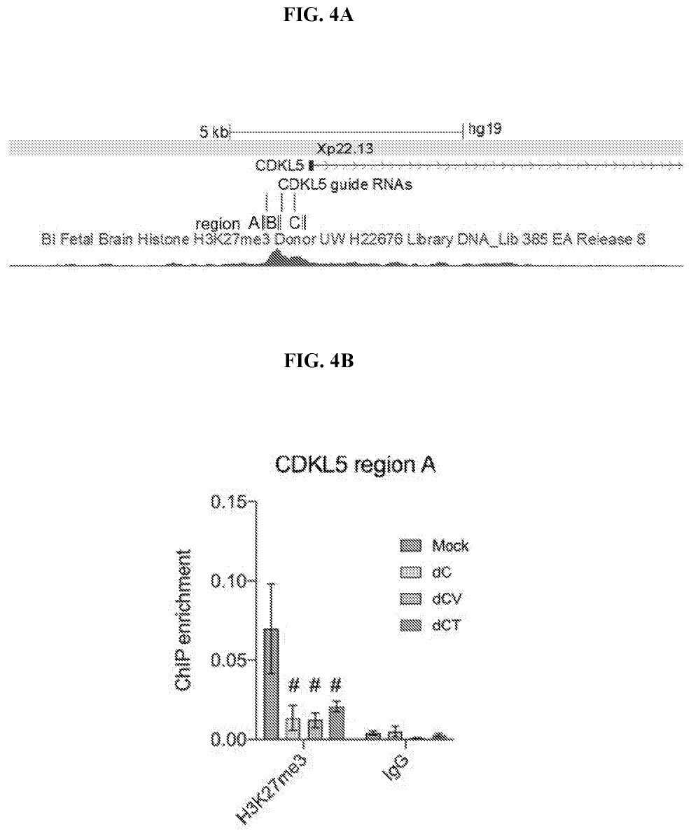

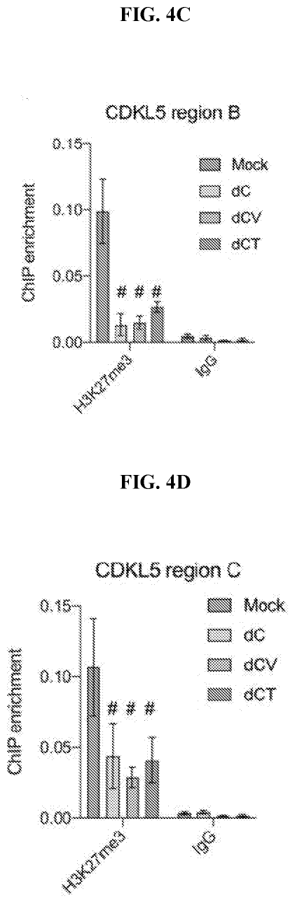

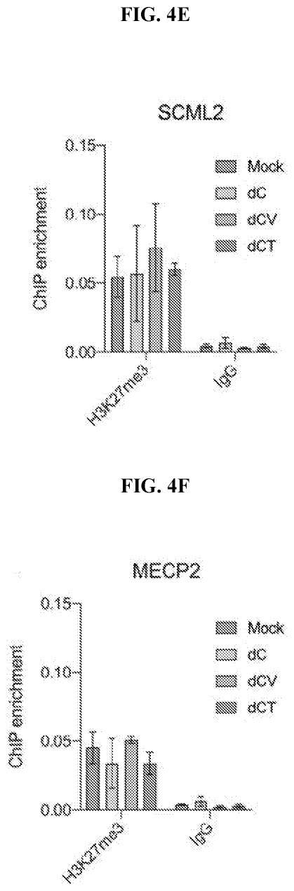

Genes that escape from XCI show a specific epigenetic signature, such as the depletion of the repressive histone mark H3K27me3. Therefore, an investigation as to whether targeted reactivation of the observed allele coincided with a remodelling of heterochromatin was conducted. ChIP-qPCR was used to test three different regions within a 1-kb fragment upstream of the transcriptional start site for changes in the H3K27me3 mark that have strong signal enrichment in brain tissue as determined by ENCODE and overlap the guide RNA target sites (FIG. 4A). When compared to mock-treated cells, treatment with dC by itself depleted H3K27me3 signal 3.5-fold in region A (p=0.0073, FIG. 4B); 2.9-fold in region B (p=0.0002, FIG. 4C) and 1.5-fold in region C (p=0.00453, FIG. 4D). There was no significant difference between treatment with dC, dC-V or dCT. To understand the effect of histone-based feedback and spreading of the depletion of the histone mark across neighboring nucleosomes in our treated cells, the signal of H3K27me3 in distal neighboring regulatory regions was investigated. ChIP-qPCR was performed on the nearest neighboring gene to the CDKL5 promoter (−70 kb) and tested for H3K27me3 signal (FIG. 4E). There were no significant differences between groups for H3K27me3 signal in the promoter region of the SCML2 gene. Therefore, the results showed that the loss of H3K27me3 remained confined to the target site and was associated with gene reactivation. In addition, no significant difference between groups was observed for H3K27me3 signal at an unrelated negative control region in the MECP2 promoter on the long arm of the X chromosome (FIG. 4F).

### Example 5: dCT Transduced Cells Showed Global Promoter Hypomethylation

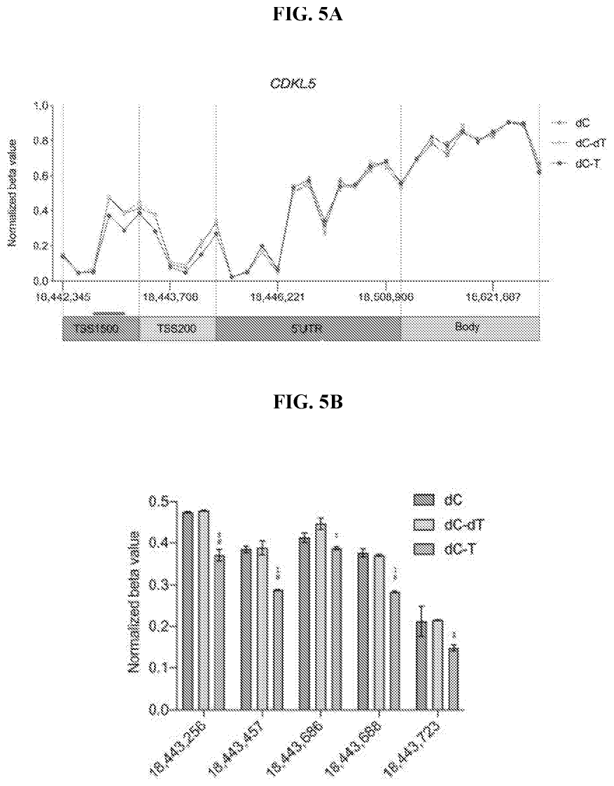

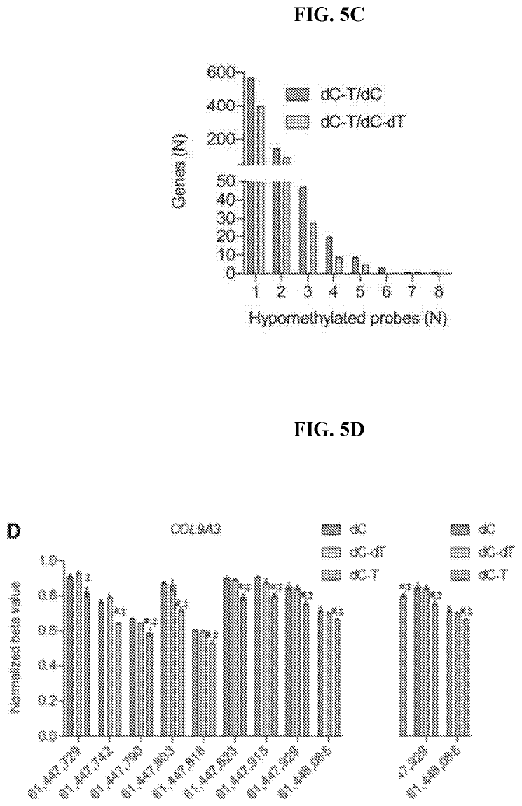

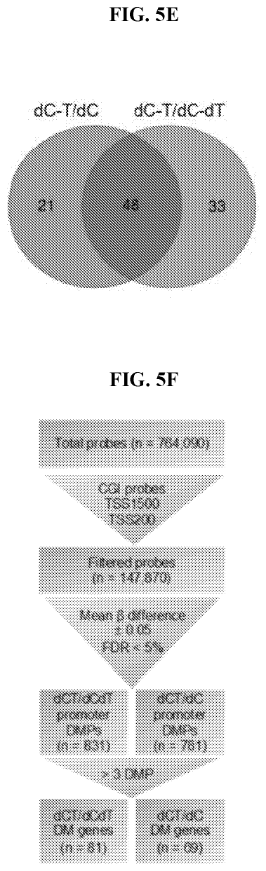

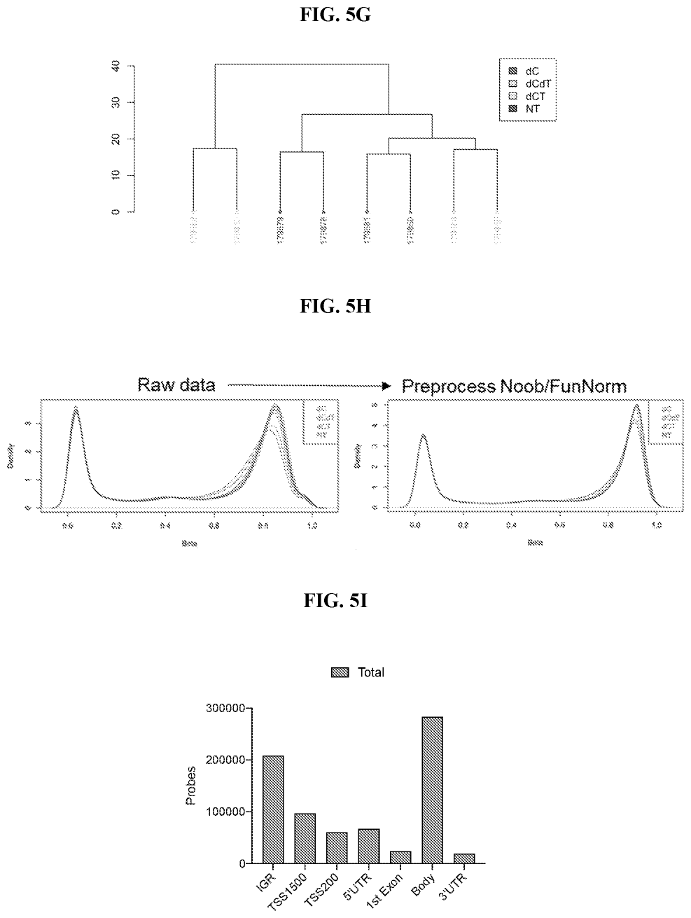

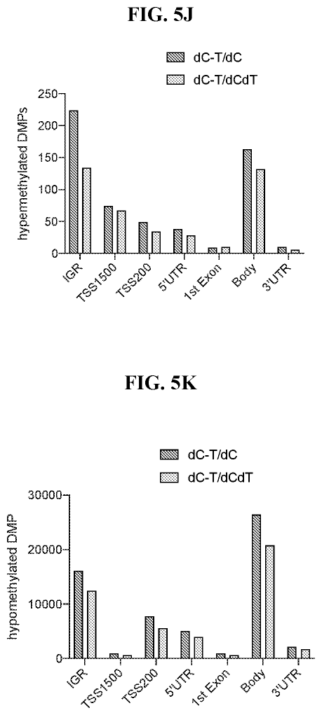

To determine on- and off-target effects of dC-T on the DNA methylome in stably transduced SH-SY5Y cells, the Illumina Infinium HumanMethylationEPIC (EPIC) array was used to interrogate 764,090 CpG sites genome-wide (Tables 2-3, FIGS. 5G-K). A smaller subset of 147,870 probes (19.4%) within CpG islands was identified. Out of this subset, 59,264 probes were further enriched that were found within 1500 and 200 bp of the TSS. For a pairwise comparisons to determine differentially methylated (DM) positions between dC-T and dC-dT or dC, a cut-off of mean difference in beta value greater than 0.05 (FDR <5%) was set. To validate these criteria, all 32 methyl-probes mapping to the CDKL5 gene without filtering for probe features was analyzed (FIGS. 5A-B). In concordance with the targeted bisulfite approach, a partial methylation of the promoter region of control treated cells (dC, dC-dT), which was modestly but significantly reduced near the sgRNA target site (FIG. 5A, line above TSS1500) in cells transduced with dC-T was identified. DM positions in any of the other genic features was not identified. These results further demonstrated the highly distinctive role of DNA methylation signatures in CGI promoters. No DM positions were identified in the nearest neighboring gene to the CDKL5 promoter (SCML2) once again confirming that the dC-T-induced DNA demethylation was targeted to the CDKL5 promoter. In total, 795 or 747 differentially hypomethylated promoters (Table 2) and 34 or 26 differentially hypermethylated (Table 3) promoters for the comparison between dC-T and dC or dC-dT, respectively were identified. Due to the small number of differentially hypermethylated sites and the fact that gene repression due to off-target hypermethylation of TET1CD was unlikely, hypermethylated sites were omitted from further analysis. The majority of differentially hypomethylated sites in gene promoters due to the introduction dC-T showed only a single DM position (568 genes when compared to dC, 402 genes when compared to dC-dT), likely not eliciting an effect on transcription (FIG. 5C). Since CDKL5 had at least 3 differentially hypomethylated sites, genes with at least 3 DM sites were considered as a differentially hypomethylated gene promoter. The gene with the highest number of DM positions was COL9A3, showing eight hypomethylated sites within the promoter (FIG. 5D). A total of 69 or 81 genes were identified when compared to dC or dC-dT respectively. Forty-eight genes were conserved between the pairwise comparisons (FIG. 5E-F).

TABLE 2
dCT_to_dCdT_hypomethylated promoter

| | Genes | N |
|---|---|---|
| 1 | 43718 | 2 |
| 2 | 43714 | 1 |
| 3 | 43716 | 1 |
| 4 | AARS2 | 1 |
| 5 | ABCA5 | 1 |
| 6 | ABCC8 | 3 |
| 7 | ABCG1 | 2 |
| 8 | ABCG2 | 1 |
| 9 | ABO | 1 |
| 10 | ACAA1 | 1 |
| 11 | ACCN4 | 2 |
| 12 | ACHE | 1 |
| 13 | ACOT4 | 1 |
| 14 | ACP1 | 1 |
| 15 | ACP5 | 2 |
| 16 | ACSF3 | 1 |
| 17 | ACSL1 | 1 |
| 18 | ACSS1 | 1 |
| 19 | ACTA1 | 1 |
| 20 | ADAM11 | 1 |
| 21 | ADAM12 | 3 |
| 22 | ADAMTS16 | 1 |
| 23 | ADD3 | 1 |
| 24 | ADM | 1 |
| 25 | ADNP | 1 |
| 26 | ADRA2C | 1 |
| 27 | AEBP2 | 1 |
| 28 | AHNAK | 2 |
| 29 | AJAP1 | 1 |
| 30 | AK5 | 1 |
| 31 | AKAP12 | 3 |
| 32 | ALDH1A2 | 1 |
| 33 | ALDH1A3 | 2 |
| 34 | ALS2CL | 3 |
| 35 | ALX1 | 4 |
| 36 | ALX4 | 2 |
| 37 | ANK1 | 1 |
| 38 | ANKDD1A | 1 |
| 39 | ANKRD30B | 2 |
| 40 | ANKS6 | 1 |
| 41 | ANXA2 | 1 |
| 42 | APOO | 1 |
| 43 | AQP5 | 1 |
| 44 | ARAP1 | 1 |
| 45 | ARFGAP3 | 1 |
| 46 | ARFRP1 | 2 |
| 47 | ARHGAP27 | 1 |
| 48 | ARHGAP6 | 1 |
| 49 | ARHGDIA | 1 |
| 50 | ARHGEF11 | 1 |
| 51 | ARRDC2 | 1 |
| 52 | ARX | 1 |
| 53 | ASAM | 4 |
| 54 | ASCL2 | 4 |
| 55 | ASS1 | 1 |
| 56 | ATOH8 | 6 |
| 57 | ATP12A | 1 |
| 58 | ATP1A1 | 1 |
| 59 | ATP1B1 | 2 |
| 60 | ATP8B1 | 1 |
| 61 | ATPAF1 | 1 |
| 62 | B3GNT2 | 1 |
| 63 | B3GNT7 | 1 |
| 64 | B4GALT1 | 1 |
| 65 | BAIAP2-AS1 | 1 |
| 66 | BCAR1 | 1 |
| 67 | BCL2 | 1 |
| 68 | BEND4 | 2 |
| 69 | BHLHA9 | 1 |
| 70 | BHLHE23 | 2 |
| 71 | BLVRA | 2 |
| 72 | BNIP3 | 1 |
| 73 | BOLL | 1 |
| 74 | BRF1 | 1 |
| 75 | BSPRY | 1 |
| 76 | BSX | 1 |
| 77 | BTG4 | 1 |
| 78 | C10orf125 | 2 |
| 79 | C10orf2 | 1 |
| 80 | C10orf93 | 1 |
| 81 | C11orf20 | 1 |
| 82 | C11orf9 | 1 |
| 83 | C11orf93 | 3 |
| 84 | C12orf62 | 1 |
| 85 | C14orf106 | 1 |
| 86 | C14orf162 | 1 |
| 87 | C14orf50 | 2 |
| 88 | C15orf29 | 2 |
| 89 | C15orf60 | 1 |
| 90 | C16orf53 | 1 |
| 91 | C16orf70 | 1 |
| 92 | C16orf74 | 1 |
| 93 | C17orf55 | 1 |
| 94 | C17orf64 | 1 |
| 95 | C18orf32 | 1 |
| 96 | C19orf34 | 1 |
| 97 | C19orf76 | 1 |
| 98 | C19orf77 | 1 |
| 99 | C1GALT1C1 | 1 |
| 100 | C1orf113 | 1 |
| 101 | C1orf133 | 1 |
| 102 | C1orf167 | 1 |
| 103 | C1orf70 | 2 |
| 104 | C1QL1 | 3 |
| 105 | C20orf166 | 2 |
| 106 | C2orf55 | 1 |
| 107 | C2orf70 | 1 |
| 108 | C4orf44 | 1 |
| 109 | C5orf32 | 1 |
| 110 | C6orf1 | 1 |
| 111 | C6orf150 | 1 |
| 112 | C7orf20 | 2 |
| 113 | C7orf26 | 1 |
| 114 | CABP1 | 1 |
| 115 | CACNA1B | 1 |
| 116 | CADM1 | 1 |
| 117 | CAST | 3 |
| 118 | CAV1 | 1 |
| 119 | CCDC134 | 1 |
| 120 | CCDC42 | 1 |
| 121 | CCDC78 | 1 |
| 122 | CD151 | 4 |
| 123 | CD274 | 1 |
| 124 | CDC42EP1 | 3 |
| 125 | CDC42EP4 | 1 |
| 126 | CDH1 | 1 |
| 127 | CDK6 | 1 |
| 128 | CDKL5 | 6 |
| 129 | CDS1 | 1 |
| 130 | CELSR2 | 1 |
| 131 | CENPV | 1 |
| 132 | CETN1 | 1 |
| 133 | CHCHD10 | 1 |
| 134 | CHD5 | 1 |
| 135 | CHRNA4 | 2 |
| 136 | CHRNB1 | 1 |
| 137 | CHST13 | 1 |
| 138 | CHST2 | 1 |
| 139 | CHST8 | 3 |
| 140 | CHST9 | 2 |
| 141 | CILP2 | 1 |
| 142 | CLCN5 | 2 |
| 143 | CLIP4 | 4 |
| 144 | CLSTN1 | 1 |
| 145 | CNTFR | 4 |
| 146 | COBL | 1 |
| 147 | COCH | 1 |
| 148 | COL11A2 | 4 |
| 149 | COL16A1 | 1 |
| 150 | COL9A2 | 1 |
| 151 | COL9A3 | 10 |
| 152 | COX17 | 2 |
| 153 | CPD | 2 |
| 154 | CPNE5 | 1 |
| 155 | CPT1B | 1 |
| 156 | CRABP2 | 3 |
| 157 | CRBN | 1 |
| 158 | CRHR1 | 1 |
| 159 | CRHR2 | 1 |
| 160 | CRYBA2 | 2 |
| 161 | CSNK1G2 | 1 |
| 162 | CSPG5 | 1 |
| 163 | CSRNP1 | 1 |
| 164 | CSRP1 | 1 |
| 165 | CTBP2 | 1 |
| 166 | CTDSP1 | 2 |
| 167 | CTPS | 1 |
| 168 | CTPS1 | 1 |
| 169 | CTPS2 | 1 |
| 170 | CXCL12 | 1 |
| 171 | CXCL2 | 1 |
| 172 | CXorf39 | 1 |
| 173 | CXorf41 | 1 |
| 174 | CYGB | 2 |
| 175 | CYHR1 | 1 |
| 176 | CYP1A1 | 3 |
| 177 | D2HGDH | 1 |
| 178 | DACT2 | 1 |
| 179 | DARS | 1 |
| 180 | DDX47 | 1 |
| 181 | DECR2 | 3 |
| 182 | DEF8 | 1 |
| 183 | DENND1B | 2 |
| 184 | DENND2D | 1 |
| 185 | DGAT1 | 1 |
| 186 | DGKZ | 2 |
| 187 | DIO3 | 3 |
| 188 | DIRC3 | 5 |
| 189 | DISP2 | 1 |
| 190 | DKFZp686O24166 | 2 |
| 191 | DLK1 | 2 |
| 192 | DMRTC2 | 1 |
| 193 | DNAJB6 | 1 |
| 194 | DNASE1L2 | 1 |
| 195 | DND1 | 1 |
| 196 | DNLZ | 1 |
| 197 | DOC2B | 1 |
| 198 | DOK7 | 2 |
| 199 | DRD1 | 1 |
| 200 | DSCAM | 1 |
| 201 | DSCAML1 | 1 |
| 202 | DSE | 1 |
| 203 | DTNB | 1 |
| 204 | DUS1L | 1 |
| 205 | DYDC2 | 1 |
| 206 | DYRK3 | 1 |
| 207 | E2F8 | 1 |
| 208 | EBF2 | 1 |
| 209 | ECEL1 | 3 |
| 210 | EDN3 | 1 |
| 211 | EEF1A1 | 2 |
| 212 | EEF1D | 1 |
| 213 | EFCAB4B | 4 |
| 214 | EFNA1 | 1 |
| 215 | EFNA4 | 3 |
| 216 | EGFLAM | 1 |
| 217 | EHBP1L1 | 2 |
| 218 | EHD2 | 1 |
| 219 | EIF1B | 1 |
| 220 | EIF4EBP3 | 1 |
| 221 | ELF4 | 1 |
| 222 | ELK3 | 1 |
| 223 | ELOVL2 | 1 |
| 224 | EMID2 | 2 |
| 225 | EMILIN3 | 1 |
| 226 | EN1 | 2 |
| 227 | ENDOD1 | 1 |
| 228 | ENTPD2 | 1 |
| 229 | EPB49 | 1 |
| 230 | EPHA2 | 1 |
| 231 | EPHA8 | 1 |
| 232 | ERICH1 | 1 |
| 233 | ETV7 | 2 |
| 234 | F2R | 1 |
| 235 | FABP5 | 3 |
| 236 | FADS6 | 1 |
| 237 | FAM100A | 1 |
| 238 | FAM125B | 1 |
| 239 | FAM134B | 1 |
| 240 | FAM156A | 1 |
| 241 | FAM165B | 1 |
| 242 | FAM19A3 | 1 |
| 243 | FAM19A4 | 1 |
| 244 | FAM59A | 3 |
| 245 | FAM69C | 1 |
| 246 | FAM78A | 2 |
| 247 | FARP1 | 3 |
| 248 | FASTK | 6 |
| 249 | FAT4 | 1 |
| 250 | FBLN1 | 1 |
| 251 | FBN1 | 1 |
| 252 | FBXO44 | 1 |
| 253 | FCHO2 | 2 |
| 254 | FGF11 | 1 |
| 255 | FGF8 | 1 |
| 256 | FGFR3 | 1 |
| 257 | FGFRL1 | 1 |
| 258 | FHL2 | 2 |
| 259 | FIGLA | 1 |
| 260 | FKBP4 | 3 |
| 261 | FLI1 | 1 |
| 262 | FLJ10357 | 1 |
| 263 | FLJ39609 | 1 |
| 264 | FLOT1 | 2 |
| 265 | FMNL1 | 1 |
| 266 | FOSL1 | 1 |
| 267 | FOXD2 | 1 |
| 268 | FOXD3 | 1 |
| 269 | FREM3 | 1 |
| 270 | FSCN2 | 4 |
| 271 | FSTL1 | 1 |
| 272 | FZD6 | 1 |
| 273 | GAD1 | 1 |
| 274 | GALNTL1 | 1 |
| 275 | GAPDHS | 1 |
| 276 | GATAD2A | 1 |
| 277 | GDF1 | 2 |
| 278 | GDF6 | 1 |
| 279 | GDF7 | 1 |
| 280 | GDPD5 | 1 |
| 281 | GJB2 | 1 |
| 282 | GJB6 | 1 |
| 283 | GLT1D1 | 1 |
| 284 | GLUL | 1 |
| 285 | GNG4 | 2 |
| 286 | GNG8 | 1 |
| 287 | GOLGA8B | 1 |
| 288 | GPC3 | 1 |
| 289 | GPR101 | 1 |
| 290 | GPR112 | 1 |
| 291 | GPR120 | 1 |
| 292 | GPR126 | 1 |
| 293 | GPR26 | 1 |
| 294 | GPRASP2 | 1 |
| 295 | GPRC5B | 1 |
| 296 | GPRC5C | 2 |
| 297 | GPS1 | 1 |
| 298 | GRM2 | 1 |
| 299 | GRM4 | 1 |
| 300 | GSC | 1 |
| 301 | GSDMD | 1 |
| 302 | GSN | 1 |
| 303 | GSTO2 | 1 |
| 304 | HAGHL | 2 |
| 305 | HCN4 | 1 |
| 306 | HDAC11 | 4 |
| 307 | HHEX | 1 |
| 308 | HHIPL1 | 4 |
| 309 | HIATL2 | 1 |
| 310 | HIC1 | 2 |
| 311 | HLCS | 4 |
| 312 | HMGA1 | 1 |
| 313 | HMX3 | 1 |
| 314 | HNRNPH1 | 1 |
| 315 | HOXA5 | 2 |
| 316 | HOXD11 | 1 |
| 317 | HOXD12 | 2 |
| 318 | HOXD4 | 1 |
| 319 | HOXD8 | 3 |
| 320 | HOXD9 | 2 |
| 321 | HR | 1 |
| 322 | HRAS | 1 |
| 323 | HRH3 | 1 |
| 324 | HS6ST1 | 3 |
| 325 | HSBP1L1 | 1 |
| 326 | HTR1A | 1 |
| 327 | HTRA4 | 1 |
| 328 | HVCN1 | 1 |
| 329 | ICOSLG | 1 |
| 330 | IFT57 | 1 |
| 331 | IGF2BP1 | 1 |
| 332 | IGFBP4 | 3 |
| 333 | IGSF21 | 1 |
| 334 | IKZF1 | 2 |
| 335 | IL27RA | 2 |
| 336 | IL28RA | 1 |
| 337 | INPP5F | 1 |
| 338 | IRF8 | 1 |
| 339 | IRS1 | 1 |
| 340 | IRX4 | 1 |
| 341 | IRX6 | 1 |
| 342 | ISG15 | 1 |
| 343 | ITM2B | 1 |
| 344 | JAZF1 | 1 |
| 345 | KCNA4 | 1 |
| 346 | KCNJ12 | 2 |
| 347 | KCNK12 | 2 |
| 348 | KCNK13 | 1 |
| 349 | KCNK15 | 1 |
| 350 | KCTD12 | 1 |
| 351 | KDM2A | 2 |
| 352 | KHDRBS3 | 1 |
| 353 | KHNYN | 3 |
| 354 | KIAA0146 | 1 |
| 355 | KIAA0562 | 1 |
| 356 | KIAA0895L | 1 |
| 357 | KIAA1161 | 2 |
| 358 | KIAA1324 | 1 |
| 359 | KIAA1609 | 1 |
| 360 | KIF17 | 4 |
| 361 | KIF2A | 1 |
| 362 | KIT | 1 |
| 363 | KITLG | 2 |
| 364 | KL | 1 |
| 365 | KLC1 | 3 |
| 366 | KLF13 | 1 |
| 367 | KLHDC7B | 1 |
| 368 | KLHL21 | 3 |
| 369 | KREMEN2 | 1 |
| 370 | KRTCAP3 | 1 |
| 371 | L3MBTL1 | 2 |
| 372 | LASS4 | 1 |
| 373 | LAYN | 1 |
| 374 | LDLRAP1 | 1 |
| 375 | LEF1 | 1 |
| 376 | LGALS3 | 1 |
| 377 | LINC01018 | 1 |
| 378 | LINC01231 | 1 |
| 379 | LIPG | 1 |
| 380 | LKAAEAR1 | 1 |
| 381 | LOC100129697 | 1 |
| 382 | LOC100130872 | 1 |
| 383 | LOC101928134 | 1 |
| 384 | LOC101929073 | 1 |
| 385 | LOC101929353 | 1 |
| 386 | LOC144571 | 2 |
| 387 | LOC200726 | 1 |
| 388 | LOC284798 | 1 |
| 389 | LOC285830 | 1 |
| 390 | LOC388965 | 1 |
| 391 | LOC389333 | 1 |
| 392 | LOC401052 | 1 |
| 393 | LOC440982 | 1 |
| 394 | LOC729467 | 1 |
| 395 | LONRF3 | 1 |
| 396 | LOXL2 | 1 |
| 397 | LOXL3 | 1 |
| 398 | LOXL4 | 3 |
| 399 | LPAR2 | 1 |
| 400 | LPIN3 | 1 |
| 401 | LRAT | 1 |
| 402 | LRRC8E | 1 |
| 403 | LTBP4 | 2 |
| 404 | LVRN | 3 |
| 405 | LY6G5C | 1 |
| 406 | LYN | 1 |
| 407 | LYPD4 | 1 |
| 408 | LYPD5 | 1 |
| 409 | MAD2L2 | 2 |
| 410 | MADCAM1 | 1 |
| 411 | MAGEB1 | 1 |
| 412 | MAOB | 1 |
| 413 | MAP3K1 | 1 |
| 414 | MAP4K5 | 1 |
| 415 | MAP6D1 | 2 |
| 416 | MAP7D2 | 1 |
| 417 | MAPK4 | 1 |
| 418 | MAPT | 2 |
| 419 | MBP | 1 |
| 420 | MCM2 | 3 |
| 421 | MDFI | 1 |
| 422 | MDGA1 | 1 |
| 423 | MDGA2 | 2 |
| 424 | ME1 | 1 |
| 425 | MECP2 | 1 |
| 426 | MEGF8 | 1 |
| 427 | MESP1 | 1 |
| 428 | MESP2 | 1 |
| 429 | METRN | 1 |
| 430 | MFSD2A | 2 |
| 431 | MGAT3 | 3 |
| 432 | MGAT5B | 1 |
| 433 | MGC16275 | 1 |
| 434 | MGC70857 | 1 |
| 435 | MGMT | 3 |
| 436 | MICAL1 | 1 |
| 437 | MIR1225 | 1 |
| 438 | MIR1306 | 1 |
| 439 | MIR149 | 1 |
| 440 | MIR1909 | 1 |
| 441 | MIR193B | 1 |
| 442 | MIR210 | 1 |
| 443 | MIR3621 | 1 |
| 444 | MIR574 | 3 |
| 445 | MIR9-3 | 1 |
| 446 | MIXL1 | 1 |
| 447 | MLPH | 1 |
| 448 | MMP15 | 1 |
| 449 | MMP17 | 1 |
| 450 | MMP23B | 1 |
| 451 | MOGAT3 | 1 |
| 452 | MPPED1 | 1 |
| 453 | MPST | 1 |
| 454 | MRGPRE | 3 |
| 455 | MRPS6 | 1 |
| 456 | MSI2 | 1 |
| 457 | MT1G | 2 |
| 458 | MT1L | 1 |
| 459 | MT2A | 1 |
| 460 | MTMR10 | 1 |
| 461 | MTSS1 | 1 |
| 462 | MTSS1L | 1 |
| 463 | MTUS1 | 1 |
| 464 | MYCBPAP | 1 |
| 465 | MYO15B | 1 |
| 466 | N6AMT2 | 1 |
| 467 | NACC2 | 1 |
| 468 | NANP | 1 |
| 469 | NAV1 | 1 |
| 470 | NBL1 | 2 |
| 471 | NCOR1 | 2 |
| 472 | NDRG2 | 1 |
| 473 | NDRG4 | 1 |
| 474 | NDUFA13 | 1 |
| 475 | NECAB1 | 2 |
| 476 | NFYB | 1 |
| 477 | NGF | 2 |
| 478 | NIPAL4 | 3 |
| 479 | NKX2-6 | 1 |
| 480 | NKX6-2 | 3 |
| 481 | NLRC5 | 1 |
| 482 | NME3 | 2 |
| 483 | NOTUM | 1 |
| 484 | NPR1 | 1 |
| 485 | NR5A2 | 1 |
| 486 | OLFM1 | 1 |
| 487 | OOEP | 1 |
| 488 | OTOP2 | 2 |
| 489 | OTOP3 | 2 |
| 490 | OTP | 1 |
| 491 | OVOL1 | 1 |
| 492 | OXCT1 | 1 |
| 493 | P2RY2 | 3 |
| 494 | P4HTM | 1 |
| 495 | PACS1 | 1 |
| 496 | PACSIN1 | 1 |
| 497 | PALD1 | 1 |
| 498 | PALM | 1 |
| 499 | PAPSS2 | 1 |
| 500 | PARP9 | 1 |
| 501 | PARVA | 2 |
| 502 | PAX2 | 1 |
| 503 | PAX6 | 3 |
| 504 | PCDH19 | 1 |
| 505 | PCGF3 | 1 |
| 506 | PCSK6 | 1 |
| 507 | PCSK9 | 2 |
| 508 | PCYT1A | 1 |
| 509 | PDE4C | 1 |
| 510 | PDLIM1 | 2 |
| 511 | PDX1 | 1 |
| 512 | PDZRN3 | 1 |
| 513 | PET117 | 1 |
| 514 | PFN4 | 3 |
| 515 | PHLDA1 | 2 |
| 516 | PKIB | 1 |
| 517 | PKN1 | 1 |
| 518 | PKP1 | 1 |
| 519 | PLBD1 | 2 |
| 520 | PLD6 | 2 |
| 521 | PLEK2 | 1 |
| 522 | PLEKHN1 | 3 |
| 523 | PNLDC1 | 2 |
| 524 | PNPLA5 | 2 |
| 525 | PNPLA6 | 1 |
| 526 | POMC | 1 |
| 527 | POU3F3 | 1 |
| 528 | PPDPF | 2 |
| 529 | PPIE | 1 |
| 530 | PPM1F | 2 |
| 531 | PPM1M | 1 |
| 532 | PPP1R13B | 2 |
| 533 | PPP1R14A | 1 |
| 534 | PPP1R3F | 1 |
| 535 | PPP3CC | 1 |
| 536 | PRDM14 | 1 |
| 537 | PRDM8 | 1 |
| 538 | PRIC285 | 1 |
| 539 | PRMT6 | 1 |
| 540 | PROK2 | 1 |
| 541 | PRR15 | 1 |
| 542 | PRR18 | 1 |
| 543 | PRR22 | 1 |
| 544 | PRRX2 | 1 |
| 545 | PRRX2-AS1 | 1 |
| 546 | PRSS23 | 1 |
| 547 | PSMB9 | 2 |
| 548 | PTF1A | 1 |
| 549 | PTPLA | 1 |
| 550 | PTPN13 | 1 |
| 551 | PTPN18 | 1 |
| 552 | PTPRJ | 1 |
| 553 | PTRF | 1 |
| 554 | PURA | 1 |
| 555 | PUS3 | 1 |
| 556 | PXYLP1 | 1 |
| 557 | PYCR2 | 1 |
| 558 | RAB32 | 1 |
| 559 | RAB4A | 1 |
| 560 | RAB6B | 1 |
| 561 | RAP1GAP | 2 |
| 562 | RARRES1 | 1 |
| 563 | RASD2 | 1 |
| 564 | RASL10A | 2 |
| 565 | RASL12 | 1 |
| 566 | RASSF1 | 2 |
| 567 | RASSF7 | 1 |
| 568 | RBM20 | 1 |
| 569 | RBM24 | 2 |
| 570 | RBM43 | 2 |
| 571 | RBM47 | 2 |
| 572 | RBP7 | 1 |
| 573 | RCAN1 | 1 |
| 574 | RGL3 | 1 |
| 575 | RGMA | 1 |
| 576 | RGS19 | 2 |
| 577 | RHBDD1 | 1 |
| 578 | RHEB | 1 |
| 579 | RLTPR | 1 |
| 580 | RNASEH2A | 2 |
| 581 | RNF165 | 1 |
| 582 | RNF213 | 1 |
| 583 | ROBO3 | 1 |
| 584 | RPL10A | 1 |
| 585 | RSPH1 | 1 |
| 586 | RTEL1 | 1 |
| 587 | RUNX3 | 3 |
| 588 | RXRA | 1 |
| 589 | RYR3 | 1 |
| 590 | S1PR4 | 1 |
| 591 | SALL1 | 2 |
| 592 | SALL3 | 1 |
| 593 | SAMD5 | 1 |
| 594 | SAP130 | 1 |
| 595 | SAP25 | 2 |
| 596 | SAT1 | 1 |
| 597 | SCARB2 | 2 |
| 598 | SCTR | 1 |
| 599 | SDC1 | 1 |
| 600 | SDHAF1 | 2 |
| 601 | SDHC | 1 |
| 602 | SEL1L3 | 2 |
| 603 | SEMA5A | 1 |
| 604 | SERF2 | 1 |
| 605 | SFRP5 | 2 |
| 606 | SGIP1 | 4 |
| 607 | SGPL1 | 2 |
| 608 | SH3BP2 | 1 |
| 609 | SH3GLB1 | 1 |
| 610 | SHB | 2 |
| 611 | SHISA9 | 1 |
| 612 | SIM2 | 1 |
| 613 | SIX2 | 2 |
| 614 | SLC10A3 | 1 |
| 615 | SLC12A4 | 1 |
| 616 | SLC16A12 | 1 |
| 617 | SLC16A3 | 1 |
| 618 | SLC16A5 | 1 |
| 619 | SLC16A6 | 1 |
| 620 | SLC22A16 | 1 |
| 621 | SLC22A23 | 1 |
| 622 | SLC25A13 | 1 |
| 623 | SLC25A39 | 2 |
| 624 | SLC26A10 | 3 |
| 625 | SLC27A3 | 1 |
| 626 | SLC2A4RG | 1 |
| 627 | SLC30A2 | 1 |
| 628 | SLC35A3 | 1 |
| 629 | SLC45A3 | 1 |
| 630 | SLC47A1 | 3 |
| 631 | SLC5A7 | 1 |
| 632 | SLC6A11 | 1 |
| 633 | SLCO4C1 | 4 |
| 634 | SLFN5 | 1 |
| 635 | SLMAP | 1 |
| 636 | SMAD3 | 1 |
| 637 | SNORD1C | 1 |
| 638 | SNPH | 2 |
| 639 | SNRPF | 1 |
| 640 | SNX31 | 1 |
| 641 | SNX9 | 1 |
| 642 | SORBS3 | 1 |
| 643 | SOX3 | 1 |
| 644 | SOX8 | 1 |
| 645 | SPG7 | 1 |
| 646 | SPINT1 | 2 |
| 647 | SPOP | 1 |
| 648 | SRD5A2 | 1 |
| 649 | SRPK3 | 1 |
| 650 | SSBP4 | 1 |
| 651 | ST6GAL2 | 1 |
| 652 | STAG2 | 1 |
| 653 | STAG3 | 1 |
| 654 | STARD8 | 1 |
| 655 | STEAP3 | 2 |
| 656 | STK38L | 1 |
| 657 | STMN1 | 1 |
| 658 | STXBP2 | 1 |
| 659 | SULF2 | 1 |
| 660 | SUSD3 | 1 |
| 661 | SV2B | 2 |
| 662 | SYNJ2 | 2 |
| 663 | SYT10 | 1 |
| 664 | SYT9 | 2 |
| 665 | TAB2 | 1 |
| 666 | TAGLN2 | 1 |
| 667 | TBX5 | 2 |
| 668 | TBXA2R | 1 |
| 669 | TCEA2 | 2 |
| 670 | TCIRG1 | 1 |
| 671 | TCONS_00029157 | 1 |
| 672 | TERC | 1 |
| 673 | TGFB3 | 1 |
| 674 | TGIF2 | 3 |
| 675 | THBS4 | 1 |
| 676 | THRB | 3 |
| 677 | TJP2 | 1 |
| 678 | TLCD2 | 2 |
| 679 | TMED1 | 1 |
| 680 | TMEM106A | 1 |
| 681 | TMEM171 | 3 |
| 682 | TMEM200B | 1 |
| 683 | TMEM238 | 1 |
| 684 | TMEM87A | 1 |
| 685 | TMEM90A | 2 |
| 686 | TMEM92 | 2 |
| 687 | TNFRSF10A | 1 |
| 688 | TNFRSF10B | 1 |
| 689 | TNK2 | 2 |
| 690 | TOX2 | 2 |
| 691 | TP53I11 | 2 |
| 692 | TP53INP1 | 3 |
| 693 | TPPP3 | 2 |
| 694 | TRH | 2 |
| 695 | TRIL | 1 |
| 696 | TRIM45 | 1 |
| 697 | TRIM65 | 3 |
| 698 | TRPC4AP | 1 |
| 699 | TSNARE1 | 1 |
| 700 | TTC22 | 1 |
| 701 | TTC23L | 1 |
| 702 | TTYH2 | 2 |
| 703 | TWIST2 | 2 |
| 704 | UBC | 1 |
| 705 | UBE2E2 | 1 |
| 706 | UCK1 | 2 |
| 707 | UCN | 3 |
| 708 | UCP1 | 2 |
| 709 | UGT8 | 1 |
| 710 | UHRF1 | 1 |
| 711 | ULK2 | 1 |
| 712 | UNC5D | 1 |
| 713 | UNKL | 1 |
| 714 | UPP1 | 3 |
| 715 | UQCRC1 | 1 |
| 716 | USH1C | 1 |
| 717 | USP44 | 1 |
| 718 | UTP11L | 1 |
| 719 | VAMP5 | 1 |
| 720 | VARS2 | 5 |
| 721 | VDAC1 | 2 |
| 722 | VENTX | 1 |
| 723 | VENTXP1 | 1 |
| 724 | VIPR2 | 1 |
| 725 | VWCE | 2 |
| 726 | WIPF3 | 2 |
| 727 | WNT2 | 2 |
| 728 | WNT9A | 2 |
| 729 | WNT9B | 1 |
| 730 | YPEL1 | 1 |
| 731 | ZBTB16 | 1 |
| 732 | ZBTB48 | 1 |
| 733 | ZC3HAV1L | 1 |
| 734 | ZFP41 | 1 |
| 735 | ZIM2 | 1 |
| 736 | ZMIZ1 | 1 |
| 737 | ZMYM2 | 1 |
| 738 | ZNF238 | 1 |
| 739 | ZNF341 | 1 |
| 740 | ZNF513 | 1 |
| 741 | ZNF518B | 1 |
| 742 | ZNF578 | 1 |
| 743 | ZNF586 | 1 |
| 744 | ZNF592 | 1 |
| 745 | ZNF703 | 1 |
| 746 | ZNF783 | 1 |
| 747 | ZNF860 | 1 |

TABLE 3
dCT_to_dCdT_hypermethylated promoter

| | Gene | N |
|---|---|---|
| 1 | CXorf39 | 1 |
| 2 | CYGB | 1 |
| 3 | DENND1B | 1 |
| 4 | DKFZp686O24166 | 1 |
| 5 | EBF2 | 1 |
| 6 | EDN3 | 1 |
| 7 | EEF1A1 | 2 |
| 8 | GJB6 | 1 |
| 9 | GPR26 | 1 |
| 10 | HIATL2 | 1 |
| 11 | IFT57 | 1 |
| 12 | IRS1 | 1 |
| 13 | LRAT | 1 |
| 14 | LVRN | 2 |
| 15 | LYPD5 | 1 |
| 16 | MAP7D2 | 1 |
| 17 | MEGF8 | 1 |
| 18 | MIR9-3 | 1 |
| 19 | MT1G | 2 |
| 20 | PFN4 | 3 |
| 21 | POU3F3 | 1 |
| 22 | PRR15 | 1 |
| 23 | UTP11L | 1 |
| 24 | VIPR2 | 1 |
| 25 | ZC3HAV1L | 1 |
| 26 | ZIM2 | 1 |

### Example 7: Specificity of CDKL5 sgRNAs and dCas9 Effector Domains

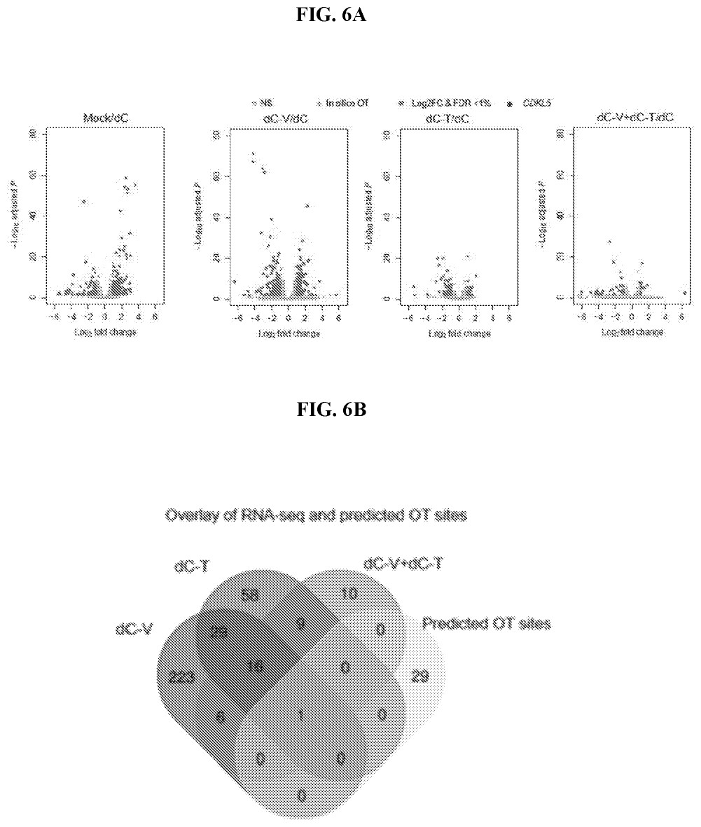

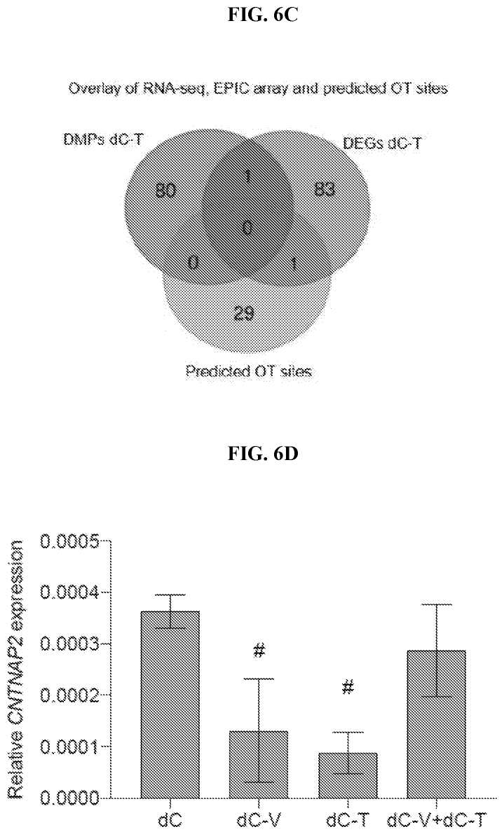

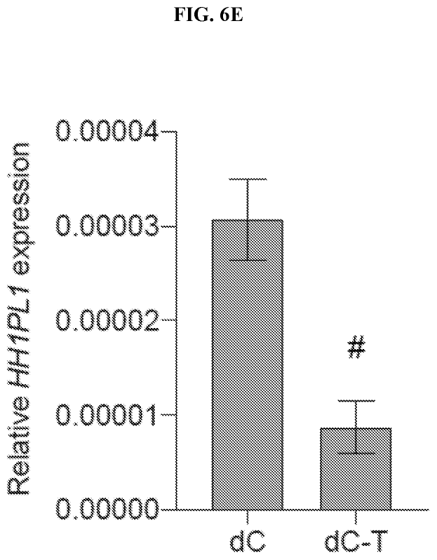

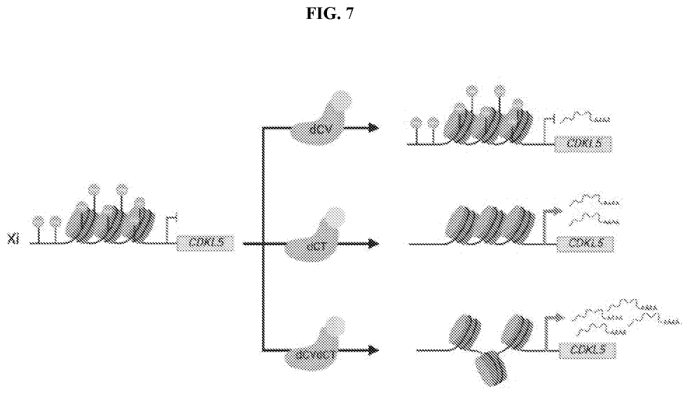

To evaluate the effect of targeting CDKL5 with dCas9 effector fusions on global gene expression, RNA-seq was performed in stably transduced SH-SY5Y. As shown in FIG. 6A and Tables 4-7, the introduction of dC alone causes 208 differentially expressed (DE) genes when compared to mock-treated cells, likely due to the introduction of the lentiviral machinery (66 up- and 142 downregulated genes). Accordingly, pairwise comparisons with dC as the control was performed. When compared to cells transduced with dC, cells transduced with dC-V or dC-V+dC-T targeted to CDKL5 specifically increased CDKL5 expression without altering expression of adjacent transcripts (nearest neighboring genes upstream and downstream of CDKL5). No significant upregulation of CDKL5 was detected in cells treated with dCas9-TET1CD.

Four genes containing heterozygous SNPs in the coding region within a ±2 Mb range of the CDKL5 target site were identified (MAP3K15, RAI2, NHS and BEND2, Tables 4-7). However, mean read counts for these genes were generally unchanged from the mock-treated group, albeit 3 out of 4 genes were lowly or not expressed. Regardless, since the mean read counts for these gene were not significantly altered, the results showed that the X-chromosomal genes were not reactivated. In total, 274 differentially expressed (DE) genes in dC-V (100 up- and 174 downregulated genes), 84 DE genes in dC-T (n=29 up- and n=55 downregulated) and 43 DE genes in dC-V+dC-T (13 up- and 30 downregulated genes) were identified. In general, a greater number of differentially downregulated genes in transduced cells was observed, which was attributed to off-target binding of the constructs as both effector domains conferred transcriptional activation to direct targets, not repression.

Although CDKL5 sgRNA sequences were designed to target a unique site in the human genome, it was possible that the sgRNAs could tolerate mismatches leading to off-target binding. To address this issue, a search for potential off-target (OT) sites with up to 3 mismatches within the sgRNA sequences using CasOFF-Finder was conducted. CasOFF-Finder scane for both nucleotide mismatches and bulges in the sequence, thereby making it a comprehensive in silico prediction tool for OT analysis. To include OT sites that fell within intergenic regions, the targets was extended by ±5 kb from the predicted OT site to include neighboring transcripts and identified a total of 30 predicted OT genes (Tables 4-7).

The majority of OT sites required at least 2 mismatches, with sgRNA 2 only being permissive for OT sites with 3 mismatches in the sequence. Out of 30 OT genes, a single target, CNTNAP2, that was downregulated in dC-V, dC-T and dC-V+dC-T in all three conditions was identified. While the predicted OT site for CNTNAP2 falls within an intronic sequence of the gene, the fact that that the differential expression was a consequence of off-target binding of the dCas9 effector domain could not be precluded. Cells transduced with dC-V showed the highest number of unique differentially expressed transcripts (n=223), followed by dC-T (n=58) and dC-V+dC-T (n=10). As shown in FIG. 6B, a total of 16 differentially expressed genes were shared between the three conditions. A gene ontology analysis did not reveal significant enrichment of terms, indicating that the set of DE genes did not share a common pathway.

To assess whether the observed global changes in DNA methylation in cells transduced with dC-T were associated with altered transcript levels, the overlap between all 81 differentially hypomethylated genes in CGI promoter regions with greater than 3 DM positions was investigated. As shown in FIG. 6C, all 84 DE genes and the predicted 30 OT genes. Overall, a single gene (HHIPL1) that showed association between differentially methylation and expression was identified (FIGS. 6D-E). However, genes overlapping the OT genes and DM positions were not identified. These results showed that the observed global DNA hypomethylation of promoters poorly correlated with gene expression.

### Experimental Discussion

A significant number of X-linked genes escape XCI and are expressed from the inactive X chromosome (16). Whether or not the epigenetic signature associated with these escapees is a cause or merely a consequence of expression from otherwise transcriptionally inert X-chromatin remains to be elucidated (17). The present disclosure demonstrates for one such epigenetic barrier in a specific gene context, that removal of CGI methylation from the promoter of the X-chromosomal gene CDKL5 by directing a fusion of the catalytic domain of TET1 to dCas9 results in reactivation of gene expression in a targeted manner. In addition, employment of a strong transcriptional activator further increased the degree of escape in a synergistic fashion, resulting in expression levels in excess of 60% of the inactive allele when compared to the active allele.

The present disclosure further demonstrates that programmable transcription using a transactivator achieved a moderate but significant CDKL5 upregulation that was achieved across several cell lines. However, the effect of the VP64 transactivator was mainly due to superactivation of the already active allele, demonstrating that the epigenetic landscape of active X-chromatin presented a chromatin state more permissive for programmable transcription. Unexpectedly, the present invention identified that binding of dCas9 with no effector was capable of reactivating CDKL5 expression from the silent allele. This may be due to the large dCas9 protein serving as a pioneer factor when constitutively expressed and targeted to transcriptionally inactive X-chromatin, thereby causing limited gene reactivation on its own. In contrast to previous studies (52, 53), the present invention did not show any hindrance of dCas9 binding to regions largely embedded in CpG-dense hypermethylated CGI promoters. However, that binding of a sgRNA outside of the methylated region on the inactive X chromosome could, at least in part, be causative for the observed effect. The limited but significant reactivation was associated with the loss of the repressive histone mark H3K27me3 in the core promoter of CDKL5. While the direct role between dCas9 binding and depletion of the histone mark is not well understood, it is possible that binding of dCas9 causes displacement of the nucleosome, resulting in the loss of H3K27me3 and enhanced chromatin accessibility (54, 55). In the present disclosure, H3K27me3 was assessed due to its role in XCI. However, future studies to investigate dCas9 effect on nucleosome rearrangement would require the assessment of multiple histone subunits. In line with previous findings (2), a spread of heterochromatin loss to the nearest neighboring gene, which may suggest a targeted effect of dCas9 binding was not observed.

Previous studies suggested that nucleosome occupancy strongly impeded binding of (d)Cas9 (56, 57). However, considering that the disclosed sgRNA design takes DNase hypersensitive sites into account, and considering the finding that the inactive X-allele is approximately 1.2 fold more compact than the active allele (58), the present disclosure demonstrates that a promoter of a gene on the inactive X-chromatin is generally targetable by dCas9. In addition, the accessibility of CDKL5 can be further attributed to the location of the gene on a chromosomal segment that is part of a younger evolutionary strata of the X chromosome (17). Indeed, the majority of facultative and constitutive escape genes are located on the short arm of the X chromosome (17). Therefore, the chromosomal location of CDKL5 might be favourable to induce an artificial escape. The fusion of VP64 to dCas9 did not further increase the observed reactivation, further supporting a steric effect primarily attributed to the large size of dCas9 that is not augmented by the addition of a small transactivator. The indirect recruitment of transcription factors by VP64 did not result in higher reactivation levels and may be due to the chromatin microenvironment, specifically the presence of DNA methylation as an epigenetic barrier that does not permit abundant transcription via VP64.

Changing the chromatin microenvironment via the introduction of TET1CD resulted in decreased DNA methylation of around 15% in the CDKL5 core promoter and significantly reactivated XCI-silenced CDKL5, thereby creating an artificial escape gene as previously defined at expression levels of at least 10% of the active allele (17). Likely due to the depletion of 5-methylcytosine substrate in the promoter of the active allele, recruitment of TET1CD to this region did not result in superactivation of the allele on the active X chromosome. Due to a lack of polymorphisms in the CDKL5 promoter of SH-SY5Y cells, the working model system of the present disclosure did not allow for testing for allele specific changes of the epigenetic signature. Rather, the working model was reliant on the assessment of total changes in DNA methylation in light of the fact that CGI methylation is highly correlative with the inactive X allele. Furthermore, a recent genome-wide assessment revealed global DNA hypomethylation of CGI promoters following TET1CD overexpression via lentiviral integration (29). However, the genome-wide assessment of promoter regions did not identify a strong correlation between reduced methylation of CpG sites in and changes in transcription by RNA-seq. This is likely because the vast majority of genes identified only contain a single differentially methylated site indicative of one CpG site, and the generally small effect size of the measured changes of DNA methylation. The change of a single CpG site in a promoter which typically contains multiple CpG sites likely would not result in biological significance, thus the lack of correlation with transcriptional activation.

Since CGI promoters on the inactive X allele frequently show higher methylation levels than on the active X allele, targeted reduction of CpG methylation is directed to a single allele, unlike the case for autosomal genes. For example, in an autosomal setting, directed epigenetic editing may confer small changes to methylation levels of both alleles. These small changes do not necessarily translate to an additive effect on transcription if neither of the alleles reaches a threshold of biological significance. However, targeting a single X-chromosomal allele of a gene has the potential to concentrate the effects of epigenetic editing that would otherwise be divided over two alleles, increasing its potential to pass this arbitrary biological threshold. Thus, a decrease of DNA methylation on the inactive X chromosome can have a broader implication for regulation of gene expression. In future studies, it will be crucial to test whether a more transient delivery of TET1CD impacts the amount of observed methylation changes. While it was suggested that the effects of dCas9-TET1CD are specific (26), the present disclosure demonstrates global DNA methylome changes (29). Similar findings have been demonstrated for genome-wide DNA methylation changes with fusions of the DNA methyltransferase DNMT3A to dCas9 (59), likely attributed to the high substrate abundance of methylated cytosines for constitutively expressed TET1CD. This highlights the need to assess transient exposure of dCas9-effectors to the CDKL5 promoter in order to reduce potential off-target effects in future studies.

Due to the strong effect of VP64 on upregulating genes in an unmethylated chromatin context, a combination of TET1CD and VP64 targeted to CDKL5 via dCas9 was assessed. A synergistic effect between removal of DNA methylation and strong transcriptional activation that resulted in a greater than 60% expression from the inactive allele was observed. Since the employment of VP64 alone did not significantly increase reactivation levels, it is most likely that the introduction of dCas9-TET1CD causes a dynamic reprograming in which methyl groups are removed from CpG dinucleotides, thus allowing for further binding of transcription factors to the inactive chromatin via an indirect recruitment from VP64. The present disclosure supports a synergistic effect between TET1CD and transactivators that have recently been supported by others (28, 29). In alternative aspect, the effect of improved transcriptional activators, such as the VP64-p65-Rta tripartite fusion (60) or the use of the SunTag (61) system can be harnessed to further potentiate the expression of XCI silenced CDKL5 in combination with TET1CD.

Interestingly, following dual expression of VP64 and TET1CD resulted in the fewest number of DE genes in RNAseq analysis. In silico analysis provided a predicted list of potential off-target genes either through base-pair mismatches or bulges in the gRNA. Only a single gene from the predicted off-target list, CNTNAP2, a gene implicated in autism-spectrum disorders (68), demonstrated differential expression following genome wide transcriptomics. Novel methodologies have been proposed to alter the binding specificities of sgRNAs in order to reduce off-target binding, such as engineering a hairpin secondary structure onto the sgRNA spacer region (66), and will be explored in future studies.

Up until recently, technical hurdles have hampered the assessment of the role of epigenetic heterogeneity in biological systems. One challenge that remains is whether the observed reactivation levels of CDKL5 are due to a limited or partial reactivation at the population-wide level or if the observed effects are specific to a fully reactivated subgroup of cells. Recent evidence suggests that there are specific populations of cells that are more responsive to targeted effects, which will then drive the phenotype at the bulk level (28). It is possible that there are different kinds of responders to the epigenetic edits in our tested culture system and future studies will need to address this mechanistic question. Most likely this biological inquiry will need to be answered at a single cell level in future studies.

Reactivation strategies hold great promise for individuals suffering from X-linked disorders. In contrast to pharmacological inhibition of DNMT1, which postulates the need for mitosis, TET1CD might be a promising tool for demethylation in quiescent tissues that have been traditionally more difficult to target, such as the brain (27). In addition, superactivation by VP64 of the already active CDKL5 allele needs to be carefully assessed due to the fact that Xp22 duplications containing the CDKL5 gene have been described as pathogenic variants (67). Interestingly, Applicant identified that epigenetic editing of dCas9-TET1 does not exceed super-physiological levels of an X-linked target gene, further making this approach favorable in the light of a dosage sensitive gene.

TABLE 4
Differential gene Expression_DESeq2_dCV_dC

| | | Log2 fold | | | | |
|---|---|---|---|---|---|---|
| | Base Mean | Change | Lfcse | Stat | Pvalue | Padj |
| ENSG00000272438 | 75.1005163 | 0.77249714 | 0.22730244 | 3.39854307 | 0.00067746 | 0.02210652 |
| ENSG00000230699 | 331.314838 | 0.80337956 | 0.19999797 | 4.01693864 | 5.90E−05 | 0.00288589 |
| ENSG00000223764 | 513.919766 | 0.94537388 | 0.13695229 | 6.90294309 | 5.09E−12 | 1.26E−09 |
| ENSG00000187634 | 1066.25504 | 1.35934148 | 0.19351993 | 7.02429712 | 2.15E−12 | 5.60E−10 |
| ENSG00000221978 | 4011.35976 | 0.30216435 | 0.09666954 | 3.12574518 | 0.00177355 | 0.04718708 |
| ENSG00000149527 | 355.820637 | −0.6117443 | 0.19585721 | −3.1234199 | 0.00178763 | 0.04737916 |
| ENSG00000142606 | 19.2151287 | −1.3757152 | 0.42886751 | −3.2077861 | 0.00133761 | 0.03806846 |
| ENSG00000171608 | 251.374075 | −0.9915808 | 0.17039503 | −5.8193058 | 5.91E−09 | 7.68E−07 |
| ENSG00000219481 | 2511.36484 | 0.429801 | 0.12964489 | 3.3152175 | 0.00091572 | 0.02834433 |
| ENSG00000070886 | 191.138666 | −0.7071598 | 0.22704409 | −3.1146367 | 0.00184172 | 0.04849616 |
| ENSG00000185436 | 13.4244455 | −1.9386401 | 0.60983881 | −3.1789386 | 0.00147815 | 0.04120305 |
| ENSG00000182749 | 261.26814 | −0.6267173 | 0.17216027 | −3.6403135 | 0.00027231 | 0.01037502 |
| ENSG00000176092 | 50.5455561 | −1.4264293 | 0.28729244 | −4.9650777 | 6.87E−07 | 5.83E−05 |
| ENSG00000060656 | 850.414466 | −0.5830075 | 0.16179368 | −3.6034011 | 0.00031408 | 0.01174786 |
| ENSG00000168528 | 207.775925 | −1.4033851 | 0.18780225 | −7.4726745 | 7.86E−14 | 2.59E−11 |
| ENSG00000220785 | 62.5325212 | −1.471762 | 0.26551483 | −5.54305 | 2.97E−08 | 3.38E−06 |
| ENSG00000183615 | 30.4313561 | −1.7496948 | 0.39227403 | −4.4603891 | 8.18E−06 | 0.00053138 |
| ENSG00000162522 | 693.212725 | −0.5379417 | 0.17260681 | −3.1165729 | 0.00182966 | 0.04836554 |
| ENSG00000160097 | 171.344295 | 0.75770485 | 0.16953068 | 4.46942619 | 7.84E−06 | 0.00051104 |
| ENSG00000171812 | 35.0090492 | −1.3670288 | 0.35503132 | −3.8504457 | 0.0001179 | 0.00520183 |
| ENSG00000183317 | 169.763339 | −0.628568 | 0.18790049 | −3.3452176 | 0.00082218 | 0.02583996 |
| ENSG00000116990 | 137.955411 | −0.6875084 | 0.20746986 | −3.3137748 | 0.00092046 | 0.02844793 |
| ENSG00000049089 | 101.540544 | −1.5436111 | 0.24218583 | −6.373664 | 1.85E−10 | 3.31E−08 |
| ENSG00000117016 | 1948.07408 | −0.5122385 | 0.14737623 | −3.4757199 | 0.00050948 | 0.01757849 |
| ENSG00000085831 | 24.8222194 | −1.8499953 | 0.39214209 | −4.7176659 | 2.39E−06 | 0.00017945 |
| ENSG00000116157 | 224.270938 | −1.1015917 | 0.18777961 | −5.8664075 | 4.45E−09 | 5.92E−07 |
| ENSG00000162407 | 227.302124 | −1.1968103 | 0.17828526 | −6.7128955 | 1.91E−11 | 4.24E−09 |
| ENSG00000134709 | 260.419574 | −0.6837188 | 0.21181313 | −3.2279342 | 0.00124688 | 0.03603262 |
| ENSG00000079739 | 1479.42399 | −0.3549829 | 0.10976969 | −3.2338879 | 0.00122117 | 0.03549036 |
| ENSG00000178965 | 215.550561 | 1.32020028 | 0.21496676 | 6.1414159 | 8.18E−10 | 1.24E−07 |
| ENSG00000154027 | 771.147547 | 0.64730674 | 0.10904752 | 5.93600596 | 2.92E−09 | 4.04E−07 |
| ENSG00000162614 | 30.4035506 | 1.20877111 | 0.37797299 | 3.19803572 | 0.00138367 | 0.03899438 |
| ENSG00000153904 | 1124.74806 | −0.7647522 | 0.14685222 | −5.207631 | 1.91E−07 | 1.91E−05 |
| ENSG00000142871 | 226.188748 | 0.81280125 | 0.15791058 | 5.14722469 | 2.64E−07 | 2.54E−05 |
| ENSG00000143013 | 215.289297 | 1.55733234 | 0.18850702 | 8.26140258 | 1.44E−16 | 7.55E−14 |
| ENSG00000137962 | 326.475887 | −0.6615784 | 0.19483776 | −3.3955347 | 0.00068495 | 0.02227983 |
| ENSG00000143036 | 29.7039746 | −1.5413893 | 0.36064996 | −4.2739205 | 1.92E−05 | 0.00110695 |
| ENSG00000235501 | 142.188258 | −0.7357623 | 0.18305032 | −4.0194536 | 5.83E−05 | 0.00286211 |
| ENSG00000237954 | 49.4200112 | −2.5496541 | 0.3302849 | −7.7195598 | 1.17E−14 | 4.42E−12 |
| ENSG00000188641 | 587.752757 | −0.7854787 | 0.15157426 | −5.1821375 | 2.19E−07 | 2.16E−05 |
| ENSG00000099260 | 36.5706791 | 1.50257577 | 0.34248783 | 4.38723839 | 1.15E−05 | 0.00070747 |
| ENSG00000060718 | 173.362961 | 0.76164448 | 0.19779604 | 3.85065582 | 0.0001178 | 0.00520183 |
| ENSG00000162631 | 78.5574294 | 0.72643617 | 0.23257831 | 3.12340458 | 0.00178772 | 0.04737916 |
| ENSG00000116299 | 132.548918 | −0.9225008 | 0.20488665 | −4.5024934 | 6.72E−06 | 0.00044906 |
| ENSG00000116396 | 116.688511 | −0.8469357 | 0.19989392 | −4.236926 | 2.27E−05 | 0.00127021 |
| ENSG00000184260 | 21.3807733 | −1.7115205 | 0.51684206 | −3.3114962 | 0.00092798 | 0.02863736 |
| ENSG00000143375 | 44.639474 | −1.000113 | 0.2985522 | −3.3498764 | 0.00080848 | 0.02548755 |
| ENSG00000197956 | 191.522187 | −1.7935013 | 0.25534976 | −7.0237045 | 2.16E−12 | 5.60E−10 |
| ENSG00000188643 | 39.3441516 | 1.47604917 | 0.35377152 | 4.1723234 | 3.02E−05 | 0.00163198 |
| ENSG00000160691 | 5930.36505 | −0.4924226 | 0.14763161 | −3.3354822 | 0.00085152 | 0.02651753 |
| ENSG00000169231 | 355.727203 | 0.50904197 | 0.13803386 | 3.68780495 | 0.0002262 | 0.00884893 |
| ENSG00000143320 | 527.431765 | −0.8420782 | 0.19303794 | −4.3622418 | 1.29E−05 | 0.00077927 |
| ENSG00000198400 | 692.709832 | 0.85900243 | 0.18968188 | 4.52864789 | 5.94E−06 | 0.00040084 |
| ENSG00000027644 | 343.378514 | 1.04954824 | 0.1912049 | 5.48912848 | 4.04E−08 | 4.47E−06 |
| ENSG00000183853 | 114.745305 | 1.12890056 | 0.24950275 | 4.52460164 | 6.05E−06 | 0.00040724 |
| ENSG00000163565 | 6.18083359 | 3.27474808 | 0.89391869 | 3.66336235 | 0.00024893 | 0.00951967 |
| ENSG00000158710 | 3326.71326 | −0.6361403 | 0.13290107 | −4.7865704 | 1.70E−06 | 0.00013001 |
| ENSG00000158769 | 113.1722 | −0.8836379 | 0.19885072 | −4.4437247 | 8.84E−06 | 0.0005653 |
| ENSG00000162755 | 53.4411614 | −1.5175311 | 0.3079957 | −4.9271178 | 8.35E−07 | 6.94E−05 |
| ENSG00000158859 | 262.427965 | −0.5608108 | 0.16171719 | −3.4678491 | 0.00052464 | 0.01795012 |
| ENSG00000143153 | 3963.35425 | −0.5784174 | 0.08874362 | −6.517848 | 7.13E−11 | 1.38E−08 |
| ENSG00000230630 | 286.12763 | 0.82944942 | 0.21170255 | 3.91799455 | 8.93E−05 | 0.00408691 |
| ENSG00000235750 | 31.4874011 | −1.554971 | 0.37241342 | −4.1753893 | 2.97E−05 | 0.00161442 |
| ENSG00000116183 | 13.6309095 | −1.9380922 | 0.50217615 | −3.8593872 | 0.00011367 | 0.005078 |
| ENSG00000143340 | 1247.03673 | −0.9035968 | 0.13779142 | −6.5577144 | 5.46E−11 | 1.13E−08 |
| ENSG00000229407 | 165.529278 | −1.0325701 | 0.17828296 | −5.7917488 | 6.97E−09 | 8.96E−07 |
| ENSG00000143333 | 849.829044 | 0.74708099 | 0.15431343 | 4.8413218 | 1.29E−06 | 0.00010308 |
| ENSG00000135829 | 7804.95605 | 0.36016903 | 0.10226981 | 3.52175333 | 0.0004287 | 0.01514898 |
| ENSG00000117266 | 254.232911 | −0.5814394 | 0.15189338 | −3.8279441 | 0.00012922 | 0.00560128 |
| ENSG00000276600 | 45.0864726 | 1.23876467 | 0.29089395 | 4.25847524 | 2.06E−05 | 0.00116654 |
| ENSG00000123689 | 51.9610231 | −1.178139 | 0.34897806 | −3.3759687 | 0.00073556 | 0.02366292 |
| ENSG00000117595 | 223.578204 | 1.08041183 | 0.15815525 | 6.83133707 | 8.41E−12 | 1.96E−09 |
| ENSG00000198570 | 186.006471 | −1.3665342 | 0.18376095 | −7.4364775 | 1.03E−13 | 3.25E−11 |
| ENSG00000230461 | 309.632859 | −0.8319628 | 0.17281017 | −4.8143164 | 1.48E−06 | 0.00011668 |
| ENSG00000152104 | 175.493759 | 0.91107076 | 0.19443802 | 4.68566166 | 2.79E−06 | 0.00020612 |
| ENSG00000196660 | 24.119524 | −2.4664167 | 0.43702635 | −5.6436339 | 1.66E−08 | 1.98E−06 |
| ENSG00000186205 | 747.6129 | −0.472508 | 0.12250272 | −3.8571226 | 0.00011473 | 0.00511409 |
| ENSG00000143674 | 222.777063 | −1.0716963 | 0.16940918 | −6.3260817 | 2.51E−10 | 4.44E−08 |
| ENSG00000135750 | 464.866573 | −0.5879712 | 0.15010417 | −3.9170878 | 8.96E−05 | 0.00409315 |
| ENSG00000183780 | 100.874409 | −0.8786935 | 0.21994091 | −3.9951345 | 6.47E−05 | 0.00312003 |
| ENSG00000180875 | 343.86164 | 2.23438796 | 0.15030124 | 14.8660646 | 5.47E−50 | 1.87E−46 |
| ENSG00000221953 | 67.655217 | −0.8210563 | 0.26359016 | −3.1148976 | 0.00184009 | 0.04849616 |
| ENSG00000115738 | 2503.56412 | 1.01212248 | 0.13866121 | 7.2992472 | 2.89E−13 | 8.34E−11 |
| ENSG00000071575 | 2429.92788 | 0.6445455 | 0.09557143 | 6.74412335 | 1.54E−11 | 3.46E−09 |
| ENSG00000132031 | 121.91475 | −1.8297283 | 0.20564518 | −8.8975014 | 5.71E−19 | 3.90E−16 |
| ENSG00000075426 | 608.189644 | 0.54286099 | 0.1675026 | 3.24091081 | 0.00119148 | 0.03487521 |
| ENSG00000049323 | 298.697547 | 0.85057775 | 0.15332553 | 5.54752866 | 2.90E−08 | 3.33E−06 |
| ENSG00000150938 | 154.566282 | 1.83463152 | 0.19670031 | 9.32703943 | 1.09E−20 | 9.28E−18 |
| ENSG00000183023 | 317.349215 | 0.73147755 | 0.16544517 | 4.42126878 | 9.81E−06 | 0.00061394 |
| ENSG00000119866 | 103.009948 | 1.15165476 | 0.22793302 | 5.05260167 | 4.36E−07 | 3.93E−05 |
| ENSG00000169604 | 2559.81655 | 0.64706992 | 0.1797601 | 3.59963048 | 0.00031867 | 0.01189778 |
| ENSG00000196975 | 303.681175 | −0.9252243 | 0.14125909 | −6.5498395 | 5.76E−11 | 1.16E−08 |
| ENSG00000144043 | 899.665797 | 0.49608003 | 0.12685306 | 3.91066667 | 9.20E−05 | 0.00419415 |
| ENSG00000163017 | 150.638098 | 1.41120579 | 0.21650226 | 6.51820343 | 7.12E−11 | 1.38E−08 |
| ENSG00000144045 | 888.856109 | 0.49836485 | 0.12593391 | 3.95735241 | 7.58E−05 | 0.00356451 |
| ENSG00000168874 | 141.952709 | 1.0196973 | 0.21981506 | 4.63888724 | 3.50E−06 | 0.00024972 |
| ENSG00000232931 | 121.166214 | 0.85598724 | 0.22350352 | 3.82986016 | 0.00012822 | 0.00558149 |
| ENSG00000158050 | 128.21304 | 1.39424329 | 0.23542128 | 5.92233326 | 3.17E−09 | 4.36E−07 |
| ENSG00000228873 | 25.803018 | 1.57019786 | 0.39163382 | 4.00935206 | 6.09E−05 | 0.002966 |
| ENSG00000235597 | 68.7680782 | −0.8145127 | 0.26041944 | −3.1276953 | 0.00176183 | 0.04693617 |
| ENSG00000115665 | 148.208812 | 0.77447285 | 0.18889015 | 4.10012293 | 4.13E−05 | 0.00212736 |
| ENSG00000175497 | 263.30137 | −1.3861259 | 0.14145985 | −9.7987232 | 1.14E−22 | 1.23E−19 |
| ENSG00000235026 | 31.7456225 | −1.3636107 | 0.34406122 | −3.9632792 | 7.39E−05 | 0.00349319 |
| ENSG00000155052 | 10.2336296 | −2.3273893 | 0.66159788 | −3.5178307 | 0.00043509 | 0.01534817 |
| ENSG00000152076 | 335.354352 | 0.62047178 | 0.16544886 | 3.75023313 | 0.00017667 | 0.00720054 |
| ENSG00000176771 | 102.435685 | −0.7449704 | 0.22848377 | −3.2604958 | 0.00111218 | 0.03278837 |
| ENSG00000150540 | 29.7334973 | 1.44921521 | 0.42239664 | 3.43093453 | 0.00060151 | 0.02014207 |
| ENSG00000168702 | 56.670944 | 1.1357534 | 0.27674264 | 4.10400578 | 4.06E−05 | 0.00211937 |
| ENSG00000115963 | 1038.96493 | −0.6808378 | 0.13515086 | −5.0376135 | 4.71E−07 | 4.16E−05 |
| ENSG00000115159 | 242.299623 | −1.7817162 | 0.17488857 | −10.187723 | 2.25E−24 | 3.07E−21 |
| ENSG00000115170 | 602.735932 | 0.40266749 | 0.12235564 | 3.29095971 | 0.00099846 | 0.03021973 |
| ENSG00000169432 | 803.237788 | 1.04294081 | 0.20234889 | 5.1541712 | 2.55E−07 | 2.46E−05 |
| ENSG00000128683 | 9.89148776 | −3.5843839 | 0.7000349 | −5.1202931 | 3.05E−07 | 2.85E−05 |
| ENSG00000091428 | 1693.26457 | 0.59237223 | 0.13184599 | 4.49291059 | 7.03E−06 | 0.00046477 |
| ENSG00000144354 | 1393.70125 | 0.34136556 | 0.10768122 | 3.17014935 | 0.00152361 | 0.0420121 |
| ENSG00000162992 | 38.6545046 | −1.1811749 | 0.31621926 | −3.7353035 | 0.00018749 | 0.00759609 |
| ENSG00000162998 | 57.6370134 | −1.9117796 | 0.27813104 | −6.873665 | 6.26E−12 | 1.49E−09 |
| ENSG00000168542 | 4165.10392 | 0.66800551 | 0.1133657 | 5.89248373 | 3.80E−09 | 5.15E−07 |
| ENSG00000204262 | 91.0074667 | 0.74035913 | 0.21402292 | 3.45925155 | 0.00054168 | 0.01837935 |
| ENSG00000151689 | 143.902812 | −1.3347677 | 0.17981794 | −7.4228838 | 1.15E−13 | 3.55E−11 |
| ENSG00000196141 | 776.601835 | 0.6862066 | 0.11103051 | 6.18034291 | 6.40E−10 | 1.01E−07 |
| ENSG00000003402 | 167.088291 | −0.8242409 | 0.18166296 | −4.5371987 | 5.70E−06 | 0.00038878 |
| ENSG00000118257 | 75.9005249 | 0.82085033 | 0.24948253 | 3.29021171 | 0.00100112 | 0.03025542 |
| ENSG00000114948 | 1272.75094 | 0.61792995 | 0.12138006 | 5.0908688 | 3.56E−07 | 3.27E−05 |
| ENSG00000115414 | 1669.50945 | 0.90122477 | 0.22327849 | 4.03632588 | 5.43E−05 | 0.00268976 |
| ENSG00000115461 | 5652.74255 | 1.36118487 | 0.17088724 | 7.96539804 | 1.65E−15 | 6.88E−13 |
| ENSG00000171951 | 3148.1728 | 0.54333057 | 0.12267456 | 4.42904039 | 9.47E−06 | 0.00059957 |
| ENSG00000163053 | 251.218806 | −0.5048124 | 0.15212136 | −3.3184843 | 0.00090507 | 0.02805729 |
| ENSG00000135899 | 47.8778709 | 1.19531363 | 0.29797971 | 4.01139274 | 6.04E−05 | 0.00294749 |
| ENSG00000188042 | 404.773974 | −0.5359039 | 0.14160676 | −3.7844515 | 0.00015405 | 0.00645865 |
| ENSG00000132329 | 241.770148 | −1.5344824 | 0.25677514 | −5.9759774 | 2.29E−09 | 3.23E−07 |
| ENSG00000176720 | 136.021614 | −0.6958058 | 0.20262148 | −3.434018 | 0.0005947 | 0.01994698 |
| ENSG00000134121 | 69.2168007 | 0.81490752 | 0.24064801 | 3.38630486 | 0.00070841 | 0.02289731 |
| ENSG00000196220 | 217.688378 | −0.6681054 | 0.18049345 | −3.7015491 | 0.00021429 | 0.00846671 |
| ENSG00000196639 | 44.106565 | 1.363336 | 0.29051737 | 4.69278658 | 2.70E−06 | 0.00020051 |
| ENSG00000163520 | 528.379697 | −0.8202021 | 0.1653617 | −4.9600488 | 7.05E−07 | 5.93E−05 |
| ENSG00000131378 | 719.129751 | −0.7687448 | 0.12541709 | −6.1295062 | 8.82E−10 | 1.32E−07 |
| ENSG00000228956 | 402.376061 | 0.41929592 | 0.13457426 | 3.11572141 | 0.00183496 | 0.04844282 |
| ENSG00000173705 | 34.9328488 | 1.54021947 | 0.32971384 | 4.6713825 | 2.99E−06 | 0.0002194 |
| ENSG00000187091 | 337.443417 | −0.6299957 | 0.14079352 | −4.4746068 | 7.66E−06 | 0.000502 |
| ENSG00000182983 | 83.4784909 | −2.6672636 | 0.30164409 | −8.8424195 | 9.37E−19 | 5.81E−16 |
| ENSG00000181585 | 74.3238403 | −0.7743555 | 0.24180613 | −3.2023814 | 0.00136296 | 0.03851691 |
| ENSG00000007402 | 2567.3274 | −0.7287859 | 0.18456054 | −3.948763 | 7.86E−05 | 0.00366118 |
| ENSG00000163932 | 223.486999 | −0.668296 | 0.15410586 | −4.3366032 | 1.45E−05 | 0.00086064 |
| ENSG00000163689 | 68.5999592 | −0.8004484 | 0.23862521 | −3.3544167 | 0.00079533 | 0.02522018 |
| ENSG00000163638 | 4307.60544 | −0.5408445 | 0.1551194 | −3.4866335 | 0.00048914 | 0.01696242 |
| ENSG00000121440 | 634.999859 | 0.46166166 | 0.12494914 | 3.6947967 | 0.00022006 | 0.00864201 |
| ENSG00000185008 | 288.228115 | −1.2066196 | 0.20324588 | −5.9367482 | 2.91E−09 | 4.04E−07 |
| ENSG00000168386 | 165.338596 | 1.15640643 | 0.19652027 | 5.88441301 | 3.99E−09 | 5.38E−07 |
| ENSG00000170017 | 4114.91898 | −0.8453398 | 0.13686196 | −6.1765869 | 6.55E−10 | 1.02E−07 |
| ENSG00000144824 | 167.235802 | 0.80225745 | 0.23301042 | 3.44301108 | 0.00057528 | 0.019327 |
| ENSG00000172020 | 1137.43219 | −0.5958128 | 0.17693233 | −3.3674615 | 0.00075864 | 0.02429061 |
| ENSG00000144843 | 12.6284706 | −2.0896558 | 0.65388028 | −3.1957773 | 0.00139455 | 0.0391929 |
| ENSG00000138495 | 128.143453 | −1.4005482 | 0.287922 | −4.864332 | 1.15E−06 | 9.29E−05 |
| ENSG00000082684 | 103.315161 | −1.5078927 | 0.24580149 | −6.1345954 | 8.54E−10 | 1.28E−07 |
| ENSG00000065534 | 344.220933 | 0.71477319 | 0.20323292 | 3.51701471 | 0.00043643 | 0.01536893 |
| ENSG00000206384 | 24.5615613 | 1.65948755 | 0.40567753 | 4.09065677 | 4.30E−05 | 0.00219475 |
| ENSG00000196353 | 31.7262009 | −1.6773728 | 0.36583529 | −4.5850492 | 4.54E−06 | 0.00031803 |
| ENSG00000154917 | 627.542444 | −0.8078423 | 0.14355357 | −5.6274623 | 1.83E−08 | 2.15E−06 |
| ENSG00000114054 | 1110.19178 | −0.4383505 | 0.1109792 | −3.9498437 | 7.82E−05 | 0.00365413 |
| ENSG00000158234 | 86.5074918 | −1.051531 | 0.24404207 | −4.3088103 | 1.64E−05 | 0.00096224 |
| ENSG00000163762 | 51.0116089 | −10.282963 | 1.2045568 | −8.5367194 | 1.38E−17 | 7.64E−15 |
| ENSG00000174899 | 13.8090112 | −3.2893654 | 0.74080102 | −4.4402819 | 8.98E−06 | 0.00057263 |
| ENSG00000169255 | 181.053948 | −1.7102807 | 0.24099114 | −7.0968612 | 1.28E−12 | 3.44E−10 |
| ENSG00000114200 | 65.2453712 | −1.6315601 | 0.25959919 | −6.2849196 | 3.28E−10 | 5.69E−08 |
| ENSG00000085276 | 32.5717915 | −1.8100428 | 0.31627967 | −5.7229184 | 1.05E−08 | 1.31E−06 |
| ENSG00000154310 | 335.125274 | −0.694236 | 0.18236941 | −3.8067568 | 0.0001408 | 0.00601417 |
| ENSG00000169760 | 116.220914 | 0.91552308 | 0.23231277 | 3.94090718 | 8.12E−05 | 0.00374903 |
| ENSG00000145198 | 111.716628 | −1.3917194 | 0.22466907 | −6.1945305 | 5.85E−10 | 9.34E−08 |
| ENSG00000073849 | 301.53194 | 0.57443403 | 0.17891626 | 3.21063055 | 0.00132444 | 0.03783896 |
| ENSG00000145012 | 53.354428 | 2.06914477 | 0.31588401 | 6.55033088 | 5.74E−11 | 1.16E−08 |
| ENSG00000180611 | 152.301745 | 0.78528628 | 0.1843658 | 4.25939245 | 2.05E−05 | 0.00116499 |
| ENSG00000163975 | 350.262103 | −0.9017179 | 0.2070315 | −4.3554621 | 1.33E−05 | 0.00079439 |
| ENSG00000145217 | 98.3998243 | 1.07547818 | 0.27057544 | 3.97478119 | 7.04E−05 | 0.00336749 |
| ENSG00000127418 | 103.602173 | 1.46340943 | 0.22202408 | 6.59121956 | 4.36E−11 | 9.20E−09 |
| ENSG00000178222 | 177.567133 | −1.0561579 | 0.16179099 | −6.5279152 | 6.67E−11 | 1.32E−08 |
| ENSG00000181215 | 42.8654341 | 1.05180353 | 0.29384328 | 3.57947111 | 0.00034429 | 0.01264664 |
| ENSG00000074211 | 771.421707 | −0.460051 | 0.11977997 | −3.8408005 | 0.00012263 | 0.00537836 |
| ENSG00000179299 | 149.653759 | −0.945836 | 0.16738056 | −5.6508114 | 1.60E−08 | 1.92E−06 |
| ENSG00000188848 | 1360.02465 | −0.6717369 | 0.16383946 | −4.0999702 | 4.13E−05 | 0.00212736 |
| ENSG00000145244 | 163.329097 | 1.79305152 | 0.18779119 | 9.54811315 | 1.32E−21 | 1.23E−18 |
| ENSG00000163293 | 68.116999 | 0.80703688 | 0.25122164 | 3.21244964 | 0.00131608 | 0.03771294 |
| ENSG00000074966 | 42.9750428 | 1.53250782 | 0.32116595 | 4.77170084 | 1.83E−06 | 0.00013894 |
| ENSG00000163071 | 72.896052 | −0.8187751 | 0.25460617 | −3.2158496 | 0.00130059 | 0.03737369 |
| ENSG00000226887 | 25.1086181 | −1.7748997 | 0.44391272 | −3.9983079 | 6.38E−05 | 0.00308578 |
| ENSG00000157404 | 534.221611 | 0.90662809 | 0.1753363 | 5.17079525 | 2.33E−07 | 2.27E−05 |
| ENSG00000109255 | 364.074564 | −0.8428809 | 0.21782782 | −3.8694823 | 0.00010907 | 0.00490817 |
| ENSG00000084093 | 744.896065 | 0.47455156 | 0.14081492 | 3.37003754 | 0.00075158 | 0.02410238 |
| ENSG00000145284 | 540.315179 | 0.62643011 | 0.12808351 | 4.89079455 | 1.00E−06 | 8.22E−05 |
| ENSG00000138640 | 158.840479 | 0.54624395 | 0.17492779 | 3.12268252 | 0.00179211 | 0.0474341 |
| ENSG00000138696 | 177.029492 | −1.1490012 | 0.18896386 | −6.0805341 | 1.20E−09 | 1.75E−07 |
| ENSG00000155011 | 1746.60472 | 0.95036619 | 0.11778014 | 8.06898527 | 7.09E−16 | 3.30E−13 |
| ENSG00000138795 | 53.0506709 | 1.10004315 | 0.289254 | 3.80303515 | 0.00014293 | 0.00606728 |
| ENSG00000005059 | 150.572355 | −1.1633587 | 0.17647127 | −6.5923407 | 4.33E−11 | 9.20E−09 |
| ENSG00000205403 | 28.7413148 | 1.34340734 | 0.35296746 | 3.80603735 | 0.00014121 | 0.00601912 |
| ENSG00000178403 | 7.76211399 | −4.8175441 | 0.9897017 | −4.8676729 | 1.13E−06 | 9.17E−05 |
| ENSG00000145349 | 2720.58692 | 0.41556241 | 0.13141711 | 3.16216366 | 0.00156602 | 0.04294996 |
| ENSG00000180801 | 1425.16258 | 0.61392887 | 0.11828252 | 5.19036015 | 2.10E−07 | 2.07E−05 |
| ENSG00000174607 | 73.665151 | −1.5204271 | 0.26531453 | −5.7306589 | 1.00E−08 | 1.26E−06 |
| ENSG00000164111 | 636.93577 | −0.6641124 | 0.14433837 | −4.6010799 | 4.20E−06 | 0.00029653 |
| ENSG00000164056 | 403.535003 | 0.5278646 | 0.12342426 | 4.27683023 | 1.90E−05 | 0.00109567 |
| ENSG00000164070 | 411.066137 | −0.7008503 | 0.165752 | −4.2283067 | 2.35E−05 | 0.00131266 |
| ENSG00000236296 | 29.1265357 | −1.2786534 | 0.39557471 | −3.2323941 | 0.00122758 | 0.03558737 |
| ENSG00000151615 | 13.6964705 | 2.66701124 | 0.66596938 | 4.00470552 | 6.21E−05 | 0.00301057 |
| ENSG00000151617 | 1972.80159 | 1.42859279 | 0.13901162 | 10.2767872 | 8.97E−25 | 1.31E−21 |
| ENSG00000280219 | 82.9412134 | 1.45818436 | 0.24397756 | 5.97671503 | 2.28E−09 | 3.23E−07 |
| ENSG00000181541 | 30.3227365 | 2.21730957 | 0.35369256 | 6.2690309 | 3.63E−10 | 6.19E−08 |
| ENSG00000145428 | 84.7531645 | −1.2010435 | 0.24093128 | −4.9850044 | 6.20E−07 | 5.35E−05 |
| ENSG00000164116 | 382.851182 | 0.93560612 | 0.18228001 | 5.13279622 | 2.85E−07 | 2.69E−05 |
| ENSG00000061918 | 397.775029 | 0.65935169 | 0.13751903 | 4.79462147 | 1.63E−06 | 0.00012583 |
| ENSG00000164125 | 33.7629789 | 1.55345828 | 0.32234201 | 4.81928584 | 1.44E−06 | 0.00011425 |
| ENSG00000168843 | 250.655119 | −0.8129437 | 0.19065068 | −4.2640482 | 2.01E−05 | 0.00114414 |
| ENSG00000218336 | 989.047012 | −0.5101628 | 0.15212308 | −3.3536187 | 0.00079762 | 0.0252231 |
| ENSG00000153404 | 437.340887 | −0.5860402 | 0.15539975 | −3.7711782 | 0.00016248 | 0.00668926 |
| ENSG00000225138 | 119.355418 | 0.88859142 | 0.24524203 | 3.62332427 | 0.00029084 | 0.01097897 |
| ENSG00000206077 | 103.038842 | −0.8741696 | 0.22043128 | −3.965724 | 7.32E−05 | 0.00346558 |
| ENSG00000250056 | 36.4597451 | −2.1960266 | 0.39480377 | −5.5623243 | 2.66E−08 | 3.09E−06 |
| ENSG00000112902 | 2339.38976 | 0.65539218 | 0.19360214 | 3.38525275 | 0.00071113 | 0.022949 |
| ENSG00000176788 | 952.227465 | −0.9954459 | 0.15985545 | −6.2271628 | 4.75E−10 | 7.84E−08 |
| ENSG00000113494 | 17.7919736 | −1.8488994 | 0.49129771 | −3.7632974 | 0.00016769 | 0.00687553 |
| ENSG00000145623 | 38.3616683 | 1.59524303 | 0.3254668 | 4.90140014 | 9.52E−07 | 7.82E−05 |
| ENSG00000016082 | 3574.99136 | −0.3150554 | 0.08561425 | −3.6799416 | 0.00023329 | 0.00903989 |
| ENSG00000164294 | 11.0140951 | 2.03601315 | 0.60042306 | 3.39096429 | 0.00069647 | 0.0225829 |
| ENSG00000145632 | 286.577628 | 0.63883366 | 0.14173879 | 4.50711954 | 6.57E−06 | 0.00044082 |
| ENSG00000113448 | 65.5823133 | −1.0356894 | 0.29374367 | −3.525827 | 0.00042216 | 0.01494368 |
| ENSG00000214944 | 97.3488142 | 0.64693008 | 0.19885593 | 3.25326019 | 0.00114089 | 0.03344213 |
| ENSG00000145703 | 87.8488735 | 1.07154864 | 0.23912368 | 4.48114811 | 7.42E−06 | 0.00048843 |
| ENSG00000164220 | 15.4782681 | 3.05776337 | 0.5357083 | 5.70788874 | 1.14E−08 | 1.41E−06 |
| ENSG00000145685 | 2265.26613 | 0.50828319 | 0.14266589 | 3.56275204 | 0.00036699 | 0.0132193 |
| ENSG00000038427 | 1105.63965 | 0.84265664 | 0.21419113 | 3.93413418 | 8.35E−05 | 0.00383899 |
| ENSG00000245526 | 15.2410883 | −1.6931367 | 0.48937125 | −3.4598207 | 0.00054054 | 0.01837102 |
| ENSG00000113532 | 9.08959969 | −2.8687566 | 0.75707726 | −3.7892522 | 0.0001511 | 0.00636119 |
| ENSG00000152503 | 279.044568 | −1.156258 | 0.15436513 | −7.4904094 | 6.87E−14 | 2.38E−11 |
| ENSG00000184838 | 835.07095 | 0.6245421 | 0.09959279 | 6.27095709 | 3.59E−10 | 6.17E−08 |
| ENSG00000248927 | 111.560857 | 0.95922465 | 0.23937837 | 4.00714838 | 6.15E−05 | 0.00298669 |
| ENSG00000229855 | 3.72327514 | 4.98271391 | 1.40462839 | 3.54735384 | 0.00038912 | 0.01391858 |
| ENSG00000113083 | 135.385085 | −0.8999789 | 0.19867754 | −4.5298473 | 5.90E−06 | 0.00039989 |
| ENSG00000113396 | 153.87229 | −0.6441312 | 0.20241228 | −3.1822736 | 0.00146124 | 0.04078705 |
| ENSG00000066583 | 10696.836 | 0.89051756 | 0.12530385 | 7.10686541 | 1.19E−12 | 3.24E−10 |
| ENSG00000145808 | 452.990741 | 1.50600926 | 0.17452283 | 8.62929683 | 6.17E−18 | 3.61E−15 |
| ENSG00000164616 | 78.8328447 | 0.72579198 | 0.23383236 | 3.10389882 | 0.00190989 | 0.04990584 |
| ENSG00000152377 | 235.457097 | −0.8841583 | 0.18372693 | −4.8123498 | 1.49E−06 | 0.00011723 |
| ENSG00000146013 | 44.6522932 | −2.9975946 | 0.37242515 | −8.0488514 | 8.36E−16 | 3.72E−13 |
| ENSG00000120322 | 44.649573 | −0.9299721 | 0.29864774 | −3.1139432 | 0.00184605 | 0.04854781 |
| ENSG00000145819 | 381.969105 | −0.5593746 | 0.15019733 | −3.7242645 | 0.00019589 | 0.00784308 |
| ENSG00000183775 | 137.40831 | 1.44771279 | 0.2030624 | 7.12939846 | 1.01E−12 | 2.79E−10 |
| ENSG00000156475 | 454.434535 | 0.85874588 | 0.14053875 | 6.11038504 | 9.94E−10 | 1.47E−07 |
| ENSG00000157510 | 102.811079 | −0.8317177 | 0.20369563 | −4.0831394 | 4.44E−05 | 0.00225575 |
| ENSG00000164591 | 220.400344 | −1.0840681 | 0.16541908 | −6.5534649 | 5.62E−11 | 1.15E−08 |
| ENSG00000135074 | 340.949329 | 0.59247734 | 0.18873364 | 3.13922491 | 0.00169395 | 0.04552178 |
| ENSG00000275038 | 82.8887579 | −1.002962 | 0.2302139 | −4.3566528 | 1.32E−05 | 0.0007924 |
| ENSG00000164438 | 308.477332 | −0.9194887 | 0.24797402 | −3.708004 | 0.0002089 | 0.00829918 |
| ENSG00000214357 | 631.076015 | −0.5458835 | 0.16707604 | −3.267276 | 0.00108588 | 0.03215204 |
| ENSG00000120149 | 497.016479 | 0.71233223 | 0.11697043 | 6.0898488 | 1.13E−09 | 1.66E−07 |
| ENSG00000145920 | 1687.58993 | −0.7773923 | 0.13162745 | −5.9060044 | 3.51E−09 | 4.78E−07 |
| ENSG00000113763 | 1426.55683 | −0.7206216 | 0.19055476 | −3.7817034 | 0.00015576 | 0.00649048 |
| ENSG00000054598 | 3112.5844 | 0.83457975 | 0.15609105 | 5.34674958 | 8.95E−08 | 9.35E−06 |
| ENSG00000021355 | 92.0775704 | −0.7693576 | 0.21702089 | −3.5450855 | 0.00039249 | 0.01397127 |
| ENSG00000260604 | 17.1382489 | 2.09020479 | 0.5813081 | 3.59569183 | 0.00032353 | 0.01205726 |
| ENSG00000124782 | 68.0545973 | 0.9744693 | 0.28052205 | 3.47377073 | 0.0005132 | 0.01767687 |
| ENSG00000111859 | 386.637165 | 0.75853338 | 0.16415998 | 4.62069614 | 3.82E−06 | 0.00027076 |
| ENSG00000124788 | 65.2862974 | 0.89746949 | 0.23881542 | 3.75800476 | 0.00017127 | 0.00699453 |
| ENSG00000112183 | 45.702263 | 1.43229905 | 0.29205845 | 4.90415204 | 9.38E−07 | 7.74E−05 |
| ENSG00000172201 | 2146.62087 | 0.40229395 | 0.1260341 | 3.19194533 | 0.00141318 | 0.03960778 |
| ENSG00000152954 | 704.567276 | −0.5396052 | 0.10711235 | −5.0377495 | 4.71E−07 | 4.16E−05 |
| ENSG00000112294 | 461.110184 | −0.409296 | 0.12972842 | −3.1550214 | 0.00160486 | 0.0438186 |
| ENSG00000168405 | 156.226029 | 1.44176124 | 0.19317455 | 7.46351548 | 8.42E−14 | 2.74E−11 |
| ENSG00000079689 | 52.5215699 | 0.90354616 | 0.27893568 | 3.23926343 | 0.00119839 | 0.03502718 |
| ENSG00000168298 | 10.3556519 | −2.7878013 | 0.81854209 | −3.4058131 | 0.00065967 | 0.02173416 |
| ENSG00000278588 | 5.87633533 | −2.8721394 | 0.88857513 | −3.2322977 | 0.00122799 | 0.03558737 |
| ENSG00000276368 | 4.19759892 | −5.3145503 | 1.43816763 | −3.6953622 | 0.00021957 | 0.00863938 |
| ENSG00000184357 | 9.70561438 | −2.4161223 | 0.73878204 | −3.2704129 | 0.00107391 | 0.03195665 |
| ENSG00000219891 | 178.3972 | −1.0799893 | 0.21119396 | −5.113732 | 3.16E−07 | 2.92E−05 |
| ENSG00000280128 | 691.474973 | 0.51423518 | 0.15571074 | 3.302503 | 0.00095826 | 0.02930645 |
| ENSG00000048545 | 95.4558053 | −0.720382 | 0.22028672 | −3.2702015 | 0.00107471 | 0.03195665 |
| ENSG00000008196 | 8624.97581 | −0.7973905 | 0.09064247 | −8.7970954 | 1.40E−18 | 8.45E−16 |
| ENSG00000151917 | 298.832667 | 0.56060962 | 0.1707543 | 3.28313623 | 0.00102659 | 0.03084294 |
| ENSG00000079841 | 86.1193399 | −0.9310725 | 0.24455442 | −3.8072202 | 0.00014054 | 0.00601417 |
| ENSG00000118407 | 75.4408608 | 1.21809594 | 0.2566595 | 4.74596081 | 2.08E−06 | 0.00015706 |
| ENSG00000065833 | 442.718968 | −0.9985074 | 0.1452658 | −6.8736582 | 6.26E−12 | 1.49E−09 |
| ENSG00000112837 | 24.706155 | −3.0803448 | 0.54275032 | −5.6754363 | 1.38E−08 | 1.67E−06 |
| ENSG00000168830 | 52.0105116 | −1.4544217 | 0.2892544 | −5.0281749 | 4.95E−07 | 4.33E−05 |
| ENSG00000132429 | 222.043414 | −0.6365577 | 0.16599356 | −3.8348337 | 0.00012565 | 0.00548142 |
| ENSG00000111885 | 533.905017 | 0.75449727 | 0.17668028 | 4.27041017 | 1.95E−05 | 0.00112136 |
| ENSG00000118523 | 7.5593593 | 2.9298074 | 0.78140184 | 3.74942475 | 0.00017724 | 0.00720943 |
| ENSG00000182747 | 105.054008 | −1.3375564 | 0.22299865 | −5.998047 | 2.00E−09 | 2.90E−07 |
| ENSG00000016402 | 107.653048 | −1.5594668 | 0.24195057 | −6.4453941 | 1.15E−10 | 2.13E−08 |
| ENSG00000236366 | 9.7665036 | 2.84152587 | 0.7296275 | 3.89448846 | 9.84E−05 | 0.00446428 |
| ENSG00000001036 | 308.705221 | −0.6514658 | 0.14499267 | −4.4930947 | 7.02E−06 | 0.00046477 |
| ENSG00000152818 | 252.245921 | 0.90141545 | 0.22166963 | 4.06648156 | 4.77E−05 | 0.00239931 |
| ENSG00000146469 | 906.476132 | 0.9084221 | 0.18111349 | 5.01576176 | 5.28E−07 | 4.60E−05 |
| ENSG00000029639 | 343.599121 | −0.6432279 | 0.12508236 | −5.1424352 | 2.71E−07 | 2.59E−05 |
| ENSG00000092820 | 1434.97982 | −0.4548299 | 0.11842847 | −3.8405454 | 0.00012276 | 0.00537836 |
| ENSG00000176381 | 17.3808124 | −2.0558867 | 0.50113939 | −4.1024249 | 4.09E−05 | 0.00212736 |
| ENSG00000164850 | 182.544241 | 1.01748449 | 0.20196175 | 5.03800588 | 4.70E−07 | 4.16E−05 |
| ENSG00000003147 | 2985.59074 | −0.3680056 | 0.11476078 | −3.2067195 | 0.00134258 | 0.03809869 |
| ENSG00000106537 | 57.5341258 | −1.1165181 | 0.3116207 | −3.5829394 | 0.00033975 | 0.01252482 |
| ENSG00000173452 | 8.76484616 | −3.8550297 | 0.82420028 | −4.6772973 | 2.91E−06 | 0.00021393 |
| ENSG00000136235 | 63.8486506 | 1.00349655 | 0.24265055 | 4.1355626 | 3.54E−05 | 0.00187199 |
| ENSG00000122585 | 220.635473 | −2.4034495 | 0.21073447 | −11.405108 | 3.94E−30 | 7.33E−27 |
| ENSG00000214870 | 35.0895126 | −1.0415506 | 0.31466796 | −3.3099988 | 0.00093296 | 0.02874765 |
| ENSG00000122574 | 94.4781546 | −0.9920516 | 0.27224229 | −3.6440027 | 0.00026843 | 0.01024644 |
| ENSG00000164619 | 74.6718287 | 2.2782545 | 0.2333908 | 9.7615438 | 1.65E−22 | 1.68E−19 |
| ENSG00000002746 | 3.06591584 | −5.2314904 | 1.45125907 | −3.6047943 | 0.0003124 | 0.01170643 |
| ENSG00000058404 | 263.599497 | −0.758085 | 0.21151498 | −3.5840723 | 0.00033828 | 0.0124931 |
| ENSG00000146674 | 5646.90033 | 1.78165824 | 0.08632134 | 20.6398355 | 1.20E−94 | 2.46E−90 |
| ENSG00000132436 | 873.350924 | 0.53178507 | 0.15580711 | 3.41309893 | 0.00064229 | 0.0212985 |
| ENSG00000165215 | 8.98123693 | −2.2716534 | 0.69646926 | −3.2616706 | 0.00110758 | 0.03273442 |
| ENSG00000223705 | 788.12693 | 0.38566203 | 0.11921483 | 3.23501714 | 0.00121635 | 0.03545101 |
| ENSG00000188372 | 32.9492657 | −1.1907255 | 0.36295211 | −3.2806684 | 0.00103561 | 0.03106842 |
| ENSG00000186088 | 109.698772 | −1.8668694 | 0.26638186 | −7.0082451 | 2.41E−12 | 6.17E−10 |
| ENSG00000005471 | 42.7664223 | −1.8250515 | 0.34801461 | −5.2441808 | 1.57E−07 | 1.60E−05 |
| ENSG00000157240 | 729.091938 | 0.47226125 | 0.14512399 | 3.25419147 | 0.00113716 | 0.03338051 |
| ENSG00000177409 | 18.8112566 | 2.37990316 | 0.49734974 | 4.78517018 | 1.71E−06 | 0.00013043 |
| ENSG00000105825 | 923.984722 | −0.7762794 | 0.13677218 | −5.675711 | 1.38E−08 | 1.67E−06 |
| ENSG00000164692 | 67.8006859 | 1.21686718 | 0.253513 | 4.8000188 | 1.59E−06 | 0.00012342 |
| ENSG00000106236 | 901.594915 | −0.6659804 | 0.17248427 | −3.8611077 | 0.00011287 | 0.0050534 |
| ENSG00000166448 | 457.44415 | −0.5893807 | 0.15004602 | −3.9279999 | 8.57E−05 | 0.00392939 |
| ENSG00000087085 | 140.105007 | −1.3130826 | 0.25764775 | −5.0964256 | 3.46E−07 | 3.19E−05 |
| ENSG00000106366 | 241.882814 | 1.44627041 | 0.1570887 | 9.206712 | 3.36E−20 | 2.75E−17 |
| ENSG00000232445 | 6.49359521 | −3.3933555 | 1.07699323 | −3.1507677 | 0.00162842 | 0.04418761 |
| ENSG00000128606 | 263.914744 | 0.89036625 | 0.17627777 | 5.05092757 | 4.40E−07 | 3.95E−05 |
| ENSG00000091129 | 7233.87922 | −0.6197185 | 0.15933491 | −3.8894085 | 0.00010049 | 0.00454868 |
| ENSG00000173114 | 1896.30582 | 1.15105697 | 0.17275483 | 6.66295113 | 2.68E−11 | 5.84E−09 |
| ENSG00000135269 | 331.219696 | −1.4054226 | 0.17540025 | −8.0126603 | 1.12E−15 | 4.78E−13 |
| ENSG00000105976 | 111.044877 | −0.7852267 | 0.21361009 | −3.6759814 | 0.00023694 | 0.00914665 |
| ENSG00000106025 | 38.3910023 | −1.6092916 | 0.31988457 | −5.0308509 | 4.88E−07 | 4.29E−05 |
| ENSG00000008311 | 32.9700182 | 1.1321468 | 0.3462412 | 3.26982113 | 0.00107616 | 0.03195665 |
| ENSG00000234224 | 798.573377 | −0.8746577 | 0.11887693 | −7.3576743 | 1.87E−13 | 5.63E−11 |
| ENSG00000179603 | 207.096354 | −1.5809589 | 0.16835166 | −9.3908126 | 5.95E−21 | 5.30E−18 |
| ENSG00000128510 | 95.6419874 | 1.05481784 | 0.23812403 | 4.42969929 | 9.44E−06 | 0.00059957 |
| ENSG00000128567 | 919.311468 | −0.452819 | 0.11901936 | −3.8045826 | 0.00014204 | 0.00604202 |
| ENSG00000221866 | 4718.91804 | 0.89506269 | 0.23674411 | 3.78071785 | 0.00015638 | 0.00649153 |
| ENSG00000181072 | 835.972528 | 1.26608085 | 0.17247546 | 7.34064336 | 2.13E−13 | 6.30E−11 |
| ENSG00000105894 | 646.919988 | 0.6753613 | 0.16661087 | 4.05352495 | 5.05E−05 | 0.00250544 |
| ENSG00000122779 | 3956.8366 | 0.60494858 | 0.12581293 | 4.80831821 | 1.52E−06 | 0.00011886 |
| ENSG00000174469 | 385.825809 | −1.2236325 | 0.22030672 | −5.5542224 | 2.79E−08 | 3.22E−06 |
| ENSG00000127399 | 176.074863 | −2.016865 | 0.24889409 | −8.1033061 | 5.35E−16 | 2.61E−13 |
| ENSG00000188707 | 63.1050841 | −1.3836744 | 0.30165316 | −4.5869713 | 4.50E−06 | 0.0003162 |
| ENSG00000101846 | 209.594902 | 0.77392395 | 0.21824443 | 3.54613378 | 0.00039093 | 0.01395878 |
| ENSG00000047648 | 63.3214326 | 0.84109778 | 0.23584209 | 3.56635989 | 0.00036197 | 0.01310431 |
| ENSG00000188158 | 35.456595 | −1.0565152 | 0.3294047 | −3.2073471 | 0.00133965 | 0.03806846 |
| ENSG00000008086 | 1624.27696 | 1.30910266 | 0.17564482 | 7.45312417 | 9.12E−14 | 2.91E−11 |
| ENSG00000130066 | 106.297961 | 0.71656605 | 0.21096453 | 3.39661859 | 0.00068224 | 0.02222711 |
| ENSG00000198947 | 32.7971026 | −1.1043064 | 0.341453 | −3.2341389 | 0.0012201 | 0.03549036 |
| ENSG00000047597 | 17.2145698 | −1.905055 | 0.53265573 | −3.5765221 | 0.0003482 | 0.01269891 |
| ENSG00000189221 | 1885.52384 | 0.6717496 | 0.10353131 | 6.48837122 | 8.68E−11 | 1.63E−08 |
| ENSG00000069535 | 388.606137 | −0.765846 | 0.15979203 | −4.7927673 | 1.64E−06 | 0.00012653 |
| ENSG00000102007 | 498.64496 | −0.792672 | 0.15496903 | −5.1150347 | 3.14E−07 | 2.92E−05 |
| ENSG00000189299 | 161.855438 | 0.68132771 | 0.18973982 | 3.59085261 | 0.0003296 | 0.01219453 |
| ENSG00000242732 | 1189.90403 | −0.5118266 | 0.1310907 | −3.9043698 | 9.45E−05 | 0.00429529 |
| ENSG00000122145 | 33.2131924 | −1.1958497 | 0.33452979 | −3.5747182 | 0.00035061 | 0.01274499 |
| ENSG00000126947 | 197.286522 | −0.7928896 | 0.17915788 | −4.4256473 | 9.62E−06 | 0.00060347 |
| ENSG00000184905 | 137.561598 | −0.8235385 | 0.20235856 | −4.0696991 | 4.71E−05 | 0.00237225 |
| ENSG00000077279 | 2923.7225 | −0.9094433 | 0.20803635 | −4.3715594 | 1.23E−05 | 0.00074896 |
| ENSG00000260802 | 336.484212 | −2.0008207 | 0.21971116 | −9.1065956 | 8.50E−20 | 6.21E−17 |
| ENSG00000131724 | 269.819839 | 0.64056906 | 0.16238574 | 3.94473722 | 7.99E−05 | 0.00370235 |
| ENSG00000174460 | 181.377695 | −1.1839664 | 0.18188658 | −6.5093663 | 7.55E−11 | 1.44E−08 |
| ENSG00000125354 | 1607.08669 | −0.5258157 | 0.09235258 | −5.6935676 | 1.24E−08 | 1.52E−06 |
| ENSG00000125675 | 856.841699 | 0.6506577 | 0.14874404 | 4.37434454 | 1.22E−05 | 0.00074374 |
| ENSG00000009694 | 30.321651 | −2.044871 | 0.42496603 | −4.8118458 | 1.50E−06 | 0.00011723 |
| ENSG00000122121 | 36.4982624 | −1.099601 | 0.32190387 | −3.4159298 | 0.00063565 | 0.02111254 |
| ENSG00000147256 | 488.353731 | −1.9938837 | 0.14448713 | −13.799732 | 2.56E−43 | 7.48E−40 |
| ENSG00000171004 | 280.445427 | −2.1597234 | 0.17566856 | −12.294308 | 9.72E−35 | 2.21E−31 |
| ENSG00000223749 | 16.7834652 | 1.64862668 | 0.48369078 | 3.40843107 | 0.00065338 | 0.02159624 |
| ENSG00000155495 | 251.826393 | 0.88747822 | 0.17452831 | 5.08501016 | 3.68E−07 | 3.36E−05 |
| ENSG00000183837 | 131.105772 | −0.7002684 | 0.17729344 | −3.9497703 | 7.82E−05 | 0.00365413 |
| ENSG00000168939 | 61.3073861 | 0.91798647 | 0.27857807 | 3.29525751 | 0.00098332 | 0.02989395 |
| ENSG00000176595 | 806.922667 | −0.5935041 | 0.15581577 | −3.8090116 | 0.00013952 | 0.00599412 |
| ENSG00000173281 | 302.315743 | −0.7513752 | 0.18268861 | −4.1128735 | 3.91E−05 | 0.00204477 |
| ENSG00000171056 | 87.6629301 | −0.8984019 | 0.25109205 | −3.5779782 | 0.00034626 | 0.0126859 |
| ENSG00000269918 | 101.042073 | 0.82617427 | 0.22251557 | 3.71288295 | 0.00020491 | 0.0081725 |
| ENSG00000164733 | 3099.48503 | 0.33461802 | 0.09648489 | 3.46808721 | 0.00052418 | 0.01795012 |
| ENSG00000036565 | 326.703684 | −0.6922071 | 0.13917605 | −4.9736077 | 6.57E−07 | 5.63E−05 |
| ENSG00000061337 | 182.656634 | 0.72054612 | 0.18838231 | 3.82491396 | 0.00013082 | 0.00564669 |
| ENSG00000168546 | 549.377509 | 1.22471913 | 0.15267023 | 8.02199048 | 1.04E−15 | 4.53E−13 |
| ENSG00000158856 | 556.267729 | −0.6509255 | 0.15795952 | −4.1208371 | 3.77E−05 | 0.00199062 |
| ENSG00000168490 | 90.6265738 | −1.30024 | 0.2368019 | −5.4908345 | 4.00E−08 | 4.47E−06 |
| ENSG00000277586 | 4529.87486 | −0.5802311 | 0.08441013 | −6.8739503 | 6.24E−12 | 1.49E−09 |
| ENSG00000120915 | 302.252436 | −0.4836022 | 0.14004148 | −3.4532783 | 0.00055382 | 0.01876011 |
| ENSG00000171320 | 398.674485 | 0.5399997 | 0.1682092 | 3.21028633 | 0.00132603 | 0.03783896 |
| ENSG00000120875 | 2837.84888 | −1.1274038 | 0.10578123 | −10.657881 | 1.60E−26 | 2.73E−23 |
| ENSG00000251191 | 21.6618319 | −1.4659791 | 0.3980546 | −3.6828592 | 0.00023063 | 0.00895397 |
| ENSG00000133878 | 228.463086 | −1.1571629 | 0.17278839 | −6.6969945 | 2.13E−11 | 4.68E−09 |
| ENSG00000168619 | 3.40854659 | −5.3763523 | 1.48798115 | −3.6131858 | 0.00030246 | 0.0113654 |
| ENSG00000104332 | 1239.06173 | 0.92318161 | 0.10280598 | 8.97984303 | 2.71E−19 | 1.91E−16 |
| ENSG00000104368 | 135.935599 | 0.70967117 | 0.21024175 | 3.37550069 | 0.00073682 | 0.02366599 |
| ENSG00000104738 | 3569.65926 | 0.29803004 | 0.0838998 | 3.55221406 | 0.000382 | 0.01370764 |
| ENSGO0000019549 | 335.184999 | 0.68139437 | 0.16141318 | 4.22142951 | 2.43E−05 | 0.00134602 |
| ENSG00000254087 | 1214.33674 | −0.9893082 | 0.11675029 | −8.4737106 | 2.38E−17 | 1.28E−14 |
| ENSG00000198846 | 614.572592 | −0.5581506 | 0.12737699 | −4.3818797 | 1.18E−05 | 0.00072292 |
| ENSG00000104313 | 87.9807537 | −1.9027674 | 0.22059541 | −8.6255983 | 6.38E−18 | 3.62E−15 |
| ENSG00000250979 | 102.628872 | −0.9255325 | 0.23969244 | −3.8613337 | 0.00011277 | 0.0050534 |
| ENSG00000121039 | 203.999663 | 1.25780225 | 0.16530685 | 7.60889391 | 2.76E−14 | 9.92E−12 |
| ENSG00000164687 | 87.9084747 | −1.0778737 | 0.26206628 | −4.1129814 | 3.91E−05 | 0.00204477 |
| ENSG00000164949 | 345.504868 | −0.5559182 | 0.12982553 | −4.2820409 | 1.85E−05 | 0.00107335 |
| ENSG00000169439 | 1740.98641 | −0.6322354 | 0.12435174 | −5.0842506 | 3.69E−07 | 3.36E−05 |
| ENSG00000104361 | 46.7945725 | −1.089497 | 0.30996667 | −3.5148845 | 0.00043995 | 0.01546612 |
| ENSG00000164920 | 3.39128041 | 4.25176969 | 1.34679812 | 3.15694656 | 0.00159431 | 0.04360895 |
| ENSG00000174417 | 35.6471726 | 1.59498275 | 0.30459731 | 5.23636511 | 1.64E−07 | 1.66E−05 |
| ENSG00000147642 | 229.856075 | −0.6456978 | 0.16369767 | −3.9444533 | 8.00E−05 | 0.00370235 |
| ENSG00000136960 | 2294.842 | 0.58326289 | 0.14311425 | 4.07550548 | 4.59E−05 | 0.00232527 |
| ENSG00000170961 | 58.1172128 | 1.87852818 | 0.27665503 | 6.79014656 | 1.12E−11 | 2.55E−09 |
| ENSG00000156804 | 99.1248091 | 0.73449229 | 0.19717775 | 3.72502627 | 0.00019529 | 0.00783477 |
| ENSG00000169427 | 40.4467807 | −1.3041016 | 0.29787899 | −4.3779575 | 1.20E−05 | 0.00073384 |
| ENSG00000184489 | 331.969762 | −0.6058375 | 0.17548643 | −3.4523327 | 0.00055576 | 0.01876385 |
| ENSG00000204791 | 63.8605302 | −1.3450467 | 0.32466374 | −4.1428916 | 3.43E−05 | 0.00181783 |
| ENSG00000120217 | 55.4053328 | 1.14731598 | 0.30994138 | 3.70171923 | 0.00021414 | 0.00846671 |
| ENSG00000178445 | 638.532752 | −0.7731362 | 0.11954772 | −6.4671765 | 9.99E−11 | 1.86E−08 |
| ENSG00000153707 | 20.2647735 | −1.4559069 | 0.42809267 | −3.4009152 | 0.00067161 | 0.02198571 |
| ENSG00000265735 | 8.9140154 | −3.2654107 | 0.78625213 | −4.1531343 | 3.28E−05 | 0.00174737 |
| ENSG00000147872 | 567.234227 | −0.6143787 | 0.13039384 | −4.7117158 | 2.46E−06 | 0.00018342 |
| ENSG00000137142 | 1125.02577 | −0.6992121 | 0.16596364 | −4.2130437 | 2.52E−05 | 0.00139323 |
| ENSG00000119139 | 1436.36363 | −0.6526105 | 0.11290138 | −5.780359 | 7.45E−09 | 9.53E−07 |
| ENSG00000107282 | 650.047025 | 0.59860676 | 0.16172155 | 3.70146567 | 0.00021436 | 0.00846671 |
| ENSG00000198963 | 2500.95921 | 0.85895517 | 0.15961733 | 5.38134043 | 7.39E−08 | 7.88E−06 |
| ENSG00000106829 | 1338.91478 | 0.42591952 | 0.11485146 | 3.70843784 | 0.00020854 | 0.00829918 |
| ENSG00000165118 | 282.11308 | −1.0620083 | 0.16868969 | −6.2956327 | 3.06E−10 | 5.35E−08 |
| ENSG00000148053 | 1534.09037 | 1.26322726 | 0.20159538 | 6.26615184 | 3.70E−10 | 6.26E−08 |
| ENSG00000213694 | 440.652299 | 0.72288047 | 0.13729861 | 5.2650241 | 1.40E−07 | 1.43E−05 |
| ENSG00000165025 | 27.0018656 | −1.7588343 | 0.41813843 | −4.2063446 | 2.60E−05 | 0.00142361 |
| ENSG00000106785 | 153.560471 | −0.8489708 | 0.19089809 | −4.4472461 | 8.70E−06 | 0.00056138 |
| ENSG00000106789 | 223.375889 | −0.5679534 | 0.15990422 | −3.5518353 | 0.00038255 | 0.01370764 |
| ENSG00000095203 | 145.730021 | 0.51976579 | 0.16384112 | 3.17237698 | 0.00151197 | 0.04180382 |
| ENSG00000165124 | 56.1930901 | 0.87633268 | 0.27862987 | 3.14514983 | 0.00166002 | 0.04492588 |
| ENSG00000198121 | 507.784129 | 0.97713261 | 0.12821744 | 7.62090255 | 2.52E−14 | 9.20E−12 |
| ENSG00000259953 | 83.4455322 | −1.105382 | 0.24976441 | −4.4256986 | 9.61E−06 | 0.00060347 |
| ENSG00000106868 | 210.836834 | −0.7791446 | 0.17022699 | −4.5770921 | 4.71E−06 | 0.00032811 |
| ENSG00000196739 | 49.5614941 | 1.07399967 | 0.28776838 | 3.73216702 | 0.00018984 | 0.00765077 |
| ENSG00000136869 | 23.5821078 | 1.26404795 | 0.40229923 | 3.14205912 | 0.00167764 | 0.04528304 |
| ENSG00000078725 | 104.380945 | 1.08086608 | 0.19787437 | 5.46238538 | 4.70E−08 | 5.17E−06 |
| ENSG00000167081 | 1103.85411 | −0.4701654 | 0.11571104 | −4.0632716 | 4.84E−05 | 0.0024266 |
| ENSG00000095370 | 459.790834 | −0.5788442 | 0.18274163 | −3.1675552 | 0.00153726 | 0.04227479 |
| ENSG00000106991 | 776.329444 | 0.85640362 | 0.13432198 | 6.37575198 | 1.82E−10 | 3.30E−08 |
| ENSG00000148357 | 42.0771739 | −0.9502887 | 0.30273831 | −3.1389773 | 0.00169539 | 0.04552178 |
| ENSG00000197859 | 189.836049 | −0.9629967 | 0.26283575 | −3.6638725 | 0.00024843 | 0.00951852 |
| ENSG00000196990 | 561.984453 | −0.9215587 | 0.17619627 | −5.2302965 | 1.69E−07 | 1.71E−05 |
| ENSG00000123454 | 5584.01389 | −1.4226795 | 0.17316111 | −8.2159293 | 2.11E−16 | 1.08E−13 |
| ENSG00000130635 | 1058.21838 | 0.70947676 | 0.21332143 | 3.32585785 | 0.00088147 | 0.02736699 |
| ENSG00000176884 | 703.700719 | −0.8853723 | 0.20003932 | −4.4259912 | 9.60E−06 | 0.00060347 |
| ENSG00000233198 | 20.9028067 | 1.39807415 | 0.40816275 | 3.42528596 | 0.00061415 | 0.02046509 |
| ENSG00000148408 | 955.842409 | −0.6462563 | 0.1890012 | −3.4193239 | 0.00062777 | 0.02088482 |
| ENSG00000142102 | 94.0778321 | 0.83357512 | 0.23905833 | 3.48691098 | 0.00048863 | 0.01696242 |
| ENSG00000177106 | 26.6302263 | −1.1352254 | 0.36265877 | −3.1302853 | 0.00174637 | 0.04658494 |
| ENSG00000130600 | 2278.61782 | −2.8739514 | 0.16677993 | −17.231998 | 1.53E−66 | 6.25E−63 |
| ENSG00000167244 | 350.661076 | −4.2252963 | 0.23599691 | −17.904033 | 1.10E−71 | 7.48E−68 |
| ENSG00000180176 | 11.9667951 | −2.2478866 | 0.59541797 | −3.7753087 | 0.00015981 | 0.00660747 |
| ENSG00000170743 | 206.880941 | 0.64827938 | 0.17844832 | 3.6328691 | 0.00028029 | 0.01061243 |
| ENSG00000148926 | 179.840819 | −0.672519 | 0.18783794 | −3.5803154 | 0.00034318 | 0.01262852 |
| ENSG00000072952 | 133.171426 | −0.7210395 | 0.21606344 | −3.3371659 | 0.00084637 | 0.02643789 |
| ENSG00000050165 | 1293.35929 | −0.5822673 | 0.10878487 | −5.3524661 | 8.68E−08 | 9.10E−06 |
| ENSG00000133816 | 97.7105008 | 0.76800025 | 0.23982097 | 3.20238985 | 0.00136292 | 0.03851691 |
| ENSG00000197702 | 102.892164 | −1.2825649 | 0.23133305 | −5.5442353 | 2.95E−08 | 3.37E−06 |
| ENSG00000133794 | 724.030193 | 0.60253982 | 0.12920693 | 4.6633707 | 3.11E−06 | 0.00022489 |
| ENSG00000110693 | 57.4742111 | −1.2220078 | 0.26761699 | −4.5662564 | 4.97E−06 | 0.0003432 |
| ENSG00000187486 | 34.7064874 | −1.3240305 | 0.33357492 | −3.9692146 | 7.21E−05 | 0.00342313 |
| ENSG00000165973 | 49.8575099 | −1.2374025 | 0.29087881 | −4.254014 | 2.10E−05 | 0.00118675 |
| ENSG00000066382 | 443.273746 | −0.6304249 | 0.12689696 | −4.9680063 | 6.76E−07 | 5.77E−05 |
| ENSG00000148948 | 9.16297642 | −2.6958072 | 0.66355533 | −4.0626713 | 4.85E−05 | 0.0024269 |
| ENSG00000134569 | 315.67573 | −0.8753071 | 0.21585712 | −4.0550301 | 5.01E−05 | 0.00249542 |
| ENSG00000134574 | 534.744597 | −0.4561694 | 0.12448072 | −3.6645791 | 0.00024775 | 0.00951009 |
| ENSG00000149131 | 42.0433377 | −1.407112 | 0.31458786 | −4.4728746 | 7.72E−06 | 0.00050447 |
| ENSG00000166801 | 55.0137524 | 1.01278097 | 0.30700177 | 3.29894176 | 0.0009705 | 0.02959231 |
| ENSG00000124942 | 6938.49386 | 0.9413736 | 0.24716934 | 3.80861806 | 0.00013975 | 0.00599412 |
| ENSG00000168539 | 173.084657 | −1.1371713 | 0.17788084 | −6.3928825 | 1.63E−10 | 2.97E−08 |
| ENSG00000176485 | 220.189862 | −1.010832 | 0.22298274 | −4.5332297 | 5.81E−06 | 0.00039485 |
| ENSG00000245532 | 1327.15948 | 0.53380557 | 0.14454233 | 3.69307439 | 0.00022156 | 0.0086841 |
| ENSG00000179292 | 317.832848 | −0.7630976 | 0.20175901 | −3.7822232 | 0.00015543 | 0.00649016 |
| ENSG00000069482 | 877.295338 | −2.2788491 | 0.24927327 | −9.1419713 | 6.13E−20 | 4.65E−17 |
| ENSG00000132749 | 187.790801 | −1.2730811 | 0.17165236 | −7.4166245 | 1.20E−13 | 3.67E−11 |
| ENSG00000168010 | 235.149006 | 0.5888301 | 0.17876366 | 3.29390265 | 0.00098807 | 0.02999385 |
| ENSG00000175567 | 617.419822 | −0.4381215 | 0.13866792 | −3.1595013 | 0.00158039 | 0.04328629 |
| ENSG00000171533 | 1526.48309 | −0.5782375 | 0.11964 | −4.8331449 | 1.34E−06 | 0.00010699 |
| ENSG00000151376 | 43.6941144 | −1.0461764 | 0.33325268 | −3.1392887 | 0.00169358 | 0.04552178 |
| ENSG00000150687 | 306.524588 | 1.405811 | 0.14097138 | 9.97231474 | 2.01E−23 | 2.58E−20 |
| ENSG00000174804 | 94.7298601 | 0.70046157 | 0.21891475 | 3.19970014 | 0.00137571 | 0.03882338 |
| ENSG00000134627 | 5.15812255 | −3.3128796 | 1.05071911 | −3.1529641 | 0.00161622 | 0.04403169 |
| ENSG00000184384 | 73.7064213 | 0.963801 | 0.25250313 | 3.81698633 | 0.00013509 | 0.0058189 |
| ENSG00000137693 | 54.1219302 | 1.18122026 | 0.26813589 | 4.40530451 | 1.06E−05 | 0.00065693 |
| ENSG00000204381 | 351.076377 | 0.844782 | 0.13722853 | 6.15602289 | 7.46E−10 | 1.15E−07 |
| ENSG00000149295 | 200.587314 | −0.7424929 | 0.17230624 | −4.309147 | 1.64E−05 | 0.00096224 |
| ENSG00000236437 | 30.7605626 | −1.1387896 | 0.34769669 | −3.2752385 | 0.00105573 | 0.03148717 |
| ENSG00000149591 | 378.668326 | 1.11538712 | 0.13728124 | 8.12483298 | 4.48E−16 | 2.24E−13 |
| ENSG00000110400 | 1066.09289 | −0.5495862 | 0.15290126 | −3.5943861 | 0.00032516 | 0.01209585 |
| ENSG00000149403 | 112.702881 | −1.1558102 | 0.22518431 | −5.1327296 | 2.86E−07 | 2.69E−05 |
| ENSG00000255248 | 895.120805 | 0.72147549 | 0.19573117 | 3.68605307 | 0.00022776 | 0.00889303 |
| ENSG00000255545 | 42.0985936 | −0.9382482 | 0.28439807 | −3.2990668 | 0.00097007 | 0.02959231 |
| ENSG00000185736 | 19.4709746 | −1.3107267 | 0.41943905 | −3.1249514 | 0.00177834 | 0.04725314 |
| ENSG00000134463 | 139.52805 | −0.9905176 | 0.21380995 | −4.6327012 | 3.61E−06 | 0.00025641 |
| ENSG00000151468 | 76.7451882 | 0.96503068 | 0.23462881 | 4.11301022 | 3.91E−05 | 0.00204477 |
| ENSG00000065809 | 2559.54356 | 0.42494044 | 0.12642299 | 3.36125913 | 0.00077588 | 0.0247251 |
| ENSG00000026025 | 1891.26677 | 0.88573758 | 0.17439216 | 5.07899879 | 3.79E−07 | 3.44E−05 |
| ENSG00000120594 | 201.133733 | 1.78014212 | 0.22703988 | 7.84065819 | 4.48E−15 | 1.73E−12 |
| ENSG00000099256 | 101.234018 | −1.2676506 | 0.24548056 | −5.1639549 | 2.42E−07 | 2.34E−05 |
| ENSG00000095739 | 1191.41474 | 0.58435836 | 0.15265252 | 3.82802945 | 0.00012917 | 0.00560128 |
| ENSG00000165757 | 42.5372107 | 0.95745529 | 0.30794178 | 3.10920874 | 0.00187589 | 0.04920607 |
| ENSG00000099250 | 160.354994 | 1.06270836 | 0.18938081 | 5.61148908 | 2.01E−08 | 2.35E−06 |
| ENSG00000177283 | 57.7473802 | 1.0451468 | 0.326863 | 3.19750718 | 0.00138621 | 0.03901217 |
| ENSG00000107562 | 44.0614137 | 1.12430651 | 0.30905819 | 3.63784737 | 0.00027493 | 0.01043598 |
| ENSG00000204175 | 15.9318685 | −2.5480581 | 0.6124735 | −4.1602749 | 3.18E−05 | 0.00169804 |
| ENSG00000128805 | 128.852477 | −0.6991017 | 0.20843329 | −3.3540787 | 0.0007963 | 0.02522018 |
| ENSG00000165633 | 182.739294 | −0.5858711 | 0.18681959 | −3.1360261 | 0.00171254 | 0.0459221 |
| ENSG00000165606 | 80.2168493 | −0.9832371 | 0.23519812 | −4.1804632 | 2.91E−05 | 0.00158302 |
| ENSG00000226389 | 4.43116604 | −4.6798887 | 1.24453489 | −3.7603516 | 0.00016967 | 0.00694309 |
| ENSG00000166228 | 939.852475 | −0.5425283 | 0.15657113 | −3.4650593 | 0.00053011 | 0.01810709 |
| ENSG00000107742 | 576.43466 | −1.1816549 | 0.16451393 | −7.1827042 | 6.83E−13 | 1.92E−10 |
| ENSG00000185737 | 61.0041287 | 0.76205695 | 0.240105 | 3.17384876 | 0.00150432 | 0.04170515 |
| ENSG00000198682 | 173.849685 | 0.61347477 | 0.18983323 | 3.23165118 | 0.00123077 | 0.03561753 |
| ENSG00000171862 | 7241.27046 | 0.36947742 | 0.118736 | 3.11175574 | 0.00185978 | 0.04884616 |
| ENSG00000107796 | 145.346625 | 0.91961522 | 0.19727361 | 4.66162317 | 3.14E−06 | 0.00022601 |
| ENSG00000119917 | 45.112253 | 0.91305465 | 0.28774719 | 3.17311405 | 0.00150813 | 0.04175426 |
| ENSG00000148677 | 89.7163902 | 2.45976537 | 0.24868171 | 9.89121946 | 4.54E−23 | 5.47E−20 |
| ENSG00000138119 | 38.6238282 | 1.18363606 | 0.37191141 | 3.18257525 | 0.00145972 | 0.04078705 |
| ENSG00000138193 | 431.352652 | 1.0505424 | 0.1798728 | 5.84047391 | 5.21E−09 | 6.83E−07 |
| ENSG00000187122 | 962.018273 | −0.9566178 | 0.18500562 | −5.17075 | 2.33E−07 | 2.27E−05 |
| ENSG00000107521 | 430.649079 | −0.478784 | 0.1423137 | −3.3642864 | 0.00076742 | 0.02453341 |
| ENSG00000119946 | 384.692257 | −0.8821252 | 0.17977392 | −4.9068585 | 9.25E−07 | 7.67E−05 |
| ENSG00000099194 | 7740.70394 | −0.4198838 | 0.13394528 | −3.134741 | 0.00172006 | 0.04606336 |
| ENSG00000235823 | 210.386201 | −0.9045229 | 0.17063386 | −5.3009579 | 1.15E−07 | 1.18E−05 |
| ENSG00000156398 | 208.918663 | 0.75880173 | 0.15332495 | 4.9489776 | 7.46E−07 | 6.26E−05 |
| ENSG00000156395 | 9.83231369 | −2.3358115 | 0.71044427 | −3.2878181 | 0.00100967 | 0.03046882 |
| ENSG00000150594 | 61.3664637 | 2.03454631 | 0.25268017 | 8.05186376 | 8.15E−16 | 3.71E−13 |
| ENSG00000148737 | 119.95062 | 0.67749074 | 0.183946 | 3.68309572 | 0.00023042 | 0.00895397 |
| ENSG00000165868 | 1024.44815 | −0.6447037 | 0.1381039 | −4.6682516 | 3.04E−06 | 0.00022118 |
| ENSG00000187164 | 448.261163 | −0.5862802 | 0.1303868 | −4.4964694 | 6.91E−06 | 0.00046046 |
| ENSG00000119973 | 22.2654684 | −3.2579451 | 0.57790951 | −5.6374659 | 1.73E−08 | 2.04E−06 |
| ENSG00000188613 | 319.726228 | −1.1537704 | 0.21345561 | −5.4052007 | 6.47E−08 | 6.93E−06 |
| ENSG00000198873 | 343.660662 | −0.8441334 | 0.14433093 | −5.8485967 | 4.96E−09 | 6.54E−07 |
| ENSG00000066468 | 211.837141 | 1.06565625 | 0.19370748 | 5.50136867 | 3.77E−08 | 4.24E−06 |
| ENSG00000166033 | 392.540148 | 0.8215438 | 0.15053516 | 5.45748789 | 4.83E−08 | 5.28E−06 |
| ENSG00000148848 | 678.219215 | −1.6765076 | 0.18889972 | −8.87512 | 6.99E−19 | 4.61E−16 |
| ENSG00000132334 | 553.175928 | 0.65125512 | 0.14246898 | 4.57120646 | 4.85E−06 | 0.00033632 |
| ENSG00000227076 | 11.6114077 | 2.45716753 | 0.69988726 | 3.5108048 | 0.00044675 | 0.01567848 |
| ENSG00000188385 | 129.617311 | −0.5814608 | 0.18110651 | −3.2106014 | 0.00132458 | 0.03783896 |
| ENSG00000111664 | 79.960979 | 0.74672694 | 0.21686898 | 3.4432169 | 0.00057484 | 0.019327 |
| ENSG00000139112 | 196.810607 | −0.4923966 | 0.1568431 | −3.1394214 | 0.00169282 | 0.04552178 |
| ENSG00000261324 | 36.0586595 | −1.318994 | 0.42106855 | −3.1324923 | 0.00173329 | 0.04635701 |
| ENSG00000197837 | 12.3447671 | −1.8816968 | 0.56412896 | −3.3355792 | 0.00085122 | 0.02651753 |
| ENSG00000084453 | 98.6224656 | −0.6986212 | 0.21985082 | −3.1777058 | 0.00148445 | 0.04126618 |
| ENSG00000121361 | 164.493102 | −0.7119582 | 0.17074874 | −4.1696251 | 3.05E−05 | 0.00164273 |
| ENSG00000111728 | 53.8112263 | −1.817966 | 0.29412162 | −6.1810009 | 6.37E−10 | 1.01E−07 |
| ENSG00000123094 | 939.036092 | 0.76182808 | 0.17477369 | 4.35894027 | 1.31E−05 | 0.00078647 |
| ENSG00000029153 | 93.6746801 | 0.79664513 | 0.23252924 | 3.42599982 | 0.00061254 | 0.02044467 |
| ENSG00000151233 | 674.323745 | 0.55313776 | 0.16333921 | 3.38643579 | 0.00070807 | 0.02289731 |
| ENSG00000139174 | 718.777878 | 0.70068107 | 0.121674 | 5.75867543 | 8.48E−09 | 1.08E−06 |
| ENSG00000184613 | 493.915011 | −0.8505901 | 0.11651138 | −7.3004898 | 2.87E−13 | 8.34E−11 |
| ENSG00000186897 | 77.3288231 | −1.1235162 | 0.26478415 | −4.2431399 | 2.20E−05 | 0.00123892 |
| ENSG00000135472 | 331.003362 | −0.7279623 | 0.15628419 | −4.6579395 | 3.19E−06 | 0.00022849 |
| ENSG00000111057 | 42.6287927 | 1.45041354 | 0.33764714 | 4.29564878 | 1.74E−05 | 0.00101823 |
| ENSG00000161638 | 36.0053856 | 1.30104566 | 0.31251067 | 4.16320396 | 3.14E−05 | 0.0016852 |
| ENSG00000139263 | 9.05624296 | 2.2576556 | 0.62396129 | 3.6182623 | 0.00029659 | 0.01117529 |
| ENSG00000153179 | 676.051775 | −0.4057481 | 0.11925435 | −3.4023753 | 0.00066803 | 0.02193878 |
| ENSG00000111490 | 494.371974 | −0.5542979 | 0.14393408 | −3.851054 | 0.00011761 | 0.00520183 |
| ENSG00000127328 | 268.979342 | −0.6057071 | 0.18200947 | −3.3278877 | 0.00087507 | 0.02720966 |
| ENSG00000139329 | 337.634661 | 1.05158084 | 0.20244289 | 5.19445675 | 2.05E−07 | 2.04E−05 |
| ENSG00000011465 | 1260.14348 | 1.67246862 | 0.14025318 | 11.92464 | 8.81E−33 | 1.80E−29 |
| ENSG00000139352 | 419.554476 | −0.7659843 | 0.16726321 | −4.5795146 | 4.66E−06 | 0.00032544 |
| ENSG00000171310 | 227.089771 | −0.5619836 | 0.17544324 | −3.2032219 | 0.00135899 | 0.03851105 |
| ENSG00000074590 | 126.923559 | 0.807529 | 0.20470406 | 3.94486066 | 7.98E−05 | 0.00370235 |
| ENSG00000151136 | 253.165693 | −1.249564 | 0.16592275 | −7.5309988 | 5.04E−14 | 1.78E−11 |
| ENSG00000139445 | 208.425737 | −0.6421852 | 0.1616363 | −3.9730257 | 7.10E−05 | 0.0033845 |
| ENSG00000111331 | 304.193388 | −1.0003117 | 0.18668949 | −5.3581578 | 8.41E−08 | 8.87E−06 |
| ENSG00000135111 | 8429.45307 | 0.59781983 | 0.12597729 | 4.74545723 | 2.08E−06 | 0.00015706 |
| ENSG00000089250 | 42.8332161 | −1.4697069 | 0.3820435 | −3.8469622 | 0.00011959 | 0.00526202 |
| ENSG00000152137 | 25.0512144 | 1.28828052 | 0.39288957 | 3.27898885 | 0.0010418 | 0.03118056 |
| ENSG00000135127 | 283.131342 | −0.5716832 | 0.15978962 | −3.5777241 | 0.0003466 | 0.0126859 |
| ENSG00000151948 | 255.025187 | −0.6928733 | 0.16369019 | −4.2328334 | 2.31E−05 | 0.00129002 |
| ENSG00000125207 | 275.826415 | −0.684731 | 0.1920182 | −3.5659691 | 0.00036251 | 0.01310431 |
| ENSG00000165480 | 470.356075 | 0.53406289 | 0.15700611 | 3.40154215 | 0.00067007 | 0.0219705 |
| ENSG00000133121 | 48.35548 | −1.1141537 | 0.31576172 | −3.5284636 | 0.00041798 | 0.01482125 |
| ENSG00000133101 | 43.6629608 | −1.3852886 | 0.32598508 | −4.249546 | 2.14E−05 | 0.00120733 |
| ENSG00000120693 | 2836.72911 | 0.52012227 | 0.14916372 | 3.48692212 | 0.00048861 | 0.01696242 |
| ENSG00000183722 | 79.0296604 | 0.93547647 | 0.22373181 | 4.18124035 | 2.90E−05 | 0.00158182 |
| ENSG00000120675 | 188.807802 | −0.6771897 | 0.18931312 | −3.5770883 | 0.00034744 | 0.01269407 |
| ENSG00000136161 | 206.091876 | 0.50277323 | 0.1552854 | 3.23773663 | 0.00120482 | 0.03516493 |
| ENSG00000184226 | 408.418256 | 0.76874738 | 0.22873624 | 3.36084648 | 0.00077704 | 0.0247251 |
| ENSG00000178695 | 2729.12734 | 1.25707719 | 0.1418735 | 8.86054981 | 7.96E−19 | 5.09E−16 |
| ENSG00000136158 | 696.45312 | −0.6313856 | 0.1443585 | −4.3737337 | 1.22E−05 | 0.00074374 |
| ENSG00000184564 | 706.344588 | 1.40670638 | 0.17841599 | 7.88441898 | 3.16E−15 | 1.24E−12 |
| ENSG00000165300 | 32.3713864 | −2.5519864 | 0.40766081 | −6.2600729 | 3.85E−10 | 6.45E−08 |
| ENSG00000224394 | 2.65943959 | 5.73536063 | 1.52324641 | 3.76522183 | 0.0001664 | 0.00683649 |
| ENSG00000139800 | 24.5554138 | 1.76569175 | 0.37903754 | 4.65835591 | 3.19E−06 | 0.00022849 |
| ENSG00000043355 | 61.3768772 | 1.33930462 | 0.24277325 | 5.51668932 | 3.45E−08 | 3.90E−06 |
| ENSG00000125266 | 1816.3175 | −0.8217977 | 0.104066 | −7.8968895 | 2.86E−15 | 1.15E−12 |
| ENSG00000204442 | 1698.86651 | −0.3719696 | 0.09073483 | −4.0995243 | 4.14E−05 | 0.00212736 |
| ENSG00000274718 | 105.845859 | −1.3310915 | 0.22244929 | −5.9837976 | 2.18E−09 | 3.12E−07 |
| ENSG00000126218 | 324.763949 | −0.5286935 | 0.14913867 | −3.5449797 | 0.00039264 | 0.01397127 |
| ENSG00000129474 | 184.128257 | 0.5619735 | 0.17231201 | 3.26137158 | 0.00110875 | 0.03273442 |
| ENSG00000100473 | 180.329873 | −0.7195304 | 0.17359948 | −4.1447727 | 3.40E−05 | 0.00180766 |
| ENSG00000129493 | 498.332842 | 0.79370552 | 0.14456889 | 5.49015446 | 4.02E−08 | 4.47E−06 |
| ENSG00000259017 | 37.315656 | −2.4498274 | 0.37719921 | −6.4947839 | 8.32E−11 | 1.58E−08 |
| ENSG00000168348 | 403.695123 | −3.0917201 | 0.17715282 | −17.452277 | 3.31E−68 | 1.69E−64 |
| ENSG00000139926 | 565.981844 | 0.40678608 | 0.12358727 | 3.29148859 | 0.00099659 | 0.03020765 |
| ENSG00000020577 | 261.2629 | −0.5981446 | 0.19101048 | −3.1314754 | 0.0017393 | 0.04645711 |
| ENSG00000131979 | 905.131557 | −1.2948008 | 0.13500463 | −9.5907879 | 8.74E−22 | 8.52E−19 |
| ENSG00000131981 | 209.424129 | −1.1133368 | 0.18730729 | −5.9439052 | 2.78E−09 | 3.90E−07 |
| ENSG00000184302 | 41.7935368 | −1.0202403 | 0.323995 | −3.1489383 | 0.00163865 | 0.04440627 |
| ENSG00000126803 | 367.472833 | −0.5495432 | 0.16161098 | −3.4004076 | 0.00067285 | 0.02199138 |
| ENSG00000070182 | 86.268933 | −1.2407894 | 0.2720721 | −4.5605171 | 5.10E−06 | 0.00035152 |
| ENSG00000197555 | 1008.87888 | −0.5853328 | 0.18417495 | −3.1781344 | 0.00148226 | 0.04126128 |
| ENSG00000184227 | 33.5540176 | −1.1835226 | 0.3755763 | −3.1512176 | 0.00162591 | 0.04418761 |
| ENSG00000119673 | 220.222994 | −1.390438 | 0.19276414 | −7.2131572 | 5.47E−13 | 1.55E−10 |
| ENSG00000119630 | 124.500941 | −1.1238045 | 0.22254925 | −5.0496889 | 4.43E−07 | 3.95E−05 |
| ENSG00000119699 | 49.5649341 | 0.95102582 | 0.26604773 | 3.57464366 | 0.00035071 | 0.01274499 |
| ENSG00000021645 | 134.759132 | 0.78400733 | 0.21274798 | 3.68514585 | 0.00022857 | 0.00890777 |
| ENSG00000119714 | 70.1232904 | −1.0503013 | 0.28644199 | −3.6667157 | 0.00024569 | 0.00944874 |
| ENSG00000100604 | 8593.89536 | −1.7799798 | 0.18109061 | −9.8292219 | 8.43E−23 | 9.58E−20 |
| ENSG00000250366 | 301.586973 | −0.5380763 | 0.15337958 | −3.508135 | 0.00045126 | 0.01580956 |
| ENSG00000140057 | 85.0509544 | −1.1604083 | 0.22663043 | −5.1202671 | 3.05E−07 | 2.85E−05 |
| ENSG00000182218 | 44.6313937 | −1.7846673 | 0.31244547 | −5.711932 | 1.12E−08 | 1.39E−06 |
| ENSG00000183092 | 1514.42165 | −1.2080436 | 0.20556996 | −5.8765572 | 4.19E−09 | 5.60E−07 |
| ENSG00000259031 | 31.9495195 | −1.6987083 | 0.38219206 | −4.4446456 | 8.80E−06 | 0.00056465 |
| ENSG00000185559 | 1605.2048 | −4.2146341 | 0.2285781 | −18.438486 | 6.45E−76 | 6.60E−72 |
| ENSG00000254656 | 1169.57968 | −3.2671826 | 0.25858884 | −12.634662 | 1.36E−36 | 3.48E−33 |
| ENSG00000221077 | 55.3196793 | −2.6149746 | 0.42016449 | −6.2236925 | 4.86E−10 | 7.95E−08 |
| ENSG00000197406 | 37.4427261 | −1.1973924 | 0.3587037 | −3.3381099 | 0.0008435 | 0.02642892 |
| ENSG00000182636 | 47.7319055 | 1.00091417 | 0.2868019 | 3.4899147 | 0.00048317 | 0.01686989 |
| ENSG00000224078 | 979.413625 | 0.64904807 | 0.17167626 | 3.78065128 | 0.00015642 | 0.00649153 |
| ENSG00000166922 | 212.210229 | −1.3919089 | 0.20003831 | −6.9582118 | 3.45E−12 | 8.70E−10 |
| ENSG00000166923 | 561.836783 | 0.77386839 | 0.16126069 | 4.79886563 | 1.60E−06 | 0.00012366 |
| ENSG00000248905 | 480.325555 | 0.74959203 | 0.22728722 | 3.29799471 | 0.00097378 | 0.02964811 |
| ENSG00000198838 | 45.5309244 | 1.23397969 | 0.33074586 | 3.73089989 | 0.0001908 | 0.00766937 |
| ENSG00000175265 | 915.601957 | 0.48942159 | 0.14267531 | 3.43031741 | 0.00060288 | 0.02015496 |
| ENSG00000215252 | 752.192855 | 0.51215027 | 0.13301538 | 3.85030867 | 0.00011797 | 0.00520183 |
| ENSG00000166073 | 236.735163 | −0.6883191 | 0.16353375 | −4.2090338 | 2.56E−05 | 0.00141436 |
| ENSG00000104081 | 549.18298 | 0.56950897 | 0.14111911 | 4.03566144 | 5.44E−05 | 0.00269087 |
| ENSG00000166145 | 42.5743607 | −1.0009252 | 0.28897 | −3.4637686 | 0.00053266 | 0.01816386 |
| ENSG00000171766 | 85.6269017 | −1.023402 | 0.21703402 | −4.715399 | 2.41E−06 | 0.0001808 |
| ENSG00000137872 | 259.338354 | −0.5484705 | 0.17644877 | −3.1083836 | 0.00188114 | 0.04928049 |
| ENSG00000140285 | 59.278884 | 1.02579492 | 0.2649428 | 3.87175991 | 0.00010805 | 0.00488025 |
| ENSG00000140284 | 31.4701904 | −1.3857682 | 0.3991136 | −3.4721148 | 0.00051638 | 0.01775637 |
| ENSG00000140416 | 1362.61801 | 1.09482316 | 0.11972006 | 9.14485956 | 5.97E−20 | 4.65E−17 |
| ENSG00000166831 | 225.311897 | −1.08244 | 0.16474506 | −6.5703944 | 5.02E−11 | 1.05E−08 |
| ENSG00000103742 | 67.3580725 | −1.3950965 | 0.25742219 | −5.4194883 | 5.98E−08 | 6.44E−06 |
| ENSG00000137834 | 532.719059 | 1.23988913 | 0.20163022 | 6.14932203 | 7.78E−10 | 1.19E−07 |
| ENSG00000137809 | 50.5178333 | 1.26620757 | 0.32018927 | 3.95455969 | 7.67E−05 | 0.00359813 |
| ENSG00000187720 | 45.223503 | 1.350013 | 0.31493497 | 4.28664056 | 1.81E−05 | 0.00105436 |
| ENSG00000129009 | 166.963065 | 0.84941533 | 0.19330303 | 4.39421641 | 1.11E−05 | 0.00068719 |
| ENSG00000137868 | 1887.7781 | 0.4511993 | 0.14066983 | 3.2075058 | 0.00133891 | 0.03806846 |
| ENSG00000140538 | 153.325231 | 1.5366654 | 0.21696744 | 7.08247012 | 1.42E−12 | 3.76E−10 |
| ENSG00000166825 | 163.700948 | −0.8078366 | 0.18529068 | −4.3598339 | 1.30E−05 | 0.00078557 |
| ENSG00000182175 | 130.282564 | −0.6791887 | 0.20539082 | −3.3068111 | 0.00094364 | 0.02894599 |
| ENSG00000140563 | 60.8301012 | −0.9209633 | 0.27008931 | −3.4098472 | 0.00064999 | 0.02151918 |
| ENSG00000182253 | 354.654633 | 0.68180562 | 0.15887198 | 4.29154092 | 1.77E−05 | 0.00103429 |
| ENSG00000140470 | 105.686944 | 1.13372895 | 0.21092804 | 5.37495608 | 7.66E−08 | 8.12E−06 |
| ENSG00000154237 | 203.095762 | −0.885372 | 0.23712892 | −3.733716 | 0.00018868 | 0.00762904 |
| ENSG00000076344 | 96.8156534 | −0.814014 | 0.25672193 | −3.1708003 | 0.0015202 | 0.04197464 |
| ENSG00000162004 | 167.876855 | 0.64098529 | 0.20226376 | 3.16905647 | 0.00152935 | 0.04211364 |
| ENSG00000162039 | 29.3439755 | 1.08726752 | 0.33387277 | 3.25653245 | 0.00112782 | 0.03315403 |
| ENSG00000127561 | 384.402877 | −0.6540937 | 0.19541045 | −3.3472808 | 0.00081608 | 0.02568784 |
| ENSG00000183971 | 111.787712 | −1.0254945 | 0.29722968 | −3.4501752 | 0.00056022 | 0.01888329 |
| ENSG00000184697 | 11.9377047 | −2.3082357 | 0.66525285 | −3.4697118 | 0.00052102 | 0.01788592 |
| ENSG00000118898 | 33.6793528 | −1.0009438 | 0.31537017 | −3.1738696 | 0.00150421 | 0.04170515 |
| ENSG00000263013 | 16.7559205 | 1.70156384 | 0.54126522 | 3.1436785 | 0.00166839 | 0.04509271 |
| ENSG00000156968 | 170.165813 | −0.8952841 | 0.19647903 | −4.5566397 | 5.20E−06 | 0.00035568 |
| ENSG00000103528 | 163.752668 | −0.5666376 | 0.17961304 | −3.1547687 | 0.00160625 | 0.0438186 |
| ENSG00000280180 | 71.1018865 | −1.3689171 | 0.25181962 | −5.4361018 | 5.45E−08 | 5.90E−06 |
| ENSG00000261010 | 4.27391463 | −4.5652019 | 1.38048715 | −3.3069499 | 0.00094318 | 0.02894599 |
| ENSG00000260153 | 9.4798296 | −1.8474627 | 0.56356395 | −3.2781775 | 0.0010448 | 0.03120663 |
| ENSG00000103546 | 1110.13289 | −0.6354925 | 0.19337434 | −3.2863329 | 0.00101501 | 0.03058483 |
| ENSG00000278928 | 309.214394 | −0.5860704 | 0.14729022 | −3.9790181 | 6.92E−05 | 0.00331579 |
| ENSG00000159713 | 149.199141 | −0.8480384 | 0.22723391 | −3.7320065 | 0.00018996 | 0.00765077 |
| ENSG00000103154 | 145.991349 | 1.7660909 | 0.22943511 | 7.69756157 | 1.39E−14 | 5.16E−12 |
| ENSG00000003249 | 1067.10577 | −0.4526685 | 0.13462595 | −3.3624162 | 0.00077264 | 0.02466167 |
| ENSG00000183688 | 894.748706 | −0.7536021 | 0.10873035 | −6.9309268 | 4.18E−12 | 1.04E−09 |
| ENSG00000083454 | 35.8834964 | −1.1615529 | 0.35642283 | −3.2589184 | 0.00111838 | 0.03292377 |
| ENSG00000108515 | 255.119942 | −0.6956288 | 0.17015606 | −4.088181 | 4.35E−05 | 0.00221278 |
| ENSG00000129250 | 440.185404 | 0.60072326 | 0.14673643 | 4.09389316 | 4.24E−05 | 0.00216973 |
| ENSG00000179111 | 51.1174922 | −1.1591315 | 0.30513716 | −3.7987228 | 0.00014544 | 0.00613562 |
| ENSG00000179277 | 66.7642074 | −1.1129834 | 0.3223653 | −3.4525535 | 0.00055531 | 0.01876385 |
| ENSG00000265519 | 33.6115655 | −1.2155018 | 0.34051764 | −3.5695708 | 0.00035757 | 0.0129713 |
| ENSG00000072310 | 654.026218 | −0.7466256 | 0.18733072 | −3.9856015 | 6.73E−05 | 0.00324035 |
| ENSG00000166482 | 2055.91698 | 0.60166955 | 0.13203714 | 4.55682059 | 5.19E−06 | 0.00035568 |
| ENSG00000109107 | 576.969647 | −0.7460971 | 0.15918812 | −4.6868891 | 2.77E−06 | 0.00020563 |
| ENSG00000131242 | 1147.67375 | −0.4404608 | 0.13977874 | −3.1511287 | 0.00162641 | 0.04418761 |
| ENSG00000270765 | 11.2539055 | −1.6257588 | 0.5056101 | −3.2154397 | 0.00130245 | 0.03737461 |
| ENSG00000271447 | 27.9320433 | −1.5238241 | 0.38265576 | −3.9822323 | 6.83E−05 | 0.00327893 |
| ENSG00000141744 | 44.3407436 | −2.3116271 | 0.40475173 | −5.7112222 | 1.12E−08 | 1.39E−06 |
| ENSG00000141753 | 1494.95716 | −0.6803248 | 0.1869395 | −3.6392779 | 0.0002734 | 0.01039747 |
| ENSG00000167920 | 98.9983987 | 0.75116484 | 0.22060433 | 3.40503217 | 0.00066156 | 0.02176137 |
| ENSG00000126561 | 29.3954495 | −1.4384824 | 0.42574809 | −3.3787173 | 0.00072825 | 0.02346451 |
| ENSG00000177469 | 274.16205 | 0.78791211 | 0.15111705 | 5.21391941 | 1.85E−07 | 1.85E−05 |
| ENSG00000214578 | 18.0271484 | −1.7723109 | 0.48211714 | −3.6761001 | 0.00023683 | 0.00914665 |
| ENSG00000197291 | 78.5473719 | −0.8960861 | 0.22065636 | −4.0610027 | 4.89E−05 | 0.00243835 |
| ENSG00000131477 | 79.2047579 | −1.2949478 | 0.26555644 | −4.8763561 | 1.08E−06 | 8.81E−05 |
| ENSG00000108828 | 3513.0979 | −0.4848806 | 0.1267232 | −3.8262972 | 0.00013009 | 0.00562694 |
| ENSG00000108830 | 612.593983 | −0.4274075 | 0.13429738 | −3.1825455 | 0.00145987 | 0.04078705 |
| ENSG00000108861 | 614.956278 | −0.4466661 | 0.11755131 | −3.7997546 | 0.00014484 | 0.00612276 |
| ENSG00000131094 | 325.411614 | −1.4580131 | 0.25833783 | −5.6438233 | 1.66E−08 | 1.98E−06 |
| ENSG00000186868 | 1366.26959 | −0.6908354 | 0.12925751 | −5.344644 | 9.06E−08 | 9.41E−06 |
| ENSG00000064300 | 399.394153 | 0.94290714 | 0.14823507 | 6.36089106 | 2.01E−10 | 3.57E−08 |
| ENSG00000108846 | 38.383169 | 1.70385411 | 0.35106804 | 4.85334444 | 1.21E−06 | 9.78E−05 |
| ENSG00000141179 | 567.987575 | −0.4824488 | 0.14979937 | −3.2206331 | 0.00127908 | 0.03691105 |
| ENSG00000008283 | 2130.34089 | −1.1974889 | 0.11578518 | −10.342333 | 4.53E−25 | 7.14E−22 |
| ENSG00000265971 | 62.1117018 | −1.1904422 | 0.30766384 | −3.869295 | 0.00010915 | 0.00490817 |
| ENSG00000159640 | 121.352346 | −1.4812143 | 0.2375392 | −6.2356625 | 4.50E−10 | 7.48E−08 |
| ENSG00000173826 | 144.184508 | −1.0281372 | 0.22886061 | −4.4924164 | 7.04E−06 | 0.00046477 |
| ENSG00000108370 | 94.6319942 | −0.7421897 | 0.2246736 | −3.303413 | 0.00095516 | 0.02925523 |
| ENSG00000075461 | 5066.4808 | −1.1323913 | 0.14338495 | −7.8975605 | 2.84E−15 | 1.15E−12 |
| ENSG00000264491 | 23.2329561 | −1.1972059 | 0.37842023 | −3.1636943 | 0.0015578 | 0.04278209 |
| ENSG00000125398 | 65.345812 | 0.94674153 | 0.25030055 | 3.78241887 | 0.00015531 | 0.00649016 |
| ENSG00000180616 | 156.262292 | 1.47715086 | 0.19749001 | 7.47962322 | 7.45E−14 | 2.50E−11 |
| ENSG00000167861 | 851.396493 | −0.7794793 | 0.15629545 | −4.9872168 | 6.13E−07 | 5.31E−05 |
| ENSG00000073350 | 115.293063 | −0.8476242 | 0.22379283 | −3.7875393 | 0.00015215 | 0.00639203 |
| ENSG00000092929 | 987.232961 | −0.9695222 | 0.1619178 | −5.9877434 | 2.13E−09 | 3.07E−07 |
| ENSG00000161544 | 667.309858 | −0.8372422 | 0.1792687 | −4.67032 | 3.01E−06 | 0.00021975 |
| ENSG00000167281 | 29.9546443 | 1.94946586 | 0.37935424 | 5.13890618 | 2.76E−07 | 2.63E−05 |
| ENSG00000266074 | 889.601922 | −0.7733657 | 0.21519032 | −3.5938685 | 0.0003258 | 0.01209793 |
| ENSG00000132205 | 73.1754873 | −1.0183713 | 0.23626589 | −4.3102765 | 1.63E−05 | 0.00096139 |
| ENSG00000088756 | 232.477404 | 0.78770191 | 0.21459951 | 3.6705671 | 0.00024201 | 0.00932502 |
| ENSG00000141441 | 565.882074 | −1.1632244 | 0.15546672 | −7.482144 | 7.31E−14 | 2.49E−11 |
| ENSG00000101489 | 609.429329 | −0.548978 | 0.14557274 | −3.7711594 | 0.00016249 | 0.00668926 |
| ENSG00000184828 | 82.934306 | −0.9568457 | 0.21520026 | −4.4463034 | 8.74E−06 | 0.00056207 |
| ENSG00000141639 | 145.502867 | −0.7869743 | 0.19813193 | −3.971971 | 7.13E−05 | 0.00339162 |
| ENSG00000041353 | 17.0780859 | −1.7859907 | 0.48045913 | −3.7172584 | 0.0002014 | 0.00804799 |
| ENSG00000141668 | 45.685524 | −1.2962667 | 0.30378135 | −4.2671043 | 1.98E−05 | 0.00113174 |
| ENSG00000101282 | 60.1866339 | −1.1303309 | 0.26862657 | −4.2078149 | 2.58E−05 | 0.00141819 |
| ENSG00000101361 | 2102.08274 | 0.35082405 | 0.10731398 | 3.26913634 | 0.00107876 | 0.03198767 |
| ENSG00000088836 | 106.741058 | −0.6874081 | 0.20965069 | −3.2788257 | 0.0010424 | 0.03118056 |
| ENSG00000205181 | 25.6174295 | −1.7475592 | 0.48108063 | −3.6325704 | 0.00028061 | 0.01061243 |
| ENSG00000089199 | 2028.44486 | −0.5940812 | 0.09556008 | −6.2168344 | 5.07E−10 | 8.24E−08 |
| ENSG00000132639 | 2996.80079 | −0.4581725 | 0.11944001 | −3.8360053 | 0.00012505 | 0.005467 |
| ENSG00000089177 | 219.165733 | −0.5200411 | 0.15478555 | −3.3597525 | 0.00078012 | 0.02478466 |
| ENSG00000204103 | 312.707527 | 1.4911281 | 0.18455809 | 8.07945123 | 6.51E−16 | 3.10E−13 |
| ENSG00000124191 | 489.704233 | −1.1086327 | 0.19053164 | −5.8186281 | 5.93E−09 | 7.68E−07 |
| ENSG00000101104 | 545.389474 | 0.45357489 | 0.14606781 | 3.10523516 | 0.00190128 | 0.04974444 |
| ENSG00000124212 | 166.092875 | −1.0090544 | 0.1853266 | −5.4447356 | 5.19E−08 | 5.65E−06 |
| ENSG00000124216 | 355.278159 | 0.98094087 | 0.15877567 | 6.17815594 | 6.49E−10 | 1.01E−07 |
| ENSG00000101115 | 69.152886 | −1.7622689 | 0.28400381 | −6.2050888 | 5.47E−10 | 8.81E−08 |
| ENSG00000054803 | 24.1796649 | −6.4334995 | 0.94508272 | −6.8073401 | 9.94E−12 | 2.29E−09 |
| ENSG00000174403 | 17.0352714 | −1.6569261 | 0.49648682 | −3.3373012 | 0.00084596 | 0.02643789 |
| ENSG00000101187 | 205.549932 | −0.6877131 | 0.17839631 | −3.8549739 | 0.00011574 | 0.005148 |
| ENSG00000092758 | 52.3181012 | −1.2268436 | 0.34413821 | −3.5649735 | 0.00036389 | 0.01313095 |
| ENSG00000196132 | 764.511785 | −0.4866825 | 0.14525738 | −3.3504835 | 0.00080671 | 0.025471 |
| ENSG00000064666 | 280.030881 | 0.75543135 | 0.18062828 | 4.18224299 | 2.89E−05 | 0.00157907 |
| ENSG00000130270 | 92.4956445 | −1.1760407 | 0.22100837 | −5.3212495 | 1.03E−07 | 1.06E−05 |
| ENSG00000167476 | 54.1644301 | −2.1152276 | 0.31827821 | −6.6458447 | 3.01E−11 | 6.49E−09 |
| ENSG00000104953 | 74.9401725 | −0.785922 | 0.22588965 | −3.4792298 | 0.00050286 | 0.01737915 |
| ENSG00000105278 | 78.1646138 | −1.2124295 | 0.25039091 | −4.8421467 | 1.28E−06 | 0.00010306 |
| ENSG00000167664 | 60.8667077 | −1.0657148 | 0.2706192 | −3.9380606 | 8.21E−05 | 0.00378523 |
| ENSG00000205744 | 120.232388 | −0.6590221 | 0.18636966 | −3.536102 | 0.00040608 | 0.01442423 |
| ENSG00000198723 | 20.8813531 | −1.7137771 | 0.41083299 | −4.1714691 | 3.03E−05 | 0.00163379 |
| ENSG00000105514 | 258.258443 | −0.6904598 | 0.21623806 | −3.1930539 | 0.00140777 | 0.03951017 |
| ENSG00000179284 | 78.2269659 | −0.8035454 | 0.24292116 | −3.3078444 | 0.00094017 | 0.02892614 |
| ENSG00000160951 | 33.4021477 | −1.4368958 | 0.39988373 | −3.5932839 | 0.00032654 | 0.01210314 |
| ENSG00000105639 | 119.482856 | −0.5972182 | 0.18186266 | −3.2838969 | 0.00102382 | 0.03080504 |
| ENSG00000130513 | 320.456526 | −1.1791059 | 0.23698139 | −4.9755209 | 6.51E−07 | 5.59E−05 |
| ENSG00000250067 | 137.323083 | 0.90049499 | 0.20480682 | 4.39680172 | 1.10E−05 | 0.00068112 |
| ENSG00000231205 | 183.23033 | −0.8572459 | 0.21296099 | −4.0253657 | 5.69E−05 | 0.00280459 |
| ENSG00000261754 | 91.7774291 | −0.8659613 | 0.25532187 | −3.3916458 | 0.00069474 | 0.02256257 |
| ENSG00000167641 | 51.4063175 | −1.81617 | 0.42537528 | −4.2695711 | 1.96E−05 | 0.00112243 |
| ENSG00000196218 | 23.1720344 | −1.5057843 | 0.41677527 | −3.6129407 | 0.00030274 | 0.0113654 |
| ENSG00000187994 | 38.9872218 | −1.2565622 | 0.33988773 | −3.6969921 | 0.00021817 | 0.00860066 |
| ENSG00000161243 | 214.574456 | −0.8023388 | 0.19938447 | −4.0240788 | 5.72E−05 | 0.00281319 |
| ENSG00000090932 | 264.750859 | −0.9974566 | 0.25187334 | −3.9601516 | 7.49E−05 | 0.0035311 |
| ENSG00000275395 | 18.2494683 | 3.69628109 | 0.53789537 | 6.87174737 | 6.34E−12 | 1.49E−09 |
| ENSG00000243137 | 29.4010299 | 1.53020832 | 0.34329482 | 4.45741745 | 8.30E−06 | 0.00053709 |
| ENSG00000125746 | 301.72452 | −0.5533983 | 0.15860822 | −3.4890896 | 0.00048467 | 0.01689322 |
| ENSG00000124440 | 96.4992964 | −0.955677 | 0.20486689 | −4.664868 | 3.09E−06 | 0.00022406 |
| ENSG00000160013 | 36.813057 | −1.3557039 | 0.3567417 | −3.8002395 | 0.00014456 | 0.00612276 |
| ENSG00000167414 | 257.61609 | −1.2303575 | 0.2956618 | −4.1613678 | 3.16E−05 | 0.00169436 |
| ENSG00000074219 | 391.268339 | 0.5136593 | 0.14445834 | 3.55576071 | 0.00037689 | 0.01355204 |
| ENSG00000204653 | 169.900402 | −0.7710543 | 0.23061805 | −3.3434256 | 0.00082751 | 0.02596753 |
| ENSG00000275183 | 29.2467052 | −1.4714434 | 0.35885946 | −4.1003333 | 4.13E−05 | 0.00212736 |
| ENSG00000131037 | 204.894485 | −0.9687742 | 0.23634168 | −4.0990409 | 4.15E−05 | 0.00212736 |
| ENSG00000105048 | 173.165797 | −0.9572987 | 0.23498806 | −4.0738184 | 4.62E−05 | 0.00233641 |
| ENSG00000080031 | 86.901619 | −0.7258848 | 0.20856836 | −3.4803209 | 0.00050081 | 0.01733781 |
| ENSG00000179954 | 55.6317859 | 1.0846854 | 0.31350082 | 3.45991242 | 0.00054035 | 0.01837102 |
| ENSG00000100302 | 19.0976883 | −1.5541959 | 0.48311005 | −3.2170639 | 0.0012951 | 0.0373207 |
| ENSG00000189060 | 5799.93036 | −0.4634447 | 0.10730366 | −4.3190015 | 1.57E−05 | 0.00092683 |
| ENSG00000177096 | 33.4478452 | −2.2197407 | 0.44748746 | −4.9604533 | 7.03E−07 | 5.93E−05 |
| ENSG00000188677 | 317.190454 | −0.6567941 | 0.15187949 | −4.3244422 | 1.53E−05 | 0.00090687 |
| ENSG00000278189 | 13.0780688 | −3.4486194 | 0.69973517 | −4.9284637 | 8.29E−07 | 6.92E−05 |
| ENSG00000280081 | 14.9529678 | 1.81640418 | 0.51991611 | 3.4936486 | 0.00047647 | 0.01666415 |
| ENSG00000154642 | 221.416533 | 0.79060934 | 0.24582641 | 3.21612853 | 0.00129933 | 0.03737369 |
| ENSG00000273492 | 22.4154376 | −1.5294511 | 0.44885919 | −3.4074184 | 0.00065581 | 0.02164157 |
| ENSG00000154734 | 2372.64189 | −0.3821058 | 0.10219933 | −3.7388286 | 0.00018488 | 0.00750524 |
| ENSG00000160179 | 150.495894 | −0.9031405 | 0.20447781 | −4.4168142 | 1.00E−05 | 0.00062482 |
| ENSG00000228709 | 459.974012 | −0.888286 | 0.21034508 | −4.2229942 | 2.41E−05 | 0.00134034 |
| ENSG00000175894 | 596.369164 | −0.7783842 | 0.17881667 | −4.3529735 | 1.34E−05 | 0.00080112 |
| ENSG00000235890 | 709.057452 | −0.9708052 | 0.23680369 | −4.0996204 | 4.14E−05 | 0.00212736 |
| ENSG00000182912 | 1286.0869 | −0.9966493 | 0.15289097 | −6.5186925 | 7.09E−11 | 1.38E−08 |
| ENSG00000233922 | 219.026482 | −0.6673587 | 0.17677285 | −3.775233 | 0.00015986 | 0.00660747 |

TABLE 5
Differential gene Expression_DESeq2_dCT_dC

| | | Log2 Fold | | | | |
|---|---|---|---|---|---|---|
| | Base Mean | Change | Lfcse | Stat | Pvalue | Padj |
| ENSG00000223764 | 513.919766 | 0.76980793 | 0.15330087 | 5.0215498 | 5.13E−07 | 0.00014386 |
| ENSG00000187634 | 1066.25504 | 1.26443382 | 0.21617229 | 5.84919485 | 4.94E−09 | 2.16E−06 |
| ENSG00000242485 | 937.600436 | 0.84197842 | 0.24250924 | 3.4719436 | 0.0005167 | 0.02318184 |
| ENSG00000160072 | 699.547282 | 0.63749214 | 0.20079995 | 3.17476243 | 0.00149959 | 0.04618309 |
| ENSG00000171608 | 251.374075 | −0.8171353 | 0.19083633 | −4.2818643 | 1.85E−05 | 0.00215788 |
| ENSG00000053372 | 1048.70128 | 0.69168747 | 0.19067769 | 3.62752167 | 0.00028615 | 0.01532705 |
| ENSG00000007968 | 639.194696 | 0.44698203 | 0.1423607 | 3.13978537 | 0.00169072 | 0.04970239 |
| ENSG00000117318 | 1362.20122 | 1.07048765 | 0.25551645 | 4.1895058 | 2.80E−05 | 0.00288784 |
| ENSG00000127423 | 102.054816 | 0.92941382 | 0.23557711 | 3.9452637 | 7.97E−05 | 0.0058991 |
| ENSG00000198830 | 7194.36677 | 0.53039221 | 0.13845409 | 3.83081642 | 0.00012772 | 0.00859646 |
| ENSG00000117748 | 1234.30582 | 0.49178296 | 0.14364896 | 3.42350526 | 0.00061819 | 0.02578337 |
| ENSG00000130770 | 1000.4386 | 0.77764761 | 0.21401401 | 3.63362947 | 0.00027946 | 0.01511636 |
| ENSG00000092853 | 835.308296 | 0.91973439 | 0.18663711 | 4.9279288 | 8.31E−07 | 0.00020266 |
| ENSG00000168389 | 269.923372 | 0.48390572 | 0.14977012 | 3.23098977 | 0.00123362 | 0.04086893 |
| ENSG00000117016 | 1948.07408 | −0.5641935 | 0.1651002 | −3.4172791 | 0.0006325 | 0.02578337 |
| ENSG00000171960 | 754.463631 | 0.62482448 | 0.18984522 | 3.29123101 | 0.0009975 | 0.03558227 |
| ENSG00000117395 | 1578.53292 | 0.66051232 | 0.18856754 | 3.50278902 | 0.00046041 | 0.02140206 |
| ENSG00000132773 | 227.758077 | 0.66942488 | 0.17450809 | 3.83606798 | 0.00012502 | 0.00845304 |
| ENSG00000132780 | 6332.75757 | 0.45771822 | 0.12440385 | 3.67929305 | 0.00023388 | 0.01343238 |
| ENSG00000162407 | 227.302124 | −0.9493862 | 0.19901447 | −4.7704383 | 1.84E−06 | 0.00035979 |
| ENSG00000116678 | 150.314744 | −0.9495469 | 0.27524424 | −3.4498338 | 0.00056093 | 0.02421955 |
| ENSG00000178965 | 215.550561 | 1.447095 | 0.23814934 | 6.07641825 | 1.23E−09 | 6.57E−07 |
| ENSG00000153904 | 1124.74806 | −0.9168102 | 0.16519596 | −5.5498342 | 2.86E−08 | 1.12E−05 |
| ENSG00000143013 | 215.289297 | 1.01913562 | 0.2120571 | 4.80594899 | 1.54E−06 | 0.00033203 |
| ENSG00000171488 | 707.048687 | −0.6188787 | 0.18949378 | −3.2659577 | 0.00109095 | 0.03747762 |
| ENSG00000137962 | 326.475887 | −1.0095764 | 0.22140045 | −4.5599564 | 5.12E−06 | 0.00081115 |
| ENSG00000143036 | 29.7039746 | −1.5222352 | 0.41188376 | −3.6957884 | 0.00021921 | 0.01304273 |
| ENSG00000237954 | 49.4200112 | −2.7360661 | 0.3903875 | −7.0085905 | 2.41E−12 | 2.39E−09 |
| ENSG00000188641 | 587.752757 | −0.6591941 | 0.1698228 | −3.8816586 | 0.00010375 | 0.00732801 |
| ENSG00000116299 | 132.548918 | −0.754858 | 0.22940468 | −3.2905085 | 0.00100006 | 0.03558843 |
| ENSG00000116396 | 116.688511 | −1.2220596 | 0.2329504 | −5.2460077 | 1.55E−07 | 5.14E−05 |
| ENSG00000092621 | 3312.3205 | 0.64433787 | 0.15908223 | 4.05034468 | 5.11E−05 | 0.00430393 |
| ENSG00000265972 | 8541.57977 | −0.5527295 | 0.1318549 | −4.1919523 | 2.77E−05 | 0.00287685 |
| ENSG00000203814 | 22.7288953 | −1.759948 | 0.49148253 | −3.5808963 | 0.00034242 | 0.01744336 |
| ENSG00000160691 | 5930.36505 | −0.7245695 | 0.1652421 | −4.3848966 | 1.16E−05 | 0.00148802 |
| ENSG00000160783 | 229.624984 | 1.042331 | 0.26479689 | 3.93634154 | 8.27E−05 | 0.00609236 |
| ENSG00000160818 | 1077.22567 | 0.72275711 | 0.19868605 | 3.63768419 | 0.0002751 | 0.01493474 |
| ENSG00000143153 | 3963.35425 | −0.4387341 | 0.09927266 | −4.4194861 | 9.89E−06 | 0.00131399 |
| ENSG00000116147 | 2240.0628 | −1.3232232 | 0.26419504 | −5.0085089 | 5.49E−07 | 0.00014835 |
| ENSG00000186283 | 539.344545 | 0.51020265 | 0.13964818 | 3.65348593 | 0.0002587 | 0.01443492 |
| ENSG00000143333 | 849.829044 | 1.04271683 | 0.17178962 | 6.06973135 | 1.28E−09 | 6.57E−07 |
| ENSG00000135829 | 7804.95605 | 0.3790856 | 0.11434844 | 3.3151793 | 0.00091584 | 0.03347217 |
| ENSG00000159176 | 579.821256 | 0.5644383 | 0.16223459 | 3.47914899 | 0.00050301 | 0.02303701 |
| ENSG00000077152 | 1335.13612 | 0.65522593 | 0.2047729 | 3.19976878 | 0.00137538 | 0.04367845 |
| ENSG00000117139 | 5294.066 | −0.5694656 | 0.16508884 | −3.4494495 | 0.00056173 | 0.02421955 |
| ENSG00000143847 | 412.507564 | −0.673461 | 0.18658983 | −3.6093124 | 0.00030701 | 0.01619072 |
| ENSG00000058668 | 874.427613 | −0.7451126 | 0.20947768 | −3.5570022 | 0.00037511 | 0.01866146 |
| ENSG00000257315 | 289.822318 | −1.0479793 | 0.31454434 | −3.3317379 | 0.00086306 | 0.03206236 |
| ENSG00000162889 | 3007.29034 | 0.52005291 | 0.13734156 | 3.7865661 | 0.00015274 | 0.00979336 |
| ENSG00000162894 | 96.626403 | 1.07392375 | 0.25491701 | 4.21283676 | 2.52E−05 | 0.00267945 |
| ENSG00000117595 | 223.578204 | 0.88397992 | 0.17692829 | 4.99626113 | 5.85E−07 | 0.00015526 |
| ENSG00000143473 | 148.877934 | −0.9436216 | 0.26588364 | −3.5490021 | 0.00038669 | 0.0190466 |
| ENSG00000143476 | 1826.89204 | 0.4559828 | 0.13953134 | 3.2679598 | 0.00108326 | 0.03738621 |
| ENSG00000230461 | 309.632859 | −0.890464 | 0.19530354 | −4.5593849 | 5.13E−06 | 0.00081115 |
| ENSG00000196660 | 24.119524 | −1.5884344 | 0.44595327 | −3.5618853 | 0.0003682 | 0.01837917 |
| ENSG00000143674 | 222.777063 | −0.6145431 | 0.18669768 | −3.2916482 | 0.00099602 | 0.03558227 |
| ENSG00000133019 | 74.6453602 | −1.3749338 | 0.30140234 | −4.5617886 | 5.07E−06 | 0.00081115 |
| ENSG00000180875 | 343.86164 | 1.04236376 | 0.17172207 | 6.07006277 | 1.28E−09 | 6.57E−07 |
| ENSG00000174371 | 1592.46943 | 0.43576552 | 0.13900854 | 3.13481109 | 0.00171965 | 0.04996048 |
| ENSG00000225234 | 112.157611 | −0.7616738 | 0.24274742 | −3.1377215 | 0.00170267 | 0.04985661 |
| ENSG00000182551 | 750.125674 | 0.50457897 | 0.15623601 | 3.22959461 | 0.00123966 | 0.0409776 |
| ENSG00000115738 | 2503.56412 | 1.10723857 | 0.15484456 | 7.15064546 | 8.64E−13 | 1.07E−09 |
| ENSG00000171848 | 2200.77166 | 0.64931334 | 0.13651121 | 4.75648369 | 1.97E−06 | 0.00037092 |
| ENSG00000071575 | 2429.92788 | 0.3718491 | 0.1071611 | 3.47000064 | 0.00052046 | 0.02324865 |
| ENSG00000151779 | 1427.46586 | −0.6080425 | 0.16615879 | −3.6594061 | 0.0002528 | 0.01424396 |
| ENSG00000115129 | 234.456911 | 0.78624126 | 0.19791795 | 3.97256169 | 7.11E−05 | 0.00554657 |
| ENSG00000171094 | 4727.26929 | −0.7202833 | 0.22425385 | −3.2119104 | 0.00131855 | 0.04256685 |
| ENSG00000158089 | 201.028778 | 0.74520599 | 0.18855407 | 3.95221377 | 7.74E−05 | 0.00580972 |
| ENSG00000279873 | 43.0997205 | −1.1538846 | 0.34620302 | −3.3329709 | 0.00085924 | 0.03206236 |
| ENSG00000225156 | 452.420714 | −0.5153001 | 0.14016066 | −3.6764957 | 0.00023646 | 0.0134764 |
| ENSG00000196975 | 303.681175 | −0.528043 | 0.15607401 | −3.3832857 | 0.00071624 | 0.02793239 |
| ENSG00000143977 | 1002.26131 | 0.66276371 | 0.21044039 | 3.14941306 | 0.00163599 | 0.04857349 |
| ENSG00000163017 | 150.638098 | 0.86651521 | 0.24592205 | 3.523536 | 0.00042583 | 0.01995209 |
| ENSG00000065911 | 1623.56559 | 0.44871115 | 0.1291569 | 3.47415541 | 0.00051246 | 0.02314402 |
| ENSG00000115350 | 195.928217 | 0.98365934 | 0.29956533 | 3.28362215 | 0.00102482 | 0.03629579 |
| ENSG00000168874 | 141.952709 | 0.99816553 | 0.24455964 | 4.081481 | 4.47E−05 | 0.00395271 |
| ENSG00000158050 | 128.21304 | 1.54191901 | 0.25992374 | 5.93219772 | 2.99E−09 | 1.39E−06 |
| ENSG00000115539 | 510.529031 | 0.58626612 | 0.17591865 | 3.33259779 | 0.00086039 | 0.03206236 |
| ENSG00000198075 | 351.465285 | −0.7910761 | 0.2275486 | −3.476515 | 0.00050798 | 0.02303701 |
| ENSG00000175497 | 263.30137 | −1.4091044 | 0.16144183 | −8.7282482 | 2.59E−18 | 7.69E−15 |
| ENSG00000152076 | 335.354352 | 0.88835786 | 0.18349553 | 4.84130512 | 1.29E−06 | 0.00029071 |
| ENSG00000076003 | 2658.18865 | 0.51145597 | 0.12594971 | 4.06079506 | 4.89E−05 | 0.00420506 |
| ENSG00000144354 | 1393.70125 | 0.48068394 | 0.12016713 | 4.00012813 | 6.33E−05 | 0.00511799 |
| ENSG00000162998 | 57.6370134 | −0.9851355 | 0.28812404 | −3.4191367 | 0.0006282 | 0.02578337 |
| ENSG00000168542 | 4165.10392 | −0.6108353 | 0.12797584 | −4.7730518 | 1.81E−06 | 0.00035979 |
| ENSG00000138411 | 163.320451 | −0.8251524 | 0.23992256 | −3.4392447 | 0.00058334 | 0.02482143 |
| ENSG00000196141 | 776.601835 | 0.46383063 | 0.12472461 | 3.7188382 | 0.00020014 | 0.01210203 |
| ENSG00000055044 | 1896.83989 | 0.66236518 | 0.17610018 | 3.7612975 | 0.00016903 | 0.01065417 |
| ENSG00000116117 | 65.0265805 | −1.0333628 | 0.32674923 | −3.1625561 | 0.00156391 | 0.04728273 |
| ENSG00000118263 | 2447.22833 | −0.5478006 | 0.13428828 | −4.0792881 | 4.52E−05 | 0.00395271 |
| ENSG00000144406 | 626.451526 | −0.7916602 | 0.2247059 | −3.5230947 | 0.00042654 | 0.01995209 |
| ENSG00000171951 | 3148.1728 | 0.47712095 | 0.13720119 | 3.47752791 | 0.00050606 | 0.02303701 |
| ENSG00000273301 | 104.498087 | −1.0356249 | 0.31587453 | −3.2785957 | 0.00104325 | 0.03659986 |
| ENSG00000187514 | 20408.7226 | 0.69575431 | 0.19899559 | 3.49633025 | 0.0004717 | 0.02185858 |
| ENSG00000168918 | 110.553766 | −1.411906 | 0.23366014 | −6.0425624 | 1.52E−09 | 7.52E−07 |
| ENSG00000157985 | 2859.85883 | −0.6823709 | 0.21327464 | −3.199494 | 0.00137669 | 0.04367845 |
| ENSG00000132323 | 445.730334 | 0.60507198 | 0.19189739 | 3.15310163 | 0.00161546 | 0.04825281 |
| ENSG00000172428 | 553.272584 | 1.00763784 | 0.30318821 | 3.32347305 | 0.00088904 | 0.03268642 |
| ENSG00000168393 | 810.168786 | 0.85459423 | 0.22698553 | 3.76497236 | 0.00016657 | 0.01054337 |
| ENSG00000150995 | 1034.41494 | −0.7107063 | 0.2080493 | −3.4160475 | 0.00063537 | 0.02582282 |
| ENSG00000134107 | 31.9094537 | −1.4393424 | 0.39883254 | −3.6088892 | 0.00030751 | 0.01619072 |
| ENSG00000196277 | 105.550395 | −0.7702413 | 0.22748137 | −3.3859533 | 0.00070931 | 0.02783919 |
| ENSG00000196220 | 217.688378 | −0.849683 | 0.20548139 | −4.1350847 | 3.55E−05 | 0.00343559 |
| ENSG00000196639 | 44.106565 | 1.08609114 | 0.32443863 | 3.3476012 | 0.00081514 | 0.03101084 |
| ENSG00000170860 | 831.048559 | 0.69254773 | 0.21460023 | 3.22715283 | 0.00125029 | 0.04114605 |
| ENSG00000131389 | 721.177494 | −0.7824407 | 0.24358621 | −3.2121716 | 0.00131736 | 0.04256685 |
| ENSG00000263740 | 23.322155 | −1.8340462 | 0.55054559 | −3.3313249 | 0.00086434 | 0.03206236 |
| ENSG00000183960 | 212.465307 | −0.9145704 | 0.22133427 | −4.1320779 | 3.59E−05 | 0.003442 |
| ENSG00000144681 | 753.216253 | −0.6047163 | 0.13842166 | −4.3686534 | 1.25E−05 | 0.0015894 |
| ENSG00000187091 | 337.443417 | −0.5262044 | 0.1579152 | −3.3321963 | 0.00086163 | 0.03206236 |
| ENSG00000163820 | 282.814221 | −0.8735319 | 0.25756217 | −3.3915379 | 0.00069502 | 0.02744799 |
| ENSG00000164045 | 875.156728 | 0.53146716 | 0.12897206 | 4.12079288 | 3.78E−05 | 0.00355466 |
| ENSG00000007402 | 2567.3274 | −0.7700611 | 0.20657154 | −3.727818 | 0.00019314 | 0.01177471 |
| ENSG00000145050 | 707.92096 | 0.57887721 | 0.18367749 | 3.15159588 | 0.00162381 | 0.04840511 |
| ENSG00000163932 | 223.486999 | −0.6402309 | 0.17376765 | −3.6844079 | 0.00022923 | 0.01333091 |
| ENSG00000163638 | 4307.60544 | −0.6155299 | 0.17360563 | −3.545564 | 0.00039177 | 0.01923312 |
| ENSG00000185008 | 288.228115 | −1.8676246 | 0.23470369 | −7.9573718 | 1.76E−15 | 3.73E−12 |
| ENSG00000170017 | 4114.91898 | −1.1437208 | 0.15344839 | −7.4534557 | 9.09E−14 | 1.35E−10 |
| ENSG00000185565 | 3158.49177 | −0.6323959 | 0.18352377 | −3.445853 | 0.00056926 | 0.02440272 |
| ENSG00000082684 | 103.315161 | −1.1160056 | 0.27139711 | −4.1120762 | 3.92E−05 | 0.00364546 |
| ENSG00000114491 | 642.82753 | 0.67426126 | 0.16639002 | 4.05229383 | 5.07E−05 | 0.00430393 |
| ENSG00000074416 | 94.6433098 | −0.890713 | 0.26154477 | −3.4055851 | 0.00066022 | 0.02661473 |
| ENSG00000196353 | 31.7262009 | −1.4041893 | 0.40680402 | −3.4517586 | 0.00055695 | 0.02415326 |
| ENSG00000154917 | 627.542444 | −0.7907312 | 0.16136158 | −4.9003688 | 9.57E−07 | 0.00022586 |
| ENSG00000163762 | 51.0116089 | −8.8381156 | 1.34792832 | −6.5568142 | 5.50E−11 | 4.09E−08 |
| ENSG00000163661 | 64.2209448 | 1.11998448 | 0.29900913 | 3.74565307 | 0.00017993 | 0.01122584 |
| ENSG00000085276 | 32.5717915 | −1.7007012 | 0.35853892 | −4.7434215 | 2.10E−06 | 0.00039073 |
| ENSG00000145198 | 111.716628 | −1.0564488 | 0.24943274 | −4.2354055 | 2.28E−05 | 0.00249528 |
| ENSG00000163918 | 793.332914 | 0.71780909 | 0.1718918 | 4.17593571 | 2.97E−05 | 0.00302353 |
| ENSG00000114315 | 101.78102 | 1.17100092 | 0.33214596 | 3.52556122 | 0.00042259 | 0.01995209 |
| ENSG00000178222 | 177.567133 | −0.9237471 | 0.18177823 | −5.0817258 | 3.74E−07 | 0.00010909 |
| ENSG00000163950 | 1395.33351 | 0.65212578 | 0.1711521 | 3.81021204 | 0.00013885 | 0.0091624 |
| ENSG00000091490 | 80.3496775 | −0.8932591 | 0.27578717 | −3.2389435 | 0.00119973 | 0.04019377 |
| ENSG00000124406 | 1346.51361 | −0.7212948 | 0.20034419 | −3.6002782 | 0.00031788 | 0.01647533 |
| ENSG00000156140 | 79.1233738 | −0.9355357 | 0.2760026 | −3.3895902 | 0.00069997 | 0.02754518 |
| ENSG00000138769 | 50.729811 | −1.2319708 | 0.34441828 | −3.5769611 | 0.00034761 | 0.01758751 |
| ENSG00000138759 | 513.401002 | −0.8587224 | 0.26331994 | −3.2611371 | 0.00110966 | 0.03794539 |
| ENSG00000138668 | 8421.02803 | 0.38778252 | 0.10240997 | 3.78657002 | 0.00015274 | 0.00979336 |
| ENSG00000184305 | 412.545857 | −0.7139081 | 0.21684973 | −3.2921787 | 0.00099414 | 0.03558227 |
| ENSG00000182168 | 2994.20419 | −0.7664923 | 0.21426037 | −3.5773874 | 0.00034705 | 0.01758751 |
| ENSG00000271474 | 269.107644 | −0.7411091 | 0.18489433 | −4.0082847 | 6.12E−05 | 0.00499877 |
| ENSG00000164032 | 2584.11854 | 0.7955587 | 0.20583354 | 3.86505868 | 0.00011106 | 0.00779271 |
| ENSG00000138795 | 53.0506709 | 1.32177346 | 0.31513722 | 4.19427915 | 2.74E−05 | 0.00286753 |
| ENSG00000164109 | 1808.52017 | 0.54539148 | 0.17289082 | 3.15454267 | 0.0016075 | 0.04811176 |
| ENSG00000164167 | 383.69077 | 0.77662385 | 0.22991896 | 3.37781559 | 0.00073064 | 0.0282293 |
| ENSG00000151617 | 1972.80159 | 0.70809269 | 0.15589264 | 4.54218169 | 5.57E−06 | 0.00085378 |
| ENSG00000137460 | 57.0640892 | −1.0129886 | 0.29957343 | −3.3814368 | 0.00072108 | 0.02793239 |
| ENSG00000168843 | 250.655119 | −1.1510733 | 0.21774166 | −5.2864174 | 1.25E−07 | 4.22E−05 |
| ENSG00000251216 | 1141.42786 | −0.6440151 | 0.17698817 | −3.6387466 | 0.00027397 | 0.01493474 |
| ENSG00000151725 | 1139.09906 | 0.60593069 | 0.17127463 | 3.5377726 | 0.00040352 | 0.01955154 |
| ENSG00000071539 | 1120.76928 | 0.45474001 | 0.11402691 | 3.98800615 | 6.66E−05 | 0.00531135 |
| ENSG00000215218 | 1621.85427 | 0.41141853 | 0.12716657 | 3.23527268 | 0.00121527 | 0.04062266 |
| ENSG00000113494 | 17.7919736 | −1.9384762 | 0.57158122 | −3.3914274 | 0.0006953 | 0.02744799 |
| ENSG00000112936 | 15556.4126 | −0.4676282 | 0.14313803 | −3.2669741 | 0.00108704 | 0.03742977 |
| ENSG00000259663 | 2173.06244 | −0.4758265 | 0.11836282 | −4.0200672 | 5.82E−05 | 0.00478149 |
| ENSG00000130449 | 653.946434 | −0.6418663 | 0.20217539 | −3.1747991 | 0.0014994 | 0.04618309 |
| ENSG00000214944 | 97.3488142 | −0.9650582 | 0.24863686 | −3.8813965 | 0.00010386 | 0.00732801 |
| ENSG00000228716 | 2220.19717 | 0.65098393 | 0.15113729 | 4.30723563 | 1.65E−05 | 0.00201553 |
| ENSG00000164176 | 65.9045405 | −1.8491083 | 0.36200861 | −5.1079125 | 3.26E−07 | 9.89E−05 |
| ENSG00000129595 | 282.507852 | −0.8120707 | 0.25895498 | −3.1359532 | 0.00171297 | 0.04986371 |
| ENSG00000113368 | 2767.97899 | 0.37677834 | 0.10226882 | 3.68419559 | 0.00022943 | 0.01333091 |
| ENSG00000066583 | 10696.836 | 0.65993757 | 0.140128 | 4.70953396 | 2.48E−06 | 0.00044497 |
| ENSG00000145833 | 2398.48358 | 0.65049682 | 0.17553877 | 3.70571593 | 0.00021079 | 0.01267995 |
| ENSG00000152377 | 235.457097 | −1.59175 | 0.21763281 | −7.3139249 | 2.59E−13 | 3.51E−10 |
| ENSG00000044115 | 3481.64199 | −0.3284454 | 0.10076638 | −3.2594737 | 0.00111619 | 0.03808106 |
| ENSG00000145819 | 381.969105 | −0.5788252 | 0.16920733 | −3.4208042 | 0.00062436 | 0.02578337 |
| ENSG00000113657 | 1357.76408 | −0.4885692 | 0.14806182 | −3.2997651 | 0.00096766 | 0.03493667 |
| ENSG00000184347 | 8511.75239 | −0.5755041 | 0.18315489 | −3.1421718 | 0.001677 | 0.04939666 |
| ENSG00000120149 | 497.016479 | 0.56770471 | 0.13112711 | 4.32942293 | 1.50E−05 | 0.00188459 |
| ENSG00000087116 | 228.336828 | 0.76847182 | 0.22630637 | 3.39571445 | 0.0006845 | 0.02722432 |
| ENSG00000054598 | 3112.5844 | 0.70552041 | 0.1745433 | 4.0420938 | 5.30E−05 | 0.00437788 |
| ENSG00000112312 | 710.099198 | 0.72628843 | 0.18380935 | 3.95131391 | 7.77E−05 | 0.00580972 |
| ENSG00000026950 | 455.546524 | −0.6655528 | 0.18862095 | −3.5285201 | 0.00041789 | 0.01995209 |
| ENSG00000137310 | 490.430255 | 0.51322309 | 0.14105616 | 3.63843087 | 0.0002743 | 0.01493474 |
| ENSG00000096433 | 128.384927 | −1.249022 | 0.33736084 | −3.7023326 | 0.00021363 | 0.01276181 |
| ENSG00000112081 | 6471.12769 | 0.58251318 | 0.18128944 | 3.21316667 | 0.0013128 | 0.04256685 |
| ENSG00000112576 | 469.900632 | 0.56593515 | 0.17736332 | 3.19082401 | 0.00141868 | 0.04433365 |
| ENSG00000180992 | 486.797143 | 0.92835901 | 0.25493299 | 3.64158053 | 0.00027097 | 0.0149284 |
| ENSG00000008196 | 8624.97581 | −0.6679235 | 0.10138842 | −6.5877689 | 4.46E−11 | 3.69E−08 |
| ENSG00000112118 | 4142.82279 | 0.44213869 | 0.08813404 | 5.0166619 | 5.26E−07 | 0.00014483 |
| ENSG00000202198 | 566.346759 | −5.3014734 | 1.38773696 | −3.8202293 | 0.00013333 | 0.00893355 |
| ENSG00000272316 | 41.6244417 | −1.5353823 | 0.37663699 | −4.0765575 | 4.57E−05 | 0.00397601 |
| ENSG00000230910 | 192.275141 | −0.5999828 | 0.19113605 | −3.1390351 | 0.00169505 | 0.04973154 |
| ENSG00000135298 | 515.032149 | −0.5798636 | 0.14623676 | −3.965238 | 7.33E−05 | 0.00565117 |
| ENSG00000079841 | 86.1193399 | −0.981586 | 0.27811153 | −3.5294688 | 0.00041639 | 0.01995209 |
| ENSG00000112367 | 487.597432 | −0.4335115 | 0.13822684 | −3.1362328 | 0.00171133 | 0.04986371 |
| ENSG00000155115 | 805.614288 | 0.77678391 | 0.21526802 | 3.60845006 | 0.00030803 | 0.01619072 |
| ENSG00000146352 | 229.15436 | −0.8885794 | 0.21759233 | −4.0836888 | 4.43E−05 | 0.00395271 |
| ENSG00000203760 | 221.053986 | 0.94664472 | 0.26709458 | 3.54423041 | 0.00039376 | 0.01926709 |
| ENSG00000118515 | 1176.5287 | 0.66851752 | 0.12172551 | 5.4920082 | 3.97E−08 | 1.52E−05 |
| ENSG00000146469 | 906.476132 | 0.70065502 | 0.20267926 | 3.45696453 | 0.0005463 | 0.02380678 |
| ENSG00000153721 | 585.426642 | −0.5241185 | 0.13375892 | −3.9183818 | 8.91E−05 | 0.00641294 |
| ENSG00000029639 | 343.599121 | −0.4685352 | 0.13973144 | −3.3531121 | 0.00079908 | 0.03047786 |
| ENSG00000185345 | 57.1503163 | −0.9741316 | 0.28419424 | −3.4276965 | 0.00060873 | 0.02550646 |
| ENSG00000164850 | 182.544241 | 0.81569307 | 0.22616348 | 3.60665239 | 0.00031017 | 0.01624584 |
| ENSG00000122687 | 1406.05674 | 0.46552775 | 0.13390384 | 3.47658259 | 0.00050785 | 0.02303701 |
| ENSG00000048052 | 4204.65126 | −0.6418913 | 0.19855121 | −3.2328754 | 0.00122551 | 0.04071354 |
| ENSG00000105855 | 41.1619749 | −1.3690612 | 0.41323574 | −3.313027 | 0.00092292 | 0.03364815 |
| ENSG00000122585 | 220.635473 | −1.1303427 | 0.22145795 | −5.1040962 | 3.32E−07 | 9.89E−05 |
| ENSG00000070882 | 190.46303 | −0.8309842 | 0.20014796 | −4.1518495 | 3.30E−05 | 0.00327051 |
| ENSG00000122574 | 94.4781546 | −0.9682824 | 0.30756568 | −3.1482133 | 0.00164272 | 0.04867616 |
| ENSG00000122641 | 177.131899 | −0.7524414 | 0.22993697 | −3.272381 | 0.00106646 | 0.03689199 |
| ENSG00000272655 | 63.4077982 | 0.85800281 | 0.26339449 | 3.25748202 | 0.00112405 | 0.0381742 |
| ENSG00000146674 | 5646.90033 | 1.01809947 | 0.09671891 | 10.5263745 | 6.53E−26 | 9.71E−22 |
| ENSG00000225648 | 371.693317 | 0.55712035 | 0.16902237 | 3.29613386 | 0.00098025 | 0.03530572 |
| ENSG00000049540 | 1513.88196 | 0.63559113 | 0.19237962 | 3.30383819 | 0.00095371 | 0.03457091 |
| ENSG00000049541 | 1368.7005 | 0.71417122 | 0.1588775 | 4.4951062 | 6.95E−06 | 0.00098508 |
| ENSG00000186088 | 109.698772 | −0.9366616 | 0.28589078 | −3.2762917 | 0.0010518 | 0.03672654 |
| ENSG00000075223 | 3666.39632 | −0.5559757 | 0.17116721 | −3.248144 | 0.0011616 | 0.03918112 |
| ENSG00000164692 | 67.8006859 | 1.15850618 | 0.28055219 | 4.12937852 | 3.64E−05 | 0.00344631 |
| ENSG00000166508 | 8960.02005 | 0.48583392 | 0.12972745 | 3.74503568 | 0.00018037 | 0.01122584 |
| ENSG00000077080 | 888.474157 | 0.52997269 | 0.16744758 | 3.16500662 | 0.00155079 | 0.04704749 |
| ENSG00000164597 | 961.894097 | −0.5603358 | 0.17236961 | −3.2507806 | 0.00115089 | 0.03899643 |
| ENSG00000173114 | 1896.30582 | 0.83483983 | 0.19326962 | 4.31956067 | 1.56E−05 | 0.00193797 |
| ENSG00000135269 | 331.219696 | −1.22014 | 0.19662974 | −6.2052668 | 5.46E−10 | 3.53E−07 |
| ENSG00000179603 | 207.096354 | −0.7311441 | 0.17859206 | −4.0939343 | 4.24E−05 | 0.00384677 |
| ENSG00000128596 | 1120.12758 | −0.6807651 | 0.2077027 | −3.2775939 | 0.00104696 | 0.03664356 |
| ENSG00000128567 | 919.311468 | −0.6651589 | 0.13441477 | −4.9485551 | 7.48E−07 | 0.00018536 |
| ENSG00000105894 | 646.919988 | 0.74304272 | 0.18597305 | 3.99543227 | 6.46E−05 | 0.00519228 |
| ENSG00000090266 | 498.833367 | 0.86264759 | 0.24458149 | 3.52703546 | 0.00042024 | 0.01995209 |
| ENSG00000159784 | 149.23514 | −1.0120132 | 0.23859093 | −4.241625 | 2.22E−05 | 0.00246334 |
| ENSG00000174469 | 385.825809 | −1.0566442 | 0.24679638 | −4.2814415 | 1.86E−05 | 0.00215788 |
| ENSG00000106462 | 1871.50657 | 0.45415596 | 0.11217322 | 4.04870211 | 5.15E−05 | 0.00430393 |
| ENSG00000197558 | 43.5577965 | −1.449795 | 0.37595224 | −3.8563276 | 0.0001151 | 0.00803831 |
| ENSG00000198947 | 32.7971026 | −1.4155624 | 0.40282702 | −3.51407 | 0.0004413 | 0.0205777 |
| ENSG00000147155 | 467.685573 | 0.71177579 | 0.20936955 | 3.39961466 | 0.00067481 | 0.02691094 |
| ENSG00000274588 | 74.3315493 | −0.9763253 | 0.30418562 | −3.2096365 | 0.00132903 | 0.04269829 |
| ENSG00000072506 | 1622.04752 | 0.72246439 | 0.22860389 | 3.16033286 | 0.00157589 | 0.0475484 |
| ENSG00000133169 | 2745.58896 | 0.77942259 | 0.2411046 | 3.23271553 | 0.0012262 | 0.04071354 |
| ENSG00000077279 | 2923.7225 | −1.1182357 | 0.23294888 | −4.800348 | 1.58E−06 | 0.00033658 |
| ENSG00000260802 | 336.484212 | −2.3876263 | 0.25318397 | −9.4304008 | 4.09E−21 | 1.52E−17 |
| ENSG00000125354 | 1607.08669 | −0.4321042 | 0.10346184 | −4.1764593 | 2.96E−05 | 0.00302353 |
| ENSG00000009694 | 30.321651 | −1.5649649 | 0.46842802 | −3.3408865 | 0.00083511 | 0.03160894 |
| ENSG00000147256 | 488.353731 | −0.5005434 | 0.15432318 | −3.2434752 | 0.00118081 | 0.03973882 |
| ENSG00000171004 | 280.445427 | −1.3422017 | 0.18933627 | −7.0889836 | 1.35E−12 | 1.55E−09 |
| ENSG00000184785 | 22.31125 | 1.74288214 | 0.50984146 | 3.41847866 | 0.00062972 | 0.02578337 |
| ENSG00000124260 | 79.3122591 | 1.13395529 | 0.29444466 | 3.85116605 | 0.00011756 | 0.00813329 |
| ENSG00000221867 | 842.616934 | 0.5479416 | 0.17069828 | 3.21000073 | 0.00132735 | 0.04269829 |
| ENSG00000198930 | 216.807444 | 0.76212296 | 0.2414772 | 3.15608666 | 0.00159901 | 0.04808737 |
| ENSG00000197172 | 639.016981 | 0.65638152 | 0.19091123 | 3.43815035 | 0.0005857 | 0.02482143 |
| ENSG00000182492 | 92.7048207 | 1.27490987 | 0.2695844 | 4.72916776 | 2.25E−06 | 0.00041401 |
| ENSG00000176595 | 806.922667 | −0.5983167 | 0.17479389 | −3.4229839 | 0.00061938 | 0.02578337 |
| ENSG00000173281 | 302.315743 | −0.7443989 | 0.2056548 | −3.6196523 | 0.000295 | 0.015728 |
| ENSG00000269918 | 101.042073 | 0.78492076 | 0.24793242 | 3.16586583 | 0.00154622 | 0.04703484 |
| ENSG00000104722 | 2094.48775 | 0.77927684 | 0.16768657 | 4.64722284 | 3.36E−06 | 0.0005623 |
| ENSG00000171320 | 398.674485 | 0.6495023 | 0.18744186 | 3.46508677 | 0.00053006 | 0.02360673 |
| ENSG00000120875 | 2837.84888 | −0.7595479 | 0.11810915 | −6.4308982 | 1.27E−10 | 8.99E−08 |
| ENSG00000156687 | 462.541799 | −0.7487925 | 0.21644965 | −3.4594304 | 0.00054132 | 0.02380678 |
| ENSG00000104738 | 3569.65926 | 0.66075568 | 0.09346931 | 7.06922589 | 1.56E−12 | 1.66E−09 |
| ENSG00000019549 | 335.184999 | 0.58706939 | 0.18066117 | 3.24956038 | 0.00115584 | 0.03907512 |
| ENSG00000254087 | 1214.33674 | −0.4074677 | 0.12945947 | −3.1474539 | 0.00164699 | 0.04870574 |
| ENSG00000104313 | 87.9807537 | −1.4904688 | 0.24166822 | −6.1674176 | 6.94E−10 | 4.13E−07 |
| ENSG00000250979 | 102.628872 | −1.3304262 | 0.27853607 | −4.7764952 | 1.78E−06 | 0.00035856 |
| ENSG00000121039 | 203.999663 | 0.77684641 | 0.18761674 | 4.14060285 | 3.46E−05 | 0.00341233 |
| ENSG00000164684 | 4033.99544 | −0.7727113 | 0.20283408 | −3.8095731 | 0.00013921 | 0.0091624 |
| ENSG00000136982 | 451.384204 | 0.62744351 | 0.18146238 | 3.45770584 | 0.0005448 | 0.02380678 |
| ENSG00000147684 | 928.496724 | 0.80392813 | 0.25513387 | 3.15100508 | 0.0016271 | 0.04840612 |
| ENSG00000178896 | 314.027821 | 0.95316643 | 0.29803537 | 3.19816548 | 0.00138305 | 0.04367845 |
| ENSG00000160957 | 946.03051 | 0.81351761 | 0.22312991 | 3.64593701 | 0.00026642 | 0.01478726 |
| ENSG00000178445 | 638.532752 | −0.5744287 | 0.13363132 | −4.2986081 | 1.72E−05 | 0.00204963 |
| ENSG00000147889 | 83.985427 | 1.25093392 | 0.34320406 | 3.64486919 | 0.00026753 | 0.01479359 |
| ENSG00000165264 | 674.393028 | 0.72371622 | 0.22028809 | 3.28531708 | 0.00101868 | 0.03616424 |
| ENSG00000107262 | 1053.12536 | 0.86876361 | 0.21845596 | 3.97683644 | 6.98E−05 | 0.00552576 |
| ENSG00000137100 | 762.22749 | 0.54750889 | 0.1689942 | 3.23980886 | 0.0011961 | 0.04016245 |
| ENSG00000221829 | 792.731761 | 0.51333965 | 0.13465958 | 3.81212869 | 0.00013778 | 0.00914925 |
| ENSG00000119139 | 1436.36363 | −0.5678164 | 0.12650622 | −4.4884463 | 7.17E−06 | 0.00100679 |
| ENSG00000135069 | 1673.25537 | 0.43449086 | 0.13612722 | 3.1917999 | 0.00141389 | 0.04427716 |
| ENSG00000148053 | 1534.09037 | 0.71706699 | 0.22571832 | 3.17682235 | 0.00148898 | 0.046047 |
| ENSG00000213694 | 440.652299 | 0.65955812 | 0.15345202 | 4.29813906 | 1.72E−05 | 0.00204963 |
| ENSG00000165244 | 531.821263 | 0.58181256 | 0.13237893 | 4.39505406 | 1.11E−05 | 0.00143246 |
| ENSG00000136943 | 209.087589 | 0.65528263 | 0.18004982 | 3.63945167 | 0.00027322 | 0.01493474 |
| ENSG00000136938 | 3938.99832 | 0.62364036 | 0.18550053 | 3.36193304 | 0.00077399 | 0.0296729 |
| ENSG00000165185 | 1197.93803 | −0.6826776 | 0.20186206 | −3.3819013 | 0.00071986 | 0.02793239 |
| ENSG00000119421 | 562.741549 | 0.62407441 | 0.19606841 | 3.18294207 | 0.00145787 | 0.04536775 |
| ENSG00000136828 | 587.502939 | −0.6632988 | 0.20074196 | −3.3042359 | 0.00095236 | 0.03457091 |
| ENSG00000136854 | 2972.62869 | −0.482256 | 0.13488004 | −3.5754434 | 0.00034963 | 0.01762989 |
| ENSG00000106991 | 776.329444 | 0.59858021 | 0.1506259 | 3.97395263 | 7.07E−05 | 0.00554657 |
| ENSG00000123454 | 5584.01389 | −0.7221269 | 0.19340917 | −3.7336746 | 0.00018871 | 0.01164733 |
| ENSG00000187796 | 268.810074 | 0.84016254 | 0.22971379 | 3.6574319 | 0.00025475 | 0.01429992 |
| ENSG00000148400 | 231.97295 | −1.1839828 | 0.34052804 | −3.4769025 | 0.00050724 | 0.02303701 |
| ENSG00000130600 | 2278.61782 | −0.6919268 | 0.18353935 | −3.76991 | 0.00016331 | 0.01038112 |
| ENSG00000167244 | 350.661076 | −2.5064145 | 0.24553069 | −10.208151 | 1.82E−24 | 9.04E−21 |
| ENSG00000166483 | 1590.30457 | 0.3819044 | 0.11807867 | 3.23432175 | 0.00121932 | 0.04066679 |
| ENSG00000133812 | 1420.92806 | −0.7869339 | 0.21119915 | −3.7260277 | 0.00019452 | 0.01181021 |
| ENSG00000050165 | 1293.35929 | −0.5809911 | 0.12212288 | −4.7574308 | 1.96E−06 | 0.00037092 |
| ENSG00000109881 | 577.857728 | 0.63159887 | 0.19958252 | 3.16460008 | 0.00155296 | 0.04704749 |
| ENSG00000066382 | 443.273746 | −0.6672194 | 0.14322885 | −4.658415 | 3.19E−06 | 0.00054017 |
| ENSG00000026508 | 193.463753 | −0.8095368 | 0.21701083 | −3.7303982 | 0.00019118 | 0.01170273 |
| ENSG00000157570 | 2075.93207 | −0.7161674 | 0.16935716 | −4.2287399 | 2.35E−05 | 0.0025331 |
| ENSG00000134569 | 315.67573 | −1.0381448 | 0.24365227 | −4.2607638 | 2.04E−05 | 0.00233114 |
| ENSG00000165916 | 1906.89858 | 0.55497284 | 0.16412189 | 3.38146751 | 0.000721 | 0.02793239 |
| ENSG00000255433 | 297.519497 | 0.89663288 | 0.24356143 | 3.68134183 | 0.00023201 | 0.01337653 |
| ENSG00000134809 | 380.53845 | 0.84385409 | 0.23825378 | 3.5418287 | 0.00039736 | 0.01933692 |
| ENSG00000189057 | 293.11634 | 0.75780995 | 0.23102645 | 3.28018693 | 0.00103738 | 0.03655391 |
| ENSG00000166900 | 132.660547 | −0.8359729 | 0.19731092 | −4.2368306 | 2.27E−05 | 0.00249528 |
| ENSG00000168496 | 1992.49991 | 0.96668632 | 0.15743403 | 6.14026293 | 8.24E−10 | 4.71E−07 |
| ENSG00000168003 | 1159.9806 | 0.71959297 | 0.18876425 | 3.81212536 | 0.00013778 | 0.00914925 |
| ENSG00000146670 | 1501.52517 | 0.58756458 | 0.13336723 | 4.40561424 | 1.05E−05 | 0.00138633 |
| ENSG00000014138 | 754.940952 | 0.56612604 | 0.12702673 | 4.45674724 | 8.32E−06 | 0.00111512 |
| ENSG00000172922 | 620.929055 | 0.8379512 | 0.23101513 | 3.62725685 | 0.00028645 | 0.01532705 |
| ENSG00000132749 | 187.790801 | −0.7360248 | 0.18785153 | −3.9181199 | 8.92E−05 | 0.00641294 |
| ENSG00000033327 | 2928.87863 | −0.7602815 | 0.20811995 | −3.6530926 | 0.0002591 | 0.01443492 |
| ENSG00000182103 | 187.050756 | 0.98634 | 0.24047714 | 4.10159564 | 4.10E−05 | 0.00375989 |
| ENSG00000150687 | 306.524588 | 0.51674741 | 0.16321367 | 3.16607921 | 0.00154509 | 0.04703484 |
| ENSG00000137727 | 53.981437 | −1.3969814 | 0.32682836 | −4.274358 | 1.92E−05 | 0.00221037 |
| ENSG00000109846 | 34.7345882 | 1.40546 | 0.39024783 | 3.60145502 | 0.00031644 | 0.01645826 |
| ENSG00000150779 | 676.874435 | 0.8549178 | 0.24473788 | 3.49319762 | 0.00047727 | 0.02200473 |
| ENSG00000188486 | 3128.38886 | 0.73477514 | 0.22768012 | 3.22722569 | 0.00124997 | 0.04114605 |
| ENSG00000149403 | 112.702881 | −1.3447072 | 0.25872176 | −5.1975033 | 2.02E−07 | 6.39E−05 |
| ENSG00000137642 | 677.959403 | −0.7819053 | 0.2056126 | −3.8028085 | 0.00014306 | 0.00933373 |
| ENSG00000154146 | 207.452709 | 1.15611337 | 0.27779668 | 4.16172487 | 3.16E−05 | 0.00317453 |
| ENSG00000149557 | 902.45893 | 0.53977949 | 0.16781856 | 3.21644689 | 0.00129789 | 0.04229943 |
| ENSG00000166105 | 107.301143 | 0.74768661 | 0.22159393 | 3.37412949 | 0.0007405 | 0.02853592 |
| ENSG00000255545 | 42.0985936 | −1.2297408 | 0.33288944 | −3.6941417 | 0.00022063 | 0.01307523 |
| ENSG00000197308 | 479.427991 | 0.89328414 | 0.26885904 | 3.32249992 | 0.00089215 | 0.03268642 |
| ENSG00000151468 | 76.7451882 | 0.95438824 | 0.25986821 | 3.67258555 | 0.00024011 | 0.01363213 |
| ENSG00000065328 | 1189.77449 | 0.54483344 | 0.13177644 | 4.13452836 | 3.56E−05 | 0.00343559 |
| ENSG00000078114 | 735.314879 | −1.1261988 | 0.19946736 | −5.6460305 | 1.64E−08 | 6.78E−06 |
| ENSG00000095739 | 1191.41474 | 0.97719979 | 0.17000826 | 5.74795472 | 9.03E−09 | 3.84E−06 |
| ENSG00000165633 | 182.739294 | −0.7836823 | 0.21311232 | −3.6773206 | 0.0002357 | 0.0134764 |
| ENSG00000122952 | 740.142453 | 0.43110353 | 0.13614499 | 3.16650315 | 0.00154284 | 0.04703484 |
| ENSG00000138336 | 891.407148 | 1.40112623 | 0.26249986 | 5.33762661 | 9.42E−08 | 3.42E−05 |
| ENSG00000156515 | 3490.71713 | −0.3534318 | 0.11141612 | −3.1721782 | 0.001513 | 0.04649979 |
| ENSG00000107742 | 576.43466 | 0.82552534 | 0.17622482 | 4.68450098 | 2.81E−06 | 0.00049112 |
| ENSG00000156113 | 609.391281 | −0.7576227 | 0.16942879 | −4.4716288 | 7.76E−06 | 0.00106916 |
| ENSG00000187122 | 962.018273 | −0.6551029 | 0.20666627 | −3.1698588 | 0.00152513 | 0.04667966 |
| ENSG00000095713 | 255.091454 | 0.67110393 | 0.20315556 | 3.30339922 | 0.0009552 | 0.03457091 |
| ENSG00000099194 | 7740.70394 | −0.6602298 | 0.14991411 | −4.4040537 | 1.06E−05 | 0.00138633 |
| ENSG00000156398 | 208.918663 | 0.6854882 | 0.17123408 | 4.00322293 | 6.25E−05 | 0.00507907 |
| ENSG00000108018 | 1665.51288 | −0.6442086 | 0.20158967 | −3.1956426 | 0.0013952 | 0.0437839 |
| ENSG00000150594 | 61.3664637 | 0.92498184 | 0.29173032 | 3.17067438 | 0.00152086 | 0.04664479 |
| ENSG00000165868 | 1024.44815 | −0.6269397 | 0.15491684 | −4.0469437 | 5.19E−05 | 0.00431215 |
| ENSG00000187164 | 448.261163 | −0.5806547 | 0.14681531 | −3.9550011 | 7.65E−05 | 0.00580972 |
| ENSG00000119973 | 22.2654684 | −1.9296219 | 0.56740605 | −3.4007778 | 0.00067194 | 0.02691094 |
| ENSG00000198873 | 343.660662 | −0.7624891 | 0.16228612 | −4.6984249 | 2.62E−06 | 0.00046427 |
| ENSG00000148848 | 678.219215 | −0.8681534 | 0.20883601 | −4.157106 | 3.22E−05 | 0.00321764 |
| ENSG00000108010 | 1095.06384 | 0.62839146 | 0.18632489 | 3.37255786 | 0.00074473 | 0.02862513 |
| ENSG00000188385 | 129.617311 | −0.890381 | 0.20962309 | −4.2475332 | 2.16E−05 | 0.00244235 |
| ENSG00000182326 | 37.9394579 | −1.3776518 | 0.3978194 | −3.463008 | 0.00053417 | 0.02371885 |
| ENSG00000139182 | 1013.40793 | −0.6187176 | 0.1629158 | −3.7977751 | 0.000146 | 0.00948367 |
| ENSG00000172572 | 84.4248088 | −1.4618384 | 0.32434421 | −4.5070588 | 6.57E−06 | 0.00094929 |
| ENSG00000084453 | 98.6224656 | −0.8356097 | 0.2509974 | −3.3291568 | 0.00087109 | 0.03215565 |
| ENSG00000111728 | 53.8112263 | −1.3055019 | 0.31971718 | −4.0833023 | 4.44E−05 | 0.00395271 |
| ENSG00000057294 | 100.9103 | −0.8360778 | 0.25625473 | −3.2626823 | 0.00110363 | 0.03782608 |
| ENSG00000173208 | 456.559256 | −0.8938114 | 0.22635649 | −3.9486892 | 7.86E−05 | 0.00584441 |
| ENSG00000018236 | 2362.82253 | −1.0226902 | 0.15589266 | −6.5602205 | 5.37E−11 | 4.09E−08 |
| ENSG00000184613 | 493.915011 | −0.5951681 | 0.12981525 | −4.5847322 | 4.55E−06 | 0.0007513 |
| ENSG00000170627 | 86.8767792 | 1.14682726 | 0.27860576 | 4.11630851 | 3.85E−05 | 0.0036017 |
| ENSG00000123374 | 502.515414 | 0.53541811 | 0.1643323 | 3.25814286 | 0.00112144 | 0.03817256 |
| ENSG00000011465 | 1260.14348 | 0.63928515 | 0.15785534 | 4.04981645 | 5.13E−05 | 0.00430393 |
| ENSG00000151136 | 253.165693 | −0.7274702 | 0.18243735 | −3.9875071 | 6.68E−05 | 0.00531135 |
| ENSG00000076248 | 1331.85909 | 0.40736001 | 0.11902937 | 3.42234855 | 0.00062083 | 0.02578337 |
| ENSG00000076555 | 497.510696 | −0.719086 | 0.20796153 | −3.4577838 | 0.00054464 | 0.02380678 |
| ENSG00000060709 | 3307.95041 | −0.5156101 | 0.15468693 | −3.333249 | 0.00085838 | 0.03206236 |
| ENSG00000196199 | 1316.71137 | 0.54631348 | 0.17160937 | 3.18347119 | 0.00145521 | 0.04536775 |
| ENSG00000165480 | 470.356075 | 0.68560117 | 0.17481106 | 3.92195524 | 8.78E−05 | 0.00639823 |
| ENSG00000133083 | 911.212212 | −0.5703831 | 0.15092076 | −3.7793546 | 0.00015724 | 0.01003809 |
| ENSG00000276644 | 148.725288 | −0.7651748 | 0.19352416 | −3.9538983 | 7.69E−05 | 0.00580972 |
| ENSG00000178695 | 2729.12734 | 0.50854294 | 0.15904103 | 3.1975581 | 0.00138596 | 0.04367845 |
| ENSG00000088387 | 260.276875 | −0.7813446 | 0.2236955 | −3.4928936 | 0.00047782 | 0.02200473 |
| ENSG00000102466 | 475.288892 | −0.5677685 | 0.17003757 | −3.3390768 | 0.00084057 | 0.03172534 |
| ENSG00000125266 | 1816.3175 | −0.8907716 | 0.11700215 | −7.6132927 | 2.67E−14 | 4.97E−11 |
| ENSG00000204442 | 1698.86651 | −0.4895261 | 0.10205228 | −4.7968174 | 1.61E−06 | 0.00033774 |
| ENSG00000274718 | 105.845859 | −1.7288267 | 0.26059207 | −6.6342262 | 3.26E−11 | 3.03E−08 |
| ENSG00000198176 | 1888.55783 | 0.47018584 | 0.11590139 | 4.05677487 | 4.98E−05 | 0.00425348 |
| ENSG00000259017 | 37.315656 | −1.8256537 | 0.40502946 | −4.5074591 | 6.56E−06 | 0.00094929 |
| ENSG00000168348 | 403.695123 | −1.9321493 | 0.18831274 | −10.260321 | 1.06E−24 | 7.91E−21 |
| ENSG00000174373 | 890.841032 | −0.7296437 | 0.22667686 | −3.2188715 | 0.00128696 | 0.04216642 |
| ENSG00000100479 | 251.080204 | 0.67380958 | 0.17536314 | 3.8423672 | 0.00012185 | 0.00831453 |
| ENSG00000020577 | 261.2629 | −0.8874652 | 0.21745567 | −4.0811318 | 4.48E−05 | 0.00395271 |
| ENSG00000131979 | 905.131557 | −0.7997083 | 0.15000209 | −5.3313144 | 9.75E−08 | 3.45E−05 |
| ENSG00000070182 | 86.268933 | −1.0543298 | 0.30485182 | −3.4584992 | 0.00054319 | 0.02380678 |
| ENSG00000274330 | 17.1569299 | 1.6527543 | 0.49506551 | 3.33845575 | 0.00084245 | 0.03172534 |
| ENSG00000119681 | 373.285888 | −0.8500122 | 0.24920091 | −3.4109515 | 0.00064737 | 0.02616731 |
| ENSG00000100604 | 8593.89536 | −1.0374267 | 0.20233992 | −5.1271478 | 2.94E−07 | 9.12E−05 |
| ENSG00000182218 | 44.6313937 | −1.4650536 | 0.34674137 | −4.2252057 | 2.39E−05 | 0.00255468 |
| ENSG00000183092 | 1514.42165 | −0.823253 | 0.22967113 | −3.5844865 | 0.00033774 | 0.01732384 |
| ENSG00000259031 | 31.9495195 | −1.7144281 | 0.43724106 | −3.9210134 | 8.82E−05 | 0.00639823 |
| ENSG00000185559 | 1605.2048 | −1.6518641 | 0.24959063 | −6.6182936 | 3.63E−11 | 3.18E−08 |
| ENSG00000254656 | 1169.57968 | −2.1558166 | 0.28664697 | −7.5208072 | 5.44E−14 | 9.00E−11 |
| ENSG00000221077 | 55.3196793 | −2.345698 | 0.47058942 | −4.9845958 | 6.21E−07 | 0.00016204 |
| ENSG00000080824 | 29725.2457 | 0.67894313 | 0.19266381 | 3.52397859 | 0.00042512 | 0.01995209 |
| ENSG00000258986 | 548.914861 | 0.95208522 | 0.19130361 | 4.97682836 | 6.46E−07 | 0.00016577 |
| ENSG00000184601 | 31.3868627 | 1.22419416 | 0.37376069 | 3.27534217 | 0.00105534 | 0.03676392 |
| ENSG00000184990 | 672.378919 | 0.93872706 | 0.27451635 | 3.419567 | 0.00062721 | 0.02578337 |
| ENSG00000185347 | 243.11557 | 1.02765337 | 0.28531534 | 3.60181609 | 0.000316 | 0.01645826 |
| ENSG00000175344 | 429.989218 | −0.6062761 | 0.17540467 | −3.4564425 | 0.00054736 | 0.02380678 |
| ENSG00000051180 | 526.852118 | 0.67484041 | 0.14959523 | 4.51110923 | 6.45E−06 | 0.00094929 |
| ENSG00000128965 | 207.156343 | 0.7768448 | 0.24155031 | 3.21607862 | 0.00129955 | 0.04229943 |
| ENSG00000137825 | 50.2762886 | 1.11159812 | 0.35223226 | 3.15586692 | 0.00160022 | 0.04808737 |
| ENSG00000128951 | 2406.59681 | 0.68649035 | 0.19112543 | 3.59183163 | 0.00032836 | 0.01690098 |
| ENSG00000138587 | 152.444254 | 0.7618604 | 0.23280379 | 3.27254288 | 0.00106585 | 0.03689199 |
| ENSG00000140416 | 1362.61801 | 0.73224651 | 0.13427484 | 5.45334117 | 4.94E−08 | 1.84E−05 |
| ENSG00000166803 | 340.428891 | 1.07561913 | 0.25006294 | 4.30139364 | 1.70E−05 | 0.00204963 |
| ENSG00000137834 | 532.719059 | 1.07922932 | 0.22530379 | 4.7901072 | 1.67E−06 | 0.00033966 |
| ENSG00000128973 | 608.332068 | 0.57071682 | 0.14864841 | 3.83937374 | 0.00012335 | 0.00837813 |
| ENSG00000259781 | 390.887013 | 0.81201833 | 0.23742214 | 3.42014577 | 0.00062588 | 0.02578337 |
| ENSG00000140365 | 976.638153 | 0.86375936 | 0.2381109 | 3.62755066 | 0.00028612 | 0.01532705 |
| ENSG00000161980 | 253.339381 | 0.77167428 | 0.23179452 | 3.32913079 | 0.00087117 | 0.03215565 |
| ENSG00000161981 | 443.672135 | 1.00496931 | 0.23883835 | 4.20773852 | 2.58E−05 | 0.00272117 |
| ENSG00000182685 | 115.470975 | 0.76128365 | 0.23802689 | 3.19830941 | 0.00138236 | 0.04367845 |
| ENSG00000162062 | 621.103782 | 0.83517622 | 0.2036697 | 4.10064057 | 4.12E−05 | 0.00375989 |
| ENSG00000118898 | 33.6793528 | −1.8358652 | 0.40082968 | −4.5801627 | 4.65E−06 | 0.00075947 |
| ENSG00000175643 | 583.354589 | 0.6774891 | 0.15807079 | 4.28598549 | 1.82E−05 | 0.00214779 |
| ENSG00000149929 | 606.917543 | 0.681939 | 0.16594262 | 4.10948684 | 3.97E−05 | 0.00366368 |
| ENSG00000179958 | 502.09622 | 0.82432078 | 0.21752677 | 3.78951414 | 0.00015094 | 0.00976202 |
| ENSG00000089280 | 8051.48337 | 0.58509767 | 0.15667208 | 3.73453697 | 0.00018806 | 0.01164733 |
| ENSG00000091651 | 607.103796 | 0.7975172 | 0.17089703 | 4.66665329 | 3.06E−06 | 0.00052952 |
| ENSG00000125148 | 44.9000027 | 1.55789865 | 0.40543406 | 3.84254504 | 0.00012177 | 0.00831453 |
| ENSG00000125170 | 4162.24254 | 0.5572408 | 0.13389568 | 4.16175328 | 3.16E−05 | 0.00317453 |
| ENSG00000181938 | 229.819668 | 0.67793174 | 0.18765138 | 3.61271916 | 0.000303 | 0.01609702 |
| ENSG00000140937 | 877.908772 | −0.540172 | 0.14474373 | −3.7319197 | 0.00019003 | 0.01168032 |
| ENSG00000103154 | 145.991349 | 2.02268453 | 0.25154751 | 8.04096424 | 8.91E−16 | 2.21E−12 |
| ENSG00000103187 | 323.892506 | 0.91201813 | 0.20356339 | 4.48026608 | 7.46E−06 | 0.00103638 |
| ENSG00000131153 | 656.796472 | 0.89952173 | 0.18568658 | 4.84430116 | 1.27E−06 | 0.00029071 |
| ENSG00000167523 | 188.06405 | 0.66965001 | 0.19626163 | 3.41202723 | 0.00064482 | 0.02613528 |
| ENSG00000129235 | 583.92011 | 0.86988115 | 0.25285566 | 3.44022815 | 0.00058122 | 0.02482143 |
| ENSG00000129255 | 781.585056 | 0.59587689 | 0.17527302 | 3.3997068 | 0.00067458 | 0.02691094 |
| ENSG00000179111 | 51.1174922 | −1.2433054 | 0.34839597 | −3.568656 | 0.00035882 | 0.01803178 |
| ENSG00000172301 | 496.16905 | 0.70102757 | 0.21790635 | 3.21710482 | 0.00129491 | 0.04229943 |
| ENSG00000267321 | 129.437949 | 0.87327784 | 0.26128064 | 3.34229824 | 0.00083088 | 0.03152883 |
| ENSG00000173991 | 58.3180863 | 0.92681516 | 0.27890441 | 3.32305665 | 0.00089037 | 0.03268642 |
| ENSG00000094804 | 869.943664 | 0.72859488 | 0.14830759 | 4.91272829 | 8.98E−07 | 0.00021549 |
| ENSG00000167920 | 98.9983987 | 0.95244498 | 0.24271466 | 3.92413451 | 8.70E−05 | 0.00637808 |
| ENSG00000108861 | 614.956278 | −0.4199559 | 0.1320699 | −3.1798 | 0.00147377 | 0.04571935 |
| ENSG00000186868 | 1366.26959 | −0.5332143 | 0.14461041 | −3.6872472 | 0.00022669 | 0.01327583 |
| ENSG00000108465 | 1821.35087 | 0.57057444 | 0.15865463 | 3.5963303 | 0.00032274 | 0.01666919 |
| ENSG00000239672 | 595.254447 | 1.06382914 | 0.2678204 | 3.97217367 | 7.12E−05 | 0.00554657 |
| ENSG00000087191 | 1942.01786 | 0.63743316 | 0.1656823 | 3.84732196 | 0.00011942 | 0.00822367 |
| ENSG00000154229 | 11045.3539 | −0.7098494 | 0.18663352 | −3.8034403 | 0.0001427 | 0.00933373 |
| ENSG00000075461 | 5066.4808 | −0.7144756 | 0.16021034 | −4.45961 | 8.21E−06 | 0.00111034 |
| ENSG00000180616 | 156.262292 | 1.15134544 | 0.22089249 | 5.21224339 | 1.87E−07 | 6.03E−05 |
| ENSG00000125450 | 964.009726 | 0.41616787 | 0.13012386 | 3.19824411 | 0.00138267 | 0.04367845 |
| ENSG00000161547 | 3572.4897 | 0.64676002 | 0.20572071 | 3.14387415 | 0.00166727 | 0.04920766 |
| ENSG00000167900 | 835.760296 | 0.96930591 | 0.19251034 | 5.03508495 | 4.78E−07 | 0.00013663 |
| ENSG00000089685 | 1727.20212 | 0.63128763 | 0.15917704 | 3.96594658 | 7.31E−05 | 0.00565117 |
| ENSG00000224877 | 917.86588 | 0.95484517 | 0.29619563 | 3.22369775 | 0.00126547 | 0.04155375 |
| ENSG00000183048 | 119.565826 | 1.04866961 | 0.31948867 | 3.28233742 | 0.0010295 | 0.03637497 |
| ENSG00000183684 | 2016.78563 | 0.88124397 | 0.23929635 | 3.68264692 | 0.00023082 | 0.01335999 |
| ENSG00000176890 | 588.137002 | 0.68922579 | 0.17758108 | 3.88118925 | 0.00010395 | 0.00732801 |
| ENSG00000080986 | 668.269042 | 0.62906952 | 0.18600849 | 3.38193989 | 0.00071976 | 0.02793239 |
| ENSG00000173482 | 140.970869 | −1.3292451 | 0.21171225 | −6.278546 | 3.42E−10 | 2.31E−07 |
| ENSG00000141441 | 565.882074 | −0.9765826 | 0.17410274 | −5.6092314 | 2.03E−08 | 8.17E−06 |
| ENSG00000166974 | 675.182292 | −0.530745 | 0.15393322 | −3.4478916 | 0.00056498 | 0.02428928 |
| ENSG00000184828 | 82.934306 | −1.015185 | 0.24573998 | −4.131135 | 3.61E−05 | 0.003442 |
| ENSG00000125835 | 2607.27947 | 0.82037717 | 0.23281659 | 3.52370579 | 0.00042556 | 0.01995209 |
| ENSG00000101361 | 2102.08274 | 0.54571463 | 0.11977113 | 4.55631209 | 5.21E−06 | 0.00081115 |
| ENSG00000088854 | 738.906445 | −0.562598 | 0.15246975 | −3.6898989 | 0.00022434 | 0.01324248 |
| ENSG00000132646 | 3349.40447 | 0.87122073 | 0.18065096 | 4.82267423 | 1.42E−06 | 0.00030985 |
| ENSG00000089199 | 2028.44486 | −0.4624342 | 0.10694522 | −4.3240292 | 1.53E−05 | 0.00191506 |
| ENSG00000125869 | 699.512665 | 0.75680214 | 0.15791217 | 4.79255109 | 1.65E−06 | 0.00033966 |
| ENSG00000101003 | 620.311777 | 0.61423161 | 0.17757843 | 3.45893141 | 0.00054232 | 0.02380678 |
| ENSG00000125968 | 1196.66942 | 1.11910003 | 0.25048065 | 4.46781039 | 7.90E−06 | 0.00107843 |
| ENSG00000101412 | 1344.81629 | 0.9013056 | 0.18209809 | 4.94956093 | 7.44E−07 | 0.00018536 |
| ENSG00000149636 | 402.47954 | 0.64129764 | 0.18053586 | 3.55218977 | 0.00038204 | 0.01887985 |
| ENSG00000204103 | 312.707527 | 1.01322919 | 0.20710462 | 4.89235429 | 9.96E−07 | 0.00023158 |
| ENSG00000101057 | 3301.17794 | 0.58238583 | 0.14275871 | 4.07951169 | 4.51E−05 | 0.00395271 |
| ENSG00000124191 | 489.704233 | −0.6690915 | 0.21208494 | −3.1548279 | 0.00160593 | 0.04811176 |
| ENSG00000158445 | 255.203877 | −0.8840059 | 0.21373521 | −4.1359862 | 3.53E−05 | 0.00343559 |
| ENSG00000124216 | 355.278159 | 0.60791364 | 0.178643 | 3.40295252 | 0.00066662 | 0.02679987 |
| ENSG00000054803 | 24.1796649 | −5.4271565 | 0.87599697 | −6.1954056 | 5.81E−10 | 3.60E−07 |
| ENSG00000101144 | 48.55399 | −1.6046206 | 0.34449964 | −4.6578295 | 3.20E−06 | 0.00054017 |
| ENSG00000130270 | 92.4956445 | −1.1886052 | 0.25172617 | −4.7218183 | 2.34E−06 | 0.00042402 |
| ENSG00000167670 | 1247.79762 | 0.78632178 | 0.14859148 | 5.29183615 | 1.21E−07 | 4.19E−05 |
| ENSG00000280239 | 22.7532849 | 1.80912674 | 0.52941651 | 3.41720875 | 0.00063267 | 0.02578337 |
| ENSG00000276043 | 1534.19026 | 0.63364712 | 0.17945889 | 3.53087611 | 0.00041419 | 0.01993855 |
| ENSG00000205744 | 120.232388 | −0.9127556 | 0.21500764 | −4.2452241 | 2.18E−05 | 0.00244235 |
| ENSG00000099783 | 5464.83442 | 0.73425565 | 0.16198722 | 4.53280002 | 5.82E−06 | 0.00087457 |
| ENSG00000198258 | 2152.44738 | 0.82647776 | 0.25993986 | 3.17949609 | 0.00147531 | 0.04571935 |
| ENSG00000161888 | 510.174599 | 0.89862706 | 0.18881165 | 4.75938348 | 1.94E−06 | 0.00037092 |
| ENSG00000104889 | 1000.50637 | 0.97653266 | 0.21709208 | 4.49824182 | 6.85E−06 | 0.00098 |
| ENSG00000105011 | 508.492535 | 0.60175125 | 0.13966832 | 4.30843045 | 1.64E−05 | 0.00201553 |
| ENSG00000123136 | 1562.21912 | 0.64280099 | 0.19636018 | 3.27358113 | 0.00106194 | 0.03689199 |
| ENSG00000105393 | 339.472345 | 0.81760884 | 0.23789844 | 3.4367978 | 0.00058863 | 0.02487484 |
| ENSG00000105639 | 119.482856 | −0.6959122 | 0.20711653 | −3.360003 | 0.00077942 | 0.02980415 |
| ENSG00000160161 | 160.686702 | −0.8488872 | 0.27069402 | −3.135966 | 0.00171289 | 0.04986371 |
| ENSG00000269416 | 339.54691 | 0.55676136 | 0.16226372 | 3.43121288 | 0.00060089 | 0.02532074 |
| ENSG00000105173 | 388.91138 | 0.5935736 | 0.17087936 | 3.47364121 | 0.00051345 | 0.02314402 |
| ENSG00000124302 | 560.471054 | 0.88562478 | 0.23903602 | 3.70498458 | 0.0002114 | 0.01267995 |
| ENSG00000011332 | 433.045432 | 1.05552553 | 0.25954641 | 4.06680838 | 4.77E−05 | 0.00412188 |
| ENSG00000125746 | 301.72452 | −0.5722012 | 0.17867924 | −3.2023933 | 0.00136291 | 0.0435984 |
| ENSG00000124440 | 96.4992964 | −0.9911026 | 0.23337901 | −4.2467513 | 2.17E−05 | 0.00244235 |
| ENSG00000105281 | 1168.99505 | 0.57324549 | 0.16717933 | 3.42892568 | 0.00060598 | 0.02546295 |
| ENSG00000142230 | 2731.46721 | 0.46645985 | 0.13754663 | 3.39128528 | 0.00069566 | 0.02744799 |
| ENSG00000142552 | 199.303656 | 0.76675578 | 0.23873334 | 3.21176669 | 0.00131921 | 0.04256685 |
| ENSG00000167747 | 881.673158 | 0.64272983 | 0.18078146 | 3.55528613 | 0.00037757 | 0.01872108 |
| ENSG00000093009 | 724.72203 | 0.82476769 | 0.13920431 | 5.92487167 | 3.13E−09 | 1.41E−06 |
| ENSG00000099901 | 3673.15568 | 0.77991822 | 0.20247097 | 3.85200026 | 0.00011716 | 0.00813329 |
| ENSG00000100024 | 27.9159568 | −1.4583038 | 0.42006906 | −3.471581 | 0.0005174 | 0.02318184 |
| ENSG00000100297 | 1649.10322 | 0.63730239 | 0.16087195 | 3.96155064 | 7.45E−05 | 0.00570959 |
| ENSG00000128283 | 159.970264 | 0.82604698 | 0.25799726 | 3.20176645 | 0.00136588 | 0.04359961 |
| ENSG00000100129 | 6351.35552 | −0.3862673 | 0.11777819 | −3.2796165 | 0.00103948 | 0.03655391 |
| ENSG00000128272 | 3378.9489 | 0.55078505 | 0.15552104 | 3.54154688 | 0.00039779 | 0.01933692 |
| ENSG00000100162 | 136.328755 | 1.06903064 | 0.23549028 | 4.5395956 | 5.64E−06 | 0.0008555 |
| ENSG00000202058 | 21.2434535 | −2.243459 | 0.69931859 | −3.2080643 | 0.00133632 | 0.04283988 |
| ENSG00000100416 | 916.810683 | 0.49944145 | 0.1452633 | 3.43818067 | 0.00058564 | 0.02482143 |
| ENSG00000025770 | 880.821129 | 0.67878568 | 0.17486319 | 3.8818101 | 0.00010368 | 0.00732801 |
| ENSG00000277437 | 53.4048221 | −3.7452416 | 1.01544642 | −3.688271 | 0.00022578 | 0.0132748 |
| ENSG00000277105 | 13850.374 | −3.4915696 | 0.97906668 | −3.5662225 | 0.00036216 | 0.01813868 |
| ENSG00000276737 | 409.786811 | −3.769321 | 1.06949845 | −3.5243819 | 0.00042447 | 0.01995209 |
| ENSG00000274735 | 322.607828 | −3.7608853 | 0.88845644 | −4.2330554 | 2.31E−05 | 0.0025031 |
| ENSG00000279718 | 56.5409922 | −0.9916965 | 0.30114366 | −3.2931011 | 0.00099089 | 0.03558227 |
| ENSG00000154734 | 2372.64189 | −0.5189089 | 0.11470966 | −4.5236717 | 6.08E−06 | 0.00090404 |
| ENSG00000154736 | 211.849603 | −1.0613665 | 0.2198382 | −4.8279438 | 1.38E−06 | 0.00030627 |
| ENSG00000159055 | 372.534032 | 0.76630863 | 0.20427096 | 3.75143207 | 0.00017583 | 0.01103558 |
| ENSG00000159259 | 510.048758 | 0.4961973 | 0.14038957 | 3.53443137 | 0.00040865 | 0.01973611 |
| ENSG00000183527 | 375.521209 | 0.52270312 | 0.16354557 | 3.19607026 | 0.00139313 | 0.0437839 |
| ENSG00000160179 | 150.495894 | −0.8244522 | 0.23019332 | −3.5815644 | 0.00034154 | 0.01744336 |
| ENSG00000228709 | 459.974012 | −0.8662752 | 0.23605303 | −3.6698332 | 0.00024271 | 0.01372735 |
| ENSG00000175894 | 596.369164 | −0.7936253 | 0.20072974 | −3.9537004 | 7.70E−05 | 0.00580972 |
| ENSG00000235890 | 709.057452 | −1.2116973 | 0.26600638 | −4.5551437 | 5.23E−06 | 0.00081115 |
| ENSG00000182912 | 1286.0869 | −1.0172503 | 0.17149546 | −5.9316459 | 3.00E−09 | 1.39E−06 |

TABLE 6
Differential gene Expression_DESeq2_dCVdCT_dC

| | | Log2 Fold | | | | |
|---|---|---|---|---|---|---|
| | Base Mean | Change | Lfcse | Stat | Pvalue | Padj |
| ENSG00000223764 | 513.919766 | 0.10910155 | 0.1408231 | 0.77474189 | 0.43849219 | 0.9694822 |
| ENSG00000187634 | 1066.25504 | 0.26490854 | 0.19532773 | 1.35622596 | 0.17502728 | 0.94056812 |
| ENSG00000242485 | 937.600436 | 0.04109376 | 0.21822692 | 0.18830746 | 0.85063564 | 0.99145366 |
| ENSG00000160072 | 699.547282 | −0.1739675 | 0.18189009 | −0.956443 | 0.33884844 | 0.96302041 |
| ENSG00000171608 | 251.374075 | −0.4030591 | 0.16829058 | −2.3950189 | 0.01661951 | 0.60525335 |
| ENSG00000053372 | 1048.70128 | 0.1041349 | 0.17179396 | 0.60616161 | 0.54440744 | 0.97648438 |
| ENSG00000007968 | 639.194696 | 0.22158022 | 0.1289021 | 1.71898077 | 0.08561787 | 0.92973396 |
| ENSG00000117318 | 1362.20122 | 0.10339611 | 0.22960672 | 0.45031831 | 0.65248094 | 0.98262846 |
| ENSG00000127423 | 102.054816 | 0.39547585 | 0.21988282 | 1.79857552 | 0.07208585 | 0.92327552 |
| ENSG00000198830 | 7194.36677 | 0.2001733 | 0.1240174 | 1.6140743 | 0.10651131 | 0.92973396 |
| ENSG00000117748 | 1234.30582 | 0.26772624 | 0.129311 | 2.07040574 | 0.03841436 | 0.81443841 |
| ENSG00000130770 | 1000.4386 | 0.15493362 | 0.19260803 | 0.80439856 | 0.42116683 | 0.9694822 |
| ENSG00000092853 | 835.308296 | 0.27129091 | 0.16879685 | 1.60720362 | 0.10800971 | 0.92973396 |
| ENSG00000168389 | 269.923372 | 0.35160485 | 0.136541 | 2.57508624 | 0.01002151 | 0.48129017 |
| ENSG00000117016 | 1948.07408 | −0.213792 | 0.1474137 | −1.4502862 | 0.14697873 | 0.9339277 |
| ENSG00000171960 | 754.463631 | 0.11289011 | 0.17134864 | 0.65883285 | 0.51000311 | 0.97158774 |
| ENSG00000117395 | 1578.53292 | 0.20147084 | 0.1693802 | 1.18945922 | 0.23425901 | 0.95658313 |
| ENSG00000132773 | 227.758077 | 0.16494205 | 0.16135103 | 1.02225589 | 0.30665981 | 0.96279754 |
| ENSG00000132780 | 6332.75757 | 0.23516091 | 0.1114675 | 2.10968139 | 0.03488581 | 0.7877789 |
| ENSG00000162407 | 227.302124 | 0.26924448 | 0.17014406 | 1.58245012 | 0.11354685 | 0.92973396 |
| ENSG00000116678 | 150.314744 | −0.0602709 | 0.23891951 | −0.2522643 | 0.8008368 | 0.98975709 |
| ENSG00000178965 | 215.550561 | 0.59259332 | 0.22058235 | 2.6864947 | 0.00722061 | 0.41123929 |
| ENSG00000153904 | 1124.74806 | −0.4531148 | 0.1468771 | −3.0849925 | 0.00203557 | 0.20012214 |
| ENSG00000143013 | 215.289297 | 0.80017152 | 0.19354513 | 4.134289 | 3.56E−05 | 0.01046357 |
| ENSG00000171488 | 707.048687 | −0.2995185 | 0.16886091 | −1.773759 | 0.07610303 | 0.92973396 |
| ENSG00000137962 | 326.475887 | −0.4236925 | 0.19514968 | −2.1711156 | 0.02992243 | 0.76376729 |
| ENSG00000143036 | 29.7039746 | −1.1463815 | 0.35469311 | −3.2320377 | 0.00122911 | 0.14448171 |
| ENSG00000237954 | 49.4200112 | −1.0893535 | 0.2990241 | −3.6430292 | 0.00026945 | 0.05048406 |
| ENSG00000188641 | 587.752757 | −0.3042693 | 0.1509957 | −2.015086 | 0.04389564 | 0.83825609 |
| ENSG00000116299 | 132.548918 | −0.1839682 | 0.19981868 | −0.9206756 | 0.35721979 | 0.96401724 |
| ENSG00000116396 | 116.688511 | −0.6477082 | 0.20050246 | −3.2304252 | 0.00123606 | 0.14449638 |
| ENSG00000092621 | 3312.3205 | 0.33707206 | 0.14263251 | 2.36322053 | 0.01811689 | 0.6253289 |
| ENSG00000265972 | 8541.57977 | −0.1415965 | 0.1178511 | −1.2014862 | 0.22956264 | 0.95487879 |
| ENSG00000203814 | 22.7288953 | −0.7957932 | 0.39852905 | −1.9968261 | 0.04584407 | 0.84643522 |
| ENSG00000160691 | 5930.36505 | −0.1988347 | 0.1476468 | −1.3466912 | 0.1780797 | 0.94056812 |
| ENSG00000160783 | 229.624984 | 0.18977057 | 0.24200113 | 0.78417227 | 0.43293903 | 0.9694822 |
| ENSG00000160818 | 1077.22567 | 0.16254477 | 0.17884887 | 0.90883867 | 0.36343529 | 0.96401724 |
| ENSG00000143153 | 3963.35425 | −0.2819938 | 0.08877232 | −3.1765964 | 0.00149014 | 0.16421838 |
| ENSG00000116147 | 2240.0628 | −0.5797846 | 0.23575895 | −2.4592262 | 0.01392369 | 0.56146632 |
| ENSG00000186283 | 539.344545 | 0.18012443 | 0.12716145 | 1.41650178 | 0.15662864 | 0.93425563 |
| ENSG00000143333 | 849.829044 | 0.43602237 | 0.15551982 | 2.80364505 | 0.00505285 | 0.34463288 |
| ENSG00000135829 | 7804.95605 | 0.27277816 | 0.1024104 | 2.66357863 | 0.00773143 | 0.42645043 |
| ENSG00000159176 | 579.821256 | 0.42778133 | 0.14655681 | 2.91887727 | 0.00351295 | 0.28401271 |
| ENSG00000077152 | 1335.13612 | 0.30360878 | 0.18383289 | 1.6515477 | 0.09862679 | 0.92973396 |
| ENSG00000117139 | 5294.066 | 0.02259587 | 0.147467 | 0.15322659 | 0.87821959 | 0.99182259 |
| ENSG00000143847 | 412.507564 | −0.4176081 | 0.16610762 | −2.5140818 | 0.01193428 | 0.52421373 |
| ENSG00000058668 | 874.427613 | −0.094336 | 0.18613459 | −0.506816 | 0.61228396 | 0.98176061 |
| ENSG00000257315 | 289.822318 | −0.1473491 | 0.27710065 | −0.5317528 | 0.59489719 | 0.97969186 |
| ENSG00000162889 | 3007.29034 | 0.09289642 | 0.12334651 | 0.75313371 | 0.45136957 | 0.97027581 |
| ENSG00000162894 | 96.626403 | 0.26714668 | 0.2387629 | 1.11887855 | 0.26319196 | 0.96256622 |
| ENSG00000117595 | 223.578204 | 0.68760595 | 0.16229199 | 4.23684464 | 2.27E−05 | 0.00750429 |
| ENSG00000143473 | 148.877934 | −0.1086743 | 0.23070724 | −0.4710484 | 0.63760614 | 0.98262846 |
| ENSG00000143476 | 1826.89204 | 0.42557852 | 0.12519404 | 3.3993512 | 0.00067546 | 0.09722477 |
| ENSG00000230461 | 309.632859 | −0.1024844 | 0.17055613 | −0.6008835 | 0.54791758 | 0.9765894 |
| ENSG00000196660 | 24.119524 | −0.8321958 | 0.36929656 | −2.2534622 | 0.02423002 | 0.70499023 |
| ENSG00000143674 | 222.777063 | −0.3199917 | 0.16541661 | −1.9344591 | 0.05305671 | 0.86173331 |
| ENSG00000133019 | 74.6453602 | −0.7431139 | 0.25674267 | −2.8943917 | 0.00379894 | 0.30105524 |
| ENSG00000180875 | 343.86164 | 1.03268203 | 0.15657868 | 6.5952914 | 4.24E−11 | 9.98E−08 |
| ENSG00000174371 | 1592.46943 | 0.29016299 | 0.12493417 | 2.32252716 | 0.02020457 | 0.64791538 |
| ENSG00000225234 | 112.157611 | −0.2856763 | 0.21163339 | −1.3498642 | 0.17705956 | 0.94056812 |
| ENSG00000182551 | 750.125674 | 0.22985713 | 0.1410905 | 1.62914669 | 0.10328197 | 0.92973396 |
| ENSG00000115738 | 2503.56412 | 0.56408418 | 0.13922222 | 4.05168222 | 5.09E−05 | 0.01361962 |
| ENSG00000171848 | 2200.77166 | 0.47956511 | 0.12258837 | 3.9119951 | 9.15E−05 | 0.02130813 |
| ENSG00000071575 | 2429.92788 | 0.36676558 | 0.09617239 | 3.81362633 | 0.00013694 | 0.029567 |
| ENSG00000151779 | 1427.46586 | −0.0014759 | 0.14789427 | −0.0099792 | 0.99203792 | 0.99924816 |
| ENSG00000115129 | 234.456911 | 0.28480098 | 0.18183854 | 1.56622999 | 0.11729478 | 0.92973396 |
| ENSG00000171094 | 4727.26929 | −0.217987 | 0.20044254 | −1.0875286 | 0.27680327 | 0.96279754 |
| ENSG00000158089 | 201.028778 | 0.93961146 | 0.16977778 | 5.53436067 | 3.12E−08 | 2.64E−05 |
| ENSG00000279873 | 43.0997205 | −0.4692347 | 0.29098745 | −1.6125601 | 0.10684012 | 0.92973396 |
| ENSG00000225156 | 452.420714 | −0.3554522 | 0.12505152 | −2.8424464 | 0.00447688 | 0.322198 |
| ENSG00000196975 | 303.681175 | −0.2730273 | 0.13856631 | −1.9703728 | 0.04879567 | 0.85207236 |
| ENSG00000143977 | 1002.26131 | 0.26310509 | 0.18913889 | 1.39106813 | 0.16420477 | 0.94056812 |
| ENSG00000163017 | 150.638098 | 0.78714607 | 0.22660598 | 3.47363326 | 0.00051346 | 0.07816078 |
| ENSG00000065911 | 1623.56559 | 0.40118217 | 0.11599719 | 3.45855086 | 0.00054309 | 0.08092419 |
| ENSG00000115350 | 195.928217 | 0.03133229 | 0.2734579 | 0.11457811 | 0.90877953 | 0.9951783 |
| ENSG00000168874 | 141.952709 | 0.33367773 | 0.22749085 | 1.46677427 | 0.14243747 | 0.9324271 |
| ENSG00000158050 | 128.21304 | 0.08812781 | 0.24929679 | 0.35350559 | 0.72370944 | 0.98450389 |
| ENSG00000115539 | 510.529031 | 0.16486157 | 0.15954114 | 1.03334834 | 0.30144091 | 0.96279754 |
| ENSG00000198075 | 351.465285 | 0.15572227 | 0.19966651 | 0.77991181 | 0.43544278 | 0.9694822 |
| ENSG00000175497 | 263.30137 | −0.4525389 | 0.13568956 | −3.3351046 | 0.00085267 | 0.11622376 |
| ENSG00000152076 | 335.354352 | 0.1139713 | 0.16919723 | 0.6736003 | 0.50056547 | 0.97128468 |
| ENSG00000076003 | 2658.18865 | 0.36393143 | 0.1130582 | 3.21897417 | 0.0012865 | 0.14718363 |
| ENSG00000144354 | 1393.70125 | 0.23222778 | 0.10842729 | 2.1417834 | 0.03221092 | 0.77572832 |
| ENSG00000162998 | 57.6370134 | −0.941783 | 0.25723544 | −3.6611714 | 0.00025106 | 0.04873649 |
| ENSG00000168542 | 4165.10392 | 0.17383754 | 0.11383553 | 1.52709391 | 0.12673766 | 0.92973396 |
| ENSG00000138411 | 163.320451 | −0.3615723 | 0.21080198 | −1.7152226 | 0.08630442 | 0.92973396 |
| ENSG00000196141 | 776.601835 | 0.63663252 | 0.11192759 | 5.68789627 | 1.29E−08 | 1.24E−05 |
| ENSG00000055044 | 1896.83989 | 0.24357604 | 0.15815339 | 1.54012531 | 0.12352981 | 0.92973396 |
| ENSG00000116117 | 65.0265805 | −0.3488121 | 0.27995583 | −1.2459542 | 0.21278121 | 0.94620299 |
| ENSG00000118263 | 2447.22833 | −0.2491948 | 0.11991325 | −2.0781254 | 0.03769781 | 0.80640746 |
| ENSG00000144406 | 626.451526 | −0.3851756 | 0.20013181 | −1.9246096 | 0.05427822 | 0.86173331 |
| ENSG00000171951 | 3148.1728 | 0.23549216 | 0.12309774 | 1.91305032 | 0.05574161 | 0.86491665 |
| ENSG00000273301 | 104.498087 | −0.665896 | 0.27838202 | −2.3920223 | 0.01675582 | 0.60708299 |
| ENSG00000187514 | 20408.7226 | 0.18264609 | 0.17804369 | 1.02584984 | 0.30496237 | 0.96279754 |
| ENSG00000168918 | 110.553766 | −0.0686662 | 0.18801429 | −0.3652181 | 0.7149486 | 0.98450389 |
| ENSG00000157985 | 2859.85883 | −0.303945 | 0.19059637 | −1.5947049 | 0.11077824 | 0.92973396 |
| ENSG00000132323 | 445.730334 | 0.05323928 | 0.17436075 | 0.30533984 | 0.76010733 | 0.98663988 |
| ENSG00000172428 | 553.272584 | −0.1482512 | 0.27356117 | −0.5419307 | 0.58786622 | 0.97969186 |
| ENSG00000168393 | 810.168786 | −0.0104087 | 0.204751 | −0.0508359 | 0.95945627 | 0.99789928 |
| ENSG00000150995 | 1034.41494 | −0.0933281 | 0.18516732 | −0.5040201 | 0.61424724 | 0.98176061 |
| ENSG00000134107 | 31.9094537 | −0.1233784 | 0.3065468 | −0.4024781 | 0.68733219 | 0.98450389 |
| ENSG00000196277 | 105.550395 | −0.6631307 | 0.20261054 | −3.2729329 | 0.00106438 | 0.13018014 |
| ENSG00000196220 | 217.688378 | −0.0292553 | 0.17819568 | −0.1641752 | 0.86959322 | 0.99145366 |
| ENSG00000196639 | 44.106565 | 0.30648592 | 0.31431184 | 0.97510142 | 0.32950996 | 0.96302041 |
| ENSG00000170860 | 831.048559 | 0.26279196 | 0.19308751 | 1.36099932 | 0.1735139 | 0.94056812 |
| ENSG00000131389 | 721.177494 | −0.1114068 | 0.21664119 | −0.5142458 | 0.60708018 | 0.98176061 |
| ENSG00000263740 | 23.322155 | −2.1274539 | 0.50315039 | −4.2282665 | 2.35E−05 | 0.00766603 |
| ENSG00000183960 | 212.465307 | −0.3503069 | 0.19399156 | −1.8057842 | 0.07095206 | 0.92112726 |
| ENSG00000144681 | 753.216253 | −0.2000776 | 0.12276085 | −1.6298157 | 0.10314045 | 0.92973396 |
| ENSG00000187091 | 337.443417 | −0.3914525 | 0.1410152 | −2.7759597 | 0.0055039 | 0.36358572 |
| ENSG00000163820 | 282.814221 | −0.1882003 | 0.22706429 | −0.8288414 | 0.40719419 | 0.9694822 |
| ENSG00000164045 | 875.156728 | 0.34063026 | 0.11659274 | 2.92153921 | 0.00348306 | 0.28401271 |
| ENSG00000007402 | 2567.3274 | −0.1041637 | 0.18439572 | −0.5648921 | 0.57214716 | 0.97865595 |
| ENSG00000145050 | 707.92096 | 0.12929629 | 0.165876 | 0.7794756 | 0.4356996 | 0.9694822 |
| ENSG00000163932 | 223.486999 | −0.2416345 | 0.15284363 | −1.5809265 | 0.11389484 | 0.92973396 |
| ENSG00000163638 | 4307.60544 | 0.12273739 | 0.15498365 | 0.79193763 | 0.42839704 | 0.9694822 |
| ENSG00000185008 | 288.228115 | −1.0505839 | 0.20401647 | −5.1495052 | 2.61E−07 | 0.00018356 |
| ENSG00000170017 | 4114.91898 | −0.5488689 | 0.13688637 | −4.0096683 | 6.08E−05 | 0.01513593 |
| ENSG00000185565 | 3158.49177 | −0.0995056 | 0.16387645 | −0.6071991 | 0.54371876 | 0.97648438 |
| ENSG00000082684 | 103.315161 | 0.124235 | 0.22857854 | 0.54351124 | 0.58677785 | 0.97969186 |
| ENSG00000114491 | 642.82753 | 0.35709206 | 0.15047901 | 2.37303576 | 0.01764256 | 0.62112951 |
| ENSG00000074416 | 94.6433098 | −0.389132 | 0.22720239 | −1.7127108 | 0.08676575 | 0.92973396 |
| ENSG00000196353 | 31.7262009 | −0.5138672 | 0.3384371 | −1.5183536 | 0.12892529 | 0.92973396 |
| ENSG00000154917 | 627.542444 | −0.3264532 | 0.14299596 | −2.282954 | 0.02243308 | 0.68336649 |
| ENSG00000163762 | 51.0116089 | −10.074482 | 1.2045568 | −8.3636418 | 6.08E−17 | 2.14E−13 |
| ENSG00000163661 | 64.2209448 | 0.60783651 | 0.28002482 | 2.17065227 | 0.02995747 | 0.76376729 |
| ENSG00000085276 | 32.5717915 | −1.6131225 | 0.31635493 | −5.0990908 | 3.41E−07 | 0.00021239 |
| ENSG00000145198 | 111.716628 | −0.6641965 | 0.21881764 | −3.0353884 | 0.00240226 | 0.22040175 |
| ENSG00000163918 | 793.332914 | 0.32988357 | 0.15521364 | 2.12535173 | 0.03355728 | 0.78026196 |
| ENSG00000114315 | 101.78102 | 0.46935121 | 0.30606784 | 1.53348753 | 0.12515578 | 0.92973396 |
| ENSG00000178222 | 177.567133 | −0.3823609 | 0.15690213 | −2.4369387 | 0.01481219 | 0.57514386 |
| ENSG00000163950 | 1395.33351 | 0.19869023 | 0.15397467 | 1.29040857 | 0.19690884 | 0.94056812 |
| ENSG00000091490 | 80.3496775 | −0.2704613 | 0.23428312 | −1.1544208 | 0.24832769 | 0.9589233 |
| ENSG00000124406 | 1346.51361 | −0.2003014 | 0.17860454 | −1.12148 | 0.26208362 | 0.96256622 |
| ENSG00000156140 | 79.1233738 | −0.1534773 | 0.23468762 | −0.6539643 | 0.5131348 | 0.97172906 |
| ENSG00000138769 | 50.729811 | −0.5497854 | 0.29235362 | −1.8805494 | 0.06003324 | 0.88595383 |
| ENSG00000138759 | 513.401002 | −0.1559224 | 0.23365674 | −0.667314 | 0.50457157 | 0.97128468 |
| ENSG00000138668 | 8421.02803 | 0.16505464 | 0.09176392 | 1.7986877 | 0.0720681 | 0.92327552 |
| ENSG00000184305 | 412.545857 | −0.1522377 | 0.19211338 | −0.7924367 | 0.4281061 | 0.9694822 |
| ENSG00000182168 | 2994.20419 | −0.2599696 | 0.19139555 | −1.3582842 | 0.17437352 | 0.94056812 |
| ENSG00000271474 | 269.107644 | −0.4307491 | 0.16367875 | −2.631674 | 0.00849653 | 0.44720937 |
| ENSG00000164032 | 2584.11854 | 0.31972594 | 0.18452407 | 1.73270594 | 0.08314795 | 0.92973396 |
| ENSG00000138795 | 53.0506709 | 1.79817075 | 0.28406817 | 6.3300678 | 2.45E−10 | 4.90E−07 |
| ENSG00000164109 | 1808.52017 | 0.3652838 | 0.15508714 | 2.35534551 | 0.01850549 | 0.63058527 |
| ENSG00000164167 | 383.69077 | 0.23719961 | 0.20822374 | 1.1391574 | 0.25463751 | 0.96002397 |
| ENSG00000151617 | 1972.80159 | 0.65673705 | 0.13986061 | 4.69565427 | 2.66E−06 | 0.0013077 |
| ENSG00000137460 | 57.0640892 | −0.4382397 | 0.2563612 | −1.7094619 | 0.08736543 | 0.92973396 |
| ENSG00000168843 | 250.655119 | −0.6947394 | 0.19170451 | −3.6240117 | 0.00029007 | 0.05217215 |
| ENSG00000251216 | 1141.42786 | 0.03782435 | 0.15732949 | 0.2404149 | 0.81000863 | 0.99092117 |
| ENSG00000151725 | 1139.09906 | 0.33980344 | 0.15407597 | 2.20542793 | 0.02742408 | 0.73265922 |
| ENSG00000071539 | 1120.76928 | 0.25246912 | 0.10306264 | 2.44966669 | 0.01429885 | 0.57261359 |
| ENSG00000215218 | 1621.85427 | 0.45765063 | 0.11408035 | 4.0116518 | 6.03E−05 | 0.01513593 |
| ENSG00000113494 | 17.7919736 | −0.6287397 | 0.45005328 | −1.397034 | 0.16240335 | 0.94056812 |
| ENSG00000112936 | 15556.4126 | 0.0898406 | 0.12796575 | 0.70206752 | 0.48263706 | 0.97128468 |
| ENSG00000259663 | 2173.06244 | −0.1023141 | 0.10554656 | −0.9693745 | 0.33235838 | 0.96302041 |
| ENSG00000130449 | 653.946434 | −0.1458385 | 0.17975582 | −0.8113142 | 0.41718525 | 0.9694822 |
| ENSG00000214944 | 97.3488142 | −0.6721842 | 0.21714253 | −3.0955899 | 0.00196422 | 0.19512158 |
| ENSG00000228716 | 2220.19717 | 0.57632438 | 0.13554587 | 4.25187714 | 2.12E−05 | 0.00723454 |
| ENSG00000164176 | 65.9045405 | 0.68703407 | 0.26592048 | 2.58360719 | 0.00977731 | 0.4782583 |
| ENSG00000129595 | 282.507852 | −0.1835399 | 0.22909306 | −0.8011587 | 0.42303975 | 0.9694822 |
| ENSG00000113368 | 2767.97899 | 0.13428904 | 0.09200094 | 1.45964865 | 0.14438666 | 0.9324271 |
| ENSG00000066583 | 10696.836 | 1.19756554 | 0.1253098 | 9.5568384 | 1.21E−21 | 8.56E−18 |
| ENSG00000145833 | 2398.48358 | 0.10434802 | 0.15763901 | 0.66194286 | 0.50800784 | 0.97128468 |
| ENSG00000152377 | 235.457097 | 0.14128184 | 0.17893587 | 0.78956689 | 0.42978075 | 0.9694822 |
| ENSG00000044115 | 3481.64199 | −0.0205157 | 0.08997801 | −0.2280083 | 0.81963981 | 0.99145366 |
| ENSG00000145819 | 381.969105 | −0.114624 | 0.14946662 | −0.7668869 | 0.44314876 | 0.9694822 |
| ENSG00000113657 | 1357.76408 | 0.13454605 | 0.13146098 | 1.02346762 | 0.3060868 | 0.96279754 |
| ENSG00000184347 | 8511.75239 | −0.1024305 | 0.16373904 | −0.6255715 | 0.53159606 | 0.97577914 |
| ENSG00000120149 | 497.016479 | −0.0843286 | 0.1216413 | −0.6932567 | 0.48814849 | 0.97128468 |
| ENSG00000087116 | 228.336828 | 0.16911545 | 0.20758232 | 0.81469098 | 0.41524922 | 0.9694822 |
| ENSG00000054598 | 3112.5844 | 0.42546324 | 0.15645282 | 2.71943482 | 0.00653936 | 0.39086519 |
| ENSG00000112312 | 710.099198 | 0.18293305 | 0.16623754 | 1.10043165 | 0.27114409 | 0.96256622 |
| ENSG00000026950 | 455.546524 | 0.03236648 | 0.16621781 | 0.19472327 | 0.84560959 | 0.99145366 |
| ENSG00000137310 | 490.430255 | 0.17017138 | 0.12874005 | 1.32182168 | 0.18622754 | 0.94056812 |
| ENSG00000096433 | 128.384927 | −0.6556235 | 0.296376 | −2.2121341 | 0.0269574 | 0.72384722 |
| ENSG00000112081 | 6471.12769 | 0.22363102 | 0.16230961 | 1.37780523 | 0.16826343 | 0.94056812 |
| ENSG00000112576 | 469.900632 | 0.11500962 | 0.1611008 | 0.71389852 | 0.47528993 | 0.97128468 |
| ENSG00000180992 | 486.797143 | 0.00210948 | 0.2306239 | 0.00914686 | 0.99270196 | 0.99929777 |
| ENSG00000008196 | 8624.97581 | −0.2267703 | 0.09055838 | −2.5041339 | 0.01227516 | 0.53223371 |
| ENSG00000112118 | 4142.82279 | 0.29903876 | 0.07917543 | 3.77691339 | 0.00015878 | 0.03359709 |
| ENSG00000202198 | 566.346759 | −6.0276847 | 1.24514722 | −4.8409414 | 1.29E−06 | 0.0007011 |
| ENSG00000272316 | 41.6244417 | −0.6152341 | 0.3102928 | −1.9827535 | 0.04739497 | 0.8477793 |
| ENSG00000230910 | 192.275141 | −0.4852156 | 0.17063648 | −2.8435627 | 0.00446122 | 0.322198 |
| ENSG00000135298 | 515.032149 | −0.1995546 | 0.12951725 | −1.5407572 | 0.1233759 | 0.92973396 |
| ENSG00000079841 | 86.1193399 | −0.5865693 | 0.24323778 | −2.4115056 | 0.01588681 | 0.58930028 |
| ENSG00000112367 | 487.597432 | −0.1631086 | 0.12287092 | −1.3274789 | 0.1843503 | 0.94056812 |
| ENSG00000155115 | 805.614288 | 0.20701261 | 0.19394337 | 1.0673869 | 0.28579716 | 0.96279754 |
| ENSG00000146352 | 229.15436 | −0.3381424 | 0.19044983 | −1.7754931 | 0.0758165 | 0.92973396 |
| ENSG00000203760 | 221.053986 | 0.1508931 | 0.24382286 | 0.61886364 | 0.53600619 | 0.97583004 |
| ENSG00000118515 | 1176.5287 | 0.41225867 | 0.11007346 | 3.74530492 | 0.00018017 | 0.03737567 |
| ENSG00000146469 | 906.476132 | 0.2820772 | 0.18262896 | 1.54453705 | 0.12245828 | 0.92973396 |
| ENSG00000153721 | 585.426642 | −0.0718349 | 0.11818522 | −0.6078162 | 0.54330938 | 0.97648438 |
| ENSG00000029639 | 343.599121 | −0.1903024 | 0.12385033 | −1.5365516 | 0.12440316 | 0.92973396 |
| ENSG00000185345 | 57.1503163 | −0.387629 | 0.24216349 | −1.6006912 | 0.10944533 | 0.92973396 |
| ENSG00000164850 | 182.544241 | 0.41994191 | 0.20758507 | 2.02298707 | 0.04307448 | 0.83718021 |
| ENSG00000122687 | 1406.05674 | 0.07649508 | 0.12077352 | 0.63337625 | 0.52648797 | 0.97419842 |
| ENSG00000048052 | 4204.65126 | −0.2478084 | 0.17746413 | −1.3963857 | 0.16259839 | 0.94056812 |
| ENSG00000105855 | 41.1619749 | −0.3739413 | 0.34566629 | −1.0817985 | 0.27934205 | 0.96279754 |
| ENSG00000122585 | 220.635473 | −1.261755 | 0.19951678 | −6.3240548 | 2.55E−10 | 4.90E−07 |
| ENSG00000070882 | 190.46303 | −0.3375687 | 0.17474425 | −1.9317871 | 0.05338579 | 0.86173331 |
| ENSG00000122574 | 94.4781546 | −0.1098413 | 0.26547317 | −0.4137566 | 0.67905241 | 0.98450389 |
| ENSG00000122641 | 177.131899 | 1.07117789 | 0.19166349 | 5.58884693 | 2.29E−08 | 2.10E−05 |
| ENSG00000272655 | 63.4077982 | 0.19811453 | 0.24955318 | 0.793877 | 0.42726704 | 0.9694822 |
| ENSG00000146674 | 5646.90033 | 0.73821544 | 0.086944 | 8.49070038 | 2.05E−17 | 1.09E−13 |
| ENSG00000225648 | 371.693317 | 0.26045086 | 0.15371386 | 1.69438757 | 0.09019166 | 0.92973396 |
| ENSG00000049540 | 1513.88196 | −0.2408659 | 0.17338965 | −1.3891594 | 0.16478428 | 0.94056812 |
| ENSG00000049541 | 1368.7005 | 0.22804556 | 0.14313707 | 1.5931971 | 0.11111598 | 0.92973396 |
| ENSG00000186088 | 109.698772 | −0.4462273 | 0.25122684 | −1.7761928 | 0.07570114 | 0.92973396 |
| ENSG00000075223 | 3666.39632 | 0.06070272 | 0.15281823 | 0.39722171 | 0.69120397 | 0.98450389 |
| ENSG00000164692 | 67.8006859 | 0.41523446 | 0.26832065 | 1.54753074 | 0.12173532 | 0.92973396 |
| ENSG00000166508 | 8960.02005 | 0.1215233 | 0.1161956 | 1.04585117 | 0.29562975 | 0.96279754 |
| ENSG00000077080 | 888.474157 | −0.0389763 | 0.15134491 | −0.257533 | 0.79676735 | 0.98858465 |
| ENSG00000164597 | 961.894097 | −0.0444178 | 0.15331562 | −0.2897151 | 0.77203421 | 0.98663988 |
| ENSG00000173114 | 1896.30582 | 0.45126935 | 0.1735288 | 2.60054431 | 0.0093076 | 0.46890358 |
| ENSG00000135269 | 331.219696 | −1.0753428 | 0.17513987 | −6.139909 | 8.26E−10 | 1.03E−06 |
| ENSG00000179603 | 207.096354 | −0.0629328 | 0.15459791 | −0.4070738 | 0.68395377 | 0.98450389 |
| ENSG00000128596 | 1120.12758 | −0.186275 | 0.185177 | −1.0059295 | 0.3144495 | 0.96279754 |
| ENSG00000128567 | 919.311468 | −0.1417358 | 0.11902106 | −1.1908464 | 0.2337139 | 0.95658313 |
| ENSG00000105894 | 646.919988 | 0.04451436 | 0.16906836 | 0.26329207 | 0.79232547 | 0.98801156 |
| ENSG00000090266 | 498.833367 | 0.03876552 | 0.2212727 | 0.17519341 | 0.86092766 | 0.99145366 |
| ENSG00000159784 | 149.23514 | −0.0082793 | 0.20338764 | −0.0407071 | 0.96752942 | 0.99796143 |
| ENSG00000174469 | 385.825809 | −1.0266373 | 0.22085511 | −4.6484651 | 3.34E−06 | 0.00156431 |
| ENSG00000106462 | 1871.50657 | 0.15333393 | 0.101152 | 1.51587647 | 0.12955059 | 0.92973396 |
| ENSG00000197558 | 43.5577965 | −0.2284486 | 0.30165359 | −0.7573209 | 0.44885764 | 0.9694822 |
| ENSG00000198947 | 32.7971026 | −0.2857067 | 0.32647931 | −0.8751144 | 0.38151168 | 0.96524935 |
| ENSG00000147155 | 467.685573 | 0.24925881 | 0.18935258 | 1.31637403 | 0.18804854 | 0.94056812 |
| ENSG00000274588 | 74.3315493 | −0.7178335 | 0.26861072 | −2.6723932 | 0.00753124 | 0.42156986 |
| ENSG00000072506 | 1622.04752 | 0.0448671 | 0.20520777 | 0.2186423 | 0.8269287 | 0.99145366 |
| ENSG00000133169 | 2745.58896 | 0.04619222 | 0.2160809 | 0.21377278 | 0.83072425 | 0.99145366 |
| ENSG00000077279 | 2923.7225 | −0.2204698 | 0.20786657 | −1.0606312 | 0.28885753 | 0.96279754 |
| ENSG00000260802 | 336.484212 | −0.368044 | 0.2129997 | −1.7279087 | 0.08400459 | 0.92973396 |
| ENSG00000125354 | 1607.08669 | −0.1543855 | 0.09223647 | −1.6738009 | 0.09416973 | 0.92973396 |
| ENSG00000009694 | 30.321651 | −2.3385809 | 0.44280543 | −5.2812831 | 1.28E−07 | 9.36E−05 |
| ENSG00000147256 | 488.353731 | −0.7627576 | 0.1393369 | −5.4741965 | 4.40E−08 | 3.44E−05 |
| ENSG00000171004 | 280.445427 | −0.2530564 | 0.16225138 | −1.5596566 | 0.11884105 | 0.92973396 |
| ENSG00000184785 | 22.31125 | 1.02581225 | 0.48163193 | 2.12986761 | 0.03318255 | 0.78008303 |
| ENSG00000124260 | 79.3122591 | 0.20385483 | 0.28013847 | 0.72769308 | 0.46680149 | 0.97128468 |
| ENSG00000221867 | 842.616934 | 0.14796624 | 0.15400932 | 0.96076158 | 0.33667206 | 0.96302041 |
| ENSG00000198930 | 216.807444 | 0.18450389 | 0.22041075 | 0.83709116 | 0.40254133 | 0.9694822 |
| ENSG00000197172 | 639.016981 | 0.28552753 | 0.17225881 | 1.65754966 | 0.0974084 | 0.92973396 |
| ENSG00000182492 | 92.7048207 | 0.25600375 | 0.25378982 | 1.00872347 | 0.31310727 | 0.96279754 |
| ENSG00000176595 | 806.922667 | −0.4071093 | 0.15612975 | −2.6075062 | 0.00912044 | 0.46556948 |
| ENSG00000173281 | 302.315743 | −0.0731501 | 0.18081439 | −0.4045592 | 0.68580158 | 0.98450389 |
| ENSG00000269918 | 101.042073 | 0.25518443 | 0.23124708 | 1.10351417 | 0.26980394 | 0.96256622 |
| ENSG00000104722 | 2094.48775 | 0.49945678 | 0.15045607 | 3.31961873 | 0.0009014 | 0.11920513 |
| ENSG00000171320 | 398.674485 | 0.3808896 | 0.17005413 | 2.23981382 | 0.02510301 | 0.71329993 |
| ENSG00000120875 | 2837.84888 | −0.6239842 | 0.10559667 | −5.9091274 | 3.44E−09 | 3.64E−06 |
| ENSG00000156687 | 462.541799 | −0.2786974 | 0.19217731 | −1.4502096 | 0.14700007 | 0.9339277 |
| ENSG00000104738 | 3569.65926 | 0.32924957 | 0.08417004 | 3.91171933 | 9.16E−05 | 0.02130813 |
| ENSG00000019549 | 335.184999 | 0.241853 | 0.1646474 | 1.46891475 | 0.14185591 | 0.9324271 |
| ENSG00000254087 | 1214.33674 | −0.307432 | 0.11585582 | −2.6535742 | 0.00796443 | 0.42871998 |
| ENSG00000104313 | 87.9807537 | −1.0651618 | 0.20754241 | −5.1322611 | 2.86E−07 | 0.00018356 |
| ENSG00000250979 | 102.628872 | −1.2301504 | 0.24746458 | −4.971016 | 6.66E−07 | 0.00039146 |
| ENSG00000121039 | 203.999663 | 0.77470617 | 0.17165203 | 4.5132362 | 6.38E−06 | 0.00275697 |
| ENSG00000164684 | 4033.99544 | −0.5007197 | 0.18133791 | −2.7612521 | 0.00575802 | 0.37258091 |
| ENSG00000136982 | 451.384204 | 0.26556325 | 0.16463717 | 1.61302127 | 0.10673989 | 0.92973396 |
| ENSG00000147684 | 928.496724 | 0.04168247 | 0.22944513 | 0.18166641 | 0.85584453 | 0.99145366 |
| ENSG00000178896 | 314.027821 | −0.0256921 | 0.27024739 | −0.0950687 | 0.92426022 | 0.99569987 |
| ENSG00000160957 | 946.03051 | −0.0099867 | 0.201135 | −0.0496517 | 0.96039991 | 0.99789928 |
| ENSG00000178445 | 638.532752 | −0.0861447 | 0.11810944 | −0.7293635 | 0.46577932 | 0.97128468 |
| ENSG00000147889 | 83.985427 | 0.6043578 | 0.31587505 | 1.9132812 | 0.05571206 | 0.86491665 |
| ENSG00000165264 | 674.393028 | 0.11996591 | 0.19866842 | 0.60384993 | 0.54594342 | 0.97648438 |
| ENSG00000107262 | 1053.12536 | 0.1577203 | 0.19661135 | 0.80219325 | 0.42244118 | 0.9694822 |
| ENSG00000137100 | 762.22749 | 0.11811211 | 0.15268509 | 0.77356672 | 0.43918706 | 0.9694822 |
| ENSG00000221829 | 792.731761 | 0.15636918 | 0.1221168 | 1.28048873 | 0.2003733 | 0.94056812 |
| ENSG00000119139 | 1436.36363 | −0.2582091 | 0.11275767 | −2.2899475 | 0.02202436 | 0.6799987 |
| ENSG00000135069 | 1673.25537 | 0.49211914 | 0.1220537 | 4.03198862 | 5.53E−05 | 0.01429004 |
| ENSG00000148053 | 1534.09037 | 0.93843831 | 0.20203209 | 4.64499629 | 3.40E−06 | 0.00156431 |
| ENSG00000213694 | 440.652299 | 0.85761219 | 0.13791233 | 6.21853175 | 5.02E−10 | 8.11E−07 |
| ENSG00000165244 | 531.821263 | 0.29768907 | 0.12073003 | 2.46574163 | 0.01367299 | 0.5585074 |
| ENSG00000136943 | 209.087589 | 0.38870714 | 0.16512712 | 2.35398722 | 0.01857325 | 0.63181895 |
| ENSG00000136938 | 3938.99832 | 0.23782175 | 0.16618306 | 1.43108302 | 0.15240642 | 0.93425563 |
| ENSG00000165185 | 1197.93803 | −0.1876855 | 0.17992526 | −1.0431305 | 0.29688786 | 0.96279754 |
| ENSG00000119421 | 562.741549 | 0.16904731 | 0.17715061 | 0.95425754 | 0.3399533 | 0.96302041 |
| ENSG00000136828 | 587.502939 | −0.2154194 | 0.17848908 | −1.2069051 | 0.22746871 | 0.95445475 |
| ENSG00000136854 | 2972.62869 | −0.1212858 | 0.12044536 | −1.0069775 | 0.31394557 | 0.96279754 |
| ENSG00000106991 | 776.329444 | 0.39704451 | 0.13604226 | 2.91853804 | 0.00351677 | 0.28401271 |
| ENSG00000123454 | 5584.01389 | −0.7750563 | 0.17304247 | −4.4789946 | 7.50E−06 | 0.00302204 |
| ENSG00000187796 | 268.810074 | 0.04005697 | 0.21031623 | 0.19046069 | 0.84894814 | 0.99145366 |
| ENSG00000148400 | 231.97295 | 0.19587662 | 0.29632548 | 0.66101848 | 0.50860046 | 0.97128468 |
| ENSG00000130600 | 2278.61782 | −1.3806772 | 0.1648348 | −8.3761269 | 5.47E−17 | 2.14E−13 |
| ENSG00000167244 | 350.661076 | −2.628348 | 0.22024922 | −11.933518 | 7.92E−33 | 1.67E−28 |
| ENSG00000166483 | 1590.30457 | 0.22626834 | 0.1062879 | 2.12882493 | 0.03326875 | 0.78008303 |
| ENSG00000133812 | 1420.92806 | −0.1592242 | 0.18820333 | −0.846022 | 0.3975405 | 0.96916243 |
| ENSG00000050165 | 1293.35929 | −0.3359161 | 0.10892506 | −3.0839191 | 0.00204293 | 0.20012214 |
| ENSG00000109881 | 577.857728 | 0.18894955 | 0.18026643 | 1.04816827 | 0.29456108 | 0.96279754 |
| ENSG00000066382 | 443.273746 | −0.2763556 | 0.12657723 | −2.1832966 | 0.02901397 | 0.75450374 |
| ENSG00000026508 | 193.463753 | −0.6666403 | 0.19270457 | −3.4593899 | 0.0005414 | 0.08092419 |
| ENSG00000157570 | 2075.93207 | −0.2479279 | 0.15109791 | −1.6408424 | 0.10083014 | 0.92973396 |
| ENSG00000134569 | 315.67573 | −0.7338448 | 0.21662879 | −3.3875684 | 0.00070515 | 0.09880993 |
| ENSG00000165916 | 1906.89858 | 0.062658 | 0.14751368 | 0.42476057 | 0.67101122 | 0.98450389 |
| ENSG00000255433 | 297.519497 | 1.04721281 | 0.21891694 | 4.78360785 | 1.72E−06 | 0.00091077 |
| ENSG00000134809 | 380.53845 | 0.10118052 | 0.21625161 | 0.46788332 | 0.63986803 | 0.98262846 |
| ENSG00000189057 | 293.11634 | 0.52127625 | 0.20926539 | 2.49098162 | 0.01273907 | 0.54239168 |
| ENSG00000166900 | 132.660547 | −0.2567302 | 0.16946725 | −1.5149253 | 0.12979133 | 0.92973396 |
| ENSG00000168496 | 1992.49991 | 0.37970718 | 0.14166663 | 2.6802866 | 0.00735591 | 0.41540737 |
| ENSG00000168003 | 1159.9806 | 0.12723912 | 0.17000156 | 0.74845856 | 0.45418361 | 0.97027581 |
| ENSG00000146670 | 1501.52517 | 0.16260608 | 0.12032036 | 1.3514428 | 0.17655363 | 0.94056812 |
| ENSG00000014138 | 754.940952 | 0.32785903 | 0.11516833 | 2.84678117 | 0.00441637 | 0.322198 |
| ENSG00000172922 | 620.929055 | 0.05830282 | 0.208777 | 0.27925883 | 0.7800462 | 0.98715916 |
| ENSG00000132749 | 187.790801 | −0.4801871 | 0.16635647 | −2.8864947 | 0.00389559 | 0.30756286 |
| ENSG00000033327 | 2928.87863 | −0.2911594 | 0.18592328 | −1.5660191 | 0.11734415 | 0.92973396 |
| ENSG00000182103 | 187.050756 | 0.33222617 | 0.22052077 | 1.50655273 | 0.13192534 | 0.92973396 |
| ENSG00000150687 | 306.524588 | 0.46409034 | 0.14901663 | 3.11435267 | 0.00184349 | 0.18574478 |
| ENSG00000137727 | 53.981437 | 0.18731118 | 0.25862889 | 0.72424695 | 0.46891414 | 0.97128468 |
| ENSG00000109846 | 34.7345882 | 1.15944144 | 0.36334531 | 3.19101806 | 0.00141772 | 0.15956184 |
| ENSG00000150779 | 676.874435 | 0.13170062 | 0.22055547 | 0.59713151 | 0.55041958 | 0.97700665 |
| ENSG00000188486 | 3128.38886 | 0.00432021 | 0.20406234 | 0.02117102 | 0.98310923 | 0.99852596 |
| ENSG00000149403 | 112.702881 | −0.3657744 | 0.21966557 | −1.6651422 | 0.09588439 | 0.92973396 |
| ENSG00000137642 | 677.959403 | −0.1644289 | 0.18251409 | −0.9009107 | 0.36763583 | 0.96401724 |
| ENSG00000154146 | 207.452709 | 0.2244436 | 0.25463624 | 0.88142834 | 0.37808602 | 0.96429199 |
| ENSG00000149557 | 902.45893 | 0.2210444 | 0.15115369 | 1.46238171 | 0.14363664 | 0.9324271 |
| ENSG00000166105 | 107.301143 | 0.20555348 | 0.20616415 | 0.99703791 | 0.31874611 | 0.96302041 |
| ENSG00000255545 | 42.0985936 | −1.0665013 | 0.29294609 | −3.6406062 | 0.000272 | 0.05048406 |
| ENSG00000197308 | 479.427991 | −0.4718882 | 0.24437682 | −1.930986 | 0.05348479 | 0.86173331 |
| ENSG00000151468 | 76.7451882 | 0.91811016 | 0.23806429 | 3.85656392 | 0.00011499 | 0.02575003 |
| ENSG00000065328 | 1189.77449 | 0.29056651 | 0.11893326 | 2.44310545 | 0.01456148 | 0.57514386 |
| ENSG00000078114 | 735.314879 | −0.4900066 | 0.17682314 | −2.7711676 | 0.00558557 | 0.36589782 |
| ENSG00000095739 | 1191.41474 | 0.685394 | 0.15296285 | 4.48078739 | 7.44E−06 | 0.00302204 |
| ENSG00000165633 | 182.739294 | 0.0095894 | 0.18444754 | 0.05198985 | 0.95853678 | 0.99789505 |
| ENSG00000122952 | 740.142453 | 0.19534633 | 0.12323447 | 1.58515982 | 0.11293003 | 0.92973396 |
| ENSG00000138336 | 891.407148 | 1.36597342 | 0.23533887 | 5.80428313 | 6.46E−09 | 6.51E−06 |
| ENSG00000156515 | 3490.71713 | 0.02006319 | 0.09946405 | 0.20171299 | 0.8401411 | 0.99145366 |
| ENSG00000107742 | 576.43466 | 0.29873802 | 0.15959602 | 1.87183877 | 0.06122891 | 0.88966453 |
| ENSG00000156113 | 609.391281 | −0.0175341 | 0.14940426 | −0.1173599 | 0.90657484 | 0.99489281 |
| ENSG00000187122 | 962.018273 | 0.04804847 | 0.18386847 | 0.26131978 | 0.79384591 | 0.98801156 |
| ENSG00000095713 | 255.091454 | 0.03945609 | 0.1867741 | 0.21125035 | 0.83269192 | 0.99145366 |
| ENSG00000099194 | 7740.70394 | −0.0662627 | 0.13394293 | −0.4947083 | 0.62080602 | 0.98262846 |
| ENSG00000156398 | 208.918663 | 0.42871258 | 0.15747833 | 2.72235915 | 0.00648177 | 0.39080799 |
| ENSG00000108018 | 1665.51288 | −0.5196655 | 0.1802889 | −2.8824041 | 0.00394653 | 0.30927664 |
| ENSG00000150594 | 61.3664637 | −0.1639681 | 0.29121365 | −0.5630509 | 0.57340021 | 0.97925466 |
| ENSG00000165868 | 1024.44815 | −0.1316877 | 0.13771842 | −0.95621 | 0.33896614 | 0.96302041 |
| ENSG00000187164 | 448.261163 | −0.3997806 | 0.1308582 | −3.0550674 | 0.0022501 | 0.21171292 |
| ENSG00000119973 | 22.2654684 | −0.6163222 | 0.46465177 | −1.3264174 | 0.18470147 | 0.94056812 |
| ENSG00000198873 | 343.660662 | −0.3124385 | 0.14285103 | −2.1871631 | 0.02873062 | 0.75043606 |
| ENSG00000148848 | 678.219215 | −0.3407258 | 0.1856086 | −1.835722 | 0.06639878 | 0.90255826 |
| ENSG00000108010 | 1095.06384 | 0.39025989 | 0.16739942 | 2.3313097 | 0.01973704 | 0.642595 |
| ENSG00000188385 | 129.617311 | −0.4262682 | 0.18210607 | −2.3407686 | 0.01924409 | 0.63922399 |
| ENSG00000182326 | 37.9394579 | −1.2616969 | 0.34844133 | −3.6209737 | 0.0002935 | 0.05217215 |
| ENSG00000139182 | 1013.40793 | −0.3403816 | 0.14535605 | −2.341709 | 0.01919567 | 0.63922399 |
| ENSG00000172572 | 84.4248088 | −0.7937712 | 0.27987828 | −2.8361301 | 0.00456638 | 0.32752579 |
| ENSG00000084453 | 98.6224656 | −0.6029991 | 0.22185153 | −2.7180301 | 0.00656719 | 0.39142286 |
| ENSG00000111728 | 53.8112263 | −0.473967 | 0.26958682 | −1.7581236 | 0.07872648 | 0.92973396 |
| ENSG00000057294 | 100.9103 | −0.4612559 | 0.22433777 | −2.056078 | 0.039775 | 0.8234826 |
| ENSG00000173208 | 456.559256 | −0.5090105 | 0.20122966 | −2.5295002 | 0.01142251 | 0.51423167 |
| ENSG00000018236 | 2362.82253 | −0.597273 | 0.13901949 | −4.2963256 | 1.74E−05 | 0.00624961 |
| ENSG00000184613 | 493.915011 | −0.4848168 | 0.11597479 | −4.1803637 | 2.91E−05 | 0.00919132 |
| ENSG00000170627 | 86.8767792 | 0.8529046 | 0.25664879 | 3.32323637 | 0.00088979 | 0.11840987 |
| ENSG00000123374 | 502.515414 | 0.25559966 | 0.1488911 | 1.71668862 | 0.08603608 | 0.92973396 |
| ENSG00000011465 | 1260.14348 | 0.43317657 | 0.14211142 | 3.04814741 | 0.00230257 | 0.21462585 |
| ENSG00000151136 | 253.165693 | −0.0448821 | 0.15938331 | −0.2815986 | 0.77825132 | 0.98663988 |
| ENSG00000076248 | 1331.85909 | 0.11969367 | 0.1074736 | 1.113703 | 0.26540662 | 0.96256622 |
| ENSG00000076555 | 497.510696 | −0.3006904 | 0.18487452 | −1.6264567 | 0.10385255 | 0.92973396 |
| ENSG00000060709 | 3307.95041 | −0.2757827 | 0.13827653 | −1.9944287 | 0.04610522 | 0.84657543 |
| ENSG00000196199 | 1316.71137 | 0.05891283 | 0.15448035 | 0.38136135 | 0.70293514 | 0.98450389 |
| ENSG00000165480 | 470.356075 | 0.50716981 | 0.15816034 | 3.20668139 | 0.00134276 | 0.15193251 |
| ENSG00000133083 | 911.212212 | 0.05078591 | 0.13365407 | 0.37998026 | 0.70396007 | 0.98450389 |
| ENSG00000276644 | 148.725288 | −0.4125196 | 0.16976797 | −2.4299024 | 0.01510289 | 0.5774324 |
| ENSG00000178695 | 2729.12734 | 0.41621384 | 0.14256994 | 2.91936599 | 0.00350744 | 0.28401271 |
| ENSG00000088387 | 260.276875 | −0.044517 | 0.19601491 | −0.2271105 | 0.82033785 | 0.99145366 |
| ENSG00000102466 | 475.288892 | 0.22957826 | 0.14926124 | 1.53809698 | 0.1240249 | 0.92973396 |
| ENSG00000125266 | 1816.3175 | −0.2129184 | 0.10363051 | −2.0545923 | 0.0399184 | 0.82403267 |
| ENSG00000204442 | 1698.86651 | −0.4294018 | 0.09138731 | −4.6987029 | 2.62E−06 | 0.0013077 |
| ENSG00000274718 | 105.845859 | −1.4098213 | 0.22709473 | −6.2080758 | 5.36E−10 | 8.11E−07 |
| ENSG00000198176 | 1888.55783 | 0.22962021 | 0.1043488 | 2.20050649 | 0.02777098 | 0.74099138 |
| ENSG00000259017 | 37.315656 | −1.0583334 | 0.34002804 | −3.1124885 | 0.00185517 | 0.18603598 |
| ENSG00000168348 | 403.695123 | −1.2359745 | 0.16419454 | −7.5275009 | 5.17E−14 | 1.56E−10 |
| ENSG00000174373 | 890.841032 | −0.1378346 | 0.20182243 | −0.68295 | 0.49463843 | 0.97128468 |
| ENSG00000100479 | 251.080204 | 0.26200597 | 0.16135844 | 1.62375123 | 0.10442891 | 0.92973396 |
| ENSG00000020577 | 261.2629 | −0.0479999 | 0.18970939 | −0.2530182 | 0.80025418 | 0.98975709 |
| ENSG00000131979 | 905.131557 | −0.0019107 | 0.13252864 | −0.0144174 | 0.98849698 | 0.99896068 |
| ENSG00000070182 | 86.268933 | −0.7240817 | 0.26789483 | −2.702858 | 0.00687461 | 0.39961507 |
| ENSG00000274330 | 17.1569299 | 1.26908697 | 0.47058436 | 2.69683203 | 0.00700026 | 0.40580395 |
| ENSG00000119681 | 373.285888 | −0.3155494 | 0.22086544 | −1.4286954 | 0.15309179 | 0.93425563 |
| ENSG00000100604 | 8593.89536 | −1.1135401 | 0.18101005 | −6.1518139 | 7.66E−10 | 1.01E−06 |
| ENSG00000182218 | 44.6313937 | −1.1179817 | 0.30157508 | −3.7071423 | 0.00020961 | 0.0419556 |
| ENSG00000183092 | 1514.42165 | −0.844362 | 0.20554165 | −4.1079852 | 3.99E−05 | 0.01096766 |
| ENSG00000259031 | 31.9495195 | −1.2391375 | 0.37581046 | −3.2972406 | 0.0009764 | 0.12445546 |
| ENSG00000185559 | 1605.2048 | −2.1799449 | 0.22390974 | −9.7358196 | 2.12E−22 | 2.24E−18 |
| ENSG00000254656 | 1169.57968 | −1.2416545 | 0.25493109 | −4.8705494 | 1.11E−06 | 0.00061967 |
| ENSG00000221077 | 55.3196793 | −1.4100692 | 0.39861654 | −3.5374078 | 0.00040408 | 0.06428443 |
| ENSG00000080824 | 29725.2457 | 0.35841835 | 0.17235634 | 2.07951939 | 0.03756964 | 0.80600298 |
| ENSG00000258986 | 548.914861 | 0.37552937 | 0.17346432 | 2.1648796 | 0.03039691 | 0.77026126 |
| ENSG00000184601 | 31.3868627 | 0.66489036 | 0.35567443 | 1.86937914 | 0.06157009 | 0.88966453 |
| ENSG00000184990 | 672.378919 | −0.1544148 | 0.24765078 | −0.6235184 | 0.53294395 | 0.97577914 |
| ENSG00000185347 | 243.11557 | −0.0561486 | 0.26086769 | −0.2152379 | 0.82958188 | 0.99145366 |
| ENSG00000175344 | 429.989218 | −0.1196778 | 0.15503731 | −0.7719292 | 0.44015634 | 0.9694822 |
| ENSG00000051180 | 526.852118 | 0.37265684 | 0.13599317 | 2.74026142 | 0.00613903 | 0.3820465 |
| ENSG00000128965 | 207.156343 | 0.46538648 | 0.21952067 | 2.12001215 | 0.03400502 | 0.78122935 |
| ENSG00000137825 | 50.2762886 | 0.62265881 | 0.32708118 | 1.9036828 | 0.05695151 | 0.8681139 |
| ENSG00000128951 | 2406.59681 | 0.30167386 | 0.17137184 | 1.76034678 | 0.07834903 | 0.92973396 |
| ENSG00000138587 | 152.444254 | 0.27808989 | 0.21419508 | 1.29830195 | 0.1941836 | 0.94056812 |
| ENSG00000140416 | 1362.61801 | 0.3524429 | 0.12162386 | 2.89781054 | 0.00375778 | 0.30004062 |
| ENSG00000166803 | 340.428891 | 0.42773627 | 0.22689109 | 1.88520523 | 0.05940213 | 0.88326758 |
| ENSG00000137834 | 532.719059 | 0.25112755 | 0.20484728 | 1.22592573 | 0.22022663 | 0.95038315 |
| ENSG00000128973 | 608.332068 | 0.21652261 | 0.13503384 | 1.60346921 | 0.1088311 | 0.92973396 |
| ENSG00000259781 | 390.887013 | 0.3269861 | 0.21476814 | 1.52250749 | 0.12788197 | 0.92973396 |
| ENSG00000140365 | 976.638153 | −0.060107 | 0.21445836 | −0.2802735 | 0.77926772 | 0.98715916 |
| ENSG00000161980 | 253.339381 | 0.16955696 | 0.21149125 | 0.80172091 | 0.42271442 | 0.9694822 |
| ENSG00000161981 | 443.672135 | 0.03642958 | 0.21699552 | 0.16788171 | 0.86667634 | 0.99145366 |
| ENSG00000182685 | 115.470975 | −0.2314262 | 0.22572464 | −1.0252587 | 0.30524112 | 0.96279754 |
| ENSG00000162062 | 621.103782 | −0.1095972 | 0.18498621 | −0.5924615 | 0.5535416 | 0.97756935 |
| ENSG00000118898 | 33.6793528 | −0.4397694 | 0.30623979 | −1.4360297 | 0.1509939 | 0.9339277 |
| ENSG00000175643 | 583.354589 | 0.31165558 | 0.1433352 | 2.17431286 | 0.02968165 | 0.76200705 |
| ENSG00000149929 | 606.917543 | 0.18031207 | 0.15057557 | 1.1974855 | 0.23111738 | 0.95572373 |
| ENSG00000179958 | 502.09622 | 0.09247055 | 0.19716934 | 0.46899053 | 0.63907641 | 0.98262846 |
| ENSG00000089280 | 8051.48337 | 0.06521591 | 0.14031969 | 0.4647666 | 0.64209861 | 0.98262846 |
| ENSG00000091651 | 607.103796 | 0.19559106 | 0.15533074 | 1.25919096 | 0.20796136 | 0.9423199 |
| ENSG00000125148 | 44.9000027 | 0.7439947 | 0.38087537 | 1.95338098 | 0.05077447 | 0.85628493 |
| ENSG00000125170 | 4162.24254 | 0.04431036 | 0.12019838 | 0.36864361 | 0.71239339 | 0.98450389 |
| ENSG00000181938 | 229.819668 | 0.25224895 | 0.17263684 | 1.4611536 | 0.14397329 | 0.9324271 |
| ENSG00000140937 | 877.908772 | −0.3842318 | 0.12933125 | −2.970912 | 0.00296917 | 0.25853758 |
| ENSG00000103154 | 145.991349 | 0.7941523 | 0.2385033 | 3.329733 | 0.00086929 | 0.11715522 |
| ENSG00000103187 | 323.892506 | 0.5198921 | 0.18545096 | 2.80339405 | 0.00505678 | 0.34463288 |
| ENSG00000131153 | 656.796472 | 0.275189 | 0.16830245 | 1.6350861 | 0.102031 | 0.92973396 |
| ENSG00000167523 | 188.06405 | 0.42695575 | 0.17940157 | 2.37988856 | 0.01731787 | 0.6160856 |
| ENSG00000129235 | 583.92011 | 0.10292859 | 0.22814813 | 0.45114806 | 0.65188284 | 0.98262846 |
| ENSG00000129255 | 781.585056 | 0.24381096 | 0.15807898 | 1.54233637 | 0.12299188 | 0.92973396 |
| ENSG00000179111 | 51.1174922 | −1.1130669 | 0.30901508 | −3.6019825 | 0.0003158 | 0.05303179 |
| ENSG00000172301 | 496.16905 | 0.0114348 | 0.19739875 | 0.0579274 | 0.95380646 | 0.99775935 |
| ENSG00000267321 | 129.437949 | 0.4429854 | 0.23943359 | 1.85013891 | 0.06429353 | 0.89893273 |
| ENSG00000173991 | 58.3180863 | 0.09961797 | 0.26687803 | 0.37327154 | 0.70894636 | 0.98450389 |
| ENSG00000094804 | 869.943664 | 0.47947377 | 0.13396667 | 3.57905283 | 0.00034484 | 0.05700396 |
| ENSG00000167920 | 98.9983987 | 0.74315315 | 0.22333709 | 3.32749538 | 0.0008763 | 0.11735269 |
| ENSG00000108861 | 614.956278 | −0.2113875 | 0.11776424 | −1.795006 | 0.07265275 | 0.92346595 |
| ENSG00000186868 | 1366.26959 | −0.194091 | 0.12898464 | −1.5047604 | 0.13238568 | 0.92973396 |
| ENSG00000108465 | 1821.35087 | −0.2405867 | 0.14306775 | −1.6816279 | 0.09264102 | 0.92973396 |
| ENSG00000239672 | 595.254447 | 0.21847413 | 0.24155497 | 0.90444891 | 0.36575741 | 0.96401724 |
| ENSG00000087191 | 1942.01786 | 0.114369 | 0.14891412 | 0.7680199 | 0.44247536 | 0.9694822 |
| ENSG00000154229 | 11045.3539 | −0.2440105 | 0.16686212 | −1.4623481 | 0.14364586 | 0.9324271 |
| ENSG00000075461 | 5066.4808 | −0.4208653 | 0.14321409 | −2.9387145 | 0.00329576 | 0.27454758 |
| ENSG00000180616 | 156.262292 | 0.53096895 | 0.20701024 | 2.56494053 | 0.01031935 | 0.48686308 |
| ENSG00000125450 | 964.009726 | 0.10488054 | 0.11769573 | 0.891116 | 0.37286694 | 0.96401724 |
| ENSG00000161547 | 3572.4897 | 0.20695768 | 0.18429832 | 1.12294933 | 0.26145901 | 0.96256622 |
| ENSG00000167900 | 835.760296 | 0.26905863 | 0.17402506 | 1.54609124 | 0.12208253 | 0.92973396 |
| ENSG00000089685 | 1727.20212 | 0.11980298 | 0.14319729 | 0.83662885 | 0.40280122 | 0.9694822 |
| ENSG00000224877 | 917.86588 | −0.0058616 | 0.26617762 | −0.0220215 | 0.98243081 | 0.9985054 |
| ENSG00000183048 | 119.565826 | −0.1171642 | 0.29644942 | −0.3952248 | 0.69267697 | 0.98450389 |
| ENSG00000183684 | 2016.78563 | 0.12190535 | 0.2146526 | 0.56791925 | 0.5700898 | 0.97809273 |
| ENSG00000176890 | 588.137002 | 0.30168047 | 0.16073557 | 1.87687433 | 0.06053532 | 0.88626863 |
| ENSG00000080986 | 668.269042 | 0.21836165 | 0.16801893 | 1.29962528 | 0.19372943 | 0.94056812 |
| ENSG00000173482 | 140.970869 | −0.4488115 | 0.17756567 | −2.5275803 | 0.01148516 | 0.51595416 |
| ENSG00000141441 | 565.882074 | −0.4808479 | 0.15398766 | −3.1226391 | 0.00179237 | 0.18233093 |
| ENSG00000166974 | 675.182292 | −0.0130867 | 0.13634286 | −0.0959838 | 0.92353347 | 0.99569987 |
| ENSG00000184828 | 82.934306 | −0.6538893 | 0.21403185 | −3.0551025 | 0.00224984 | 0.21171292 |
| ENSG00000125835 | 2607.27947 | 0.03798674 | 0.20873987 | 0.18198124 | 0.85559744 | 0.99145366 |
| ENSG00000101361 | 2102.08274 | 0.18117959 | 0.10788305 | 1.67940736 | 0.09307268 | 0.92973396 |
| ENSG00000088854 | 738.906445 | −0.0951501 | 0.13527763 | −0.703369 | 0.48182581 | 0.97128468 |
| ENSG00000132646 | 3349.40447 | 0.37415017 | 0.16197519 | 2.30992266 | 0.02089244 | 0.66157976 |
| ENSG00000089199 | 2028.44486 | −0.347687 | 0.09566941 | −3.6342546 | 0.00027879 | 0.0512941 |
| ENSG00000125869 | 699.512665 | 0.42043807 | 0.14282315 | 2.94376702 | 0.00324244 | 0.27224915 |
| ENSG00000101003 | 620.311777 | 0.42742383 | 0.16010918 | 2.66957724 | 0.00759468 | 0.42177386 |
| ENSG00000125968 | 1196.66942 | −0.0280856 | 0.22547338 | −0.1245627 | 0.90086975 | 0.99474038 |
| ENSG00000101412 | 1344.81629 | 0.19732817 | 0.16408712 | 1.20258172 | 0.22913823 | 0.95445475 |
| ENSG00000149636 | 402.47954 | 0.18243322 | 0.16447072 | 1.10921398 | 0.26733788 | 0.96256622 |
| ENSG00000204103 | 312.707527 | 0.19095551 | 0.19120692 | 0.99868514 | 0.31794724 | 0.96279754 |
| ENSG00000101057 | 3301.17794 | 0.16118452 | 0.12814535 | 1.2578258 | 0.20845476 | 0.9423199 |
| ENSG00000124191 | 489.704233 | −0.2269958 | 0.18862065 | −1.2034513 | 0.22880173 | 0.95445475 |
| ENSG00000158445 | 255.203877 | −0.2688332 | 0.18738444 | −1.4346615 | 0.15138358 | 0.9339277 |
| ENSG00000124216 | 355.278159 | 0.04686198 | 0.16402772 | 0.28569551 | 0.77511135 | 0.98663988 |
| ENSG00000054803 | 24.1796649 | −2.6049882 | 0.50740764 | −5.133916 | 2.84E−07 | 0.00018356 |
| ENSG00000101144 | 48.55399 | −1.8673004 | 0.31487404 | −5.9303091 | 3.02E−09 | 3.37E−06 |
| ENSG00000130270 | 92.4956445 | −0.7382357 | 0.21784803 | −3.3887647 | 0.00070208 | 0.09880993 |
| ENSG00000167670 | 1247.79762 | 0.2590725 | 0.13421422 | 1.93029095 | 0.0535708 | 0.86173331 |
| ENSG00000280239 | 22.7532849 | 0.63388466 | 0.50842516 | 1.24676098 | 0.21248515 | 0.94608273 |
| ENSG00000276043 | 1534.19026 | 0.23078829 | 0.16128597 | 1.43092601 | 0.15245142 | 0.93425563 |
| ENSG00000205744 | 120.232388 | −0.4754966 | 0.18707825 | −2.5416988 | 0.01103152 | 0.50944234 |
| ENSG00000099783 | 5464.83442 | 0.15337458 | 0.1451777 | 1.05646099 | 0.29075765 | 0.96279754 |
| ENSG00000198258 | 2152.44738 | 0.00906974 | 0.23304084 | 0.03891912 | 0.96895487 | 0.99796143 |
| ENSG00000161888 | 510.174599 | 0.26072265 | 0.17160437 | 1.51932411 | 0.12868093 | 0.92973396 |
| ENSG00000104889 | 1000.50637 | 0.14939857 | 0.19562156 | 0.76371222 | 0.44503876 | 0.9694822 |
| ENSG00000105011 | 508.492535 | 0.23692308 | 0.12747069 | 1.85864754 | 0.06307711 | 0.89393747 |
| ENSG00000123136 | 1562.21912 | −0.0018301 | 0.17650759 | −0.0103686 | 0.9917272 | 0.99915206 |
| ENSG00000105393 | 339.472345 | 0.05673895 | 0.21631484 | 0.26229799 | 0.79309171 | 0.98801156 |
| ENSG00000105639 | 119.482856 | −0.6120881 | 0.18492117 | −3.3099949 | 0.00093298 | 0.12037108 |
| ENSG00000160161 | 160.686702 | −0.6778684 | 0.24125684 | −2.8097375 | 0.00495819 | 0.34284437 |
| ENSG00000269416 | 339.54691 | 0.30783494 | 0.1478261 | 2.08241263 | 0.0373048 | 0.80450709 |
| ENSG00000105173 | 388.91138 | 0.26403937 | 0.15536367 | 1.69949236 | 0.08922645 | 0.92973396 |
| ENSG00000124302 | 560.471054 | 0.11826319 | 0.21629216 | 0.5467752 | 0.58453318 | 0.97969186 |
| ENSG00000011332 | 433.045432 | 0.06655107 | 0.23544038 | 0.28266634 | 0.77743262 | 0.98663988 |
| ENSG00000125746 | 301.72452 | −0.3897162 | 0.1592941 | −2.4465203 | 0.01442427 | 0.57261359 |
| ENSG00000124440 | 96.4992964 | −0.5963667 | 0.2030197 | −2.9374819 | 0.00330889 | 0.2745604 |
| ENSG00000105281 | 1168.99505 | 0.0804705 | 0.15071729 | 0.53391686 | 0.59339906 | 0.97969186 |
| ENSG00000142230 | 2731.46721 | 0.20895398 | 0.12344512 | 1.6926872 | 0.09051502 | 0.92973396 |
| ENSG00000142552 | 199.303656 | −0.0406611 | 0.21952019 | −0.1852271 | 0.85305091 | 0.99145366 |
| ENSG00000167747 | 881.673158 | 0.04936715 | 0.16329587 | 0.30231717 | 0.76241029 | 0.98663988 |
| ENSG00000093009 | 724.72203 | 0.33752276 | 0.12681742 | 2.66148581 | 0.00777966 | 0.42645043 |
| ENSG00000099901 | 3673.15568 | 0.19665008 | 0.18143211 | 1.08387694 | 0.27841937 | 0.96279754 |
| ENSG00000100024 | 27.9159568 | −1.1135486 | 0.36160379 | −3.0794715 | 0.00207368 | 0.2012708 |
| ENSG00000100297 | 1649.10322 | 0.14522839 | 0.14478781 | 1.00304288 | 0.31584017 | 0.96279754 |
| ENSG00000128283 | 159.970264 | 0.33172317 | 0.23603595 | 1.40539256 | 0.15990458 | 0.93901912 |
| ENSG00000100129 | 6351.35552 | 0.00738734 | 0.10523472 | 0.07019867 | 0.94403553 | 0.99699764 |
| ENSG00000128272 | 3378.9489 | 0.27211054 | 0.1394185 | 1.95175351 | 0.05096748 | 0.85628493 |
| ENSG00000100162 | 136.328755 | 0.15377922 | 0.22082824 | 0.69637481 | 0.48619413 | 0.97128468 |
| ENSG00000202058 | 21.2434535 | −3.4772339 | 0.6889469 | −5.0471726 | 4.48E−07 | 0.00027107 |
| ENSG00000100416 | 916.810683 | −0.0377248 | 0.13167796 | −0.2864928 | 0.77450072 | 0.98663988 |
| ENSG00000025770 | 880.821129 | 0.07351208 | 0.15808084 | 0.46502838 | 0.64191113 | 0.98262846 |
| ENSG00000277437 | 53.4048221 | −4.3616282 | 0.92575931 | −4.7114062 | 2.46E−06 | 0.00126961 |
| ENSG00000277105 | 13850.374 | −3.7359464 | 0.87572583 | −4.2661142 | 1.99E−05 | 0.00689946 |
| ENSG00000276737 | 409.786811 | −4.1856208 | 0.95803671 | −4.3689566 | 1.25E−05 | 0.00463425 |
| ENSG00000274735 | 322.607828 | −3.5733746 | 0.79355053 | −4.5030208 | 6.70E−06 | 0.00283507 |
| ENSG00000279718 | 56.5409922 | −0.4562904 | 0.25764509 | −1.7710037 | 0.07656009 | 0.92973396 |
| ENSG00000154734 | 2372.64189 | −0.181198 | 0.10233728 | −1.7705962 | 0.07662787 | 0.92973396 |
| ENSG00000154736 | 211.849603 | −0.6338525 | 0.19320165 | −3.2807818 | 0.0010352 | 0.1288456 |
| ENSG00000159055 | 372.534032 | −0.0273056 | 0.18661563 | −0.1463199 | 0.88366889 | 0.99281445 |
| ENSG00000159259 | 510.048758 | 0.29548622 | 0.12747487 | 2.31799588 | 0.02044954 | 0.65251636 |
| ENSG00000183527 | 375.521209 | 0.25103341 | 0.14870492 | 1.68813121 | 0.09138605 | 0.92973396 |
| ENSG00000160179 | 150.495894 | −0.0435039 | 0.19922442 | −0.2183661 | 0.82714385 | 0.99145366 |
| ENSG00000228709 | 459.974012 | −0.3256038 | 0.20961332 | −1.5533546 | 0.12033844 | 0.92973396 |
| ENSG00000175894 | 596.369164 | −0.543398 | 0.17905007 | −3.0348942 | 0.0024062 | 0.22040175 |
| ENSG00000235890 | 709.057452 | −0.6104192 | 0.23675801 | −2.5782408 | 0.00993048 | 0.48082139 |
| ENSG00000182912 | 1286.0869 | −0.5044724 | 0.15264589 | −3.3048541 | 0.00095026 | 0.12185769 |

TABLE 7
Differential gene Expression_DESeq2_dC_Mock

| | | Log2 Fold | | | | |
|---|---|---|---|---|---|---|
| | Base Mean | Change | Lfcse | Stat | Pvalue | Padj |
| ENSG00000227232 | 150.313092 | −0.6637818 | 0.2201574 | −3.0150331 | 0.00256951 | 0.04770747 |
| ENSG00000228794 | 421.20101 | 0.42393973 | 0.14148654 | 2.99632562 | 0.00273254 | 0.0498616 |
| ENSG00000221978 | 4011.35976 | −0.3568485 | 0.1083594 | −3.2931936 | 0.00099056 | 0.02344469 |
| ENSG00000171680 | 539.748302 | −0.7637991 | 0.23686928 | −3.2245594 | 0.00126167 | 0.02798757 |
| ENSG00000049249 | 11.137498 | 3.05237817 | 0.69290435 | 4.40519413 | 1.06E−05 | 0.00065939 |
| ENSG00000171608 | 251.374075 | −0.6182372 | 0.18656927 | −3.3137139 | 0.00092066 | 0.0222241 |
| ENSG00000116661 | 127.72285 | −0.8718779 | 0.24665415 | −3.5348196 | 0.00040805 | 0.0124098 |
| ENSG00000177000 | 373.463612 | −0.4942737 | 0.15028511 | −3.288907 | 0.00100577 | 0.02363921 |
| ENSG00000070886 | 191.138666 | −0.9506329 | 0.25467523 | −3.7327261 | 0.00018942 | 0.00666895 |
| ENSG00000249087 | 124.251797 | −0.723928 | 0.22711975 | −3.1874285 | 0.00143544 | 0.03075569 |
| ENSG00000007968 | 639.194696 | 0.69034806 | 0.14061242 | 4.90958086 | 9.13E−07 | 8.28E−05 |
| ENSG00000169504 | 3161.94974 | 0.55711554 | 0.17282768 | 3.2235319 | 0.0012662 | 0.0280515 |
| ENSG00000127423 | 102.054816 | 0.70556844 | 0.23455072 | 3.00817008 | 0.00262826 | 0.0483224 |
| ENSG00000117748 | 1234.30582 | 0.45715662 | 0.14313938 | 3.19378647 | 0.0014042 | 0.03043329 |
| ENSG00000134684 | 2095.07881 | 0.37255086 | 0.11130168 | 3.34721692 | 0.00081627 | 0.02052171 |
| ENSG00000092853 | 835.308296 | 0.91142759 | 0.18609809 | 4.89756559 | 9.70E−07 | 8.71E−05 |
| ENSG00000183317 | 169.763339 | −0.7525365 | 0.20976424 | −3.587535 | 0.00033382 | 0.01051003 |
| ENSG00000116990 | 137.955411 | −0.722962 | 0.23059006 | −3.1352697 | 0.00171696 | 0.0347695 |
| ENSG00000132780 | 6332.75757 | 0.53919787 | 0.12424234 | 4.33988806 | 1.43E−05 | 0.00085786 |
| ENSG00000085999 | 325.676153 | 0.521018 | 0.16537993 | 3.15043059 | 0.0016303 | 0.03349066 |
| ENSG00000123473 | 504.833466 | 0.61162574 | 0.18990419 | 3.22070691 | 0.00127875 | 0.02821894 |
| ENSG00000162374 | 10995.5619 | 0.5332 | 0.1569422 | 3.39742905 | 0.00068022 | 0.01806474 |
| ENSG00000169213 | 192.784416 | 0.6366386 | 0.20769573 | 3.06524641 | 0.00217491 | 0.04184601 |
| ENSG00000085840 | 357.351495 | 0.84352107 | 0.16627919 | 5.07292036 | 3.92E−07 | 4.00E−05 |
| ENSG00000116133 | 1258.35855 | 0.7276945 | 0.12826784 | 5.67324211 | 1.40E−08 | 2.24E−06 |
| ENSG00000162407 | 227.302124 | −1.2332801 | 0.19863204 | −6.208868 | 5.34E−10 | 1.15E−07 |
| ENSG00000162607 | 1756.01494 | 0.65180864 | 0.16593353 | 3.92813098 | 8.56E−05 | 0.00359611 |
| ENSG00000172380 | 750.651241 | 0.67028482 | 0.20084746 | 3.33728296 | 0.00084602 | 0.0210512 |
| ENSG00000024526 | 760.935777 | 0.66962508 | 0.21213703 | 3.15656854 | 0.00159637 | 0.03307749 |
| ENSG00000172260 | 196.886238 | 0.77155651 | 0.22426731 | 3.44034313 | 0.00058098 | 0.01603119 |
| ENSG00000178965 | 215.550561 | 1.2174429 | 0.23760781 | 5.12374941 | 3.00E−07 | 3.20E−05 |
| ENSG00000162614 | 30.4035506 | 1.75610244 | 0.40102143 | 4.37907384 | 1.19E−05 | 0.00073816 |
| ENSG00000142871 | 226.188748 | 1.71192707 | 0.16864262 | 10.1512123 | 3.27E−24 | 3.27E−21 |
| ENSG00000143013 | 215.289297 | −0.8542431 | 0.23033505 | −3.7086977 | 0.00020833 | 0.0072594 |
| ENSG00000197147 | 789.695287 | 0.62936817 | 0.1889682 | 3.33055062 | 0.00086674 | 0.02137885 |
| ENSG00000162664 | 1022.08734 | 0.60869157 | 0.15761332 | 3.86192988 | 0.00011249 | 0.00450245 |
| ENSG00000223745 | 331.552952 | −0.6510476 | 0.15866507 | −4.1032823 | 4.07E−05 | 0.00200359 |
| ENSG00000237954 | 49.4200112 | −2.2651139 | 0.357457 | −6.3367452 | 2.35E−10 | 5.46E−08 |
| ENSG00000228971 | 282.287961 | −0.7247427 | 0.20674774 | −3.5054442 | 0.00045585 | 0.01348914 |
| ENSG00000099260 | 36.5706791 | 2.00301986 | 0.36203884 | 5.53261047 | 3.15E−08 | 4.50E−06 |
| ENSG00000156876 | 473.837278 | 0.6729561 | 0.17552947 | 3.83386399 | 0.00012615 | 0.00491465 |
| ENSG00000162692 | 19.5290366 | 2.97446941 | 0.47228842 | 6.29799352 | 3.02E−10 | 6.73E−08 |
| ENSG00000060718 | 173.362961 | 1.33086064 | 0.21521833 | 6.1837699 | 6.26E−10 | 1.31E−07 |
| ENSG00000184371 | 88.1023102 | 1.43386743 | 0.26377725 | 5.43590256 | 5.45E−08 | 7.23E−06 |
| ENSG00000116396 | 116.688511 | −0.8577976 | 0.22182262 | −3.8670429 | 0.00011016 | 0.00441954 |
| ENSG00000116774 | 73.1656435 | 0.83557761 | 0.25032969 | 3.33790849 | 0.00084412 | 0.0210512 |
| ENSG00000183508 | 36.5158056 | 1.53956072 | 0.33886134 | 4.54333539 | 5.54E−06 | 0.00037737 |
| ENSG00000092621 | 3312.3205 | 0.54052143 | 0.15894843 | 3.40060876 | 0.00067236 | 0.017912 |
| ENSG00000265972 | 8541.57977 | −0.9091396 | 0.13185748 | −6.8948658 | 5.39E−12 | 1.91E−09 |
| ENSG00000162836 | 280.765018 | −0.5545233 | 0.16024495 | −3.4604731 | 0.00053923 | 0.01517527 |
| ENSG00000203814 | 22.7288953 | −1.915018 | 0.4836941 | −3.959151 | 7.52E−05 | 0.00327289 |
| ENSG00000184678 | 208.124549 | −0.7327504 | 0.21564337 | −3.3979733 | 0.00067887 | 0.0180571 |
| ENSG00000143401 | 4332.62512 | 0.6869099 | 0.16035718 | 4.28362434 | 1.84E−05 | 0.00103665 |
| ENSG00000197747 | 13.5573855 | 2.25415276 | 0.59308008 | 3.80075617 | 0.00014426 | 0.00546431 |
| ENSG00000143578 | 311.463247 | −0.7392861 | 0.16916428 | −4.3702258 | 1.24E−05 | 0.00076039 |
| ENSG00000160691 | 5930.36505 | −0.6437534 | 0.16505204 | −3.9003055 | 9.61E−05 | 0.00394413 |
| ENSG00000143320 | 527.431765 | 0.68914494 | 0.2103635 | 3.27597196 | 0.00105299 | 0.02437823 |
| ENSG00000143303 | 585.345042 | −0.4499134 | 0.1415403 | −3.178695 | 0.0014794 | 0.03146036 |
| ENSG00000198400 | 692.709832 | 0.66613096 | 0.21159912 | 3.14807993 | 0.00164347 | 0.03360198 |
| ENSG00000027644 | 343.378514 | 0.71457166 | 0.21316864 | 3.35214246 | 0.00080189 | 0.02028758 |
| ENSG00000183853 | 114.745305 | 1.24854982 | 0.27488559 | 4.54207082 | 5.57E−06 | 0.00037812 |
| ENSG00000158710 | 3326.71326 | 0.48400903 | 0.14776335 | 3.27556879 | 0.00105449 | 0.02437979 |
| ENSG00000162745 | 76.6681789 | 1.3479287 | 0.23616632 | 5.70753998 | 1.15E−08 | 1.87E−06 |
| ENSG00000000460 | 431.863001 | 0.94026256 | 0.18170363 | 5.17470426 | 2.28E−07 | 2.53E−05 |
| ENSG00000135829 | 7804.95605 | 0.56988884 | 0.11416421 | 4.99183451 | 5.98E−07 | 5.73E−05 |
| ENSG00000198860 | 1040.35608 | 0.44392992 | 0.14314835 | 3.10118772 | 0.00192746 | 0.03785765 |
| ENSG00000135842 | 35.8103349 | 1.49227821 | 0.32876046 | 4.53910492 | 5.65E−06 | 0.00038144 |
| ENSG00000118193 | 874.150692 | 0.68181048 | 0.22322992 | 3.05429696 | 0.00225589 | 0.04296565 |
| ENSG00000159176 | 579.821256 | 1.32314377 | 0.15923754 | 8.30924549 | 9.63E−17 | 6.29E−14 |
| ENSG00000163431 | 32.0031836 | 1.29349513 | 0.34407049 | 3.75938992 | 0.00017033 | 0.00611093 |
| ENSG00000228288 | 139.8182 | −0.9494663 | 0.26825797 | −3.5393778 | 0.00040107 | 0.01228553 |
| ENSG00000143847 | 412.507564 | −0.5749402 | 0.18424131 | −3.1205822 | 0.00180494 | 0.0362483 |
| ENSG00000163545 | 27.8850321 | 1.50273587 | 0.37450452 | 4.01259736 | 6.01E−05 | 0.00273107 |
| ENSG00000143486 | 698.474981 | −0.3911026 | 0.12878259 | −3.0369217 | 0.00239008 | 0.04471839 |
| ENSG00000076356 | 3493.01779 | 0.76604316 | 0.2236546 | 3.42511689 | 0.00061453 | 0.01668585 |
| ENSG00000117595 | 223.578204 | 0.74559593 | 0.17550286 | 4.24834065 | 2.15E−05 | 0.00119433 |
| ENSG00000198570 | 186.006471 | 0.85152957 | 0.18160242 | 4.6889771 | 2.75E−06 | 0.00020709 |
| ENSG00000170385 | 288.733445 | 0.87375254 | 0.17012205 | 5.13603356 | 2.81E−07 | 3.01E−05 |
| ENSG00000143476 | 1826.89204 | 0.93667905 | 0.13861023 | 6.7576471 | 1.40E−11 | 4.67E−09 |
| ENSG00000152104 | 175.493759 | 0.74678412 | 0.21534978 | 3.46777281 | 0.00052479 | 0.01491742 |
| ENSG00000117724 | 8065.2183 | 0.58870061 | 0.18374877 | 3.2038344 | 0.0013561 | 0.02963326 |
| ENSG00000092969 | 111.687784 | 1.7902849 | 0.24989244 | 7.16422198 | 7.82E−13 | 3.24E−10 |
| ENSG00000163050 | 1333.12144 | −0.6211247 | 0.14924878 | −4.1616736 | 3.16E−05 | 0.00162461 |
| ENSG00000181218 | 462.029319 | −0.9047315 | 0.24573815 | −3.6816892 | 0.00023169 | 0.00797534 |
| ENSG00000196890 | 47.9719811 | −0.9038781 | 0.29048618 | −3.1116045 | 0.00186074 | 0.03706184 |
| ENSG00000168264 | 3247.99992 | −0.3467926 | 0.10949528 | −3.1671919 | 0.00153919 | 0.03248756 |
| ENSG00000180875 | 343.86164 | 1.79262445 | 0.16553909 | 10.8290099 | 2.51E−27 | 3.55E−24 |
| ENSG00000174371 | 1592.46943 | 0.78325359 | 0.1381069 | 5.67135724 | 1.42E−08 | 2.25E−06 |
| ENSG00000115738 | 2503.56412 | 0.67571675 | 0.15490756 | 4.36206435 | 1.29E−05 | 0.00077809 |
| ENSG00000171848 | 2200.77166 | 0.91511968 | 0.13595545 | 6.73102627 | 1.68E−11 | 5.50E−09 |
| ENSG00000178295 | 1124.30805 | 0.79887298 | 0.20281737 | 3.93887855 | 8.19E−05 | 0.0034993 |
| ENSG00000138092 | 519.557341 | 0.59065146 | 0.13136316 | 4.49632483 | 6.91E−06 | 0.00045586 |
| ENSG00000234072 | 502.042261 | −0.5036832 | 0.15293976 | −3.2933439 | 0.00099003 | 0.02344469 |
| ENSG00000075426 | 608.189644 | 0.69903797 | 0.18603365 | 3.75758888 | 0.00017156 | 0.00614208 |
| ENSG00000213626 | 602.273167 | 0.50729917 | 0.12255366 | 4.13940443 | 3.48E−05 | 0.0017431 |
| ENSG00000172954 | 648.833712 | 0.58968987 | 0.17123002 | 3.44384627 | 0.0005735 | 0.01589605 |
| ENSG00000049323 | 298.697547 | 1.38749911 | 0.16708683 | 8.30406004 | 1.01E−16 | 6.32E−14 |
| ENSG00000150938 | 154.566282 | 1.54830906 | 0.21677751 | 7.14238794 | 9.17E−13 | 3.62E−10 |
| ENSG00000152133 | 616.472508 | 0.6345193 | 0.1921129 | 3.30284589 | 0.00095709 | 0.02292036 |
| ENSG00000225156 | 452.420714 | −0.6807842 | 0.13915071 | −4.8924235 | 9.96E−07 | 8.85E−05 |
| ENSG00000116016 | 1420.29983 | 0.58391104 | 0.18015543 | 3.24115154 | 0.00119048 | 0.02693657 |
| ENSG00000234690 | 24.2823373 | −1.3547506 | 0.43973104 | −3.0808618 | 0.00206402 | 0.04014463 |
| ENSG00000095002 | 2650.98225 | 0.60293791 | 0.15515966 | 3.88591926 | 0.00010194 | 0.00414863 |
| ENSG00000116062 | 4600.0304 | 0.47250769 | 0.11864368 | 3.9825779 | 6.82E−05 | 0.00304441 |
| ENSG00000143942 | 90.5527248 | 0.88578912 | 0.27987016 | 3.1650002 | 0.00155083 | 0.03253097 |
| ENSG00000173209 | 578.604221 | −0.4908499 | 0.14338254 | −3.4233588 | 0.00061852 | 0.01674059 |
| ENSG00000169764 | 581.489125 | 0.59139536 | 0.18199184 | 3.24957082 | 0.00115579 | 0.02618666 |
| ENSG00000115902 | 499.90418 | 0.63465382 | 0.16646755 | 3.81247759 | 0.00013758 | 0.00527337 |
| ENSG00000144043 | 899.665797 | 0.53133469 | 0.14090489 | 3.77087477 | 0.00016268 | 0.00596246 |
| ENSG00000163017 | 150.638098 | 3.61118462 | 0.22147877 | 16.3048792 | 9.11E−60 | 7.73E−56 |
| ENSG00000065911 | 1623.56559 | 0.65675424 | 0.12837781 | 5.11579257 | 3.12E−07 | 3.29E−05 |
| ENSG00000115363 | 69.9481129 | 1.78688643 | 0.27951501 | 6.39281026 | 1.63E−10 | 3.95E−08 |
| ENSG00000168874 | 141.952709 | 0.8919877 | 0.2427904 | 3.67390019 | 0.00023888 | 0.00810745 |
| ENSG00000068615 | 224.10288 | 0.86969583 | 0.16928564 | 5.13744609 | 2.78E−07 | 3.01E−05 |
| ENSG00000121152 | 917.008981 | 0.6353623 | 0.11746495 | 5.40895196 | 6.34E−08 | 8.21E−06 |
| ENSG00000115073 | 886.521455 | −0.5305383 | 0.15698402 | −3.3795687 | 0.000726 | 0.0188571 |
| ENSG00000170500 | 450.084957 | 0.72235175 | 0.21440136 | 3.36915654 | 0.00075399 | 0.01949267 |
| ENSG00000228528 | 23.003013 | −1.5967425 | 0.52119272 | −3.0636316 | 0.00218668 | 0.04188258 |
| ENSG00000115665 | 148.208812 | 0.69510753 | 0.20866021 | 3.33128938 | 0.00086445 | 0.02135322 |
| ENSG00000169679 | 1067.58024 | 0.45955537 | 0.14899353 | 3.08439825 | 0.00203964 | 0.03987644 |
| ENSG00000169607 | 486.582032 | 0.83667321 | 0.18262295 | 4.58142418 | 4.62E−06 | 0.00032655 |
| ENSG00000175497 | 263.30137 | −0.6001694 | 0.14962434 | −4.0111751 | 6.04E−05 | 0.00273408 |
| ENSG00000076003 | 2658.18865 | 0.84116108 | 0.12538506 | 6.70862262 | 1.96E−11 | 6.17E−09 |
| ENSG00000150540 | 29.7334973 | 1.86654912 | 0.45570218 | 4.09598461 | 4.20E−05 | 0.0020618 |
| ENSG00000144227 | 25.9240804 | 1.44387161 | 0.42956943 | 3.36120665 | 0.00077603 | 0.01983963 |
| ENSG00000187123 | 361.932957 | 0.63369814 | 0.19389597 | 3.26823789 | 0.00108219 | 0.02495221 |
| ENSG00000123610 | 128.542357 | −0.8607161 | 0.25790237 | −3.3373718 | 0.00084575 | 0.0210512 |
| ENSG00000115159 | 242.299623 | 0.66759936 | 0.17003131 | 3.92633193 | 8.63E−05 | 0.00361403 |
| ENSG00000152253 | 385.895688 | 0.63396833 | 0.20035698 | 3.16419382 | 0.00155513 | 0.03257065 |
| ENSG00000128683 | 9.89148776 | −4.2622578 | 0.90019687 | −4.7348063 | 2.19E−06 | 0.00017307 |
| ENSG00000071967 | 128.511473 | 0.66127811 | 0.20902919 | 3.1635683 | 0.00155848 | 0.03257065 |
| ENSG00000128708 | 801.274149 | 0.5711266 | 0.1892714 | 3.01750073 | 0.00254868 | 0.04737259 |
| ENSG00000091436 | 817.824625 | 0.46939545 | 0.15336212 | 3.06070016 | 0.0022082 | 0.04219952 |
| ENSG00000144354 | 1393.70125 | 0.57179937 | 0.11941201 | 4.78845777 | 1.68E−06 | 0.00013778 |
| ENSG00000170144 | 9873.18697 | 0.51643637 | 0.13106784 | 3.94022202 | 8.14E−05 | 0.00348854 |
| ENSG00000138448 | 1033.00414 | 0.5524299 | 0.18220202 | 3.0319636 | 0.00242968 | 0.04535947 |
| ENSG00000168542 | 4165.10392 | 1.3293586 | 0.12627596 | 10.527408 | 6.46E−26 | 7.83E−23 |
| ENSG00000144395 | 127.157434 | 0.65995025 | 0.19653175 | 3.35798294 | 0.00078513 | 0.01994571 |
| ENSG00000163535 | 683.565942 | 0.71582617 | 0.22383108 | 3.19806429 | 0.00138353 | 0.03009362 |
| ENSG00000155755 | 1144.77411 | 0.63654884 | 0.16526558 | 3.85167222 | 0.00011731 | 0.0046464 |
| ENSG00000155760 | 1477.73117 | −0.7506468 | 0.13283859 | −5.6508189 | 1.60E−08 | 2.51E−06 |
| ENSG00000118257 | 75.9005249 | 1.06103105 | 0.27138223 | 3.90972925 | 9.24E−05 | 0.00382444 |
| ENSG00000114948 | 1272.75094 | 0.44613447 | 0.13534929 | 3.29617144 | 0.00098012 | 0.02331927 |
| ENSG00000115414 | 1669.50945 | 1.64752198 | 0.24894149 | 6.61810938 | 3.64E−11 | 1.06E−08 |
| ENSG00000279348 | 252.769425 | 0.98608283 | 0.26288266 | 3.7510379 | 0.0001761 | 0.00625206 |
| ENSG00000260804 | 562.956932 | 0.8726238 | 0.1877949 | 4.6466853 | 3.37E−06 | 0.00024996 |
| ENSG00000115461 | 5652.74255 | 0.80037889 | 0.19104809 | 4.18941061 | 2.80E−05 | 0.00148318 |
| ENSG00000163516 | 606.7158 | −0.435645 | 0.14431769 | −3.018653 | 0.00253901 | 0.04724455 |
| ENSG00000237732 | 191.314287 | −0.8100997 | 0.26572246 | −3.0486684 | 0.00229858 | 0.0435345 |
| ENSG00000163082 | 260.376734 | −1.3085142 | 0.22002352 | −5.9471559 | 2.73E−09 | 4.98E−07 |
| ENSG00000123983 | 1337.59815 | 0.61307394 | 0.15423927 | 3.97482394 | 7.04E−05 | 0.00310447 |
| ENSG00000182600 | 25.9080797 | −1.5858636 | 0.48770164 | −3.2517085 | 0.00114714 | 0.02602526 |
| ENSG00000168918 | 110.553766 | −0.9905351 | 0.21769391 | −4.5501278 | 5.36E−06 | 0.00036984 |
| ENSG00000123485 | 794.516979 | 0.41382936 | 0.11383417 | 3.63537042 | 0.00027758 | 0.00911133 |
| ENSG00000132329 | 241.770148 | −0.9330081 | 0.28244154 | −3.3033672 | 0.00095531 | 0.02292036 |
| ENSG00000134121 | 69.2168007 | −0.9453448 | 0.29462463 | −3.2086413 | 0.00133364 | 0.02924009 |
| ENSG00000180914 | 89.3478068 | 0.7449285 | 0.23083498 | 3.22710401 | 0.0012505 | 0.02784906 |
| ENSG00000070950 | 622.871059 | 0.6430411 | 0.18755519 | 3.42854332 | 0.00060683 | 0.01652953 |
| ENSG00000196220 | 217.688378 | −0.6582922 | 0.20039978 | −3.2848947 | 0.0010202 | 0.02380998 |
| ENSG00000214021 | 196.442187 | −0.5924056 | 0.18082948 | −3.2760453 | 0.00105272 | 0.02437823 |
| ENSG00000144554 | 834.147088 | 0.5576491 | 0.16636101 | 3.35204212 | 0.00080218 | 0.02028758 |
| ENSG00000196639 | 44.106565 | 1.11950867 | 0.31800946 | 3.52036274 | 0.00043096 | 0.01289831 |
| ENSG00000263740 | 23.322155 | −2.000924 | 0.54435914 | −3.6757424 | 0.00023716 | 0.0080653 |
| ENSG00000129810 | 196.192927 | 0.69413517 | 0.21066729 | 3.29493568 | 0.00098444 | 0.02336501 |
| ENSG00000173705 | 34.9328488 | 1.17078923 | 0.36289611 | 3.22623799 | 0.00125429 | 0.02786035 |
| ENSG00000229589 | 98.1446269 | −0.7441202 | 0.24134171 | −3.0832641 | 0.00204743 | 0.03997111 |
| ENSG00000144655 | 41.0360996 | 1.07912257 | 0.30803075 | 3.50329497 | 0.00045954 | 0.01351542 |
| ENSG00000163808 | 809.907456 | 0.6038675 | 0.16861069 | 3.58143065 | 0.00034172 | 0.01067947 |
| ENSG00000164045 | 875.156728 | 0.69479646 | 0.12771604 | 5.44016599 | 5.32E−08 | 7.11E−06 |
| ENSG00000270441 | 40.8459775 | −1.2436356 | 0.37781809 | −3.2916254 | 0.0009961 | 0.02351022 |
| ENSG00000164062 | 1833.36451 | −0.4121921 | 0.12868505 | −3.2031082 | 0.00135953 | 0.02965451 |
| ENSG00000230454 | 50.2110006 | −1.0898874 | 0.30184587 | −3.6107414 | 0.00030532 | 0.00977609 |
| ENSG00000012171 | 107.144656 | −1.1470862 | 0.26206115 | −4.3771699 | 1.20E−05 | 0.00074193 |
| ENSG00000164082 | 145.409554 | 0.87868374 | 0.21856205 | 4.02029413 | 5.81E−05 | 0.00270244 |
| ENSG00000163932 | 223.486999 | −0.625807 | 0.17041167 | −3.6723246 | 0.00024035 | 0.00814134 |
| ENSG00000144730 | 3416.42255 | −0.4083582 | 0.13113692 | −3.1139832 | 0.0018458 | 0.03685085 |
| ENSG00000163376 | 92.3624215 | 1.11766912 | 0.25885787 | 4.31769411 | 1.58E−05 | 0.00092263 |
| ENSG00000185008 | 288.228115 | −1.2700994 | 0.22707279 | −5.5933581 | 2.23E−08 | 3.32E−06 |
| ENSG00000057019 | 915.80867 | 0.48918103 | 0.14877154 | 3.28813572 | 0.00100853 | 0.02363921 |
| ENSG00000168386 | 165.338596 | 2.34410012 | 0.20871021 | 11.2313629 | 2.86E−29 | 4.85E−26 |
| ENSG00000154175 | 82.9068122 | −1.2740219 | 0.32145828 | −3.9632576 | 7.39E−05 | 0.00322534 |
| ENSG00000163507 | 633.826231 | 0.84889732 | 0.17436605 | 4.86847834 | 1.12E−06 | 9.74E−05 |
| ENSG00000144824 | 167.235802 | 1.77331576 | 0.25295779 | 7.01032272 | 2.38E−12 | 8.77E−10 |
| ENSG00000091986 | 104.099881 | 1.17060441 | 0.23864086 | 4.90529746 | 9.33E−07 | 8.42E−05 |
| ENSG00000121579 | 2105.59893 | 0.45014974 | 0.1287389 | 3.49661008 | 0.00047121 | 0.01371601 |
| ENSG00000185565 | 3158.49177 | −0.551308 | 0.18317549 | −3.0097261 | 0.00261483 | 0.04823232 |
| ENSG00000163430 | 2754.92815 | 0.79076915 | 0.12837183 | 6.15998972 | 7.27E−10 | 1.49E−07 |
| ENSG00000051341 | 428.482131 | 0.89330631 | 0.23481733 | 3.8042606 | 0.00014223 | 0.00543576 |
| ENSG00000173193 | 52.1800177 | 0.85478478 | 0.28445798 | 3.00495975 | 0.00265616 | 0.04878251 |
| ENSG00000206527 | 514.672239 | 0.56680178 | 0.18245494 | 3.10653015 | 0.00189297 | 0.03752769 |
| ENSG00000065534 | 344.220933 | 1.21230559 | 0.22442352 | 5.4018652 | 6.60E−08 | 8.31E−06 |
| ENSG00000082781 | 140.579635 | 0.72672239 | 0.20867305 | 3.48258868 | 0.00049659 | 0.0142833 |
| ENSG00000173706 | 208.19034 | 1.25219745 | 0.20437393 | 6.12699217 | 8.96E−10 | 1.77E−07 |
| ENSG00000073111 | 2721.55265 | 0.5513073 | 0.13723013 | 4.01739254 | 5.88E−05 | 0.00272101 |
| ENSG00000114626 | 218.588082 | −0.6000959 | 0.17532536 | −3.4227558 | 0.0006199 | 0.01675105 |
| ENSG00000206384 | 24.5615613 | 1.74711476 | 0.43587521 | 4.00829116 | 6.12E−05 | 0.00276032 |
| ENSG00000163710 | 29.8041431 | −1.2167449 | 0.38310154 | −3.1760377 | 0.00149302 | 0.03163106 |
| ENSG00000144891 | 30.9187812 | −1.6244817 | 0.49949824 | −3.2522271 | 0.00114505 | 0.0260126 |
| ENSG00000163762 | 51.0116089 | −10.517562 | 1.46961191 | −7.1566936 | 8.26E−13 | 3.34E−10 |
| ENSG00000163661 | 64.2209448 | 1.42498423 | 0.29152695 | 4.88800169 | 1.02E−06 | 9.00E−05 |
| ENSG00000113810 | 3427.02789 | 0.60888598 | 0.16095112 | 3.78304916 | 0.00015492 | 0.00577796 |
| ENSG00000169255 | 181.053948 | 1.34226839 | 0.23833334 | 5.63189518 | 1.78E−08 | 2.75E−06 |
| ENSG00000114200 | 65.2453712 | −1.7562115 | 0.29207373 | −6.0129045 | 1.82E−09 | 3.40E−07 |
| ENSG00000085276 | 32.5717915 | −3.7419146 | 0.48312474 | −7.7452349 | 9.54E−15 | 4.91E−12 |
| ENSG00000114346 | 1589.61263 | 0.74633495 | 0.17983876 | 4.15002278 | 3.32E−05 | 0.00169926 |
| ENSG00000169760 | 116.220914 | 1.01616911 | 0.25491445 | 3.98631421 | 6.71E−05 | 0.00300479 |
| ENSG00000145198 | 111.716628 | −1.2859736 | 0.24809272 | −5.1834396 | 2.18E−07 | 2.45E−05 |
| ENSG00000145012 | 53.354428 | 2.39774782 | 0.33952184 | 7.06213132 | 1.64E−12 | 6.32E−10 |
| ENSG00000180611 | 152.301745 | 0.75071122 | 0.20264291 | 3.70460138 | 0.00021172 | 0.0073626 |
| ENSG00000114315 | 101.78102 | 1.01011815 | 0.33084799 | 3.05311862 | 0.00226476 | 0.04308497 |
| ENSG00000072274 | 3124.46183 | 0.71133793 | 0.16596189 | 4.28615219 | 1.82E−05 | 0.00103179 |
| ENSG00000119227 | 93.266148 | −0.8794025 | 0.25853396 | −3.4014972 | 0.00067018 | 0.01788196 |
| ENSG00000163975 | 350.262103 | −0.776967 | 0.23034077 | −3.3731198 | 0.00074322 | 0.01925554 |
| ENSG00000186777 | 49.3564808 | 1.19327385 | 0.30266105 | 3.94260792 | 8.06E−05 | 0.00346695 |
| ENSG00000215375 | 123.299254 | −0.7755077 | 0.22110542 | −3.5074117 | 0.00045249 | 0.01346583 |
| ENSG00000127415 | 250.274729 | −0.9901775 | 0.282309 | −3.5074245 | 0.00045247 | 0.01346583 |
| ENSG00000168936 | 973.452981 | −0.5639523 | 0.18745928 | −3.0083992 | 0.00262628 | 0.0483224 |
| ENSG00000123933 | 2082.48058 | −0.6215733 | 0.19438476 | −3.1976441 | 0.00138555 | 0.03009362 |
| ENSG00000181215 | 42.8654341 | 0.99495942 | 0.31989379 | 3.11028052 | 0.0018691 | 0.0371412 |
| ENSG00000178163 | 154.166418 | 0.89027927 | 0.20724488 | 4.29578407 | 1.74E−05 | 0.00099464 |
| ENSG00000109805 | 1193.94897 | 0.6574218 | 0.14661293 | 4.48406418 | 7.32E−06 | 0.000478 |
| ENSG00000038210 | 141.410532 | 0.75686134 | 0.22719484 | 3.33133158 | 0.00086432 | 0.02135322 |
| ENSG00000091490 | 80.3496775 | 0.73224621 | 0.24013353 | 3.04932932 | 0.00229353 | 0.04348736 |
| ENSG00000163394 | 20.7713188 | 1.71931147 | 0.4581163 | 3.7530022 | 0.00017473 | 0.00622158 |
| ENSG00000163697 | 669.364213 | 0.64470179 | 0.1648342 | 3.91121378 | 9.18E−05 | 0.0038103 |
| ENSG00000188848 | 1360.02465 | 0.63208427 | 0.18125792 | 3.48720914 | 0.00048809 | 0.01408653 |
| ENSG00000145248 | 1013.77448 | −0.5209142 | 0.16934084 | −3.0761287 | 0.00209707 | 0.04053227 |
| ENSG00000134853 | 3144.89091 | −0.6586824 | 0.14729086 | −4.4719842 | 7.75E−06 | 0.00050388 |
| ENSG00000174799 | 376.976233 | 0.67087975 | 0.16720239 | 4.01238126 | 6.01E−05 | 0.00273107 |
| ENSG00000084092 | 896.427562 | −0.5301643 | 0.17052297 | −3.1090495 | 0.0018769 | 0.03725267 |
| ENSG00000138758 | 2767.63087 | 0.60244786 | 0.1596693 | 3.77309771 | 0.00016123 | 0.0059352 |
| ENSG00000138675 | 35.4675086 | 1.66648931 | 0.3392134 | 4.91280504 | 8.98E−07 | 8.19E−05 |
| ENSG00000189308 | 441.599082 | 0.62458409 | 0.15417676 | 4.05109097 | 5.10E−05 | 0.00242331 |
| ENSG00000184305 | 412.545857 | −0.7240724 | 0.21516972 | −3.3651221 | 0.0007651 | 0.01967229 |
| ENSG00000163110 | 495.239317 | 0.8016529 | 0.16593508 | 4.83112379 | 1.36E−06 | 0.00011462 |
| ENSG00000155011 | 1746.60472 | −0.5625389 | 0.13352174 | −4.2130884 | 2.52E−05 | 0.00136575 |
| ENSG00000138795 | 53.0506709 | −1.499016 | 0.41420877 | −3.6189867 | 0.00029576 | 0.00957793 |
| ENSG00000138658 | 416.850572 | 0.73760204 | 0.23211495 | 3.17774463 | 0.00148425 | 0.03148474 |
| ENSG00000172403 | 1413.67295 | 0.67954889 | 0.21468925 | 3.16526735 | 0.00154941 | 0.03253097 |
| ENSG00000145390 | 194.320344 | 1.02979448 | 0.23315177 | 4.41684168 | 1.00E−05 | 0.00062948 |
| ENSG00000138735 | 279.385187 | 0.96220143 | 0.2190535 | 4.39254067 | 1.12E−05 | 0.00069641 |
| ENSG00000164109 | 1808.52017 | 0.66402326 | 0.17247327 | 3.85000683 | 0.00011811 | 0.00465977 |
| ENSG00000164111 | 636.93577 | 0.5668139 | 0.1567384 | 3.61630529 | 0.00029884 | 0.00958654 |
| ENSG00000145386 | 1106.26545 | 0.53084141 | 0.12798843 | 4.14757335 | 3.36E−05 | 0.00170725 |
| ENSG00000142731 | 692.62433 | 0.82082697 | 0.18257604 | 4.49580888 | 6.93E−06 | 0.00045586 |
| ENSG00000151012 | 36.0945069 | 1.10363165 | 0.34185458 | 3.22836587 | 0.001245 | 0.02779946 |
| ENSG00000061918 | 397.775029 | 0.68946993 | 0.15172777 | 4.54412474 | 5.52E−06 | 0.00037737 |
| ENSG00000168843 | 250.655119 | −0.6556833 | 0.21126985 | −3.1035346 | 0.00191224 | 0.03777728 |
| ENSG00000245213 | 39.943521 | −1.0656272 | 0.32935042 | −3.2355423 | 0.00121412 | 0.02722932 |
| ENSG00000237125 | 9731.51257 | −0.340198 | 0.10838193 | −3.1388817 | 0.00169594 | 0.03442594 |
| ENSG00000250043 | 135.983413 | −0.6442353 | 0.21281782 | −3.0271681 | 0.00246857 | 0.0460347 |
| ENSG00000251216 | 1141.42786 | −0.7002063 | 0.17631762 | −3.9712782 | 7.15E−05 | 0.00313476 |
| ENSG00000151725 | 1139.09906 | 0.73402981 | 0.17059102 | 4.30286321 | 1.69E−05 | 0.00097321 |
| ENSG00000071539 | 1120.76928 | 0.57103576 | 0.11301158 | 5.05289625 | 4.35E−07 | 4.40E−05 |
| ENSG00000215218 | 1621.85427 | 0.97963414 | 0.12597902 | 7.7761687 | 7.48E−15 | 4.09E−12 |
| ENSG00000112977 | 897.322228 | 0.40825379 | 0.12800561 | 3.18934287 | 0.00142597 | 0.03070895 |
| ENSG00000145569 | 338.413013 | 0.97534294 | 0.18549373 | 5.25809116 | 1.46E−07 | 1.72E−05 |
| ENSG00000250448 | 624.290076 | −0.7689172 | 0.20469589 | −3.7563881 | 0.00017238 | 0.00615862 |
| ENSG00000113407 | 2855.49085 | 0.4632001 | 0.12101262 | 3.82770082 | 0.00012935 | 0.00501141 |
| ENSG00000039560 | 636.953704 | 0.70468761 | 0.17477204 | 4.03203861 | 5.53E−05 | 0.00258501 |
| ENSG00000113594 | 264.760356 | 0.70649651 | 0.23242993 | 3.03961071 | 0.00236884 | 0.04456678 |
| ENSG00000145623 | 38.3616683 | 1.79278978 | 0.35109015 | 5.10635171 | 3.28E−07 | 3.42E−05 |
| ENSG00000112936 | 15556.4126 | −0.8629623 | 0.14314754 | −6.0284815 | 1.66E−09 | 3.16E−07 |
| ENSG00000198865 | 176.825873 | 0.86434955 | 0.21787301 | 3.96721712 | 7.27E−05 | 0.00318042 |
| ENSG00000250722 | 52.3311978 | 0.95223317 | 0.27731171 | 3.43380082 | 0.00059518 | 0.01631701 |
| ENSG00000112972 | 747.663213 | 0.58791546 | 0.14788077 | 3.9756047 | 7.02E−05 | 0.00310236 |
| ENSG00000259663 | 2173.06244 | −0.598649 | 0.11799534 | −5.0734973 | 3.91E−07 | 4.00E−05 |
| ENSG00000016082 | 3574.99136 | −0.5231539 | 0.0957384 | −5.4644097 | 4.64E−08 | 6.36E−06 |
| ENSG00000164171 | 189.689659 | 0.82523777 | 0.23984169 | 3.44076037 | 0.00058008 | 0.01603119 |
| ENSG00000123219 | 483.620787 | 0.84731377 | 0.20441778 | 4.14501013 | 3.40E−05 | 0.00172131 |
| ENSG00000145675 | 912.731472 | 0.53692333 | 0.17309319 | 3.10193222 | 0.00192262 | 0.03780632 |
| ENSG00000131711 | 10876.2162 | 0.70915528 | 0.1279467 | 5.54258374 | 2.98E−08 | 4.29E−06 |
| ENSG00000171617 | 494.843977 | 1.11330999 | 0.13377399 | 8.32232011 | 8.63E−17 | 5.86E−14 |
| ENSG00000145703 | 87.8488735 | 0.96840954 | 0.26321913 | 3.67910009 | 0.00023406 | 0.00802418 |
| ENSG00000145685 | 2265.26613 | 0.50244416 | 0.15922743 | 3.15551263 | 0.00160216 | 0.03311658 |
| ENSG00000113273 | 641.656968 | 0.74340659 | 0.15418606 | 4.82149035 | 1.42E−06 | 0.00011912 |
| ENSG00000228716 | 2220.19717 | 0.92950201 | 0.15061307 | 6.17145629 | 6.77E−10 | 1.40E−07 |
| ENSG00000038427 | 1105.63965 | 0.78685839 | 0.23908825 | 3.29107927 | 0.00099804 | 0.0235232 |
| ENSG00000164176 | 65.9045405 | −0.9555302 | 0.31763536 | −3.0082615 | 0.00262747 | 0.0483224 |
| ENSG00000245526 | 15.2410883 | −1.8257443 | 0.55187875 | −3.3082344 | 0.00093886 | 0.02257111 |
| ENSG00000133302 | 647.474414 | 0.65532392 | 0.19484649 | 3.36328315 | 0.00077021 | 0.01974399 |
| ENSG00000113083 | 135.385085 | 0.76820882 | 0.20116162 | 3.81886371 | 0.00013407 | 0.00518253 |
| ENSG00000113368 | 2767.97899 | 0.41670104 | 0.10187088 | 4.09048221 | 4.30E−05 | 0.00210524 |
| ENSG00000064651 | 425.065248 | 0.55554183 | 0.17431193 | 3.18705566 | 0.00143729 | 0.03075569 |
| ENSG00000113396 | 153.87229 | 1.03578932 | 0.20791022 | 4.98190671 | 6.30E−07 | 6.00E−05 |
| ENSG00000170606 | 4602.42671 | 0.40582978 | 0.12990706 | 3.12400105 | 0.0017841 | 0.03591476 |
| ENSG00000145833 | 2398.48358 | 0.61443327 | 0.17533111 | 3.50441669 | 0.00045761 | 0.01348914 |
| ENSG00000164616 | 78.8328447 | 0.97736989 | 0.25322814 | 3.85964169 | 0.00011355 | 0.00453412 |
| ENSG00000120708 | 14.9320533 | 1.72601215 | 0.51798742 | 3.33215074 | 0.00086178 | 0.02135322 |
| ENSG00000271824 | 35.9891942 | 1.86709886 | 0.39506309 | 4.72607767 | 2.29E−06 | 0.00017737 |
| ENSG00000146013 | 44.6522932 | −1.0234232 | 0.3145265 | −3.2538535 | 0.00113851 | 0.02593357 |
| ENSG00000228672 | 84.3361262 | −0.9913691 | 0.28789458 | −3.4435144 | 0.00057421 | 0.01589605 |
| ENSG00000113070 | 183.892498 | 1.74756525 | 0.19413463 | 9.00182134 | 2.22E−19 | 1.79E−16 |
| ENSG00000113108 | 340.549821 | −0.5874831 | 0.15261191 | −3.8495235 | 0.00011835 | 0.00465977 |
| ENSG00000113657 | 1357.76408 | 0.53353429 | 0.14546137 | 3.66787603 | 0.00024457 | 0.00821865 |
| ENSG00000178776 | 17.25202 | 3.02778106 | 0.59106821 | 5.1225578 | 3.01E−07 | 3.20E−05 |
| ENSG00000113140 | 2152.72916 | 0.97668594 | 0.10437417 | 9.3575443 | 8.16E−21 | 6.92E−18 |
| ENSG00000145907 | 3028.78286 | 0.46537837 | 0.14862232 | 3.1312819 | 0.00174045 | 0.03516124 |
| ENSG00000164574 | 290.331487 | 0.61483797 | 0.18015081 | 3.41290699 | 0.00064274 | 0.01725835 |
| ENSG00000135074 | 340.949329 | 1.05125698 | 0.20795994 | 5.05509377 | 4.30E−07 | 4.37E−05 |
| ENSG00000172548 | 15.9517673 | 2.49560152 | 0.51572627 | 4.83900409 | 1.30E−06 | 0.00011168 |
| ENSG00000164330 | 172.432536 | −0.5925625 | 0.19340796 | −3.0637961 | 0.00218548 | 0.04188258 |
| ENSG00000040275 | 852.643276 | 0.72460802 | 0.17921087 | 4.04332628 | 5.27E−05 | 0.00249034 |
| ENSG00000175309 | 354.770726 | −0.5546946 | 0.16237656 | −3.4160999 | 0.00063525 | 0.0171386 |
| ENSG00000087116 | 228.336828 | 0.84429909 | 0.22424987 | 3.76499255 | 0.00016655 | 0.00602379 |
| ENSG00000233937 | 59.0548755 | −1.0937113 | 0.30947189 | −3.5341218 | 0.00040913 | 0.01242036 |
| ENSG00000168994 | 51.6992626 | 1.07545298 | 0.29515578 | 3.64367924 | 0.00026877 | 0.00892563 |
| ENSG00000270504 | 21.8859549 | 1.90192884 | 0.45115408 | 4.21569687 | 2.49E−05 | 0.00135438 |
| ENSG00000260604 | 17.1382489 | 3.03661646 | 0.61230347 | 4.95933244 | 7.07E−07 | 6.67E−05 |
| ENSG00000111859 | 386.637165 | 1.10821683 | 0.18030992 | 6.1461779 | 7.94E−10 | 1.60E−07 |
| ENSG00000212802 | 145.054541 | −0.8601221 | 0.26424199 | −3.2550545 | 0.00113371 | 0.02585886 |
| ENSG00000007944 | 172.170355 | −0.816473 | 0.18513196 | −4.4102217 | 1.03E−05 | 0.00064664 |
| ENSG00000124795 | 5115.73526 | 0.59970202 | 0.181612 | 3.3021057 | 0.00095962 | 0.02292036 |
| ENSG00000172201 | 2146.62087 | −1.1609992 | 0.14278042 | −8.1313614 | 4.24E−16 | 2.57E−13 |
| ENSG00000152954 | 704.567276 | −0.4963422 | 0.11882772 | −4.1769903 | 2.95E−05 | 0.00153559 |
| ENSG00000274267 | 9.3778659 | −4.6784126 | 1.28537261 | −3.6397326 | 0.00027292 | 0.00901367 |
| ENSG00000180573 | 149.588882 | −0.950396 | 0.21153562 | −4.4928413 | 7.03E−06 | 0.00046048 |
| ENSG00000168298 | 10.3556519 | −3.8835243 | 1.01308601 | −3.8333609 | 0.0001264 | 0.00491465 |
| ENSG00000197409 | 8.29760672 | −2.3814634 | 0.75236633 | −3.1652977 | 0.00154924 | 0.03253097 |
| ENSG00000276966 | 12.0539898 | −3.0078246 | 0.93385109 | −3.2208825 | 0.00127797 | 0.02821894 |
| ENSG00000184357 | 9.70561438 | −3.245701 | 0.89208343 | −3.638338 | 0.0002744 | 0.00904198 |
| ENSG00000137310 | 490.430255 | 0.67154189 | 0.13910739 | 4.82750699 | 1.38E−06 | 0.00011615 |
| ENSG00000168394 | 14.7028695 | 1.69883977 | 0.4953332 | 3.42969093 | 0.00060427 | 0.01648625 |
| ENSG00000096060 | 503.984936 | 0.64915024 | 0.17243646 | 3.76457651 | 0.00016683 | 0.00602379 |
| ENSG00000124762 | 1643.99052 | −0.6182079 | 0.15236331 | −4.0574589 | 4.96E−05 | 0.00237148 |
| ENSG00000124587 | 854.585389 | −0.7647543 | 0.17847478 | −4.2849434 | 1.83E−05 | 0.00103396 |
| ENSG00000044090 | 1232.25664 | −0.5835654 | 0.15134944 | −3.8557487 | 0.00011538 | 0.00459608 |
| ENSG00000008196 | 8624.97581 | −0.5677007 | 0.1011835 | −5.6106051 | 2.02E−08 | 3.05E−06 |
| ENSG00000112118 | 4142.82279 | 0.61736063 | 0.08772204 | 7.03769132 | 1.95E−12 | 7.37E−10 |
| ENSG00000202198 | 566.346759 | −5.42873 | 1.38668627 | −3.9148942 | 9.04E−05 | 0.00377109 |
| ENSG00000112208 | 212.237884 | 0.63245787 | 0.18058213 | 3.50232807 | 0.00046121 | 0.01351771 |
| ENSG00000146143 | 441.454969 | 0.69464481 | 0.16126664 | 4.30743035 | 1.65E−05 | 0.00095659 |
| ENSG00000230910 | 192.275141 | −1.2516479 | 0.19559659 | −6.3991292 | 1.56E−10 | 3.84E−08 |
| ENSG00000135298 | 515.032149 | −0.4877499 | 0.14392386 | −3.3889443 | 0.00070162 | 0.01843117 |
| ENSG00000118407 | 75.4408608 | 1.29493667 | 0.2805343 | 4.61596555 | 3.91E−06 | 0.00028323 |
| ENSG00000065609 | 3020.65834 | −0.4109937 | 0.12357974 | −3.3257369 | 0.00088185 | 0.02158038 |
| ENSG00000203877 | 459.938972 | −0.9668886 | 0.2367462 | −4.0840721 | 4.43E−05 | 0.0021518 |
| ENSG00000112837 | 24.706155 | −2.4756248 | 0.53216642 | −4.6519748 | 3.29E−06 | 0.0002447 |
| ENSG00000135318 | 28.0498738 | 1.37879126 | 0.43583248 | 3.16358079 | 0.00155841 | 0.03257065 |
| ENSG00000146278 | 954.306545 | −0.4230271 | 0.14049196 | −3.0110412 | 0.00260354 | 0.04818102 |
| ENSG00000118412 | 789.235539 | 0.68134511 | 0.19664737 | 3.46480672 | 0.00053061 | 0.0150075 |
| ENSG00000146263 | 618.483005 | 0.70482112 | 0.20389496 | 3.45678532 | 0.00054666 | 0.01534913 |
| ENSG00000184486 | 214.087184 | −0.6929866 | 0.1679675 | −4.1257185 | 3.70E−05 | 0.00183385 |
| ENSG00000135596 | 1851.24624 | −0.5715852 | 0.15144003 | −3.7743335 | 0.00016044 | 0.0059187 |
| ENSG00000111885 | 533.905017 | 0.83007561 | 0.19628127 | 4.22901092 | 2.35E−05 | 0.00128907 |
| ENSG00000118515 | 1176.5287 | 1.06658191 | 0.12037525 | 8.86047505 | 7.97E−19 | 5.88E−16 |
| ENSG00000118513 | 130.754024 | 0.89367122 | 0.21948039 | 4.0717588 | 4.67E−05 | 0.00225587 |
| ENSG00000171408 | 347.278409 | 0.58920353 | 0.16639486 | 3.5409961 | 0.00039862 | 0.0122325 |
| ENSG00000029363 | 4677.37809 | 0.62684582 | 0.18883266 | 3.31958375 | 0.00090152 | 0.02188663 |
| ENSG00000051620 | 46.3131638 | −1.1666442 | 0.32603592 | −3.5782688 | 0.00034588 | 0.01076981 |
| ENSG00000118495 | 364.227032 | −0.6147821 | 0.17755922 | −3.4624061 | 0.00053537 | 0.01509171 |
| ENSG00000120256 | 423.229688 | 0.61393009 | 0.18709056 | 3.28145949 | 0.00103271 | 0.02403999 |
| ENSG00000131016 | 4998.17369 | 0.49594629 | 0.15932063 | 3.11288179 | 0.0018527 | 0.03694519 |
| ENSG00000146469 | 906.476132 | 0.91433745 | 0.2017947 | 4.53102817 | 5.87E−06 | 0.00039216 |
| ENSG00000112029 | 967.627312 | 0.68516835 | 0.14051248 | 4.87620974 | 1.08E−06 | 9.51E−05 |
| ENSG00000078269 | 182.866354 | 1.20445359 | 0.21077641 | 5.71436634 | 1.10E−08 | 1.83E−06 |
| ENSG00000026297 | 176.18014 | −0.8438583 | 0.27418591 | −3.0776866 | 0.00208614 | 0.04037148 |
| ENSG00000164850 | 182.544241 | 0.68801641 | 0.22467811 | 3.06223157 | 0.00219693 | 0.04203154 |
| ENSG00000164638 | 2955.51358 | −0.6439128 | 0.180405 | −3.5692627 | 0.00035799 | 0.01106566 |
| ENSG00000003147 | 2985.59074 | −0.7054629 | 0.12846535 | −5.4914642 | 3.99E−08 | 5.64E−06 |
| ENSG00000006468 | 321.62806 | 0.6443321 | 0.18262218 | 3.52822485 | 0.00041836 | 0.0126777 |
| ENSG00000173452 | 8.76484616 | −2.9967376 | 0.77944109 | −3.8447263 | 0.00012069 | 0.00472993 |
| ENSG00000122566 | 22454.8179 | 0.39083235 | 0.11191375 | 3.49226401 | 0.00047894 | 0.01389349 |
| ENSG00000255690 | 744.770647 | −0.6351112 | 0.1480364 | −4.2902364 | 1.78E−05 | 0.0010164 |
| ENSG00000106066 | 21.0391635 | 1.36076094 | 0.44020713 | 3.0911833 | 0.00199361 | 0.03910311 |
| ENSG00000106105 | 4400.30784 | 0.56415584 | 0.10387851 | 5.43091983 | 5.61E−08 | 7.38E−06 |
| ENSG00000164619 | 74.6718287 | 1.06222536 | 0.26439031 | 4.01764097 | 5.88E−05 | 0.00272101 |
| ENSG00000011426 | 1657.71398 | 0.61812924 | 0.17639289 | 3.50427527 | 0.00045785 | 0.01348914 |
| ENSG00000164543 | 624.32484 | 0.54936894 | 0.17026889 | 3.22647858 | 0.00125324 | 0.02786035 |
| ENSG00000105968 | 4061.78086 | 0.36800262 | 0.11569985 | 3.18066619 | 0.00146937 | 0.0312863 |
| ENSG00000146674 | 5646.90033 | 0.52960291 | 0.09681834 | 5.47006794 | 4.50E−08 | 6.26E−06 |
| ENSG00000136205 | 329.545343 | 0.83618366 | 0.23346419 | 3.58163572 | 0.00034145 | 0.01067947 |
| ENSG00000132436 | 873.350924 | 0.72347195 | 0.17316039 | 4.17804533 | 2.94E−05 | 0.00153526 |
| ENSG00000132437 | 1589.67415 | 0.4126647 | 0.12012991 | 3.43515382 | 0.00059222 | 0.01626203 |
| ENSG00000146648 | 312.531085 | 1.29741016 | 0.27279526 | 4.75598489 | 1.97E−06 | 0.00015883 |
| ENSG00000154978 | 855.14167 | 0.4078751 | 0.11686006 | 3.49028651 | 0.0004825 | 0.01397282 |
| ENSG00000196247 | 382.109054 | 0.8342881 | 0.21754525 | 3.83500952 | 0.00012556 | 0.00490955 |
| ENSG00000234215 | 34.4721496 | −1.241653 | 0.37202257 | −3.3375744 | 0.00084513 | 0.0210512 |
| ENSG00000009954 | 5676.65284 | 0.41775373 | 0.12798151 | 3.26417239 | 0.00109784 | 0.02524445 |
| ENSG00000176428 | 420.83628 | −0.9276468 | 0.25696098 | −3.6100687 | 0.00030612 | 0.00978303 |
| ENSG00000165171 | 51.0651218 | −1.0960503 | 0.31325145 | −3.4989473 | 0.0004671 | 0.01366667 |
| ENSG00000049540 | 1513.88196 | 0.99440863 | 0.19181531 | 5.18419848 | 2.17E−07 | 2.45E−05 |
| ENSG00000223705 | 788.12693 | −0.5429563 | 0.13533087 | −4.0120654 | 6.02E−05 | 0.00273107 |
| ENSG00000153993 | 24.6255783 | 1.87728755 | 0.44157633 | 4.25133188 | 2.13E−05 | 0.00118624 |
| ENSG00000105810 | 1551.90477 | 0.7753039 | 0.19666196 | 3.94231758 | 8.07E−05 | 0.00346695 |
| ENSG00000177409 | 18.8112566 | 1.79692638 | 0.54574956 | 3.29258423 | 0.00099271 | 0.02346284 |
| ENSG00000105825 | 923.984722 | 0.89181469 | 0.14825765 | 6.01530289 | 1.80E−09 | 3.39E−07 |
| ENSG00000164692 | 67.8006859 | 1.26861427 | 0.27486149 | 4.6154674 | 3.92E−06 | 0.00028323 |
| ENSG00000242265 | 7634.37657 | 1.05389115 | 0.1615857 | 6.52218077 | 6.93E−11 | 1.84E−08 |
| ENSG00000158560 | 151.932174 | −1.132983 | 0.22965716 | −4.9333668 | 8.08E−07 | 7.58E−05 |
| ENSG00000105880 | 480.449047 | −0.8820554 | 0.18020173 | −4.8948221 | 9.84E−07 | 8.79E−05 |
| ENSG00000070669 | 3219.23762 | 0.57211473 | 0.13612728 | 4.2027927 | 2.64E−05 | 0.00140692 |
| ENSG00000197093 | 231.972871 | −0.7238876 | 0.2201119 | −3.2887254 | 0.00100642 | 0.02363921 |
| ENSG00000213420 | 3432.12066 | −0.6972303 | 0.19376615 | −3.5983081 | 0.00032029 | 0.01019773 |
| ENSG00000106366 | 241.882814 | 2.04043833 | 0.16960532 | 12.0305087 | 2.46E−33 | 4.63E−30 |
| ENSG00000128606 | 263.914744 | 0.66241835 | 0.19602278 | 3.3792926 | 0.00072673 | 0.0188571 |
| ENSG00000173114 | 1896.30582 | 0.73433287 | 0.19305661 | 3.80371777 | 0.00014254 | 0.00543576 |
| ENSG00000071243 | 331.965595 | 0.6830828 | 0.21747146 | 3.14102268 | 0.00168359 | 0.03425722 |
| ENSG00000106034 | 22.7403345 | 1.61831048 | 0.487037 | 3.32276705 | 0.00089129 | 0.02173789 |
| ENSG00000273329 | 251.465269 | −0.5965393 | 0.16369226 | −3.6442733 | 0.00026815 | 0.0089225 |
| ENSG00000128510 | 95.6419874 | 2.99571874 | 0.24078853 | 12.4412853 | 1.56E−35 | 3.31E−32 |
| ENSG00000106484 | 95.2173734 | 1.95404839 | 0.27009775 | 7.23459714 | 4.67E−13 | 1.98E−10 |
| ENSG00000122786 | 3855.50149 | 0.99196911 | 0.13260731 | 7.48050114 | 7.40E−14 | 3.31E−11 |
| ENSG00000105894 | 646.919988 | 0.8462744 | 0.18497236 | 4.57513987 | 4.76E−06 | 0.00033511 |
| ENSG00000106462 | 1871.50657 | 0.45617502 | 0.11168784 | 4.08437494 | 4.42E−05 | 0.0021518 |
| ENSG00000197558 | 43.5577965 | −1.1107939 | 0.35093877 | −3.1652072 | 0.00154973 | 0.03253097 |
| ENSG00000127399 | 176.074863 | 1.04849452 | 0.24407105 | 4.29585773 | 1.74E−05 | 0.00099464 |
| ENSG00000188707 | 63.1050841 | 1.46864612 | 0.27178181 | 5.40376908 | 6.53E−08 | 8.31E−06 |
| ENSG00000187260 | 1073.16574 | −0.8402094 | 0.24824805 | −3.3845558 | 0.00071294 | 0.01867054 |
| ENSG00000239911 | 326.037054 | −1.1413048 | 0.25539642 | −4.4687583 | 7.87E−06 | 0.00050959 |
| ENSG00000261455 | 407.047706 | −0.7262598 | 0.21263814 | −3.4154729 | 0.00063671 | 0.01715084 |
| ENSG00000196584 | 1119.30822 | 0.85371094 | 0.19743546 | 4.32400012 | 1.53E−05 | 0.00090477 |
| ENSG00000273344 | 270.005613 | −0.7543578 | 0.19979063 | −3.7757418 | 0.00015953 | 0.00591105 |
| ENSG00000146918 | 1797.81344 | 0.65980332 | 0.12824946 | 5.14468697 | 2.68E−07 | 2.95E−05 |
| ENSG00000101846 | 209.594902 | 0.98179148 | 0.24060972 | 4.08043154 | 4.50E−05 | 0.00217954 |
| ENSG00000101871 | 268.327857 | 0.94321939 | 0.1989142 | 4.7418405 | 2.12E−06 | 0.00016794 |
| ENSG00000205542 | 2200.4592 | 0.68096974 | 0.21952636 | 3.10199535 | 0.00192221 | 0.03780632 |
| ENSG00000181544 | 78.7732903 | 1.20982076 | 0.26798717 | 4.51447261 | 6.35E−06 | 0.00042242 |
| ENSG00000184368 | 405.584229 | 0.70312707 | 0.14279045 | 4.92418836 | 8.47E−07 | 7.81E−05 |
| ENSG00000101868 | 972.284706 | 0.72296358 | 0.15636506 | 4.62356208 | 3.77E−06 | 0.00027646 |
| ENSG00000069535 | 388.606137 | 1.09689742 | 0.16748227 | 6.54933446 | 5.78E−11 | 1.58E−08 |
| ENSG00000102317 | 1214.26155 | 0.55719701 | 0.1479488 | 3.76614753 | 0.00016579 | 0.00602379 |
| ENSG00000274588 | 74.3315493 | −1.4313745 | 0.30733307 | −4.6574045 | 3.20E−06 | 0.00024045 |
| ENSG00000184194 | 745.256551 | −0.5944957 | 0.13828966 | −4.2989164 | 1.72E−05 | 0.00098734 |
| ENSG00000072501 | 7025.99821 | 0.64654519 | 0.18278208 | 3.53724615 | 0.00040432 | 0.01234057 |
| ENSG00000090889 | 1603.39257 | 0.51207941 | 0.10822244 | 4.73173047 | 2.23E−06 | 0.0001749 |
| ENSG00000186871 | 426.577887 | 0.88104685 | 0.18226944 | 4.83376064 | 1.34E−06 | 0.00011368 |
| ENSG00000102384 | 280.699886 | 0.82897478 | 0.20652448 | 4.01392979 | 5.97E−05 | 0.00273107 |
| ENSG00000147224 | 897.070383 | 0.47314214 | 0.13595978 | 3.48001542 | 0.00050138 | 0.01437247 |
| ENSG00000101888 | 592.095764 | 0.79925656 | 0.21049201 | 3.79708741 | 0.00014641 | 0.00552114 |
| ENSG00000102024 | 803.268593 | 0.67318667 | 0.16949921 | 3.97162133 | 7.14E−05 | 0.00313476 |
| ENSG00000147251 | 532.353302 | 0.61030171 | 0.18226556 | 3.34842024 | 0.00081274 | 0.02046312 |
| ENSG00000131724 | 269.819839 | 0.90722718 | 0.1781497 | 5.09249902 | 3.53E−07 | 3.66E−05 |
| ENSG00000101972 | 2298.27995 | 0.5901637 | 0.19651015 | 3.00322246 | 0.00267137 | 0.04900882 |
| ENSG00000009694 | 30.321651 | −2.9459339 | 0.51420602 | −5.7290926 | 1.01E−08 | 1.70E−06 |
| ENSG00000147256 | 488.353731 | −2.50365 | 0.16626093 | −15.058559 | 3.03E−51 | 8.58E−48 |
| ENSG00000171004 | 280.445427 | −0.7022365 | 0.18134784 | −3.8723181 | 0.00010781 | 0.0043352 |
| ENSG00000076716 | 44.8994688 | 1.590063 | 0.31570321 | 5.03657536 | 4.74E−07 | 4.65E−05 |
| ENSG00000147257 | 273.936329 | 0.53917998 | 0.16829994 | 3.20368492 | 0.00135681 | 0.02963326 |
| ENSG00000223749 | 16.7834652 | 2.07979932 | 0.51110333 | 4.06923453 | 4.72E−05 | 0.00227397 |
| ENSG00000184785 | 22.31125 | 1.67368911 | 0.50539332 | 3.31165656 | 0.00092745 | 0.02235637 |
| ENSG00000178947 | 132.980602 | −0.7032732 | 0.21134322 | −3.3276355 | 0.00087586 | 0.02154117 |
| ENSG00000022267 | 2339.81849 | 0.42850026 | 0.10908526 | 3.92812246 | 8.56E−05 | 0.00359611 |
| ENSG00000155495 | 251.826393 | 1.0127693 | 0.19169083 | 5.28334781 | 1.27E−07 | 1.52E−05 |
| ENSG00000029993 | 1815.22451 | 0.46856137 | 0.14875959 | 3.14978941 | 0.00163388 | 0.03349066 |
| ENSG00000124260 | 79.3122591 | 1.47291768 | 0.28798512 | 5.1145618 | 3.14E−07 | 3.29E−05 |
| ENSG00000154319 | 3307.72056 | 0.56820397 | 0.15658754 | 3.62866666 | 0.00028489 | 0.00929465 |
| ENSG00000036565 | 326.703684 | 0.56642818 | 0.1458492 | 3.88365639 | 0.0001029 | 0.00417743 |
| ENSG00000168490 | 90.6265738 | −0.8686863 | 0.25527223 | −3.4029803 | 0.00066655 | 0.01781318 |
| ENSG00000134013 | 232.211985 | 0.97218699 | 0.18490693 | 5.25770986 | 1.46E−07 | 1.72E−05 |
| ENSG00000104722 | 2094.48775 | 0.80175824 | 0.16742192 | 4.78884872 | 1.68E−06 | 0.00013778 |
| ENSG00000184661 | 445.957135 | 0.51644238 | 0.15797878 | 3.26906159 | 0.00107905 | 0.02491353 |
| ENSG00000120885 | 664.325743 | 0.73718524 | 0.17535651 | 4.20392278 | 2.62E−05 | 0.00140433 |
| ENSG00000168077 | 207.62523 | 1.0665369 | 0.22597803 | 4.71964853 | 2.36E−06 | 0.00018059 |
| ENSG00000171320 | 398.674485 | 1.10919835 | 0.18457831 | 6.00936446 | 1.86E−09 | 3.44E−07 |
| ENSG00000120875 | 2837.84888 | −0.7694555 | 0.1177232 | −6.5361415 | 6.31E−11 | 1.70E−08 |
| ENSG00000133863 | 583.588964 | 1.0329462 | 0.24688535 | 4.18391042 | 2.87E−05 | 0.00150775 |
| ENSG00000147526 | 195.14228 | 0.59827 | 0.18010926 | 3.32170591 | 0.00089469 | 0.02178318 |
| ENSG00000168615 | 488.055742 | 0.95776965 | 0.1708123 | 5.6071467 | 2.06E−08 | 3.09E−06 |
| ENSG00000176907 | 23.7310903 | 2.86636447 | 0.55375574 | 5.17622526 | 2.26E−07 | 2.53E−05 |
| ENSG00000104332 | 1239.06173 | 0.87612871 | 0.11416659 | 7.67412515 | 1.67E−14 | 8.31E−12 |
| ENSG00000147536 | 200.362456 | 0.68489844 | 0.17020893 | 4.02386895 | 5.72E−05 | 0.00266904 |
| ENSG00000104368 | 135.935599 | 0.80137304 | 0.23128034 | 3.46494229 | 0.00053035 | 0.0150075 |
| ENSG00000104738 | 3569.65926 | 0.72321488 | 0.09313343 | 7.76536268 | 8.14E−15 | 4.32E−12 |
| ENSG00000137563 | 599.174708 | 0.61216277 | 0.1989057 | 3.07765324 | 0.00208638 | 0.04037148 |
| ENSG00000185697 | 300.167738 | 0.5589311 | 0.18233648 | 3.06538283 | 0.00217392 | 0.04184601 |
| ENSG00000121039 | 203.999663 | 2.74345992 | 0.17120838 | 16.024098 | 8.67E−58 | 3.68E−54 |
| ENSG00000123119 | 165.257532 | 1.08948765 | 0.23048186 | 4.72699959 | 2.28E−06 | 0.00017737 |
| ENSG00000175305 | 101.00205 | 0.83383904 | 0.23217511 | 3.59142306 | 0.00032888 | 0.01039301 |
| ENSG00000156466 | 37.2041576 | 1.12528161 | 0.3267569 | 3.44378838 | 0.00057362 | 0.01589605 |
| ENSG00000132561 | 221.289444 | 1.79517367 | 0.20126459 | 8.91947112 | 4.69E−19 | 3.61E−16 |
| ENSG00000253948 | 28.2858716 | −1.7762482 | 0.42528557 | −4.1766012 | 2.96E−05 | 0.00153559 |
| ENSG00000136960 | 2294.842 | −1.3975054 | 0.16211067 | −8.620687 | 6.66E−18 | 4.71E−15 |
| ENSG00000136982 | 451.384204 | 0.6281883 | 0.18030473 | 3.4840368 | 0.00049391 | 0.01423036 |
| ENSG00000170961 | 58.1172128 | 1.73677692 | 0.30036462 | 5.78222862 | 7.37E−09 | 1.28E−06 |
| ENSG00000156802 | 1592.79788 | 0.88074336 | 0.16182896 | 5.44243344 | 5.26E−08 | 7.08E−06 |
| ENSG00000173334 | 349.28764 | −0.7231585 | 0.15828925 | −4.5685891 | 4.91E−06 | 0.00034432 |
| ENSG00000136997 | 2309.85625 | −0.835458 | 0.15816169 | −5.282303 | 1.28E−07 | 1.52E−05 |
| ENSG00000155897 | 100.520548 | −0.7602105 | 0.24392276 | −3.1166034 | 0.00182947 | 0.0366543 |
| ENSG00000104419 | 467.856785 | −0.4825066 | 0.15805732 | −3.0527314 | 0.00226769 | 0.04308497 |
| ENSG00000008513 | 95.9529264 | 0.77034338 | 0.22571235 | 3.41294298 | 0.00064265 | 0.01725835 |
| ENSG00000198576 | 196.625325 | −0.8722778 | 0.24202 | −3.6041559 | 0.00031317 | 0.00998962 |
| ENSG00000253716 | 152.861664 | −0.9108288 | 0.25861338 | −3.5219708 | 0.00042835 | 0.01288851 |
| ENSG00000181085 | 181.574075 | −0.9107819 | 0.23165518 | −3.9316277 | 8.44E−05 | 0.00357267 |
| ENSG00000261150 | 13.3959835 | 2.2901365 | 0.64037075 | 3.57626658 | 0.00034854 | 0.01083081 |
| ENSG00000255182 | 17.9517938 | −1.6992865 | 0.52886321 | −3.2130927 | 0.00131314 | 0.02886525 |
| ENSG00000107249 | 27.2717019 | 1.14949393 | 0.38144385 | 3.01353375 | 0.00258224 | 0.04785135 |
| ENSG00000120217 | 55.4053328 | 2.20237577 | 0.32874902 | 6.6992619 | 2.09E−11 | 6.37E−09 |
| ENSG00000147872 | 567.234227 | 0.53489555 | 0.14011276 | 3.81760756 | 0.00013475 | 0.00518934 |
| ENSG00000086062 | 279.975529 | 0.80249569 | 0.1700155 | 4.72013249 | 2.36E−06 | 0.00018059 |
| ENSG00000186638 | 233.617423 | 0.7165172 | 0.22493743 | 3.18540666 | 0.00144551 | 0.03081691 |
| ENSG00000165304 | 1044.48316 | 0.66080479 | 0.15959908 | 4.14040482 | 3.47E−05 | 0.00174065 |
| ENSG00000198963 | 2500.95921 | 0.72465596 | 0.1782632 | 4.06508997 | 4.80E−05 | 0.00230169 |
| ENSG00000148019 | 1360.06368 | 0.46078565 | 0.13726302 | 3.35695411 | 0.00078806 | 0.01999015 |
| ENSG00000135069 | 1673.25537 | 0.5874367 | 0.13546809 | 4.33634746 | 1.45E−05 | 0.00086261 |
| ENSG00000178966 | 560.6875 | 0.65072342 | 0.1947175 | 3.34188464 | 0.00083212 | 0.02085822 |
| ENSG00000213694 | 440.652299 | 0.99551576 | 0.15047439 | 6.61584847 | 3.69E−11 | 1.06E−08 |
| ENSG00000165244 | 531.821263 | 0.74323257 | 0.13045289 | 5.69732523 | 1.22E−08 | 1.97E−06 |
| ENSG00000136943 | 209.087589 | 0.79658957 | 0.1767536 | 4.50677984 | 6.58E−06 | 0.00043631 |
| ENSG00000136824 | 1711.42671 | 0.90336662 | 0.18671195 | 4.83829038 | 1.31E−06 | 0.00011168 |
| ENSG00000148219 | 544.968063 | 0.54553289 | 0.15597096 | 3.49765685 | 0.00046936 | 0.01370932 |
| ENSG00000056558 | 18.8684889 | 1.90767834 | 0.44893014 | 4.249388 | 2.14E−05 | 0.00119266 |
| ENSG00000148175 | 73.0443308 | 1.18550519 | 0.22860321 | 5.18586409 | 2.15E−07 | 2.45E−05 |
| ENSG00000185585 | 25.3231372 | 1.47035811 | 0.41925703 | 3.50705652 | 0.00045309 | 0.01346583 |
| ENSG00000171097 | 375.216523 | −0.4894798 | 0.15888624 | −3.0806937 | 0.00206519 | 0.04014463 |
| ENSG00000130635 | 1058.21838 | 1.49230494 | 0.23742286 | 6.28543064 | 3.27E−10 | 7.21E−08 |
| ENSG00000187609 | 244.271925 | −1.1414752 | 0.24533829 | −4.6526582 | 3.28E−06 | 0.0002447 |
| ENSG00000069696 | 39.1422222 | −1.2604635 | 0.40473378 | −3.1143027 | 0.0018438 | 0.03685085 |
| ENSG00000177106 | 26.6302263 | −1.4952835 | 0.41630368 | −3.5918095 | 0.00032839 | 0.01039301 |
| ENSG00000255284 | 55.6755143 | −1.1129422 | 0.29503007 | −3.772301 | 0.00016175 | 0.0059413 |
| ENSG00000226416 | 70.7908207 | −1.2563021 | 0.34488502 | −3.6426695 | 0.00026983 | 0.00894323 |
| ENSG00000232987 | 12.1257601 | −2.3079323 | 0.65127788 | −3.5436983 | 0.00039456 | 0.01212975 |
| ENSG00000130600 | 2278.61782 | −0.7296088 | 0.18331531 | −3.9800757 | 6.89E−05 | 0.00306052 |
| ENSG00000167244 | 350.661076 | −2.2959962 | 0.24171072 | −9.4989423 | 2.12E−21 | 1.89E−18 |
| ENSG00000110628 | 109.044391 | −0.8385433 | 0.25726993 | −3.259391 | 0.00111652 | 0.02556989 |
| ENSG00000167325 | 2385.72357 | 0.570695 | 0.11954127 | 4.77404144 | 1.81E−06 | 0.00014732 |
| ENSG00000132256 | 29.9547956 | 1.2999292 | 0.39531055 | 3.28837464 | 0.00100768 | 0.02363921 |
| ENSG00000166483 | 1590.30457 | 0.49529361 | 0.11734922 | 4.2206807 | 2.44E−05 | 0.00133333 |
| ENSG00000133816 | 97.7105008 | 2.5512945 | 0.24645738 | 10.351869 | 4.10E−25 | 4.64E−22 |
| ENSG00000197702 | 102.892164 | 0.86154598 | 0.21918437 | 3.93069072 | 8.47E−05 | 0.00357561 |
| ENSG00000133794 | 724.030193 | 0.47810959 | 0.14372375 | 3.32658718 | 0.00087917 | 0.02156661 |
| ENSG00000187486 | 34.7064874 | −1.2910611 | 0.36827161 | −3.5057309 | 0.00045536 | 0.01348914 |
| ENSG00000270607 | 236.454747 | −0.6504999 | 0.19786744 | −3.2875541 | 0.00101062 | 0.02365542 |
| ENSG00000198168 | 378.520914 | 0.56906986 | 0.18001719 | 3.16119725 | 0.00157122 | 0.03279657 |
| ENSG00000066382 | 443.273746 | −0.9098965 | 0.14268592 | −6.3769187 | 1.81E−10 | 4.26E−08 |
| ENSG00000085063 | 1156.79497 | 0.51309656 | 0.16076918 | 3.19151073 | 0.00141531 | 0.03059591 |
| ENSG00000166016 | 182.792179 | −0.6836295 | 0.21937883 | −3.1162057 | 0.00183194 | 0.0366605 |
| ENSG00000026508 | 193.463753 | 1.52944934 | 0.19304153 | 7.92290325 | 2.32E−15 | 1.31E−12 |
| ENSG00000175097 | 8.72479401 | −5.6233641 | 1.61234436 | −3.4876942 | 0.0004872 | 0.01408495 |
| ENSG00000148948 | 9.16297642 | −2.0727224 | 0.67237979 | −3.0826661 | 0.00205155 | 0.03997111 |
| ENSG00000134574 | 534.744597 | −0.4677698 | 0.13837009 | −3.3805702 | 0.00072336 | 0.01885615 |
| ENSG00000255433 | 297.519497 | 1.35862129 | 0.24057537 | 5.64738314 | 1.63E−08 | 2.54E−06 |
| ENSG00000189057 | 293.11634 | 1.41291786 | 0.22732708 | 6.21535225 | 5.12E−10 | 1.11E−07 |
| ENSG00000168496 | 1992.49991 | 0.77350092 | 0.15730674 | 4.91715067 | 8.78E−07 | 8.06E−05 |
| ENSG00000124942 | 6938.49386 | 0.92811072 | 0.27628532 | 3.35924739 | 0.00078155 | 0.01988443 |
| ENSG00000068831 | 158.527156 | −0.778033 | 0.25738259 | −3.0228657 | 0.00250393 | 0.04664298 |
| ENSG00000146670 | 1501.52517 | 0.53649945 | 0.13296625 | 4.0348544 | 5.46E−05 | 0.00256835 |
| ENSG00000014138 | 754.940952 | 0.65860349 | 0.12579213 | 5.23564947 | 1.64E−07 | 1.91E−05 |
| ENSG00000172803 | 88.0177775 | −1.2306521 | 0.25246691 | −4.8745084 | 1.09E−06 | 9.54E−05 |
| ENSG00000179292 | 317.832848 | −0.6826633 | 0.22439999 | −3.0421718 | 0.00234878 | 0.04428752 |
| ENSG00000006534 | 36.0429775 | −1.2438689 | 0.38989092 | −3.1902998 | 0.00142125 | 0.03064633 |
| ENSG00000132749 | 187.790801 | −0.7183159 | 0.18432572 | −3.8969921 | 9.74E−05 | 0.00397305 |
| ENSG00000110092 | 10822.9374 | 0.47270897 | 0.14974407 | 3.15677924 | 0.00159522 | 0.03307749 |
| ENSG00000162344 | 8.22492835 | −2.6982592 | 0.79504262 | −3.3938547 | 0.00068916 | 0.01824312 |
| ENSG00000171631 | 12.5499529 | 2.66453887 | 0.68806167 | 3.87252912 | 0.00010771 | 0.0043352 |
| ENSG00000165490 | 526.032174 | 1.05333618 | 0.21210189 | 4.96618017 | 6.83E−07 | 6.47E−05 |
| ENSG00000151376 | 43.6941144 | 1.2246468 | 0.31406476 | 3.89934487 | 9.65E−05 | 0.00394413 |
| ENSG00000150687 | 306.524588 | 2.51389875 | 0.1493168 | 16.8360076 | 1.33E−63 | 2.26E−59 |
| ENSG00000174804 | 94.7298601 | 1.07924183 | 0.23627807 | 4.56767669 | 4.93E−06 | 0.0003444 |
| ENSG00000149218 | 134.294609 | 0.88668164 | 0.19511765 | 4.54434359 | 5.51E−06 | 0.00037737 |
| ENSG00000184384 | 73.7064213 | 1.21602457 | 0.27359012 | 4.44469471 | 8.80E−06 | 0.00055733 |
| ENSG00000137727 | 53.981437 | −1.2451976 | 0.31147278 | −3.9977734 | 6.39E−05 | 0.00287058 |
| ENSG00000204381 | 351.076377 | 0.95007794 | 0.15079029 | 6.30065732 | 2.96E−10 | 6.71E−08 |
| ENSG00000109846 | 34.7345882 | 1.75089487 | 0.37913543 | 4.61812512 | 3.87E−06 | 0.00028202 |
| ENSG00000250303 | 35.8326941 | 1.27998522 | 0.36295248 | 3.52659173 | 0.00042095 | 0.01273341 |
| ENSG00000149591 | 378.668326 | 2.36498973 | 0.14642678 | 16.1513466 | 1.11E−58 | 6.29E−55 |
| ENSG00000184232 | 228.593209 | −0.8191808 | 0.22312914 | −3.6713303 | 0.00024129 | 0.0081568 |
| ENSG00000149403 | 112.702881 | −1.6899822 | 0.2594754 | −6.5130728 | 7.36E−11 | 1.92E−08 |
| ENSG00000154146 | 207.452709 | 0.89768879 | 0.27745922 | 3.23539004 | 0.00121477 | 0.02722932 |
| ENSG00000149548 | 165.127765 | 0.87042815 | 0.21153252 | 4.1148668 | 3.87E−05 | 0.00191669 |
| ENSG00000149554 | 904.593061 | 0.71108612 | 0.15041951 | 4.7273529 | 2.27E−06 | 0.00017737 |
| ENSG00000067082 | 427.66281 | 0.55352082 | 0.14120436 | 3.91999801 | 8.85E−05 | 0.0037012 |
| ENSG00000173848 | 730.843201 | 0.5993366 | 0.13475051 | 4.44775032 | 8.68E−06 | 0.00055359 |
| ENSG00000065328 | 1189.77449 | 0.89559939 | 0.13056965 | 6.85916996 | 6.93E−12 | 2.40E−09 |
| ENSG00000026025 | 1891.26677 | 0.68867838 | 0.19477511 | 3.53576169 | 0.0004066 | 0.01238784 |
| ENSG00000120594 | 201.133733 | 0.97010269 | 0.25412535 | 3.817418 | 0.00013486 | 0.00518934 |
| ENSG00000078114 | 735.314879 | −0.9071396 | 0.19759563 | −4.5908888 | 4.41E−06 | 0.00031603 |
| ENSG00000204682 | 261.920637 | −0.8106903 | 0.22347255 | −3.6276954 | 0.00028596 | 0.00929651 |
| ENSG00000099256 | 101.234018 | 0.81229758 | 0.23936164 | 3.39359972 | 0.0006898 | 0.01824312 |
| ENSG00000231976 | 72.5228575 | −1.0478135 | 0.29302633 | −3.5758339 | 0.00034911 | 0.01083081 |
| ENSG00000120539 | 841.50772 | 0.70043998 | 0.16881402 | 4.14918143 | 3.34E−05 | 0.0017004 |
| ENSG00000099250 | 160.354994 | 1.6241131 | 0.20482304 | 7.92934781 | 2.20E−15 | 1.29E−12 |
| ENSG00000107562 | 44.0614137 | 2.05072694 | 0.32132329 | 6.38212979 | 1.75E−10 | 4.17E−08 |
| ENSG00000197444 | 1277.77564 | −0.4322421 | 0.13790465 | −3.134355 | 0.00172232 | 0.03483651 |
| ENSG00000107984 | 1076.45706 | 0.51630491 | 0.16720393 | 3.0878755 | 0.00201593 | 0.03945827 |
| ENSG00000122952 | 740.142453 | 0.71617495 | 0.13445235 | 5.32660781 | 1.00E−07 | 1.23E−05 |
| ENSG00000165443 | 1913.70516 | 0.40562016 | 0.13123026 | 3.09090424 | 0.00199548 | 0.03910311 |
| ENSG00000182010 | 595.322064 | 0.9244915 | 0.19227852 | 4.80808524 | 1.52E−06 | 0.00012676 |
| ENSG00000138346 | 590.503348 | 0.83712257 | 0.19747747 | 4.23907883 | 2.24E−05 | 0.0012366 |
| ENSG00000165655 | 1191.04131 | −0.7389305 | 0.23519288 | −3.1418064 | 0.00167909 | 0.03420668 |
| ENSG00000156113 | 609.391281 | −0.8499586 | 0.16830144 | −5.0502157 | 4.41E−07 | 4.42E−05 |
| ENSG00000198682 | 173.849685 | 0.74831536 | 0.20847095 | 3.58954268 | 0.00033126 | 0.01044881 |
| ENSG00000227268 | 258.014062 | −0.6182314 | 0.16851555 | −3.6686905 | 0.0002438 | 0.00820877 |
| ENSG00000107796 | 145.346625 | 2.23125607 | 0.20692458 | 10.7829437 | 4.14E−27 | 5.41E−24 |
| ENSG00000148677 | 89.7163902 | 2.63502198 | 0.26648284 | 9.88814899 | 4.69E−23 | 4.42E−20 |
| ENSG00000138160 | 1800.09155 | 0.7961207 | 0.18225619 | 4.36814071 | 1.25E−05 | 0.00076492 |
| ENSG00000138119 | 38.6238282 | 2.12875269 | 0.39451724 | 5.39584196 | 6.82E−08 | 8.51E−06 |
| ENSG00000119969 | 623.015488 | 0.64566398 | 0.20802466 | 3.10378583 | 0.00191062 | 0.03777728 |
| ENSG00000107438 | 139.939758 | 0.73702742 | 0.20084637 | 3.66960779 | 0.00024292 | 0.00819563 |
| ENSG00000236552 | 1346.47352 | −0.7615224 | 0.25407153 | −2.9972757 | 0.00272404 | 0.04986004 |
| ENSG00000235823 | 210.386201 | −0.6479344 | 0.18687442 | −3.4672185 | 0.00052587 | 0.01492322 |
| ENSG00000166169 | 365.208985 | −0.5657283 | 0.15098301 | −3.7469666 | 0.00017899 | 0.00634111 |
| ENSG00000156374 | 250.990399 | 0.53742495 | 0.17128181 | 3.13766502 | 0.00170299 | 0.03452784 |
| ENSG00000065613 | 797.764833 | 0.54324059 | 0.17018094 | 3.19213529 | 0.00141225 | 0.03056876 |
| ENSG00000108055 | 3311.41942 | 0.54986911 | 0.18102134 | 3.03759282 | 0.00238476 | 0.04467869 |
| ENSG00000198825 | 1396.03695 | 0.89811178 | 0.1516897 | 5.92071702 | 3.21E−09 | 5.79E−07 |
| ENSG00000166033 | 392.540148 | 1.10953338 | 0.16563517 | 6.69865815 | 2.10E−11 | 6.37E−09 |
| ENSG00000227076 | 11.6114077 | 2.51738633 | 0.7390651 | 3.40617669 | 0.0006588 | 0.01763368 |
| ENSG00000188385 | 129.617311 | −0.6639573 | 0.20142752 | −3.2962589 | 0.00097982 | 0.02331927 |
| ENSG00000171798 | 80.4366859 | −0.9174844 | 0.27045002 | −3.392436 | 0.00069274 | 0.01828277 |
| ENSG00000165828 | 27.9524411 | −1.268981 | 0.39801629 | −3.1882639 | 0.0014313 | 0.03071888 |
| ENSG00000111206 | 1324.98409 | 0.65044413 | 0.20089621 | 3.23771226 | 0.00120492 | 0.02719088 |
| ENSG00000130038 | 9.33931173 | 2.20797123 | 0.63985758 | 3.45072295 | 0.00055909 | 0.01560479 |
| ENSG00000111247 | 587.447978 | 0.63058447 | 0.19369468 | 3.25555915 | 0.00113169 | 0.02584769 |
| ENSG00000111653 | 1007.32674 | −0.4910197 | 0.14682352 | −3.3442853 | 0.00082495 | 0.02070913 |
| ENSG00000139182 | 1013.40793 | −0.6151654 | 0.16205506 | −3.7960271 | 0.00014703 | 0.0055325 |
| ENSG00000111341 | 52.5591407 | 1.48716829 | 0.32435927 | 4.5849416 | 4.54E−06 | 0.00032379 |
| ENSG00000172572 | 84.4248088 | −1.0453147 | 0.31100227 | −3.3611158 | 0.00077628 | 0.01983963 |
| ENSG00000004700 | 471.471902 | 0.50703802 | 0.1567036 | 3.23565004 | 0.00121366 | 0.02722932 |
| ENSG00000121361 | 164.493102 | −0.58957 | 0.18772776 | −3.1405583 | 0.00168626 | 0.03427049 |
| ENSG00000111728 | 53.8112263 | −1.158527 | 0.30769296 | −3.7652048 | 0.00016641 | 0.00602379 |
| ENSG00000060982 | 2072.64907 | 0.72426246 | 0.17406251 | 4.1609331 | 3.17E−05 | 0.00162497 |
| ENSG00000211455 | 966.70334 | 0.61111384 | 0.19881597 | 3.07376639 | 0.00211375 | 0.0408081 |
| ENSG00000029153 | 93.6746801 | 1.3096821 | 0.24957145 | 5.247724 | 1.54E−07 | 1.80E−05 |
| ENSG00000064763 | 95.9401665 | 1.82435552 | 0.26199656 | 6.96328051 | 3.32E−12 | 1.20E−09 |
| ENSG00000151233 | 674.323745 | 0.63064482 | 0.18159734 | 3.47276472 | 0.00051513 | 0.01471667 |
| ENSG00000173157 | 39.5357031 | 1.24455093 | 0.36093308 | 3.44814868 | 0.00056444 | 0.01572841 |
| ENSG00000139636 | 551.366813 | −0.5428625 | 0.13516544 | −4.0162816 | 5.91E−05 | 0.00272177 |
| ENSG00000161800 | 1838.65717 | 0.46280158 | 0.12176137 | 3.80089016 | 0.00014418 | 0.00546431 |
| ENSG00000050426 | 977.905555 | −0.4243176 | 0.11671552 | −3.6354855 | 0.00027746 | 0.00911133 |
| ENSG00000139629 | 199.645897 | 1.12130893 | 0.17312966 | 6.47670049 | 9.38E−11 | 2.37E−08 |
| ENSG00000050438 | 1376.0608 | 0.67381185 | 0.20303589 | 3.31868343 | 0.00090443 | 0.02192594 |
| ENSG00000161835 | 85.5576125 | −1.0521646 | 0.3113539 | −3.3793205 | 0.00072665 | 0.0188571 |
| ENSG00000111057 | 42.6287927 | 2.65409095 | 0.35083773 | 7.56501 | 3.88E−14 | 1.88E−11 |
| ENSG00000012822 | 1008.26603 | −0.5823158 | 0.16207507 | −3.5928768 | 0.00032705 | 0.01037382 |
| ENSG00000161638 | 36.0053856 | 1.57920114 | 0.33431937 | 4.72363047 | 2.32E−06 | 0.0001787 |
| ENSG00000170627 | 86.8767792 | 1.19928009 | 0.27482521 | 4.36379207 | 1.28E−05 | 0.00077472 |
| ENSG00000182796 | 250.103145 | −0.7497622 | 0.19059624 | −3.933772 | 8.36E−05 | 0.0035566 |
| ENSG00000111602 | 2330.86351 | 0.54470319 | 0.11886176 | 4.58266125 | 4.59E−06 | 0.00032598 |
| ENSG00000174099 | 31.3441604 | −1.6514605 | 0.44201165 | −3.7362376 | 0.00018679 | 0.00659022 |
| ENSG00000127324 | 140.764352 | −0.9219979 | 0.26184428 | −3.5211689 | 0.00042965 | 0.01289831 |
| ENSG00000139278 | 37.1707977 | 1.36219565 | 0.3940954 | 3.45651241 | 0.00054721 | 0.01534913 |
| ENSG00000165891 | 191.42975 | 0.85454995 | 0.21083114 | 4.05324346 | 5.05E−05 | 0.00240785 |
| ENSG00000070961 | 861.520988 | 0.64626753 | 0.18335396 | 3.52469898 | 0.00042396 | 0.01280191 |
| ENSG00000011465 | 1260.14348 | −0.9417459 | 0.16164969 | −5.8258439 | 5.68E−09 | 1.00E−06 |
| ENSG00000198431 | 3689.05075 | 0.53310322 | 0.12966139 | 4.11150317 | 3.93E−05 | 0.00193918 |
| ENSG00000136010 | 285.746265 | 0.75536488 | 0.21856313 | 3.45604899 | 0.00054816 | 0.01535017 |
| ENSG00000074590 | 126.923559 | 1.14469769 | 0.22270228 | 5.14003578 | 2.75E−07 | 3.01E−05 |
| ENSG00000151136 | 253.165693 | −1.0094735 | 0.18207407 | −5.5443011 | 2.95E−08 | 4.28E−06 |
| ENSG00000111249 | 2138.04096 | 0.58952871 | 0.17385785 | 3.39086619 | 0.00069672 | 0.01833079 |
| ENSG00000111271 | 469.367801 | −0.4929724 | 0.14237378 | −3.4625222 | 0.00053514 | 0.01509171 |
| ENSG00000135111 | 8429.45307 | 0.43799149 | 0.14079473 | 3.1108515 | 0.00186549 | 0.03711292 |
| ENSG00000111445 | 523.0625 | 0.54048876 | 0.1481737 | 3.64767004 | 0.00026463 | 0.00882271 |
| ENSG00000139725 | 32.6110633 | −1.3768146 | 0.43060523 | −3.197394 | 0.00138675 | 0.03009362 |
| ENSG00000212694 | 179.992774 | −0.592367 | 0.18686728 | −3.1699876 | 0.00152445 | 0.03221667 |
| ENSG00000184445 | 1555.51616 | 0.62133307 | 0.18678282 | 3.32650014 | 0.00087944 | 0.02156661 |
| ENSG00000188026 | 308.012575 | −0.7007029 | 0.2224295 | −3.1502248 | 0.00163145 | 0.03349066 |
| ENSG00000184992 | 699.226999 | 0.49352315 | 0.13299678 | 3.71079029 | 0.00020661 | 0.00721446 |
| ENSG00000060709 | 3307.95041 | −0.5441322 | 0.15442156 | −3.5236803 | 0.0004256 | 0.0128284 |
| ENSG00000165480 | 470.356075 | 0.91441714 | 0.17288454 | 5.28917816 | 1.23E−07 | 1.49E−05 |
| ENSG00000127863 | 32.8970768 | 1.92658888 | 0.35283879 | 5.4602525 | 4.75E−08 | 6.45E−06 |
| ENSG00000151849 | 763.813102 | 0.71568143 | 0.17000838 | 4.20968319 | 2.56E−05 | 0.00138208 |
| ENSG00000120694 | 4611.94632 | 0.40997906 | 0.11822301 | 3.46784481 | 0.00052465 | 0.01491742 |
| ENSG00000139618 | 493.652989 | 1.04987054 | 0.22008656 | 4.77026191 | 1.84E−06 | 0.00014939 |
| ENSG00000133119 | 1158.95582 | 0.73103943 | 0.17652928 | 4.14117959 | 3.46E−05 | 0.00174065 |
| ENSG00000180660 | 3822.48715 | −0.723923 | 0.09585084 | −7.5525997 | 4.27E−14 | 2.01E−11 |
| ENSG00000120693 | 2836.72911 | 0.50959869 | 0.16656584 | 3.05944292 | 0.00221749 | 0.04232937 |
| ENSG00000150907 | 102.104456 | 0.78057465 | 0.22486798 | 3.47125749 | 0.00051803 | 0.01477465 |
| ENSG00000139687 | 1440.1359 | 0.65297468 | 0.20739506 | 3.1484582 | 0.00164134 | 0.033599 |
| ENSG00000136108 | 2343.70149 | 0.52931489 | 0.17315756 | 3.0568397 | 0.00223684 | 0.04265073 |
| ENSG00000139734 | 414.985469 | 0.66792881 | 0.1651838 | 4.04354904 | 5.26E−05 | 0.00249034 |
| ENSG00000276644 | 148.725288 | −1.4979981 | 0.20112352 | −7.44815 | 9.47E−14 | 4.12E−11 |
| ENSG00000152193 | 228.433775 | 0.77018487 | 0.21975496 | 3.50474393 | 0.00045705 | 0.01348914 |
| ENSG00000102580 | 324.321811 | 0.67752254 | 0.18257079 | 3.71101276 | 0.00020643 | 0.00721446 |
| ENSG00000043355 | 61.3768772 | −1.0434058 | 0.31054976 | −3.3598666 | 0.0007798 | 0.0198815 |
| ENSG00000204442 | 1698.86651 | −0.4993717 | 0.10133223 | −4.9280635 | 8.30E−07 | 7.71E−05 |
| ENSG00000274718 | 105.845859 | −1.0229629 | 0.24316303 | −4.2069012 | 2.59E−05 | 0.00139138 |
| ENSG00000187498 | 850.036516 | 0.6612711 | 0.18565064 | 3.56191118 | 0.00036816 | 0.01135956 |
| ENSG00000126218 | 324.763949 | −0.6553885 | 0.16648355 | −3.936656 | 8.26E−05 | 0.00352298 |
| ENSG00000198176 | 1888.55783 | 0.35579883 | 0.11558878 | 3.07814339 | 0.00208295 | 0.04037148 |
| ENSG00000100968 | 332.668205 | −0.546531 | 0.17195728 | −3.1782952 | 0.00148144 | 0.03146434 |
| ENSG00000259017 | 37.315656 | −1.1188867 | 0.37336892 | −2.9967324 | 0.0027289 | 0.04986004 |
| ENSG00000168348 | 403.695123 | −0.9792201 | 0.18062966 | −5.4211477 | 5.92E−08 | 7.73E−06 |
| ENSG00000100479 | 251.080204 | 0.74612559 | 0.1729305 | 4.31459799 | 1.60E−05 | 0.00093244 |
| ENSG00000100504 | 80.1519347 | 0.9338382 | 0.24246931 | 3.85136667 | 0.00011746 | 0.0046464 |
| ENSG00000073712 | 2037.89378 | 0.47664659 | 0.13410863 | 3.55418295 | 0.00037916 | 0.01167743 |
| ENSG00000131979 | 905.131557 | −0.6572079 | 0.14852435 | −4.4249168 | 9.65E−06 | 0.00060865 |
| ENSG00000198554 | 771.995885 | 0.79197895 | 0.15838431 | 5.00036239 | 5.72E−07 | 5.58E−05 |
| ENSG00000131981 | 209.424129 | −0.8945364 | 0.20621326 | −4.3379191 | 1.44E−05 | 0.00086214 |
| ENSG00000184302 | 41.7935368 | −1.3947294 | 0.3699302 | −3.7702502 | 0.00016308 | 0.00596452 |
| ENSG00000179841 | 150.955423 | 1.42714152 | 0.24630554 | 5.79419168 | 6.87E−09 | 1.20E−06 |
| ENSG00000126803 | 367.472833 | 0.69313432 | 0.17410228 | 3.98119032 | 6.86E−05 | 0.0030542 |
| ENSG00000100678 | 851.706968 | 0.65249624 | 0.18191625 | 3.58679476 | 0.00033477 | 0.01052038 |
| ENSG00000205683 | 29.2826784 | 1.28215894 | 0.37403444 | 3.42791677 | 0.00060823 | 0.01654118 |
| ENSG00000119599 | 196.596188 | −0.5881898 | 0.18183953 | −3.2346643 | 0.00121786 | 0.02722932 |
| ENSG00000119699 | 49.5649341 | 1.21782045 | 0.28569619 | 4.26264153 | 2.02E−05 | 0.00113522 |
| ENSG00000100604 | 8593.89536 | −1.3089533 | 0.20232087 | −6.4696899 | 9.82E−11 | 2.45E−08 |
| ENSG00000165943 | 1379.51242 | −0.3530255 | 0.11521297 | −3.0641123 | 0.00218317 | 0.04188258 |
| ENSG00000012963 | 1019.46674 | 0.48018041 | 0.13136392 | 3.65534484 | 0.00025684 | 0.00859668 |
| ENSG00000188488 | 17.5705134 | 1.6260162 | 0.46515175 | 3.49566827 | 0.00047288 | 0.01374094 |
| ENSG00000235706 | 116.450069 | −0.889234 | 0.27340726 | −3.2524154 | 0.00114429 | 0.0260126 |
| ENSG00000168398 | 18.573313 | 2.26845895 | 0.4822722 | 4.7036901 | 2.56E−06 | 0.00019443 |
| ENSG00000100749 | 481.845805 | 0.59647744 | 0.19871799 | 3.00162779 | 0.0026854 | 0.04921305 |
| ENSG00000182218 | 44.6313937 | −1.9743896 | 0.35401004 | −5.5772134 | 2.44E−08 | 3.58E−06 |
| ENSG00000066629 | 488.522303 | 0.57302579 | 0.14643613 | 3.91314492 | 9.11E−05 | 0.00378921 |
| ENSG00000140105 | 1356.91621 | 0.46861322 | 0.10838661 | 4.32353434 | 1.54E−05 | 0.00090477 |
| ENSG00000259031 | 31.9495195 | −1.2609829 | 0.41156258 | −3.063891 | 0.00218478 | 0.04188258 |
| ENSG00000185559 | 1605.2048 | −1.6650843 | 0.24923726 | −6.6807198 | 2.38E−11 | 7.08E−09 |
| ENSG00000258913 | 76.2186512 | 0.77732668 | 0.25033722 | 3.10511834 | 0.00190203 | 0.03766328 |
| ENSG00000198826 | 1327.79111 | 0.64557014 | 0.1631254 | 3.95750847 | 7.57E−05 | 0.00328704 |
| ENSG00000166923 | 561.836783 | 2.74233616 | 0.1743686 | 15.7272366 | 9.84E−56 | 3.34E−52 |
| ENSG00000176454 | 786.081012 | −0.6857511 | 0.1443834 | −4.7495143 | 2.04E−06 | 0.00016322 |
| ENSG00000137801 | 56.3403825 | 1.64130375 | 0.28734227 | 5.71201631 | 1.12E−08 | 1.84E−06 |
| ENSG00000166073 | 236.735163 | 0.74436704 | 0.17044458 | 4.3672086 | 1.26E−05 | 0.00076544 |
| ENSG00000156970 | 1412.04556 | 0.56114707 | 0.15453151 | 3.63127938 | 0.00028202 | 0.00922134 |
| ENSG00000137812 | 1086.25955 | 0.74073483 | 0.20477455 | 3.61731876 | 0.00029767 | 0.00958533 |
| ENSG00000245849 | 206.843649 | −0.7519849 | 0.20001791 | −3.7595878 | 0.00017019 | 0.00611093 |
| ENSG00000051180 | 526.852118 | 0.74794428 | 0.14818283 | 5.04744213 | 4.48E−07 | 4.44E−05 |
| ENSG00000137804 | 2219.341 | 0.47162158 | 0.1430943 | 3.29587957 | 0.00098114 | 0.02331927 |
| ENSG00000092470 | 703.11765 | 0.69017125 | 0.12628659 | 5.46511898 | 4.63E−08 | 6.36E−06 |
| ENSG00000259520 | 15.9892856 | −1.9082959 | 0.58099043 | −3.2845566 | 0.00102143 | 0.02380998 |
| ENSG00000166147 | 2206.83093 | 1.23509162 | 0.25063093 | 4.92792977 | 8.31E−07 | 7.71E−05 |
| ENSG00000244879 | 2590.51986 | −0.678329 | 0.16257009 | −4.1725325 | 3.01E−05 | 0.00155851 |
| ENSG00000069869 | 213.609931 | 0.66820968 | 0.18070236 | 3.69784708 | 0.00021744 | 0.00754578 |
| ENSG00000128923 | 382.87676 | 0.5947485 | 0.18456061 | 3.22251039 | 0.00127073 | 0.02811501 |
| ENSG00000182718 | 832.938598 | 1.31630423 | 0.17530837 | 7.5085076 | 5.98E−14 | 2.74E−11 |
| ENSG00000259370 | 10.2791561 | 2.18937726 | 0.69439078 | 3.15294688 | 0.00161631 | 0.0332874 |
| ENSG00000140416 | 1362.61801 | 1.89289949 | 0.13210021 | 14.3292696 | 1.44E−46 | 3.48E−43 |
| ENSG00000166803 | 340.428891 | 0.80293924 | 0.24985351 | 3.21364005 | 0.00131064 | 0.02886525 |
| ENSG00000174442 | 869.801891 | 0.66551352 | 0.18285115 | 3.63964634 | 0.00027301 | 0.00901367 |
| ENSG00000137834 | 532.719059 | 0.89432512 | 0.22492455 | 3.97611158 | 7.01E−05 | 0.00310236 |
| ENSG00000188779 | 71.4729901 | −1.0476328 | 0.33584519 | −3.1193919 | 0.00181225 | 0.03635206 |
| ENSG00000128973 | 608.332068 | 0.55849328 | 0.14761533 | 3.78343685 | 0.00015468 | 0.00577796 |
| ENSG00000137809 | 50.5178333 | 2.28968767 | 0.33876924 | 6.75884169 | 1.39E−11 | 4.67E−09 |
| ENSG00000137807 | 1007.54603 | 0.52038052 | 0.14099855 | 3.69067999 | 0.00022366 | 0.00773 |
| ENSG00000187720 | 45.223503 | 1.8228732 | 0.33748165 | 5.40139948 | 6.61E−08 | 8.31E−06 |
| ENSG00000179335 | 1111.1811 | −0.3407479 | 0.10366008 | −3.2871659 | 0.00101201 | 0.02365543 |
| ENSG00000178802 | 688.361928 | −0.4987439 | 0.14737895 | −3.384092 | 0.00071414 | 0.0186733 |
| ENSG00000140398 | 201.461396 | −0.8412845 | 0.19512031 | −4.3116193 | 1.62E−05 | 0.00094185 |
| ENSG00000140400 | 698.996233 | −0.6428611 | 0.14394873 | −4.4659031 | 7.97E−06 | 0.00051447 |
| ENSG00000103888 | 94.6706449 | 1.56996573 | 0.30561704 | 5.13703597 | 2.79E−07 | 3.01E−05 |
| ENSG00000140525 | 2292.67873 | 0.71676113 | 0.1550373 | 4.62315296 | 3.78E−06 | 0.00027646 |
| ENSG00000166825 | 163.700948 | −0.7908059 | 0.20527635 | −3.8523967 | 0.00011697 | 0.0046464 |
| ENSG00000242498 | 347.783964 | 0.77302787 | 0.16755981 | 4.61344426 | 3.96E−06 | 0.00028479 |
| ENSG00000197299 | 672.836057 | 0.91627999 | 0.16706355 | 5.48461931 | 4.14E−08 | 5.81E−06 |
| ENSG00000198901 | 2157.55581 | 0.40847913 | 0.109717 | 3.72302512 | 0.00019685 | 0.00690195 |
| ENSG00000140450 | 753.946078 | −0.4327365 | 0.13584068 | −3.1856181 | 0.00144445 | 0.03081691 |
| ENSG00000182253 | 354.654633 | 0.87461543 | 0.17508946 | 4.99524891 | 5.88E−07 | 5.67E−05 |
| ENSG00000172366 | 544.407633 | −0.8397349 | 0.26660325 | −3.1497548 | 0.00163408 | 0.03349066 |
| ENSG00000162004 | 167.876855 | −0.8075295 | 0.23690607 | −3.4086483 | 0.00065286 | 0.01750231 |
| ENSG00000103227 | 110.518964 | −0.9399698 | 0.2948323 | −3.1881508 | 0.00143186 | 0.03071888 |
| ENSG00000206053 | 2383.39823 | 0.37793458 | 0.11011234 | 3.43226357 | 0.00059857 | 0.01635694 |
| ENSG00000172382 | 270.247468 | −0.9210656 | 0.23741309 | −3.8795905 | 0.00010463 | 0.00423774 |
| ENSG00000276791 | 58.978889 | −1.0162383 | 0.29250728 | −3.4742326 | 0.00051232 | 0.01466107 |
| ENSG00000006327 | 156.091245 | 0.84588502 | 0.25253379 | 3.34959145 | 0.00080931 | 0.02043744 |
| ENSG00000008517 | 39.6159825 | 1.9052153 | 0.35867165 | 5.31186472 | 1.09E−07 | 1.32E−05 |
| ENSG00000263013 | 16.7559205 | 1.79959496 | 0.57995461 | 3.1029928 | 0.00191574 | 0.03780251 |
| ENSG00000103540 | 1493.85269 | 0.58815588 | 0.19354335 | 3.03888451 | 0.00237456 | 0.04462487 |
| ENSG00000167191 | 97.6006923 | 0.89023424 | 0.24933905 | 3.5703763 | 0.00035647 | 0.01103882 |
| ENSG00000140743 | 382.483775 | 0.41577875 | 0.1350763 | 3.07810285 | 0.00208323 | 0.04037148 |
| ENSG00000077238 | 59.2354456 | 1.345658 | 0.25759898 | 5.22384837 | 1.75E−07 | 2.02E−05 |
| ENSG00000149922 | 56.7482566 | −1.035694 | 0.31241835 | −3.315087 | 0.00091615 | 0.02214672 |
| ENSG00000261474 | 68.1034157 | −0.8678462 | 0.27509063 | −3.1547646 | 0.00160628 | 0.03316121 |
| ENSG00000260267 | 116.426551 | −0.7710878 | 0.24507457 | −3.1463394 | 0.00165328 | 0.03376194 |
| ENSG00000260153 | 9.4798296 | −2.5276911 | 0.68694337 | −3.6796208 | 0.00023358 | 0.00802403 |
| ENSG00000171241 | 232.729567 | 0.89869624 | 0.16630191 | 5.4040042 | 6.52E−08 | 8.31E−06 |
| ENSG00000091651 | 607.103796 | 0.66499942 | 0.17037979 | 3.90304162 | 9.50E−05 | 0.00391891 |
| ENSG00000278928 | 309.214394 | 0.64755739 | 0.15639852 | 4.14043173 | 3.47E−05 | 0.00174065 |
| ENSG00000125148 | 44.9000027 | 1.4563197 | 0.40269775 | 3.61640884 | 0.00029872 | 0.00958654 |
| ENSG00000125170 | 4162.24254 | 0.69361754 | 0.13366843 | 5.18909018 | 2.11E−07 | 2.42E−05 |
| ENSG00000181938 | 229.819668 | 0.79978924 | 0.18485549 | 4.32656468 | 1.51E−05 | 0.00089866 |
| ENSG00000067955 | 1123.17191 | 0.37488954 | 0.11863791 | 3.15994716 | 0.00157798 | 0.03289715 |
| ENSG00000103044 | 111.559614 | 0.68809971 | 0.21819913 | 3.15354011 | 0.00161303 | 0.03326018 |
| ENSG00000181019 | 216.656421 | 1.20646721 | 0.21457121 | 5.62268926 | 1.88E−08 | 2.87E−06 |
| ENSG00000168411 | 1450.61635 | 0.59540248 | 0.14390533 | 4.13745946 | 3.51E−05 | 0.00174762 |
| ENSG00000171724 | 1009.10779 | 0.53841415 | 0.16895613 | 3.18670975 | 0.00143901 | 0.03075569 |
| ENSG00000103154 | 145.991349 | 0.83572962 | 0.25823632 | 3.23629778 | 0.00121091 | 0.02722932 |
| ENSG00000103187 | 323.892506 | 1.30427791 | 0.20082096 | 6.49472994 | 8.32E−11 | 2.14E−08 |
| ENSG00000131153 | 656.796472 | 0.73278019 | 0.18531481 | 3.95424511 | 7.68E−05 | 0.00332372 |
| ENSG00000103257 | 1801.56092 | 0.67247362 | 0.19816772 | 3.39345693 | 0.00069016 | 0.01824312 |
| ENSG00000187741 | 527.760446 | 0.5233368 | 0.16172736 | 3.23591992 | 0.00121251 | 0.02722932 |
| ENSG00000132386 | 837.517737 | −0.4639859 | 0.15243102 | −3.043907 | 0.00233527 | 0.04408185 |
| ENSG00000132535 | 1001.34567 | −0.4615012 | 0.14138206 | −3.2642133 | 0.00109768 | 0.02524445 |
| ENSG00000072818 | 73.0373444 | −1.0854018 | 0.28207347 | −3.8479399 | 0.00011912 | 0.00467913 |
| ENSG00000179111 | 51.1174922 | −1.5544975 | 0.34852651 | −4.4601987 | 8.19E−06 | 0.00052635 |
| ENSG00000133026 | 7009.33249 | 0.46758363 | 0.14072454 | 3.32268731 | 0.00089155 | 0.02173789 |
| ENSG00000221926 | 235.049676 | −0.5809418 | 0.1743871 | −3.3313347 | 0.00086431 | 0.02135322 |
| ENSG00000175061 | 4331.12919 | −0.4597105 | 0.15134395 | −3.0375219 | 0.00238532 | 0.04467869 |
| ENSG00000108448 | 407.284459 | −0.6379269 | 0.15686363 | −4.066761 | 4.77E−05 | 0.00229172 |
| ENSG00000128487 | 455.870894 | 0.59617365 | 0.13408028 | 4.44639315 | 8.73E−06 | 0.00055501 |
| ENSG00000109084 | 1108.3879 | 0.38633342 | 0.1289584 | 2.99579871 | 0.00273727 | 0.04989418 |
| ENSG00000076382 | 1122.73639 | 0.41021868 | 0.11730983 | 3.49688257 | 0.00047073 | 0.01371601 |
| ENSG00000176658 | 344.519368 | 1.03460503 | 0.15728525 | 6.57788979 | 4.77E−11 | 1.33E−08 |
| ENSG00000108691 | 123.597105 | 3.04163387 | 0.29802103 | 10.2061049 | 1.86E−24 | 1.97E−21 |
| ENSG00000277161 | 351.106286 | 0.66751185 | 0.21929273 | 3.04393059 | 0.00233509 | 0.04408185 |
| ENSG00000141736 | 321.398637 | −0.5847693 | 0.17243517 | −3.3912415 | 0.00069577 | 0.01833079 |
| ENSG00000094804 | 869.943664 | 0.9884758 | 0.14707133 | 6.72106371 | 1.80E−11 | 5.78E−09 |
| ENSG00000131747 | 9271.95076 | 0.61624934 | 0.16283897 | 3.78440939 | 0.00015407 | 0.00577182 |
| ENSG00000187595 | 15.0418627 | −1.7801745 | 0.53900775 | −3.3026882 | 0.00095763 | 0.02292036 |
| ENSG00000167925 | 394.639173 | −0.5944849 | 0.1881667 | −3.1593521 | 0.0015812 | 0.03292395 |
| ENSG00000177469 | 274.16205 | 1.75534705 | 0.16183937 | 10.8462302 | 2.08E−27 | 3.21E−24 |
| ENSG00000108785 | 187.247507 | −0.738168 | 0.22044598 | −3.3485208 | 0.00081244 | 0.02046312 |
| ENSG00000012048 | 1016.29395 | 1.02895712 | 0.17896028 | 5.74963969 | 8.94E−09 | 1.52E−06 |
| ENSG00000108309 | 1180.49887 | −0.5595829 | 0.18587037 | −3.0106085 | 0.00260725 | 0.04819717 |
| ENSG00000182963 | 1427.41123 | 0.41332417 | 0.12302403 | 3.35970266 | 0.00078026 | 0.0198815 |
| ENSG00000181513 | 144.205114 | −0.7113506 | 0.21990731 | −3.2347748 | 0.00121739 | 0.02722932 |
| ENSG00000263412 | 108.161436 | −0.7618659 | 0.23367703 | −3.260337 | 0.0011128 | 0.02551919 |
| ENSG00000108821 | 311.371996 | 1.01631157 | 0.20177616 | 5.03682678 | 4.73E−07 | 4.65E−05 |
| ENSG00000136444 | 1385.83244 | −0.4492516 | 0.13338547 | −3.3680701 | 0.00075696 | 0.01949267 |
| ENSG00000136449 | 50.126548 | −1.2257386 | 0.31407826 | −3.9026535 | 9.51E−05 | 0.00391891 |
| ENSG00000006282 | 820.380311 | −0.6418439 | 0.17455513 | −3.6770268 | 0.00023597 | 0.00805711 |
| ENSG00000108846 | 38.383169 | 1.4578579 | 0.38504234 | 3.78622754 | 0.00015295 | 0.00574245 |
| ENSG00000229980 | 50.455427 | −0.9925319 | 0.30455478 | −3.2589603 | 0.00111821 | 0.02557423 |
| ENSG00000166292 | 1479.80896 | −0.7418894 | 0.17166768 | −4.32166 | 1.55E−05 | 0.00090933 |
| ENSG00000141179 | 567.987575 | −0.5749332 | 0.16717266 | −3.439158 | 0.00058353 | 0.0160754 |
| ENSG00000182628 | 892.28329 | 0.55390453 | 0.175519 | 3.1558095 | 0.00160053 | 0.03311658 |
| ENSG00000136492 | 1209.83971 | 0.99401439 | 0.21424493 | 4.6396169 | 3.49E−06 | 0.00025754 |
| ENSG00000008283 | 2130.34089 | 0.57517432 | 0.12675423 | 4.53771305 | 5.69E−06 | 0.00038144 |
| ENSG00000182481 | 5298.71756 | 0.48401078 | 0.12797965 | 3.7819356 | 0.00015561 | 0.00579115 |
| ENSG00000180616 | 156.262292 | 1.344574 | 0.21676756 | 6.20283779 | 5.55E−10 | 1.18E−07 |
| ENSG00000172794 | 24.75624 | 1.27470017 | 0.39094757 | 3.26053998 | 0.001112 | 0.02551919 |
| ENSG00000109065 | 1025.22782 | −0.4899183 | 0.14732091 | −3.3255178 | 0.00088254 | 0.02158038 |
| ENSG00000167861 | 851.396493 | −0.5752463 | 0.17388568 | −3.3081867 | 0.00093902 | 0.02257111 |
| ENSG00000073350 | 115.293063 | −0.7863422 | 0.24781072 | −3.1731567 | 0.00150791 | 0.0319068 |
| ENSG00000266714 | 192.477017 | −1.1567489 | 0.231436 | −4.9981374 | 5.79E−07 | 5.61E−05 |
| ENSG00000108469 | 636.333023 | −0.485531 | 0.14704571 | −3.3019052 | 0.00096031 | 0.02292036 |
| ENSG00000141524 | 272.789463 | −0.6831584 | 0.22694553 | −3.0102306 | 0.00261049 | 0.04820466 |
| ENSG00000167900 | 835.760296 | 0.80644506 | 0.19222894 | 4.19523234 | 2.73E−05 | 0.00145012 |
| ENSG00000035862 | 6038.5496 | 0.6238341 | 0.10250167 | 6.08608726 | 1.16E−09 | 2.23E−07 |
| ENSG00000167280 | 907.977976 | −0.5575413 | 0.15836013 | −3.5207177 | 0.00043038 | 0.01289831 |
| ENSG00000169660 | 635.178202 | −0.5355946 | 0.17027418 | −3.1454831 | 0.00165813 | 0.03382022 |
| ENSG00000176890 | 588.137002 | 0.6329387 | 0.17683283 | 3.57930535 | 0.00034451 | 0.0107469 |
| ENSG00000173482 | 140.970869 | −1.1702074 | 0.20328855 | −5.7563863 | 8.59E−09 | 1.47E−06 |
| ENSG00000168461 | 553.109296 | 0.44347717 | 0.13165614 | 3.36845037 | 0.00075592 | 0.01949267 |
| ENSG00000132872 | 2031.82376 | 0.93006392 | 0.1936971 | 4.80164094 | 1.57E−06 | 0.00013027 |
| ENSG00000184828 | 82.934306 | −0.7604082 | 0.23469458 | −3.2399905 | 0.00119534 | 0.02701047 |
| ENSG00000166845 | 330.908877 | 0.82415394 | 0.22779203 | 3.61801048 | 0.00029688 | 0.00957793 |
| ENSG00000081923 | 48.1451714 | 1.59407772 | 0.32880576 | 4.84808339 | 1.25E−06 | 0.00010738 |
| ENSG00000125895 | 275.406847 | −0.9805899 | 0.24255571 | −4.0427411 | 5.28E−05 | 0.00249034 |
| ENSG00000088836 | 106.741058 | −0.8489418 | 0.23461259 | −3.6184834 | 0.00029633 | 0.00957793 |
| ENSG00000101265 | 166.938115 | 0.67506883 | 0.18445901 | 3.65972262 | 0.00025249 | 0.00846784 |
| ENSG00000132646 | 3349.40447 | 0.65523033 | 0.18059167 | 3.62824233 | 0.00028536 | 0.00929465 |
| ENSG00000089199 | 2028.44486 | −0.3736416 | 0.10622914 | −3.5173173 | 0.00043593 | 0.01302424 |
| ENSG00000125885 | 876.440311 | 0.66489385 | 0.15684682 | 4.23912864 | 2.24E−05 | 0.0012366 |
| ENSG00000101384 | 158.199451 | 0.69308633 | 0.20190623 | 3.43271385 | 0.00059757 | 0.01635614 |
| ENSG00000101003 | 620.311777 | 0.6707518 | 0.17656359 | 3.79892477 | 0.00014533 | 0.00549258 |
| ENSG00000088325 | 2963.97072 | 0.42670292 | 0.11739773 | 3.63467788 | 0.00027833 | 0.0091182 |
| ENSG00000101412 | 1344.81629 | 0.60457888 | 0.18204333 | 3.32107127 | 0.00089673 | 0.0218015 |
| ENSG00000088340 | 82.1369088 | −1.1506074 | 0.28530861 | −4.032852 | 5.51E−05 | 0.00258319 |
| ENSG00000214078 | 1416.09704 | −0.3762675 | 0.12020409 | −3.1302389 | 0.00174664 | 0.03524436 |
| ENSG00000118707 | 616.734141 | −0.3678197 | 0.11784997 | −3.1210844 | 0.00180186 | 0.03622942 |
| ENSG00000149636 | 402.47954 | 0.74934096 | 0.17892553 | 4.18800473 | 2.81E−05 | 0.00148774 |
| ENSG00000101347 | 282.759015 | 0.56044786 | 0.15030695 | 3.7286889 | 0.00019248 | 0.00676265 |
| ENSG00000080839 | 267.126924 | 0.8943639 | 0.20450769 | 4.37325315 | 1.22E−05 | 0.00075264 |
| ENSG00000198959 | 123.91911 | 1.45646712 | 0.22087656 | 6.59403198 | 4.28E−11 | 1.21E−08 |
| ENSG00000101445 | 869.035542 | 0.55428989 | 0.1725042 | 3.21319657 | 0.00131266 | 0.02886525 |
| ENSG00000101057 | 3301.17794 | 0.67826197 | 0.14250532 | 4.75955552 | 1.94E−06 | 0.00015679 |
| ENSG00000124207 | 3776.12209 | 0.53117001 | 0.16458024 | 3.22742265 | 0.00124911 | 0.02784906 |
| ENSG00000054803 | 24.1796649 | −3.8707505 | 0.63070872 | −6.1371445 | 8.40E−10 | 1.68E−07 |
| ENSG00000101144 | 48.55399 | −2.263001 | 0.35779233 | −6.3249008 | 2.53E−10 | 5.81E−08 |
| ENSG00000125531 | 170.580113 | −1.0737157 | 0.25665118 | −4.1835604 | 2.87E−05 | 0.00150775 |
| ENSG00000115257 | 96.2326578 | −0.9264635 | 0.2775558 | −3.3379361 | 0.00084403 | 0.0210512 |
| ENSG00000167670 | 1247.79762 | 0.61791958 | 0.14833543 | 4.16569113 | 3.10E−05 | 0.00160112 |
| ENSG00000276043 | 1534.19026 | 0.65871229 | 0.17908499 | 3.67821043 | 0.00023488 | 0.00803598 |
| ENSG00000105519 | 144.396519 | −0.9071235 | 0.25046437 | −3.6217666 | 0.0002926 | 0.00949405 |
| ENSG00000205744 | 120.232388 | −0.9555768 | 0.21057513 | −4.5379375 | 5.68E−06 | 0.00038144 |
| ENSG00000125734 | 463.606055 | −0.7045864 | 0.18693516 | −3.7691484 | 0.00016381 | 0.00597802 |
| ENSG00000090661 | 354.69383 | −0.6183691 | 0.16428508 | −3.7640007 | 0.00016722 | 0.00602476 |
| ENSG00000167772 | 66.8737929 | 1.23361078 | 0.31632705 | 3.89979544 | 9.63E−05 | 0.00394413 |
| ENSG00000105088 | 1544.84935 | −0.5986957 | 0.18662227 | −3.2080616 | 0.00133633 | 0.02926129 |
| ENSG00000130816 | 4685.61518 | 0.41217198 | 0.13677715 | 3.01345639 | 0.0025829 | 0.04785135 |
| ENSG00000090339 | 9.80900729 | 2.50966557 | 0.62507691 | 4.01497083 | 5.95E−05 | 0.00272681 |
| ENSG00000105376 | 65.5694082 | −1.0979846 | 0.27826712 | −3.9457935 | 7.95E−05 | 0.00343442 |
| ENSG00000130176 | 35.4912277 | 1.92883282 | 0.34536391 | 5.58492874 | 2.34E−08 | 3.45E−06 |
| ENSG00000179284 | 78.2269659 | −0.9292453 | 0.27140796 | −3.4237955 | 0.00061753 | 0.01674041 |
| ENSG00000141854 | 75.9943981 | −1.0040655 | 0.26671437 | −3.7645721 | 0.00016683 | 0.00602379 |
| ENSG00000105011 | 508.492535 | 0.73744967 | 0.13794922 | 5.34580523 | 9.00E−08 | 1.12E−05 |
| ENSG00000011243 | 444.541633 | −0.51959 | 0.15425916 | −3.3682929 | 0.00075635 | 0.01949267 |
| ENSG00000131351 | 228.722665 | 0.6100986 | 0.19072616 | 3.19881977 | 0.00137991 | 0.03006052 |
| ENSG00000130304 | 292.331681 | −0.7276522 | 0.21627124 | −3.3645351 | 0.00076673 | 0.01968436 |
| ENSG00000105639 | 119.482856 | −0.9819559 | 0.20683138 | −4.7476159 | 2.06E−06 | 0.00016399 |
| ENSG00000105643 | 211.593241 | −0.588099 | 0.19625128 | −2.9966631 | 0.00272952 | 0.04986004 |
| ENSG00000105649 | 822.035493 | −0.4757171 | 0.15640805 | −3.0415127 | 0.00235393 | 0.04433533 |
| ENSG00000130513 | 320.456526 | −1.1041259 | 0.26403363 | −4.1817623 | 2.89E−05 | 0.00151504 |
| ENSG00000105696 | 1012.50211 | −0.6966752 | 0.21245156 | −3.2792192 | 0.00104095 | 0.02419847 |
| ENSG00000160161 | 160.686702 | −0.988265 | 0.26882287 | −3.6762683 | 0.00023667 | 0.00806486 |
| ENSG00000184635 | 210.390309 | 0.89871372 | 0.22859382 | 3.93148738 | 8.44E−05 | 0.00357267 |
| ENSG00000197124 | 133.311216 | 0.77883687 | 0.22600833 | 3.44605375 | 0.00056884 | 0.01582486 |
| ENSG00000213988 | 81.6262511 | 0.7430199 | 0.24183007 | 3.07248769 | 0.00212283 | 0.04093677 |
| ENSG00000231205 | 183.23033 | 0.84279417 | 0.22327993 | 3.77460785 | 0.00016026 | 0.0059187 |
| ENSG00000268119 | 84.5783584 | 0.94699132 | 0.25234343 | 3.75278772 | 0.00017488 | 0.00622158 |
| ENSG00000197020 | 194.687678 | 1.07457897 | 0.23595254 | 4.55421651 | 5.26E−06 | 0.00036421 |
| ENSG00000197134 | 77.2148368 | 1.22135317 | 0.29033298 | 4.20673239 | 2.59E−05 | 0.00139138 |
| ENSG00000196081 | 150.604933 | 0.86883536 | 0.20031986 | 4.33724031 | 1.44E−05 | 0.00086214 |
| ENSG00000269416 | 339.54691 | 0.66251344 | 0.16008201 | 4.13858773 | 3.49E−05 | 0.00174417 |
| ENSG00000196172 | 287.268708 | 0.76584798 | 0.23368247 | 3.27730177 | 0.00104804 | 0.02433008 |
| ENSG00000213967 | 91.8853631 | 0.92174235 | 0.24246033 | 3.80162135 | 0.00014375 | 0.00546431 |
| ENSG00000153879 | 1348.94628 | 0.39627135 | 0.12337475 | 3.21193247 | 0.00131845 | 0.02894458 |
| ENSG00000089351 | 3151.45123 | −0.6871756 | 0.18349536 | −3.744921 | 0.00018045 | 0.00637968 |
| ENSG00000236144 | 209.245094 | −0.6219678 | 0.17304614 | −3.5942307 | 0.00032535 | 0.01033935 |
| ENSG00000099338 | 31.6891693 | −1.4297893 | 0.40823638 | −3.5023564 | 0.00046116 | 0.01351771 |
| ENSG00000105204 | 820.110744 | −0.7194732 | 0.20830026 | −3.4540199 | 0.0005523 | 0.01544065 |
| ENSG00000090006 | 804.300645 | −0.7925508 | 0.21446152 | −3.6955386 | 0.00021942 | 0.00759914 |
| ENSG00000123815 | 596.001597 | −0.7594043 | 0.19592655 | −3.8759642 | 0.0001062 | 0.00429112 |
| ENSG00000243137 | 29.4010299 | 1.20078017 | 0.37654594 | 3.18893406 | 0.00142798 | 0.03071343 |
| ENSG00000176531 | 197.957664 | −0.8337297 | 0.24632476 | −3.384677 | 0.00071262 | 0.01867054 |
| ENSG00000130202 | 596.135072 | −1.1973632 | 0.20388191 | −5.8728271 | 4.28E−09 | 7.65E−07 |
| ENSG00000213889 | 122.333681 | −0.6829968 | 0.21857177 | −3.1248169 | 0.00177916 | 0.03585785 |
| ENSG00000125746 | 301.72452 | −0.7120987 | 0.17731342 | −4.0160451 | 5.92E−05 | 0.00272177 |
| ENSG00000142230 | 2731.46721 | 0.45798773 | 0.13728678 | 3.33599287 | 0.00084995 | 0.02111817 |
| ENSG00000105327 | 597.783535 | −1.2637109 | 0.27700802 | −4.5620013 | 5.07E−06 | 0.00035239 |
| ENSG00000105373 | 10037.4906 | −0.7326643 | 0.24151204 | −3.0336555 | 0.0024161 | 0.04515555 |
| ENSG00000105516 | 270.780513 | −0.9029832 | 0.27218169 | −3.317575 | 0.00090803 | 0.02198173 |
| ENSG00000198464 | 538.719611 | 0.56350169 | 0.18278353 | 3.08289086 | 0.00205 | 0.03997111 |
| ENSG00000198300 | 517.788473 | 0.65962327 | 0.21714134 | 3.03776 | 0.00238344 | 0.04467869 |
| ENSG00000099326 | 562.990333 | −0.6476158 | 0.16987383 | −3.8123338 | 0.00013766 | 0.00527337 |
| ENSG00000274602 | 335.233436 | −0.5228974 | 0.16558575 | −3.1578647 | 0.00158929 | 0.03301139 |
| ENSG00000093009 | 724.72203 | 0.84251893 | 0.13828457 | 6.09264596 | 1.11E−09 | 2.17E−07 |
| ENSG00000234409 | 272.222873 | −0.6752436 | 0.18490831 | −3.6517752 | 0.00026043 | 0.00869992 |
| ENSG00000184117 | 3074.35027 | −0.3645044 | 0.11514471 | −3.1656203 | 0.00154753 | 0.03253097 |
| ENSG00000100297 | 1649.10322 | 0.61534647 | 0.16053659 | 3.83306051 | 0.00012656 | 0.00491465 |
| ENSG00000100345 | 4577.06237 | 0.66281596 | 0.21754481 | 3.04680199 | 0.0023129 | 0.04375687 |
| ENSG00000100353 | 3295.2046 | −0.3698626 | 0.11922086 | −3.1023311 | 0.00192003 | 0.03780632 |
| ENSG00000189060 | 5799.93036 | −0.4531704 | 0.11987535 | −3.780347 | 0.00015661 | 0.00581547 |
| ENSG00000184381 | 89.5811258 | −0.866921 | 0.26045601 | −3.3284736 | 0.00087323 | 0.02150764 |
| ENSG00000244627 | 56.3750776 | −0.9368251 | 0.27705432 | −3.381377 | 0.00072124 | 0.01882979 |
| ENSG00000202058 | 21.2434535 | −2.5838403 | 0.70043872 | −3.6888884 | 0.00022524 | 0.00776881 |
| ENSG00000093000 | 2073.89854 | 0.40282729 | 0.13196824 | 3.0524563 | 0.00226977 | 0.04308497 |
| ENSG00000075218 | 1247.47627 | 0.40596073 | 0.12854647 | 3.15808525 | 0.00158809 | 0.03301139 |
| ENSG00000277437 | 53.4048221 | −4.6083693 | 1.03459676 | −4.4542661 | 8.42E−06 | 0.00053907 |
| ENSG00000276104 | 46.1261027 | −4.3521794 | 1.08757422 | −4.0017309 | 6.29E−05 | 0.00283047 |
| ENSG00000278047 | 49.2431991 | −3.4960437 | 1.00420648 | −3.4813992 | 0.0004988 | 0.01432261 |
| ENSG00000277105 | 13850.374 | −3.3635253 | 0.97902356 | −3.4355918 | 0.00059126 | 0.01626203 |
| ENSG00000276737 | 409.786811 | −4.5202827 | 1.07123489 | −4.2196933 | 2.45E−05 | 0.00133487 |
| ENSG00000275783 | 46.209722 | −5.1996409 | 1.46961435 | −3.5380989 | 0.00040302 | 0.01232294 |
| ENSG00000278189 | 13.0780688 | −4.3956298 | 0.90219579 | −4.8721463 | 1.10E−06 | 9.61E−05 |
| ENSG00000274735 | 322.607828 | −2.8185171 | 0.88324839 | −3.1910809 | 0.00141742 | 0.03060248 |
| ENSG00000154734 | 2372.64189 | −0.5368687 | 0.11422705 | −4.7000137 | 2.60E−06 | 0.00019708 |
| ENSG00000159259 | 510.048758 | 0.69841476 | 0.13830662 | 5.04975639 | 4.42E−07 | 4.42E−05 |
| ENSG00000175894 | 596.369164 | −0.7142216 | 0.19931295 | −3.5834179 | 0.00033913 | 0.01063769 |
| ENSG00000182912 | 1286.0869 | −0.7244085 | 0.17027033 | −4.2544613 | 2.10E−05 | 0.00117363 |

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that the materials, methods, and examples provided here are representative of preferred embodiments, are exemplary, and are not intended as limitations on the scope of the invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Unless otherwise defined, all technical and scientific terms used herein have the same meaning as commonly understood by one of ordinary skill in the art to which this invention belongs.

All patents, patent applications, provisional applications, and publications referred to or cited herein are incorporated by reference in their entirety, including all figures and tables, to the extent they are not inconsistent with the explicit teachings of this specification. In case of conflict, the present specification, including definitions, will control.

### REFERENCES

1. Cavalli, G. and Heard, E. (2019) Advances in epigenetics link genetics to the environment and disease. Nature, 571, 489-499.
2. Allshire, R. C. and Madhani, H. D. (2017) Ten principles of heterochromatin formation and function. Nat. Rev. Mol. Cell Biol., 19, 229.
3. Bonev, B. and Cavalli, G. (2016) Organization and function of the 3D genome. Nat. Rev. Genet., 17, 661-678.
4. Horvath, S. (2013) DNA methylation age of human tissues and cell types. Genome Biol., 14, R115.
5. Feinberg, A. P. (2018) The Key Role of Epigenetics in Human Disease Prevention and Mitigation. N. Engl. J. Med., 378, 1323-1334.
6. Disteche, C. M. (2012) Dosage compensation of the sex chromosomes. Annu. Rev. Genet., 46, 537-560.
7. Zhang, L.-F., Huynh, K. D. and Lee, J. T. (2007) Perinucleolar targeting of the inactive X during S phase: evidence for a role in the maintenance of silencing. Cell, 129, 693-706.
8. Chen, C.-K., Blanco, M., Jackson, C., Aznauryan, E., Ollikainen, N., Surka, C., Chow, A., Cerase, A., McDonel, P. and Guttman, M. (2016) Xist recruits the X chromosome to the nuclear lamina to enable chromosome-wide silencing. Science (80-.)., 354, 468 LP-472.
9. Penny, G. D., Kay, G. F., Sheardown, S. A., Rastan, S. and Brockdorff, N. (1996) Requirement for Xist in X chromosome inactivation. Nature, 379, 131-137.
10. Holoch, D. and Moazed, D. (2015) RNA-mediated epigenetic regulation of gene expression. Nat. Rev. Genet., 16, 71.
11. McHugh, C. A., Chen, C.-K., Chow, A., Surka, C. F., Tran, C., McDonel, P., Pandya-Jones, A., Blanco, M., Burghard, C., Moradian, A., et al. (2015) The Xist lncRNA interacts directly with SHARP to silence transcription through HDAC3. Nature, 521, 232-236.
12. Cotton, A. M., Price, E. M., Jones, M. J., Balaton, B. P., Kobor, M. S. and Brown, C. J. (2015) Landscape of DNA methylation on the X chromosome reflects CpG density, functional chromatin state and X-chromosome inactivation. Hum. Mol. Genet., 24, 1528-1539.
13. Heard, E., Rougeulle, C., Arnaud, D., Avner, P., Allis, C. D. and Spector, D. L. (2001) Methylation of histone H3 at Lys-9 is an early mark on the X chromosome during X inactivation. Cell, 107, 727-738.
14. Plath, K., Fang, J., Mlynarczyk-Evans, S. K., Cao, R., Worringer, K. A., Wang, H., de la Cruz, C. C., Otte, A. P., Panning, B. and Zhang, Y. (2003) Role of histone H3 lysine 27 methylation in X inactivation. Science, 300, 131-135.
15. Costanzi, C. and Pehrson, J. R. (1998) Histone macroH2A1 is concentrated in the inactive X chromosome of female mammals. Nature, 393, 599-601.
16. Tukiainen, T., Villani, A.-C., Yen, A., Rivas, M. A., Marshall, J. L., Satija, R., Aguirre, M., Gauthier, L., Fleharty, M., Kirby, A., et al. (2017) Landscape of X chromosome inactivation across human tissues. Nature, 550, 244.
17. Balaton, B. P. and Brown, C. J. (2016) Escape Artists of the X Chromosome. Trends Genet., 32, 348-359.
18. Berletch, J. B., Ma, W., Yang, F., Shendure, J., Noble, W. S., Disteche, C. M. and Deng, X. (2015) Escape from X Inactivation Varies in Mouse Tissues. PLOS Genet., 11, e1005079.
19. Bennett-Baker, P. E., Wilkowski, J. and Burke, D. T. (2003) Age-associated activation of epigenetically repressed genes in the mouse. Genetics, 165, 2055-2062.
20. Sharp, A. J., Stathaki, E., Migliavacca, E., Brahmachary, M., Montgomery, S. B., Dupre, Y. and Antonarakis, S. E. (2011) DNA methylation profiles of human active and inactive X chromosomes. Genome Res., 21, 1592-1600.
21. Carrette, L. L. G., Wang, C.-Y., Wei, C., Press, W., Ma, W., Kelleher, R. J. and Lee, J. T. (2018) A mixed modality approach towards Xi reactivation for Rett syndrome and other X-linked disorders. Proc. Natl. Acad. Sci., 115, E668 LP-E675.
22. Sripathy, S., Leko, V., Adrianse, R. L., Loe, T., Foss, E. J., Dalrymple, E., Lao, U., Gatbonton-Schwager, T., Carter, K. T., Payer, B., et al. (2017) Screen for reactivation of MeCP2 on the inactive X chromosome identifies the BMP/TGF-β superfamily as a regulator of XIST expression. Proc. Natl. Acad. Sci., 114, 1619 LP-1624.
23. Bhatnagar, S., Zhu, X., Ou, J., Lin, L., Chamberlain, L., Zhu, L. J., Wajapeyee, N. and Green, M. R. (2014) Genetic and pharmacological reactivation of the mammalian inactive X chromosome. Proc. Natl. Acad. Sci. U.S.A, 111, 12591-12598.
24. Stricker, S. H., Koferle, A. and Beck, S. (2017) From profiles to function in epigenomics. Nat. Rev. Genet., 18, 51-66.
25. Thakore, P. I., Black, J. B., Hilton, I. B. and Gersbach, C. A. (2016) Editing the epigenome: technologies for programmable transcription and epigenetic modulation. Nat. Methods, 13, 127-137.
26. Liu, X. S., Wu, H., Ji, X., Stelzer, Y., Wu, X., Czauderna, S., Shu, J., Dadon, D., Young, R. A. and Jaenisch, R. (2016) Editing DNA Methylation in the Mammalian Genome. Cell, 167, 233-247.e17.
27. Liu, X. S., Wu, H., Krzisch, M., Wu, X., Graef, J., Muffat, J., Hnisz, D., Li, C. H., Yuan, B., Xu, C., et al. (2018) Rescue of Fragile X Syndrome Neurons by DNA Methylation Editing of the FMR1 Gene. Cell, 172, 979-992.e6.
28. Baumann, V., Wiesbeck, M., Breunig, C. T., Braun, J. M., Koferle, A., Ninkovic, J., Gotz, M. and Stricker, S. H. (2019) Targeted removal of epigenetic barriers during transcriptional reprogramming. Nat. Commun., 10, 2119.
29. Josipović, G., Tadić, V., Klasić, M., Zanki, V., Bečeheli, I., Chung, F., Ghantous, A., Keser, T., Madunid, J., Bošković, M., et al. (2019) Antagonistic and synergistic epigenetic modulation using orthologous CRISPR/dCas9-based modular system. Nucleic Acids Res., 10.1093/nar/gkz709.
30. Kalscheuer, V. M., Tao, J., Donnelly, A., Hollway, G., Schwinger, E., Kubart, S., Menzel, C., Hoeltzenbein, M., Tommerup, N., Eyre, H., et al. (2003) Disruption of the serine/threonine kinase 9 gene causes severe X-linked infantile spasms and mental retardation. Am. J. Hum. Genet., 72, 1401-1411.
31. Weaving, L. S., Christodoulou, J., Williamson, S. L., Friend, K. L., McKenzie, O. L. D., Archer, H., Evans, J., Clarke, A., Pelka, G. J., Tam, P. P. L., et al. (2004) Mutations of CDKL5 cause a severe neurodevelopmental disorder with infantile spasms and mental retardation. Am. J. Hum. Genet., 75, 1079-1093.
32. Montague, T. G., Cruz, J. M., Gagnon, J. A., Church, G. M. and Valen, E. (2014) CHOPCHOP: a CRISPR/Cas9 and TALEN web tool for genome editing. Nucleic Acids Res., 42, W401-7.
33. Mali, P., Yang, L., Esvelt, K. M., Aach, J., Guell, M., DiCarlo, J. E., Norville, J. E. and Church, G. M. (2013) RNA-guided human genome engineering via Cas9. Science, 339, 823-826.
34. Joung, J., Konermann, S., Gootenberg, J. S., Abudayyeh, O. O., Platt, R. J., Brigham, M. D., Sanjana, N. E. and Zhang, F. (2017) Genome-scale CRISPR-Cas9 knockout and transcriptional activation screening. Nat. Protoc., 12, 828-863.
35. Krishna, A., Biryukov, M., Trefois, C., Antony, P. M. A., Hussong, R., Lin, J., Heininiemi, M., Glusman, G., Köglsberger, S., Boyd, O., et al. (2014) Systems genomics evaluation of the SH-SY5Y neuroblastoma cell line as a model for Parkinson's disease. BMC Genomics, 15, 1154.
36. Pollock, K., Dahlenburg, H., Nelson, H., Fink, K. D., Cary, W., Hendrix, K., Annett, G., Torrest, A., Deng, P., Gutierrez, J., et al. (2016) Human Mesenchymal Stem Cells Genetically Engineered to Overexpress Brain-derived Neurotrophic Factor Improve Outcomes in Huntington's Disease Mouse Models. Mol. Ther., 24, 965-977.
37. Li, L.-C. and Dahiya, R. (2002) MethPrimer: designing primers for methylation PCRs. Bioinformatics, 18, 1427-1431.
38. Krueger, F. and Andrews, S. R. (2011) Bismark: a flexible aligner and methylation caller for Bisulfite-Seq applications. Bioinformatics, 27, 1571-1572.
39. Guo, W., Zhu, P., Pellegrini, M., Zhang, M. Q., Wang, X. and Ni, Z. (2018) CGmapTools improves the precision of heterozygous SNV calls and supports allele-specific methylation detection and visualization in bisulfite-sequencing data. Bioinformatics, 34, 381-387.
40. O'Geen, H., Bates, S. L., Carter, S. S., Nisson, K. A., Halmai, J., Fink, K. D., Rhie, S. K., Farnham, P. J. and Segal, D. J. (2019) Ezh2-dCas9 and KRAB-dCas9 enable engineering of epigenetic memory in a context-dependent manner. Epigenetics Chromatin, 12, 26.
41. Aryee, M. J., Jaffe, A. E., Corrada-Bravo, H., Ladd-Acosta, C., Feinberg, A. P., Hansen, K. D. and Irizarry, R. A. (2014) Minfi: a flexible and comprehensive Bioconductor package for the analysis of Infinium DNA methylation microarrays. Bioinformatics, 30, 1363-1369.
42. Fortin, J.-P., Triche, T. J. J. and Hansen, K. D. (2017) Preprocessing, normalization and integration of the Illumina HumanMethylationEPIC array with minfi. Bioinformatics, 33, 558-560.
43. Triche, T. J. J., Weisenberger, D. J., Van Den Berg, D., Laird, P. W. and Siegmund, K. D. (2013) Low-level processing of Illumina Infinium DNA Methylation BeadArrays. Nucleic Acids Res., 41, e90.
44. Fortin, J.-P., Labbe, A., Lemire, M., Zanke, B. W., Hudson, T. J., Fertig, E. J., Greenwood, C. M. and Hansen, K. D. (2014) Functional normalization of 450 k methylation array data improves replication in large cancer studies. Genome Biol., 15, 503.
45. Tian, Y., Morris, T. J., Webster, A. P., Yang, Z., Beck, S., Feber, A. and Teschendorff, A. E. (2017) ChAMP: updated methylation analysis pipeline for Illumina BeadChips. Bioinformatics, 33, 3982-3984.
46. Smyth, G. K. (2004) Linear models and empirical bayes methods for assessing differential expression in microarray experiments. Stat. Appl. Genet. Mol. Biol., 3, Article3.
47. Wettenhall, J. M. and Smyth, G. K. (2004) limmaGUI: a graphical user interface for linear modeling of microarray data. Bioinformatics, 20, 3705-3706.
48. Love, M. I., Huber, W. and Anders, S. (2014) Moderated estimation of fold change and dispersion for RNA-seq data with DESeq2. Genome Biol., 15, 550.
49. Bae, S., Park, J. and Kim, J.-S. (2014) Cas-OFFinder: a fast and versatile algorithm that searches for potential off-target sites of Cas9 RNA-guided endonucleases. Bioinformatics, 30, 1473-1475.
50. Perez-Pinera, P., Kocak, D. D., Vockley, C. M., Adler, A. F., Kabadi, A. M., Polstein, L. R., Thakore, P. I., Glass, K. A., Ousterout, D. G., Leong, K. W., et al. (2013) RNA-guided gene activation by CRISPR-Cas9-based transcription factors. Nat. Methods, 10, 973-976.
51. Xu, X., Tao, Y., Gao, X., Zhang, L., Li, X., Zou, W., Ruan, K., Wang, F., Xu, G. and Hu, R. (2016) A CRISPR-based approach for targeted DNA demethylation. Cell Discov., 2, 16009.
52. Cano-Rodriguez, D., Gjaltema, R. A. F., Jilderda, L. J., Jellema, P., Dokter-Fokkens, J., Ruiters, M. H. J. and Rots, M. G. (2016) Writing of H3K4Me3 overcomes epigenetic silencing in a sustained but context-dependent manner. Nat. Commun., 7, 12284.
53. Verkuijl, S. A. and Rots, M. G. (2019) The influence of eukaryotic chromatin state on CRISPR-Cas9 editing efficiencies. Curr. Opin. Biotechnol., 55, 68-73.
54. Thakore, P. I., D'Ippolito, A. M., Song, L., Safi, A., Shivakumar, N. K., Kabadi, A. M., Reddy, T. E., Crawford, G. E. and Gersbach, C. A. (2015) Highly specific epigenome editing by CRISPR-Cas9 repressors for silencing of distal regulatory elements. Nat. Methods, 12, 1143.
55. Chen, F., Ding, X., Feng, Y., Seebeck, T., Jiang, Y. and Davis, G. D. (2017) Targeted activation of diverse CRISPR-Cas systems for mammalian genome editing via proximal CRISPR targeting. Nat. Commun., 8, 14958.
56. Kim, D. and Kim, J.-S. (2018) DIG-seq: a genome-wide CRISPR off-target profiling method using chromatin DNA. Genome Res., 28, 1894-1900.
57. Yarrington, R. M., Verma, S., Schwartz, S., Trautman, J. K. and Carroll, D. (2018) Nucleosomes inhibit target cleavage by CRISPR-Cas9 in vivo. Proc. Natl. Acad. Sci. U.S.A., 115, 9351-9358.
58. Jegu, T., Aeby, E. and Lee, J. T. (2017) The X chromosome in space. Nat. Rev. Genet., 18, 377-389.
59. Galonska, C., Charlton, J., Mattei, A. L., Donaghey, J., Clement, K., Gu, H., Mohammad, A. W., Stamenova, E. K., Cacchiarelli, D., Klages, S., et al. (2018) Genome-wide tracking of dCas9-methyltransferase footprints. Nat. Commun., 9, 597.
60. Chavez, A., Scheiman, J., Vora, S., Pruitt, B. W., Tuttle, M., P R Iyer, E., Lin, S., Kiani, S., Guzman, C. D., Wiegand, D. J., et al. (2015) Highly efficient Cas9-mediated transcriptional programming. Nat. Methods, 12, 326-328.
61. Tanenbaum, M. E., Gilbert, L. A., Qi, L. S., Weissman, J. S. and Vale, R. D. (2014) A protein-tagging system for signal amplification in gene expression and fluorescence imaging. Cell, 159, 635-646.
62. Alarcon, M., Abrahams, B. S., Stone, J. L., Duvall, J. A., Perederiy, J. V, Bomar, J. M., Sebat, J., Wigler, M., Martin, C. L., Ledbetter, D. H., et al. (2008) Linkage, association, and gene-expression analyses identify CNTNAP2 as an autism-susceptibility gene. Am. J. Hum. Genet., 82, 150-159.
63. Kocak, D. D., Josephs, E. A., Bhandarkar, V., Adkar, S. S., Kwon, J. B. and Gersbach, C. A. (2019) Increasing the specificity of CRISPR systems with engineered RNA secondary structures. Nat. Biotechnol., 37, 657-666.
64. Szafranski, P., Golla, S., Jin, W., Fang, P., Hixson, P., Matalon, R., Kinney, D., Bock, H., Craigen, W., Smith, J. L., et al. (2015) Neurodevelopmental and neurobehavioral characteristics in males and females with CDKL5 duplications. Eur. J. Hum. Genet., 23 (7), 915-921.
65. Mari F., Azimonti S., Bertani I., Bolognese F., Colombo E., Caselli R., Scala E., Longo I., Grosso S., Pescucci C., Ariani F., Hayek G., Balestri P., Bergo A., Badaracco G., Zappella M., Broccoli V., Renieri A., Kilstrup-Nielsen C., Landsberger N. (2005) CDKL5 belongs to the same molecular pathway of MeCP2 and it is responsible for the early-onset seizure variant of Rett syndrome. Hum. Mol. Genet. 14:1935-1946(2005).
66 Morgan L Maeder, James F Angstman, Marcy E Richardson, Samantha J Linder, Vincent M Cascio, Shengdar Q Tsai, Quan H Ho, Jeffry D Sander, Deepak Reyon, Bradley E Bernstein, Joseph F Costello, Miles F Wilkinson, J Keith Joung (2013) Targeted DNA demethylation and activation of endogenous genes using programmable TALE-TET1 fusion proteins. Nat Biotechnol. 31(12):1137-42.
67 Roger R. Beerli, David J. Segal, Birgit Dreier, and Carlos F. Barbas III (1998) Toward controlling gene expression at will: Specific regulation of the erbB-2/HER-2 promoter by using polydactyl zinc finger proteins constructed from modular building blocks. PNAS 95 (25):14628-14633.
68. Mamta Tahiliani, Kian Peng Koh, Yinghua Shen, William A. Pastor, Hozefa Bandukwala, Yevgeny Brudno, Suneet Agarwal, Lakshminarayan M. Iyer, David R. Liu, L. Aravind, and Anjana Rao (2009) Science 324(5929): 930-935.

1. A gene editing system comprising:
(i) a first nucleotide molecule encoding a dCas9-Ten-Eleven Translocation methylcytosine dioxygenase 1 catalytic domain (TET1CD) fusion protein; and
(ii) a second nucleotide molecule encoding at least one small guide RNA (sgRNA), comprising: a scaffold region and a spacer region, wherein the spacer region hybridizes to a nucleotide sequence complementary to a target sequence adjacent to a 5′-end of a protospacer adjacent motif (PAM), and wherein the target sequence and the PAM are located within 1 kilobase (kb) of the transcriptional start site (TSS) of the CDKL5 gene.

2. The system of claim 1, further comprising a third nucleotide molecule encoding a dCas9 protein fused to at least one transcriptional activator.

3. The system of claim 2, wherein the at least one transcriptional activator comprises VP64.

4. The system of claim 1, wherein the target sequence for the sgRNA comprises AGAGCATCGGACCGAAGCGG, or
GGGGGAGAACATACTCGGGG, or CCCAGGTTGCTAGGGCTTGG.

5-6. (canceled)

7. The system of claim 1, wherein the at least one sgRNA comprises a first sgRNA, a second sgRNA, and a third sgRNA, wherein the target sequence for the first sgRNA comprises AGAGCATCGGACCGAAGCGG, wherein the target sequence for the second sgRNA comprises GGGGGAGAACATACTCGGGG, and wherein the target sequence for the third sgRNA comprises CCCAGGTTGCTAGGGCTTGG.

8. The system of claim 2, wherein the first nucleotide molecule, the second nucleotide molecule, and the third nucleotide molecule are integrated into one or more viral or plasmid vectors.

9. The system of claim 8, wherein the viral vector is a lentiviral vector, an adeno-associated viral (AAV) vector, or an adenoviral vector.

10. A vector encoding a sgRNA, wherein the sgRNA comprises a scaffold region and a spacer region, wherein the spacer region hybridizes to a nucleotide sequence complementary to a target sequence comprising AGAGCATCGGACCGAAGCGG, or
GGGGGAGAACATACTCGGGG, or CCCAGGTTGCTAGGGCTTGG.

11-12. (canceled)

13. A vector encoding a first sgRNA and a second sgRNA, wherein the first sgRNA comprises a scaffold region and a spacer region, wherein the spacer region of the first sgRNA hybridizes to a nucleotide sequence complementary to a target sequence comprising AGAGCATCGGACCGAAGCGG, or
GGGGGAGAACATACTCGGGG or CCCAGGTTGCTAGGGCTTGG,
wherein the second sgRNA comprises a scaffold region and a spacer region, and wherein the spacer region of the second sgRNA hybridizes to a nucleotide sequence complementary to a different target sequence comprising GGGGGAGAACATACTCGGGG, or
AGAGCATCGGACCGAAGCGG, or CCCAGGTTGCTAGGGCTTGG.

14-15. (canceled)

16. A vector encoding a first sgRNA, a second sgRNA, and a third sgRNA, wherein the first sgRNA comprises a scaffold region and a spacer region, wherein the spacer region of the first sgRNA hybridizes to a nucleotide sequence complementary to a target sequence comprising AGAGCATCGGACCGAAGCGG, wherein the second sgRNA comprises a scaffold region and a spacer region, wherein the spacer region of the second sgRNA hybridizes to a nucleotide sequence complementary to a target sequence comprising GGGGGAGAACATACTCGGGG, wherein the third sgRNA comprises a scaffold region and a spacer region, and wherein the spacer region of the third sgRNA hybridizes to a nucleotide sequence complementary to a target sequence comprising CCCAGGTTGCTAGGGCTTGG.

17-23. (canceled)

24. A host cell comprising the system of claim 1.

25-29. (canceled)

30. A pharmaceutical composition comprising the host cell of claim 24 and a carrier, optionally a pharmaceutically acceptable carrier or excipient.

31. A method for increasing CDKL5 gene expression in a cell or subject comprising administering to the cell or subject the system of claim 1.

32. The method of claim 31, wherein the cell or subject is in need of increased CDLK5 gene expression.

33. The method of claim 32, wherein cell or subject has a methylated or a hypermethylated CDKL5 promoter region as compared to a CDKL5 promoter on a non-silenced X-chromosome.

34. The method of claim 33, wherein the CDKL5 promoter region in the cell or subject is located on a silenced X-chromosomal allele of the subject.

35. The method of claim 31, wherein the subject has been diagnosed with CDKL5 deficiency disorder (CDD) or the cell is isolated from a subject having been diagnosed with CDD.

36. The method of claim 31, wherein the cell is a neuronal cell.

37-41. (canceled)

42. A method for treating or preventing CDD in a subject in need thereof comprising administering to the subject the system of claim 1.

43-49. (canceled)

50. A kit comprising the system of claim 1 and optional instructions for use.
